(19) United States
(12) Patent Application Publication
(10) Pub. No.: US 2021/0379131 A1
(43) Pub. Date: Dec. 9, 2021

(54) METHODS AND SYSTEMS FOR INDUSTRIAL PROCESSES OF CANNABIS PRODUCTS

(71) Applicant: HEXO OPERATIONS INC., Ottawa (CA)

(72) Inventors: Max Alsayar, Ottawa (CA); Denis Keseris, Baie D'Urfe (CA); Stephan Georgiev, Saint-Hubert (CA); Bryce Dorin, Ottawa (CA); Jeff Leuschner, Ottawa (CA); David Walters, Stittsville (CA)

(21) Appl. No.: 17/407,957

(22) Filed: Aug. 20, 2021

Related U.S. Application Data
(63) Continuation of application No. 17/429,924.

(30) Foreign Application Priority Data
Feb. 11, 2019 (CA) 3033404
Jun. 28, 2019 (CA) 3048298

(51) Int. Cl. A61K 36/185; A24B 15/167; A23L 33/105; A23L 2/52; A23D 7/005; B65B 3/04; B65B 61/26; B65C 9/46

(52) U.S. Cl. CPC A61K 36/185; A24B 15/167; A23L 33/105; A23L 2/52; A23D 7/0053; A23V 2002/00; B65B 61/26; B65C 9/46; A61K 2236/17; A61K 2236/15; B65B 3/04

(57) ABSTRACT

The present application relates to the production of cannabis products, particularly on a large scale, such as at an industrial level. Cannabis is typically a controlled and regulated substance, and so inventory control, security, and traceability of the cannabis may need to be provided. A human-based manual and/or labour-intensive implementation is not scalable, and is therefore infeasible at an industrial level. Disclosed herein are computer systems for inventory control, security systems, and computer methods for facilitating traceability from a cannabis product back to a batch of cannabis plants.

### FIELD

This disclosure relates generally to information and communication technology (ICT) for the cannabis industry. In particular, in some embodiments, the disclosure relates to systems and methods for tracing cannabinoid-containing substances through complex industrial cultivation, extraction, manufacturing and distribution chains.

### BACKGROUND

While the legal market for cannabis-based consumer products is gaining momentum, historically, the clandestine nature of the cannabis industry has largely suppressed innovation, and led to a market characterised by unsophisticated and small-scale production processes, as well as underdeveloped consumer product safety and characterisation standards.

Even in jurisdictions in which medical cannabis has been legal for some time, most governments impose strict controls on cannabinoid-containing substances, for at least the reasons of undermining the financial success of organized crime (e.g. by reducing the amount of cannabis-based products in the black/grey market) and ensuring public safety (e.g. by restricting access to psychotropic substances). As a result, cannabis producers and processors have been subject to stringent record keeping requirements, particularly in regard to tracking the provenance and chain of custody of cannabinoid-containing substances.

While these requirements are quite onerous, compliance has not historically posed a significant technical problem because the size of the medical cannabis market has kept the demand for cannabis-based products relatively limited. As a result, cannabis producers and processors have had no need to implement industrial-scale processes.

Moreover, the limited diversity of legally-available cannabis-based consumer products (e.g. restricted to cannabis flower, seeds and oils, in Canada) has also helped cannabis producers and processors comply with the record keeping requirements imposed by public health organizations, in that the limited consumer product diversity effectively limits record-keeping requirements. Accordingly, many cannabis producers and processors have relied on manual and/or labour-intensive systems and methods of record keeping.

More recently however, rapidly evolving changes in cannabis legislation in many jurisdictions around the world are contributing to a quantum leap in both the demand for, and the variety of (e.g. edibles, concentrates, etc.), cannabis-based consumer products. This entirely new market for complex and sophisticated cannabis-based products will need to be supported by equally complex and sophisticated industrial cultivation, extraction, manufacturing and distribution processes.

As such, attempts at scaling known manual and/or labour-intensive systems and methods of tracking cannabinoid-containing substances to meet this demand can lead to dilatory, ineffective and unsafe solutions. Applying these known solutions to this new and unique industrial environment provides significant financial and technical drawbacks. Moreover, scaled versions of known solutions are clearly also susceptible to human error and data-security risks, which in turn put the public's safety at risk and leave legal production, manufacturing and distributions chains open to misappropriation by organized crime.

For these and other reasons, there is a clear need for technical improvements in data-communication network and computer-based systems and methods for tracking cannabinoid-containing substances through complex industrial cultivation, extraction, manufacturing and distribution chains.

### SUMMARY

In accordance with various aspects of this disclosure, the provenance and chain of custody of cannabinoid-containing substances are tracked and/or traced through industrial cultivation, extraction, manufacturing and distribution processes by way of information and communication technology methods and systems.

In accordance with an aspect, this disclosure relates to a method that comprises the step of providing a database in which is stored information associated with a plurality of cannabis plants and a plurality of cannabis products. The method also comprises the steps of assigning a batch identifier to a batch of the plurality of cannabis plants and processing plant material from a portion of the cannabis plants in the batch using a first process to produce a plurality of units of a first cannabis product. The method also comprises the step of processing plant material from another portion of the cannabis plants in the batch using a second process to produce a plurality of units of a second cannabis product. The method also comprises the steps of assigning a first lot identifier to a lot of the plurality of units of the first cannabis product and a second lot identifier to a lot of the plurality of units of the second cannabis product and modifying the database to include information conveying the batch identifier, the first lot identifier and the second lot identifier, wherein the first lot identifier and the second lot identifier are each associated with the batch identifier.

In accordance with another aspect, this disclosure relates to a processor-readable storage medium, having processor-executable instructions stored thereon, which, when executed by a processor, cause a computing device comprising the processor to implement a system configured to implement a database configured to store information associated with a plurality of cannabis plants and a plurality of cannabis products. The system is further configured to assign a batch identifier to a batch of the plurality of cannabis plants and receive processing information relating to the processing of plant material from a portion of the cannabis plants in the batch using a first process to produce a plurality of units of a first cannabis product. The system is further configured to receive processing information relating to the processing of plant material from another portion of the cannabis plants in the batch using a second process to produce a plurality of units of a second cannabis product. The system is further configured to assign, using the processing information, a first lot identifier to a lot of the plurality of units of the first cannabis product and a second lot identifier to a lot of the plurality of units of the second cannabis product. The system is further configured to modify the database to include information relating to the batch identifier, the first lot identifier and the second lot identifier, wherein the first lot identifier and the second lot identifier are each associated with the batch identifier.

In accordance with yet another aspect, this disclosure relates to a method that comprises the steps of providing a database in which is stored information associated with a plurality of cannabis plants and a plurality of cannabis products and assigning a batch identifier to a batch of the plurality of cannabis plants. The method further comprises the steps of extracting cannabinoids from the plant material of a portion of the cannabis plants in the batch using an extraction method to produce a cannabis extract and assigning an extract identifier to the cannabis extract. The method further comprises processing an amount of the cannabis extract to produce a plurality of units of a cannabis product and assigning a lot identifier to a lot of the plurality of units of the cannabis product. The method further comprises the step of modifying the database to include information relating to the batch identifier, the extract identifier and the lot identifier, wherein the lot identifier is associated with the extract identifier and the extract identifier is associated with the batch identifier.

In accordance with yet another aspect, this disclosure relates to a processor-readable storage medium, having processor-executable instructions stored thereon, which, when executed by a processor, cause a computing device comprising the processor to implement a system configured to implement a database configured to store information associated with a plurality of cannabis plants and a plurality of cannabis products. The system being further configured to assign a batch identifier to a batch of the plurality of cannabis plants. The system being further configured to receive extraction information relating to the extraction of cannabinoids from the plant material of a portion of the cannabis plants in the batch using an extraction method to produce cannabis extract and assign an extract identifier to the cannabis extract. The system being further configured to receive processing information related to the processing of an amount of the cannabis extract to produce a plurality of units of a cannabis product and assign a lot identifier to a lot of the plurality of units of the cannabis product. The system being further configured to modify the database to include information relating to the batch identifier, the extract identifier and the lot identifier, wherein the lot identifier is associated with the extract identifier and the extract identifier is associated with the batch identifier.

In accordance with yet another aspect, this disclosure relates to a method of labelling cannabis products in an automated manufacturing process. The method comprises processing a portion of a first amount of cannabinoid-containing substance to sequentially produce a first plurality of units of a cannabis product, the first amount of cannabinoid-containing substance being associated with a first cannabinoid-containing substance identifier. The method further comprises determining a last unit of cannabis product produced in the first plurality of units and processing a portion of a second amount of cannabinoid-containing substance to sequentially produce a second plurality of units of a cannabis product, the second amount of cannabinoid-containing substance being associated with a second cannabinoid-containing substance identifier. The method further comprises labelling the first and second pluralities of units of cannabis product by controlling an automated labelling system to label units of cannabis product with label information conveying a first lot identifier associated with the first cannabinoid-containing substance identifier until the last unit of cannabis product has been labelled, and to label units of cannabis product with label information conveying a second lot identifier associated with the second cannabinoid-containing substance identifier thereafter.

In accordance with yet another aspect, this disclosure relates to a method for applying an indicia to containers filled with cannabis-infused beverage. The method comprises providing a marking station to mark with an indicia containers filled with cannabis-infused beverage, the indicia being indicative of a particular amount of a cannabinoid-containing substance derived from cannabis plant material and containing one or more cannabinoids, from which the cannabis-infused beverage is prepared, the marking station configured to receive a succession of containers filled with cannabis-infused beverage, the succession of containers being arranged in successive sets, where each set of containers is filled with cannabis-infused beverage made from a respective amount of the cannabinoid-containing substance. The method also comprises applying at each container from a first set a first indicia associated with a first amount of cannabinoid-containing substance from which the cannabis-infused beverage dispensed in the first set of containers is made. The method also comprises detecting in the succession of containers a transition from a first set of containers to a second set of containers, wherein the first set of containers is filled with cannabis-infused beverage prepared from a first amount of cannabinoid-containing substance and the second set of containers is filled with cannabis-infused beverage prepared from a second amount of cannabinoid-containing substance. The method also comprises controlling the marking station to apply a first indicia to the last container of the set in the succession, wherein the first indicia is associated with the first amount, and a second indicia to the next container in the succession of containers which is the first container of the second set, the second indicia being associated with the second amount.

In accordance with yet another aspect, this disclosure relates to a method of identifying a lot of cannabis products for recall. The method comprises providing a database in which is stored information associated with a plurality of batches of cannabis plants, each batch being associated with a batch identifier, and a plurality of lots of cannabis products, each lot being associated with a lot identifier, wherein each batch identifier in the database is associated with at least one lot identifier. The method also comprises determining, using a lot identifier associated with a defective cannabis product, at least one suspect batch identifier associated with the lot identifier. The method also comprises determining, for each archived cannabis material sample associated with the at least one suspect batch identifier, whether the archived cannabis material sample is defective and determining all lot identifiers in the database associated with each archived cannabis material sample that is found to be defective.

In accordance with yet another aspect, this disclosure relates to a method of identifying a lot of cannabis products for recall. The method comprises providing a database in which is stored information associated with a plurality of batches of cannabis plants, each batch being associated with a batch identifier, and a plurality of lots of cannabis products, each lot being associated with a lot identifier, wherein each batch identifier in the database is associated with at least one lot identifier. The method also comprises providing a graphical user interface implemented on a computer system to enable a user to input a suspect lot identifier associated with a defective cannabis product. The method also comprises providing a database search module implemented on the computer system, the database search module being configured to determine, in response to a user inputting a suspect lot identifier, at least one suspect batch identifier associated with the suspect lot identifier in the database and all lot identifiers associated with the at least one suspect batch identifier in the database and inputting a suspect lot identifier into the graphical user interface.

In accordance with yet another aspect, this disclosure relates to a system for identifying a lot of cannabis products for recall. The system comprises a database in which is stored information associated with a plurality of batches of cannabis plants, each batch being associated with a batch identifier, and a plurality of lots of cannabis products, each lot being associated with a lot identifier, wherein each batch identifier in the database is associated with at least one lot identifier. The system also comprises a graphical user interface implemented on a computer system to enable a user to input a suspect lot identifier associated with a defective cannabis product. The system also comprises a database search module implemented on the computer system, the database search module being configured to determine, in response to a user inputting a suspect lot identifier through the graphical user interface, at least one suspect batch identifier associated with the suspect lot identifier in the database and recall lot identifiers associated with the at least one suspect batch identifier in the database.

In accordance with yet another aspect, this disclosure relates to a method for dynamically generating a hierarchal dataset having a tree structure, representative of a process flow to transform a batch of cannabis plants into a range of cannabis products. The method comprises recording on a computer readable storage medium a batch identifier associated with the batch of cannabis plants, the batch identifier distinguishing the batch of cannabis plants among a plurality of batches of cannabis plants, wherein the batch identifier is a root level of the hierarchal dataset. The method also comprises processing a first portion of the batch of cannabis plants using a first process to produce a plurality of units of first cannabis products and recording on the computer readable storage medium a first lot number associated with the first cannabis products. The method also comprises processing a second portion of the batch of cannabis plants using a second process, to produce a plurality of units of a second cannabis product and recording on the computer readable storage medium a second lot number associated with the second cannabis products. The method also comprises linking the first and second lot numbers to the batch identifier in the hierarchal dataset, whereby the first lot number forms a first branch of the hierarchal data set ascending from the root node and the second lot number forms a second branch of the hierarchal data structure ascending from the root node.

In accordance with yet another aspect, this disclosure relates to a method for bottling a cannabis-infused beverage. The method comprises providing a filling line including a filling station, a container marking station and a control device, the control device configured to control an operation of the container marking station. The method also comprises filling containers at the filling station with cannabis-infused beverage supplied from a master batch of cannabis-infused beverage, the master batch being prepared from an amount of cannabis-containing substance derived from cannabis plant material, the cannabis-containing substance containing one or more cannabinoids, the master batch including a quantity of cannabis-infused beverage to fill a plurality of containers, the filling station being configured to perform a supply switch from a first master batch to a second master batch of cannabis-infused beverage, whereby a first set of containers is filled with cannabis-infused beverage drawn from the first master batch and a second set of containers is filled with cannabis infused beverage drawn from the second master batch. The method also comprises applying an indicia on each container at the marking station, the indicia being indicative of the master batch of the cannabis-infused beverage supplying the filling station when the container is filled by the filling station. The method also comprises controlling with the control device the operation of the marking station such that when a supply switch is performed from the first master batch to the second master batch a marking switchover from a first indicia to a second indicia is performed by the marking station such that containers filled with cannabis-infused beverage drawn from the first master batch are marked with a first indicia associated with the first master batch, and containers with cannabis-infused beverage drawn from the second master batch are marked with a second indicia associated with the second master batch.

In accordance with yet another aspect, this disclosure relates to a method for manufacturing and packaging a cannabis-infused consumable product made from a cannabis-containing substance. The method comprises providing multiple amounts of cannabis-containing substance, each amount derived from cannabis plant material, the cannabis-containing substance containing one or more cannabinoids, each amount of cannabis-containing substance being associated with an identifier allowing distinguishing one amount from another amount. The method further comprises providing a control device having a machine-readable storage and storing in the machine-readable storage identifiers associated with respective ones of the amounts of cannabis-containing substance. The method further comprises diluting each amount of cannabis-containing substance with a diluting agent to produce a master batch of consumable product and dispensing the master batch into a set of packages, each package holding a portion of the master batch. The method further comprises applying an indicia on individual packages. The step of applying an indicia further comprises feeding a stream of individual packages to a marking unit and distinguishing in the stream between individual packages holding a consumable product made from different amounts of cannabis-containing substance and controlling the marking unit with the control device to apply to each individual package an indicia derived from the identifier of the respective amount from which the consumable product in the package was made.

In accordance with yet another aspect, this disclosure relates to a method for manufacturing and packaging a cannabis-infused consumable product made from a cannabis-containing substance. The method comprises providing multiple amounts of a cannabis-containing substance, each amount derived from cannabis plant material, the cannabis-containing substance containing one or more cannabinoids. The method comprises providing a control device having a machine-readable storage and storing in the machine-readable storage identifiers associated with respective ones of the amounts of cannabis-containing substance, the identifiers allowing distinguishing one amount from another amount. The method further comprises diluting each amount of cannabis-containing substance with a diluting agent to produce respective master batches of consumable product and dispensing the master batches into respective sets of individual packages, each package of a given set holding a portion of the respective master batch. The method further comprises feeding a stream of individual packages to a marking unit, the stream being arranged in an order determined by which master batch is the source of the consumable product held in each individual package. The method further comprises, under control of the control device, synchronizing the operation of the marking unit with the order in which the stream of individual packages is arranged such that each individual package receives an indicia associated with the particular amount from which the consumable product in the package is made.

In accordance with yet another aspect, this disclosure relates to a method for manufacturing and packaging a cannabis-infused consumable product made from a cannabis-containing substance. The method comprises providing multiple amounts of cannabis-containing substance, each amount derived from cannabis plant material, the cannabis-containing substance containing one or more cannabinoids and providing a control device having a machine-readable storage. The method further comprises diluting each amount of cannabis-containing substance with a diluting agent to produce respective master batches of consumable product. The method further comprises, for each master batch, dispensing the master batch into a set of individual packages, each package holding a portion of the master batch and withholding from dispensing into an individual package a residual volume of consumable product from the master batch that is less than the volume of consumable product required to fill the individual package to capacity. The method further comprises, for one or more master batches, determining the number of individual packages filled to capacity from the master batch in the respective set of individual packages, storing the number in the machine-readable storage, feeding a stream of individual packages to a marking unit and controlling with the control device the marking unit including deriving from the machine readable storage the number and operating the marking unit a corresponding number of times to apply to each individual package in the set an indicia linked to the particular amount of cannabis-containing substance from which the consumable product in the package is made.

In accordance with yet another aspect, this disclosure relates to a method of creating video content. The method comprises receiving video images of a cannabis operations area in which cannabis material is being processed and receiving processing information associated with the processing being carried out in in the cannabis operations area. The method further comprises generating metadata using at least some of the processing information and generating a video record by combining the video images and the metadata.

These and other aspects of this disclosure will now become apparent to those of ordinary skill in the art upon review of a description of embodiments in conjunction with accompanying drawings.

### BRIEF DESCRIPTION OF THE DRAWINGS

For a more complete understanding of the present disclosure, reference is now made to the following description taken in conjunction with the accompanying drawings, in which:

FIG. 1 is a flow diagram illustrating an example process for producing cannabis products;

FIG. 2 is a block diagram illustrating an example system for producing cannabis products;

FIG. 3 illustrates example formats of machine-readable code for a label;

FIGS. 4A-4N are block diagrams illustrating an example system implementing an inventory control system (ICS);

FIG. 5 is a block diagram illustrating an example implementation of a barcode scanner in communication with an ICS server;

FIG. 6 is a flow chart illustrating an example method according to one embodiment;

FIG. 7 illustrates operation of a machine for generating labels, according to one embodiment;

FIG. 8 is a flow diagram illustrating an example method of labelling cannabis products in an automated manufacturing process.

FIG. 9 is a flow diagram illustrating an example method for drying and/or curing a cannabis material;

FIG. 10 is a flow diagram illustrating an example method for milling cannabis material;

FIG. 11 is a flow diagram illustrating an example method for producing pre-rolled cannabis cigarettes with a cone filling machine;

FIG. 12 is a flow diagram illustrating an example process for producing cannabis extracts and other cannabis products;

FIG. 13 is a flow diagram illustrating an example method for decarboxylation of a cannabis product;

FIG. 14 is a flow diagram illustrating an example method for supercritical fluid extraction with CO₂;

FIG. 15 is a flow diagram illustrating an example method for resin packaging;

FIG. 16 is a flow diagram illustrating an example process for oil formulation;

FIG. 17 is a flow diagram illustrating an example method for oil packaging;

FIG. 18 is a flow diagram illustrating an example method according to another embodiment.

FIG. 19 is a flow diagram illustrating an example method for cannabis product irradiation;

FIG. 20 is a flow diagram illustrating an example method for final packaging;

FIG. 21 illustrates an example of a lot record;

FIG. 22 illustrates an example of an extract record;

FIG. 23 illustrates an example of an extraction process record;

FIG. 24 is a block diagram of a cannabis producer and a cannabis processor, according to one embodiment;

FIG. 25 is a schematic illustrating an example of traceability from a cannabis-infused consumer product back to a batch of cannabis plants;

FIGS. 26-28 are block diagrams of a system for producing cannabis-infused beverages, according to one embodiment;

FIG. 29 is a flow diagram illustrating an example method of producing cannabis-infused beverages, according to one embodiment;

FIG. 30 is a flow diagram illustrating an example method for applying an indicia to containers filled with cannabis-infused beverage, according to another embodiment;

FIG. 31 is a flow diagram illustrating an example method of producing a cannabis-infused consumer product, according to one embodiment.

FIG. 32 illustrates a system for identifying a lot of cannabis products for recall, according to one embodiment;

FIG. 33 is a flow diagram illustrating an example method of identifying a lot of cannabis products for recall;

FIG. 34 is a flow diagram illustrating another example of a method of identifying a lot of cannabis products for recall; and

FIG. 35 is a flow diagram illustrating an example method of creating video content.

### DETAILED DESCRIPTION

For illustrative purposes, specific example embodiments will be explained in greater detail below in conjunction with the figures. It should be appreciated, however, that the present disclosure provides many applicable concepts that can be embodied in any of a wide variety of specific contexts. The specific embodiments discussed are merely illustrative and do not limit the scope of the present disclosure. For example, embodiments could include additional, different, or fewer features than shown in the drawings. In the flow diagrams illustrated in the accompanying figures, a rectangle generally annotates a step, apparatus, device, location or operation, and a pentagon generally annotates an input, product or output.

The present disclosure relates, in part, to the production and traceability of cannabis products. Cannabis products could be any goods that are produced from cannabis or hemp, which include plants, plant material, oils, resins, drinks, food additives, edibles, creams, aerosol sprays and vaporization substances, for example. These cannabis products could be used for medical and/or recreational purposes. Cannabis products could include active substances such as cannabinoids. However, the cannabis products described herein might not always include an active substance. As used herein, the term “cannabinoid” is generally understood to include any chemical compound that acts upon a cannabinoid receptor. Cannabinoids could include endocannabinoids (produced naturally by humans and animals), phytocannabinoids (found in cannabis and some other plants), and synthetic cannabinoids (manufactured artificially). For the purpose of this specification, the expression “cannabinoid” means a compound such as cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetra hydrocannabinolic acid-C4 (THCA-C4), delta-9-tetrahydrocannabinol-C4, delta-9-tetra hydrocannabivarin (THCV), delta-9-tetrahydrocannabiorcol (THC-C1), delta-7-cis-iso tetrahydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-H HCV), cannabiripsol (CBR), trihydroxy-delta-9-tetrahydrocannabinol (triOH-THC), cannabinol propyl variant (CBNV), and derivatives thereof.

For the purpose of this specification, the expressions “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-I-yl)-5-pentyl-I,3-benzenediol); (2) Δ4-cannabidiol (2-(6-isopropenyl-3-methyl-4-cyclohexen-I-yl)-5-pentyl-I,3-benzenediol); (3) Δ3-cannabidiol (2-(6-isopropenyl-3-methyl-3-cyclohexen-I-yl)-5-pentyl-I,3-benzenediol); (4) Δ3,7-cannabidiol (2-(6-isopropenyl-3-methylenecyclohex-I-yl)-5-pentyl-I,3-benzenediol); (5) Δ2-cannabidiol (2-(6-isopropenyl-3-methyl-2-cyclohexen-I-yl)-5-pentyl-I,3-benzenediol); (6) Δ1-cannabidiol (2-(6-isopropenyl-3-methyl-I-cyclohexen-I-yl)-5-pentyl-I,3-benzenediol); and (7) Δ6-cannabidiol (2-(6-isopropenyl-3-methyl-6-cyclohexen-I-yl)-5-pentyl-I,3-benzenediol).

A cannabis product could comprise a cannabinoid in its pure or isolated form or a source material comprising the cannabinoid. Examples of source materials comprising cannabinoids include, but are not limited to, cannabis or hemp plant material (for example, flowers, seeds, trichomes, and kief), milled cannabis or hemp plant material, extracts obtained from cannabis or hemp plant material (for example, resins, waxes and concentrates), and distilled extracts. In some embodiments, pure or isolated cannabinoids and/or source materials comprising cannabinoids could be combined with water, lipids, hydrocarbons (for example, butane), ethanol, acetone, isopropanol, or mixtures thereof.

Examples of phytocannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ⁹-THC), delta-9-tetrahydrocannabinolic acid A (THCA-A), delta-9-tetrahydrocannabionolic acid B (THCA-B), delta-9-tetra hydrocannabinolic acid-C4 (THCA-C4), delta-9-tetrahydrocannabinol-C4, delta-9-tetra hydrocannabivarin (THCV), delta-9-tetrahydrocannabiorcol (THC-C1), delta-7-cis-iso tetrahydrocannabivarin, delta-8-tetrahydrocannabinol (Δ⁸-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-H HCV), cannabiripsol (CBR), trihydroxy-delta-9-tetrahydrocannabinol (triOH-THC), cannabinol propyl variant (CBNV), and derivatives thereof.

Examples of synthetic cannabinoids include, but are not limited to, naphthoylindoles, naphthylmethylindoles, naphthoylpyrroles, naphthylmethylindenes, phenylacetylindoles, cyclohexylphenols, tetramethylcyclopropylindoles, adamantoylindoles, indazole carboxamides, and quinolinyl esters.

A cannabinoid may be in an acid form or a non-acid form, the latter also being referred to as the decarboxylated form since the non-acid form can be generated by decarboxylating the acid form. Within the context of the present disclosure, where reference is made to a particular cannabinoid, the cannabinoid can be in its acid or non-acid form, or be a mixture of both acid and non-acid forms.

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ⁹-THC) and delta-8-tetrahydrocannabinol (Δ⁸-THC) produce the effects associated with cannabis by binding to the CB1 cannabinoid receptors in the brain.

In some embodiments, the cannabinoid is cannabidiol (CBD). The terms “cannabidiol” or “CBD” are generally understood to refer to one or more of the following compounds, and, unless a particular other stereoisomer or stereoisomers are specified, includes the compound “Δ²-cannabidiol.” These compounds are: (1) Δ⁵-cannabidiol (2-(6-isopropenyl-3-methyl-5-cyclohexen-I-yl)-5-pentyl-I,3-benzenediol); (2) Δ⁴-cannabidiol (2-(6-isopropenyl-3-methyl-4-cyclohexen-I-yl)-5-pentyl-I,3-benzenediol); (3) Δ³-cannabidiol (2-(6-isopropenyl-3-methyl-3-cyclohexen-I-yl)-5-pentyl-I,3-benzenediol); (4) Δ^{3,7}-cannabidiol (2-(6-isopropenyl-3-methylenecyclohex-I-yl)-5-pentyl-I,3-benzenediol); (5) Δ²-cannabidiol (2-(6-isopropenyl-3-methyl-2-cyclohexen-I-yl)-5-pentyl-I,3-benzenediol); (6) Δ¹-cannabidiol (2-(6-isopropenyl-3-methyl-I-cyclohexen-I-yl)-5-pentyl-I,3-benzenediol); and (7) Δ⁶-cannabidiol (2-(6-isopropenyl-3-methyl-6-cyclohexen-I-yl)-5-pentyl-I,3-benzenediol).

In some embodiments, a cannabis product is produced by a cannabis producer. A cannabis producer refers to any entity (e.g. individual or organization) that cultivates and/or processes cannabis to produce a cannabis product. A cannabis producer may sometimes be called a licensed producer.

Process Overview

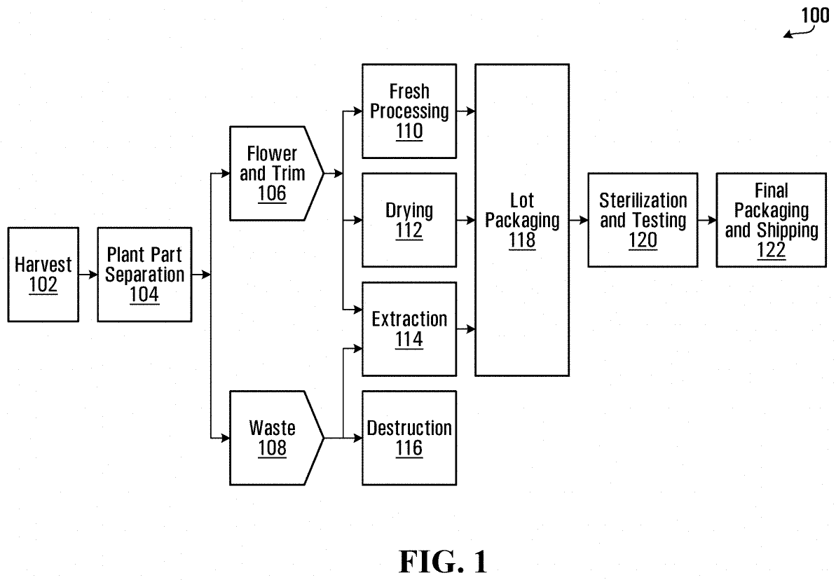

FIG. 1 is a flow diagram illustrating an example process 100 for producing cannabis products. Process 100 provides an overview of cannabis product production. Illustrative examples of various processes for cannabis product production are also described in detail elsewhere herein.

Process 100 includes an operation 102 of harvesting at least one batch of cannabis plants. A “batch” refers to a group or set of cannabis plants. Each batch of cannabis plants could be assigned a unique identifier (ID), which is referred to herein as a batch number. In general, a batch number could include alphanumeric characters and/or other symbols. By way of example, three different batches could be identified by the batch numbers “Batch-51”, “Batch-52” and “Batch-53”. In some embodiments, individual cannabis plants are also assigned unique IDs, which are referred to herein as plant numbers. Although referred to herein by way of example as numbers, IDs associated with plants, and/or other IDs herein, could include alphanumeric characters and/or other symbols.

A batch of cannabis plants could be cultivated in a particular grow area. In some embodiments, a grow area is defined as an area that cultivates similar cannabis plants. Grow areas could be provided in greenhouses or other structures that support the cultivation of cannabis plants. Grow areas could be contiguous areas, but this need not be the case in all embodiments. For example, grow areas that include multiple non-adjacent areas are also possible. In some embodiments, a grow area could be controlled to provide particular and/or consistent growth conditions. Cannabis plants that are cultivated in a grow area could be from the same types of seeds, or be from the same mother plant. A mother plant is a plant grown for the purpose of taking cuttings or offsets in order to grow more of the same plant. A grow area could instead cultivate plants from multiple different seed types and/or multiple different mother plants.

Several different batches of cannabis plants could be cultivated and/or harvested in parallel. For example, a greenhouse could be divided into several different grow areas, each grow area being used to cultivate a respective batch of cannabis plants. These batches of cannabis plants could be used for producing different cannabis products. Multiple cannabis products could be produced from a single batch of cannabis plants. Different batches of cannabis plants could also or instead be combined to produce a single cannabis product.

The cannabis plants that are harvested in operation 102 are sent to operation 104 for plant part separation, which divides the plants into flower and trim 106, and waste 108. Cannabis flower could also be referred to as bud, and is typically harvested from mature cannabis plants. Trim includes the leaves of the cannabis plant that are separated from the flower and stems. Trim could be harvested before the flower, while the plants mature. Waste from plant part separation could include stalks, stems and leaves that were not separated into trim, for example. In some embodiments, plant part separation includes manually cutting or otherwise removing the leaves and/or buds from the cannabis plants. However, an automated plant part separation process could also or instead be used.

At least a portion of the flower and trim 106 could be sent for fresh processing at operation 110, drying at operation 112, and/or extraction at operation 114. At least a portion of the waste 108 could also or instead be sent for extraction at operation 114. The remainder of the waste 108 could be sent for destruction at operation 116. Destruction of cannabis waste could include, for example, burning the waste.

Fresh processing at operation 110 could be used to produce fresh cannabis products. Fresh cannabis could include flower or trim that has not been dried or cured. In some embodiments, fresh processing could include sealing the cannabis after plant part separation to help prevent or reduce drying in the ambient atmosphere. Harvested flower or trim could also or instead be rapidly transported to a packaging process, at operation 118 for example, to help prevent or reduce drying before packaging.

Operation 112 is labelled in the drawing as drying, but could include drying and/or curing at least a selection of the cannabis flower and trim 106. In some embodiments, drying cannabis material could include the use of a commercial dehydrator system. Drying could also or instead be performed in the ambient environment. Curing includes a prolonged process of removing moisture from cannabis plant products under controlled conditions. Curing could act to preserve cannabis plant products and/or increase the concentration of some cannabinoids in the cannabis plant products.

The extraction process at operation 114 could be used to generate cannabis products such as resins. Operation 114 could also include other processes such as drying, curing, decarboxylation, winterization, distillation and/or product formulation processes. Product formulation processes, such as oil formulation processes and liquid formulation processes, could be used to produce cannabis products such as cannabis oils, vaporization substances, emulsions, food additives, edibles, drinks and oral sprays, for example. Examples of extraction, drying, curing, decarboxylation, winterization, distillation and product formulation processes are discussed in greater detail elsewhere herein.

The cannabis products produced in operations 110, 112, 114 are sent for lot packaging at operation 118. “Lots” refer to groups or sets of cannabis products. Cannabis products in the same lot have similar properties in some embodiments. For example, a lot could be a single type of cannabis product that is produced from the same batch of cannabis plants and/or by the same process or processes. Each lot is assigned a respective ID, for example, “Lot-5368”, “Lot-5369” and “Lot-5370”. A lot ID could also be referred to as a lot number. In general, a lot number could include alphanumeric characters and/or other symbols. In some embodiments, one batch of cannabis plants could produce one lot of cannabis products. In other embodiments, one batch of cannabis plants could produce multiple lots of cannabis products, which could be the same type of cannabis product or include multiple different types of cannabis products. For example, one batch of cannabis plants could be processed to produce lots of dry cannabis, fresh cannabis, and/or cannabis oil.

In some embodiments, lots are assigned as follows: a particular cannabis product originating from one batch of cannabis plants is assigned one lot number; each different cannabis product originating from that same batch is assigned a respective different lot number; and any cannabis products originating from different batches are assigned respective different lot numbers. The lot number assigned to each cannabis product is used for all units of that cannabis product.

Other methods of lot assignment are also possible. For example, two or more batches of cannabis plants could be mixed together. In some embodiments, lot numbers could be assigned to such mixed-batch cannabis products as follows: a particular cannabis product originating from one mixture of two or more batches of cannabis plants is assigned one lot number; each different cannabis product originating from that same mixture is assigned a respective different lot number; and any cannabis products originating from a different mixture of two or more batches are assigned respective different lot numbers. The lot number assigned to each cannabis product is used for all units of that cannabis product.

Packaging at operation 118 could include transferring lots of cannabis products into holding containers. The phrase “holding container”, as used herein, refers to any container in which cannabis products are or could be contained. Holding containers include containers that are used for storing products before, during and after processing, as well as containers that store products for sale. In some embodiments, holding containers are used to seal cannabis products from their environment. In some embodiments, holding containers could provide a form of child-resistance, tamper proofing and/or tamper detection. Examples of holding containers include jars, bins, vessels, bags, packets, boxes, bottles and cartridges (for vaporization devices, for example), any of which could be made out of wood, paper, cardboard, plastic, glass and/or metal, for example. Some holding containers, such as jars and bottles, could be sealed with caps or lids. In some embodiments, caps include tamper-proof induction seals. Holding containers could also or instead be sealed with one or more of: foil seals, heat seals, induction seals and shrink wrap, for example. A single holding container of cannabis product could be referred to as a unit.

During operation 118, labels could be applied to the holding containers. A label could associate a holding container with a specific cannabis product. Labels could be applied before, while and/or after filling the holding containers with cannabis products. Although labels could include material that is glued or otherwise attached to a holding container, this might not always be the case. For example, a label could be formed on or into a holding container, or be printed directly onto the surface of a holding container. In general, markings could be applied to any of various types of containers and/or packages. In some embodiments, marking involves printing or otherwise producing labels and affixing labels to containers and/or packages. In other embodiments, marking could also or instead involve printing or otherwise forming markings directly on containers and/or packages. As such, features disclosed herein in the context of labels or labelling could be applied more generally to other types of marking.

A label could include a written description of the holding container and/or the product in the holding container. A label could also or instead include a unique identifier that distinguishes a holding container from other holding containers. Examples of unique identifiers include letters, numbers, symbols, machine-readable code, and combinations thereof. The unique identifier could encode a description of a holding container and/or a product in the holding container. The following is a non-exhaustive list of information types, any one or more of which could be included in a description of a holding container and/or a product in a holding container:
plant number;
batch number;
lot number;
cannabis producer name, telephone number and/or email address;
cannabis producer number, which is a unique ID assigned to a specific cannabis producer;
customer name, telephone number and/or email address;
shipping information;
Global Trade Item Number (GTIN);
product name;
cannabinoid concentration;
product type;
product composition;
unit or case number;
processing date(s);
packaging date(s);
safety information;
regulatory information;
expiration or “best before” date;
product/container weight;
product/container volume; and
unit size.

In some embodiments, a machine for making labels, such as a label maker, could be used in operation 118 to generate labels for and/or apply labels to holding containers. During a first time period, the label maker could generate labels for a particular cannabis product originating from a particular batch of cannabis plants. Later, during a second time period when a cannabis product originating from a new batch of cannabis plants is being packaged, the label maker could update the labels being generated such that they map back to the new batch of cannabis plants. In other embodiments, the label maker could be replaced with a machine that generates cannabis holding containers that already include labels.

In process 100, packaged lots of cannabis products are sent for sterilization and testing at operation 120. Sterilization could be performed to remove and/or kill undesirable biological agents, such as bacteria and fungi. Irradiation is one example of a sterilization process, which is discussed in greater detail elsewhere herein. Testing could be performed to determine or confirm the composition of the cannabis products. For example, testing could determine the uniformity of a product, the safety of a product, and/or the amount(s) and type(s) of cannabinoid(s) in a product. For example, the concentration of tetrahydrocannabinol (THC) and/or cannabidiol (CBD) could be determined through testing. Testing cannabis products could also include sampling cannabis products. As discussed in more detail below, in some embodiments, only certain holding containers for a lot of products could be sampled, and in other embodiments, each holding container in a lot could be sampled.

Although lot packaging at operation 118 is illustrated before sterilization and testing at operation 120, this might not always be the case. For example, cannabis products might not be released for lot packaging until the products have been tested and the results are deemed satisfactory.

Final packaging and shipping occurs at operation 122. Operation 122 could also be referred to as picking, packaging and shipping (PPS). In some embodiments, final packaging includes packing multiple holding containers into larger packages for transportation. In general, cannabis products from multiple lots could be packaged together in operation 122. Final packaging could also or instead include removing cannabis products from one holding container and adding them to another holding container. Operation 122 could further include updating and/or adding labels on holding containers and/or packages. Final packaging could prepare cannabis products for shipping, such as by protecting and insulating the cannabis products. After final packaging, cannabis products could be released for sale, which could include shipping the products to customers and/or storing the products in a particular area until they are shipped. In some embodiments, shipping could be performed using a courier service. The term “customer”, as used herein, includes any individual or organization that receives a cannabis product from a cannabis producer or processor. Examples of customers include end users of a cannabis product, distributors of cannabis products, and producers of other cannabis products. Each customer could be assigned a unique customer ID.

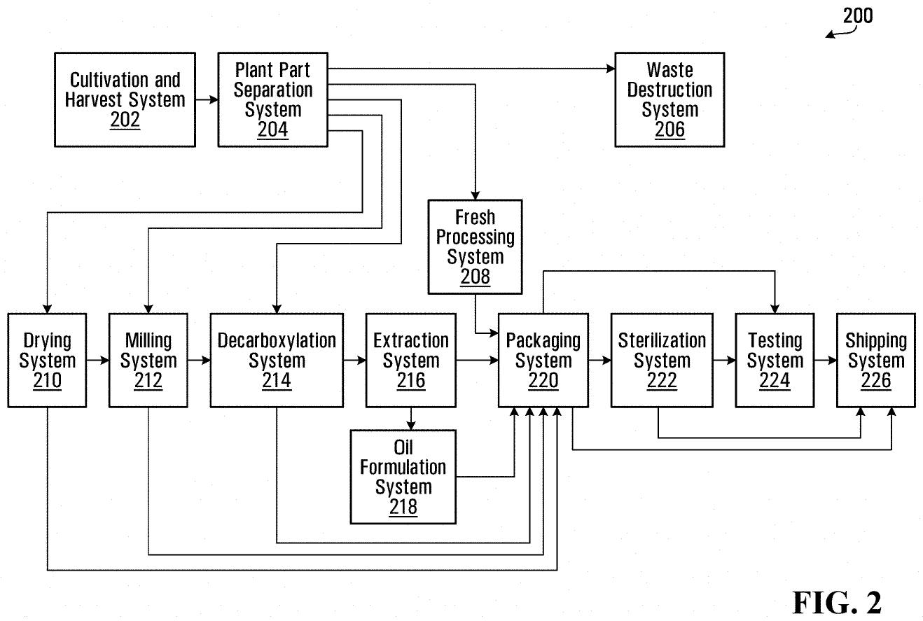

FIG. 2 is a block diagram illustrating an example system 200 for producing cannabis products. In some embodiments, the system 200 could be used to implement any or all of the operations 102, 104, 110, 112, 114, 116, 118, 120, 122 of FIG. 1. The system 200 includes a cultivation and harvest system 202, a plant part separation system 204, a waste destruction system 206, a fresh processing system 208, a drying system 210, a milling system 212, a decarboxylation system 214, an extraction system 216, an oil formulation system 218, a packaging system 220, a sterilization system 222, a testing system 224, and a shipping system 226. Various functions that could be performed by the systems 202, 204, 206, 208, 210, 212, 214, 216, 218, 220, 222, 224, 226, as well as various components and devices that could be included in these systems, are described elsewhere herein.

FIG. 2 illustrates various connections between the systems 202, 204, 206, 208, 210, 212, 214, 216, 218, 220, 222, 224, 226. In general, each of these connections indicates a transfer of cannabis products between two different systems, and/or a means for transferring cannabis products between two different systems. Transfers of cannabis product could include physical transfers and/or logical transfers. An example of a physical transfer includes moving a holding container of cannabis product from one facility to a different facility. Vehicles, carts and/or trollies could be used to physically transfer the holding containers. An example of a logical transfer includes processing a cannabis product using one system and then processing the same cannabis product using another system, even if the cannabis product does not actually move location. These transfers, whether physical or logical, could include manual and/or automated transfers.

FIG. 2 includes a connection between the cultivation and harvest system 202 and the plant part separation system 204, which could enable the transfer of harvested cannabis plants from the cultivation and harvest system to the plant part separation system in holding containers, for example.

The plant part separation system 204 is connected to the waste destruction system 206, which could enable the transfer of waste material, in holding containers for example, from the plant part separation system to the waste destruction system. The plant part separation system 204 is further connected to the fresh processing system 208, the drying system 210, the milling system 212 and the decarboxylation system 214. These connections could enable transfers of cannabis flower and/or trim, in holding containers for example, from the plant part separation system 204 to the fresh processing system 208, the drying system 210, the milling system 212 and/or the decarboxylation system 214.

Plant material could also or instead be serially processed through some or all of the drying system 210, the milling system 212, and the decarboxylation system 214. The drying system 210 is connected to the milling system 212 for transferring dry cannabis to the milling system, in holding containers for example. The milling system 212 is connected to the decarboxylation system 214, which could enable transfers of milled cannabis to the decarboxylation system, in holding containers for example. The decarboxylation system 214 is connected to the extraction system 216 for transferring decarboxylated cannabis to the extraction system, in holding containers for example. In some embodiments, plant material from the plant part separation system 204 is not processed through the drying system 210, the milling system 212, the decarboxylation system 214, and is instead transferred to the extraction system 216.

In the illustrated embodiment, the extraction system 216 is connected to the oil formulation system 218, which could enable transfer of cannabis extract to the oil formulation system, in holding containers for example.

The fresh processing system 208, the drying system 210, the milling system 212, the decarboxylation system 214, the extraction system 216 and the oil formulation system 218 are connected to the packaging system 220. Any one or more of different types of cannabis products produced by these systems could be transferred from these systems to the packaging system 220, in the same or one or more different types of holding containers, for example.

The packaging system 220 is connected to the sterilization system 222, the testing system 224 and the shipping system 226. Packaged cannabis products could be transferred, in packages and/or holding containers for example, from the packaging system 220 to any of the sterilization system 222, the testing system 224 and/or the shipping system 226.

In some embodiments, packaged cannabis products are sterilized and then tested, and transfer of sterilized cannabis products from the sterilization system 222 to the testing system 224 is illustrated in FIG. 2. Sterilized cannabis products could instead be shipped without testing, and transfer of sterilized cannabis products from the sterilization system 222 to the shipping system 226 is also illustrated. These transfers could involve transferring cannabis products in packages and/or holding containers.

The testing system 224 is also connected to the shipping system 226, which could enable the transfer of tested cannabis products to the shipping system, in holding containers for example.

The systems and connections illustrated in FIG. 2 represent an example embodiment. Other embodiments could include more, fewer and/or different systems, with similar and/or different interconnections.

Inventory Control System

An inventory control system (ICS) could be used to record, log, track and/or monitor cannabis products throughout cultivation, harvesting, processing, sales, shipping, and/or other operations. By way of example, an ICS could record cannabis products throughout operations 102, 104, 110, 112, 114, 116, 118, 120, 122 of FIG. 1. An ICS could also or instead be connected to or otherwise have access to any or all of the systems 202, 204, 206, 208, 210, 212, 214, 216, 218, 220, 222, 224, 226 of FIG. 2, to provide information to and/or record information from these systems. An ICS could also or instead record any or all transfers of cannabis product within and/or between the systems 202, 204, 206, 208, 210, 212, 214, 216, 218, 220, 222, 224, 226. In general, an ICS could enable traceability of any or all cannabis-containing substance through at least part of a production process, including traceability to lot level, batch level, or even plant level. This could include plants in cultivation, cannabis products in processing, cannabis products in storage, and/or cannabis products that have been released for sale or sold. By way of example, in the event of a recall, an ICS could be used to determine the status and/or location of all cannabis products that fall within the scope of the recall.

For example, transfers of cannabis-containing substances between original holding containers and new holding containers could be recorded in the ICS, using a transfer action for example. A transfer action could begin by recording information from labels on original holding containers in the ICS, for example, and designating them as labels for “source” containers. If the new holding containers have pre-existing labels, then these labels could be recorded in the ICS and designated as labels for “target” holding containers for a transfer. Alternatively, if the new holding containers do not have labels, then labels could be created by the ICS, designated as labels for target holding containers, and applied to the new holding containers. Once the transfer is complete, the ICS could record that the new holding containers now contain the transferred cannabis-containing substances. For example, a record associated with a transferred substance in the ICS could be updated to indicate that the new holding containers contain the substance.

In the case of holding containers being moved, information related to the holding containers in an ICS could be updated to include such information as any one or more of: date of transfer, time of transfer, originating location, and/or destination.

Cannabis products and the processes that are used to produce cannabis products could be recorded and tracked in an ICS in the form of “records”. A record in an ICS generally relates to a particular combination of variables, functions, and/or data structures. Records could help provide a convenient and logical organization of information within an ICS. Multiple types of records could be available in an ICS, where each record corresponds to a specific type of cannabis product or process, for example. Record types could include, for example, extraction process records, extract records, oil container records, lab sample records and/or oil jar records. Records are discussed by way of example in further detail elsewhere herein.

An ICS could create, store and/or update records as cannabis products are produced, processed, transported and sold. For example, a record could be created or updated in an ICS to record a batch of plants, a lot of product, or a certain instance of a process. Any measurements and/or data that are related to the batch, lot or process could be added to the corresponding record in an ICS. Special circumstances for a cannabis product or process, such as deviation from a standard operating procedure, could also be recorded in a record in an ICS. As such, records could provide a consolidated source of information for a product. Records could be created and/or updated by entering information into forms, logs and/or tables associated with an ICS. When a record is created, it could be assigned a unique ID to distinguish it from other records stored in an ICS, which could be referred to as a record ID. Multiple different records could also be interrelated. For example, a record for a process could be associated with a record for a product that is produced by that process.

Records and/or other information that is stored in an ICS could be recorded or updated using “actions”. For example, if cannabis product is transferred from one or more holding containers to one or more new holding containers, or a holding container is transferred from one location to a different location, then this transfer could be recorded in an ICS using a “transfer” action. Another example of an action in an ICS is a “request”, which could include a request to receive and/or view information that is stored on the ICS for example.

An ICS could use labels that are applied to holding containers and/or other packaging to help record and track cannabis products. In some embodiments, holding containers with pre-existing labels could be used. For example, a label with a unique identifier could be applied to a holding container without any knowledge of the cannabis product that will be later held in that holding container. Once the cannabis product that has been or will be transferred to the holding container is known, the label could be recorded in an ICS along with information relating to that cannabis product. Such information could include a cannabis producer number, lot number, batch number and/or plant number, for example. Other information could also or instead be recorded in the ICS, such as the date, time and location of the transfer. The recorded information could be stored in the ICS in the form of a record, for example. Recording information in the ICS could occur before, while, and/or after the cannabis product is transferred to the holding container.

In some embodiments, unique identifiers for holding container labels could be generated by an ICS. For example, after the cannabis product that will be transferred to a holding container is determined, a unique identifier could be generated by the ICS for that holding container. Labels and/or unique identifiers could be generated by the ICS using a “create new label” action. Generating a unique identifier could include generating a lot number for the product that was or will be transferred to the holding container. The unique identifier could be recorded in the ICS by adding the unique identifier to a record associated with the cannabis product. The unique identifier could be printed directly onto a label on the holding container, or printed onto a label that is later applied to the holding container. The unique identifier could indicate the cannabis producer number, lot number, batch number and/or plant number of the product, for example. In some embodiments, holding containers include both pre-existing and ICS generated labels. An ICS itself might not generate or affix labels to containers or packages, but could provide label information to a labelling machine or equipment, for example.

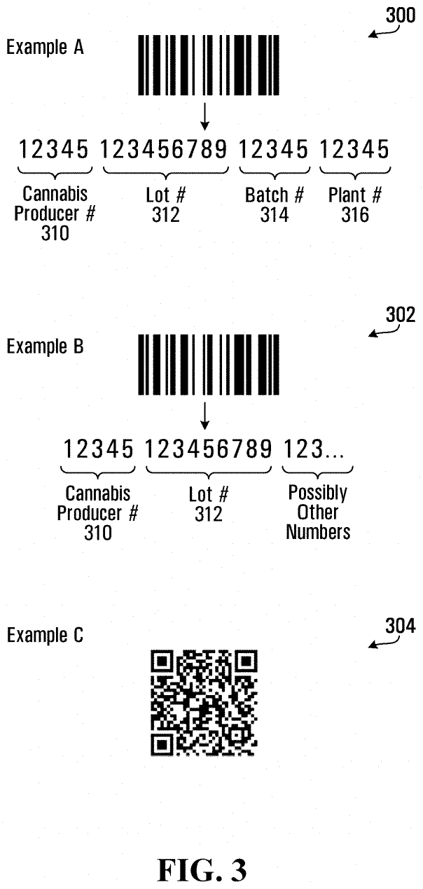

Unique identifiers on labels could include machine-readable code. FIG. 3 illustrates example formats of machine-readable code for a label. In Example A, a machine-readable code 300 is a linear barcode that encodes a number in a pattern that is readable by a machine. Specifically, in this example the machine readable-code 300 encodes a cannabis producer number 310, a lot number 312, a batch number 314 and a plant number 316. In some embodiments, the lot number 312 could be part of the GTIN or be provided in addition to the GTIN. For example, all units of the same cannabis product from the same producer could each include a barcode encoding the same GTIN corresponding to the cannabis product, but different lot numbers are assigned and also included as part of the barcode for different lots of the cannabis product.

Not all of the information shown in Example A of FIG. 3 need necessarily be encoded by the machine-readable code 300. For example, in some embodiments the particular plants from which the cannabis product that is in a container might not be known, and the plant number 316 might not be encoded.

In Example B, the lot number 312 and the producer number 310 are encoded by a machine-readable code 302. Additional information could also be included as part of the number that is encoded by the machine-readable code 302. For example, digits could be reserved for future tracking use, for internal use by regulatory authorities, and/or for internal use by cannabis producers. There could also or instead be digits that convey other types of information, such as manufacture date of the cannabis product and the expiry or ‘best before’ date of the product, for example.

The machine-readable codes 302, 304 are illustrated in FIG. 3 as linear or one-dimensional bar codes, but this is only an example. Alternatively, a barcode could be a matrix barcode or two-dimensional barcode, such as a quick response (QR) code. In Example C, a machine-readable code 304 is a QR code that could convey the same information as the machines-readable codes 300, 302, and/or different information. In some embodiments, a QR code could be used to navigate to a website hosted by an ICS, and the website could provide information related to the cannabis product.

Machine-readable codes could also or instead be carried by “smart” labels such as radio frequency identification (RFID) chips or tags. An RFID chip could be integrated into a holding container or label, for example, and encode a unique identifier and/or information related to the holding container and/or its contents.

Machine-readable codes that encode information represent one illustrative example of how information could be conveyed in markings on a label, container, or package. The information itself could be included in markings, for example. These examples, and/or other types of encoding or marking, could be used to convey any of various types of information.

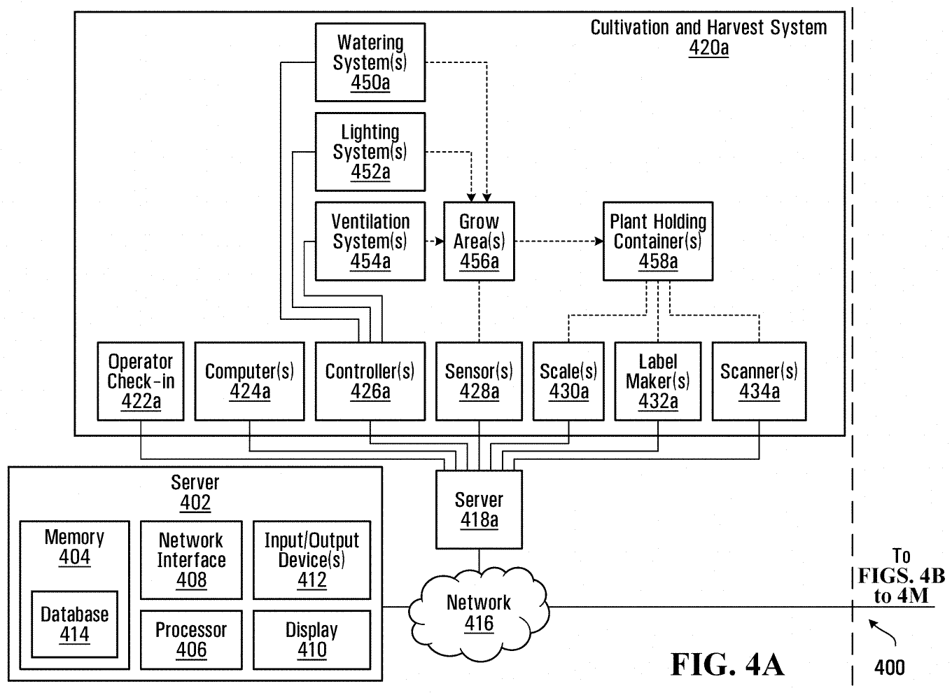

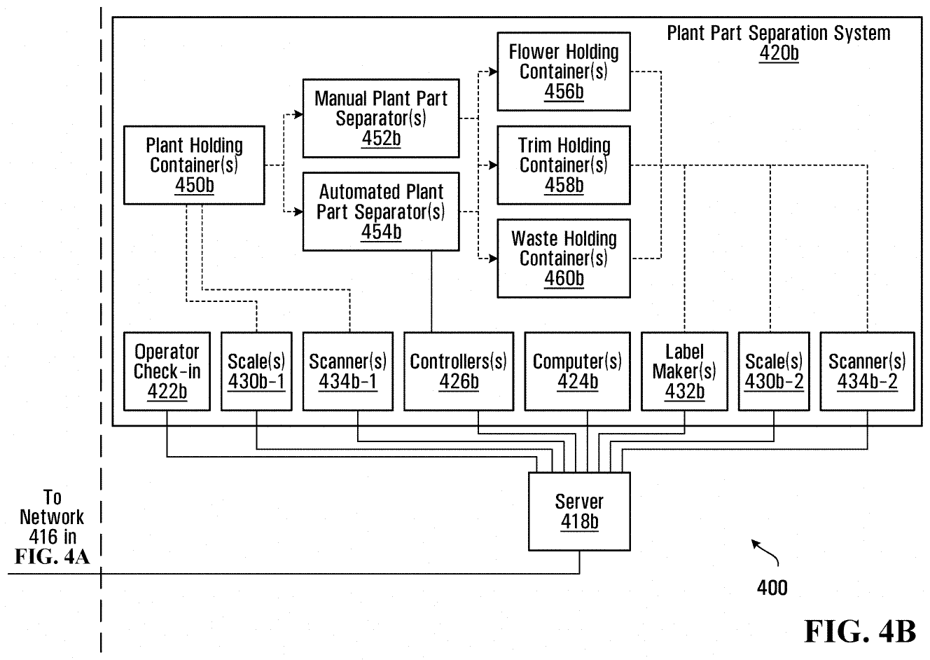

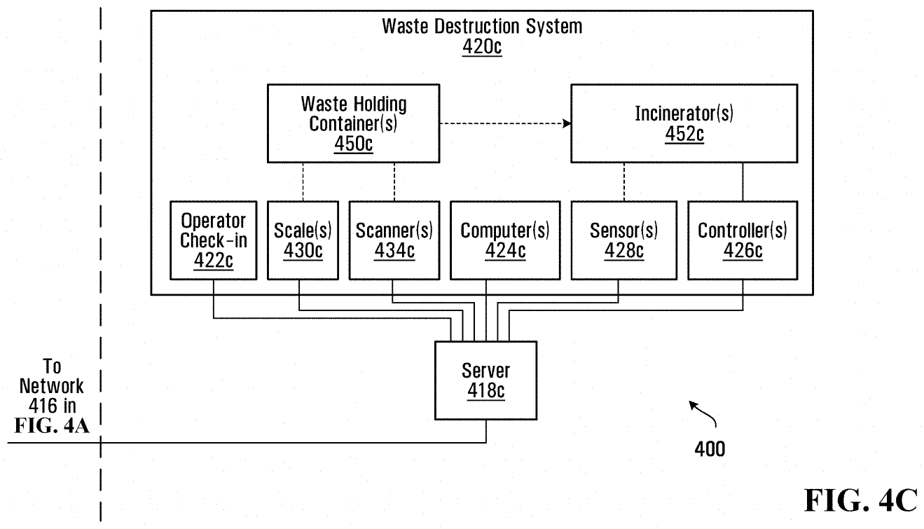

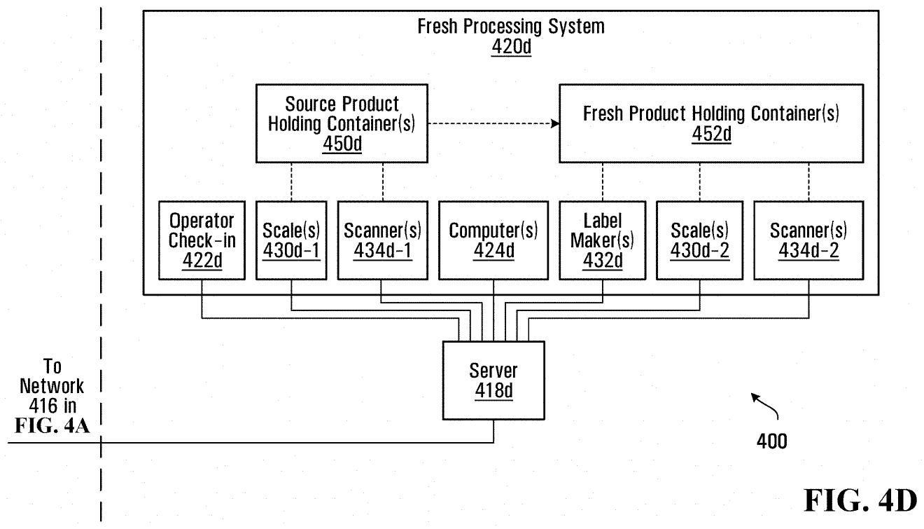

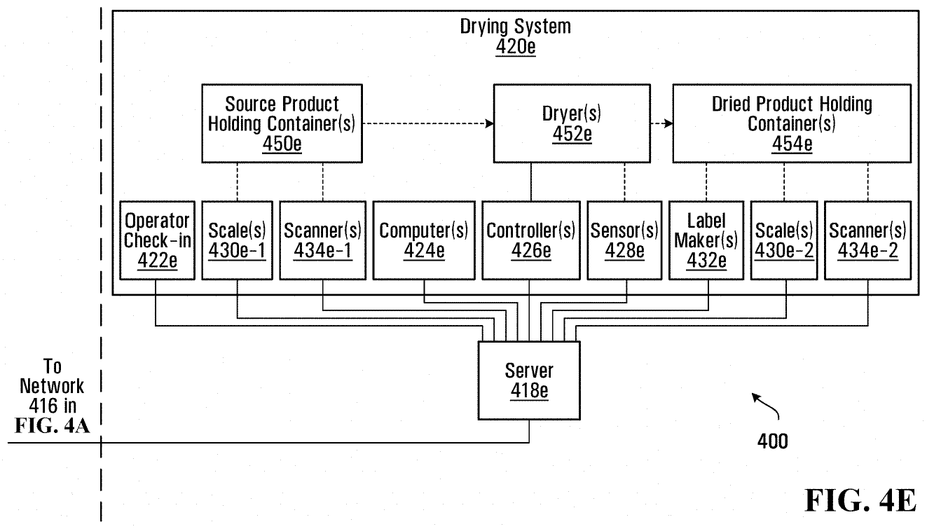

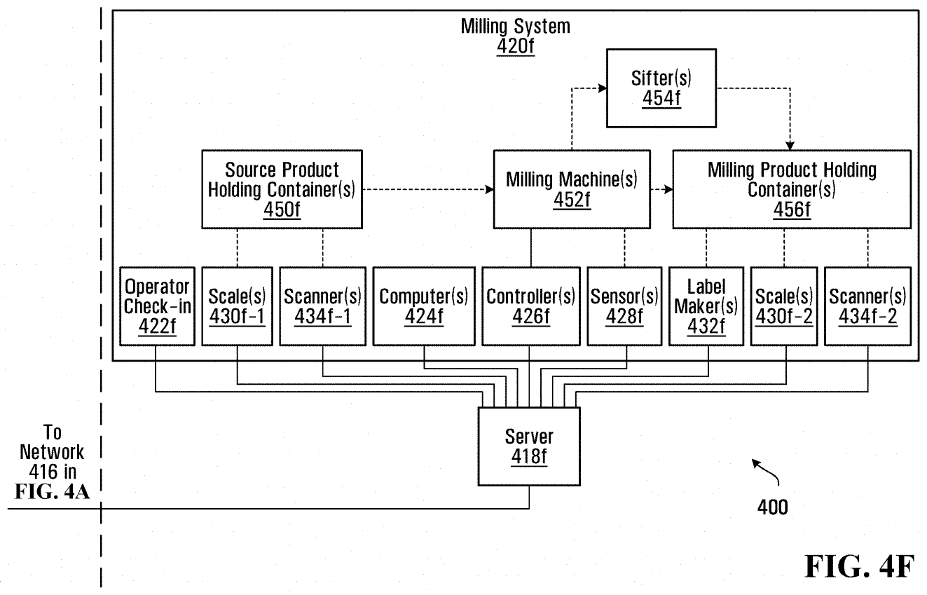

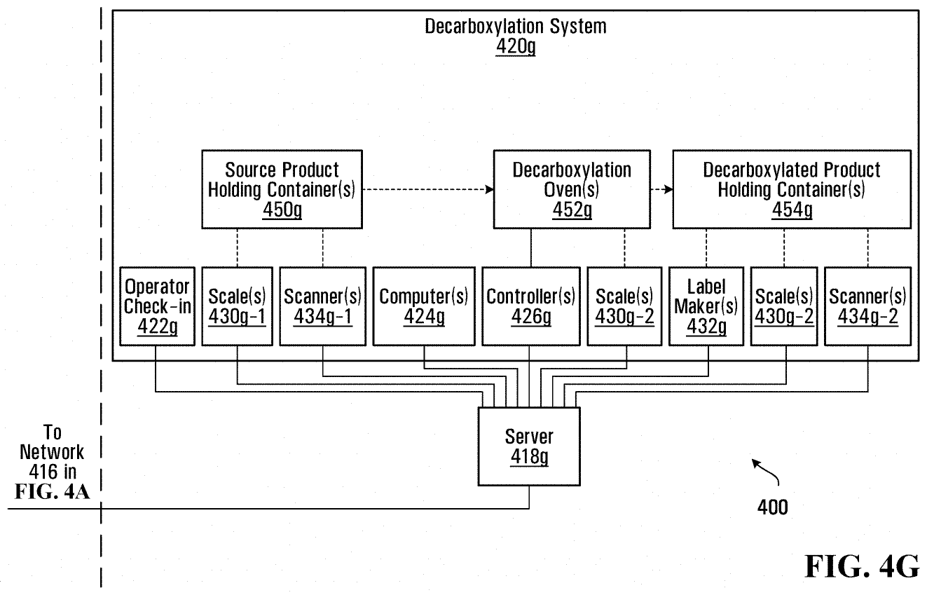

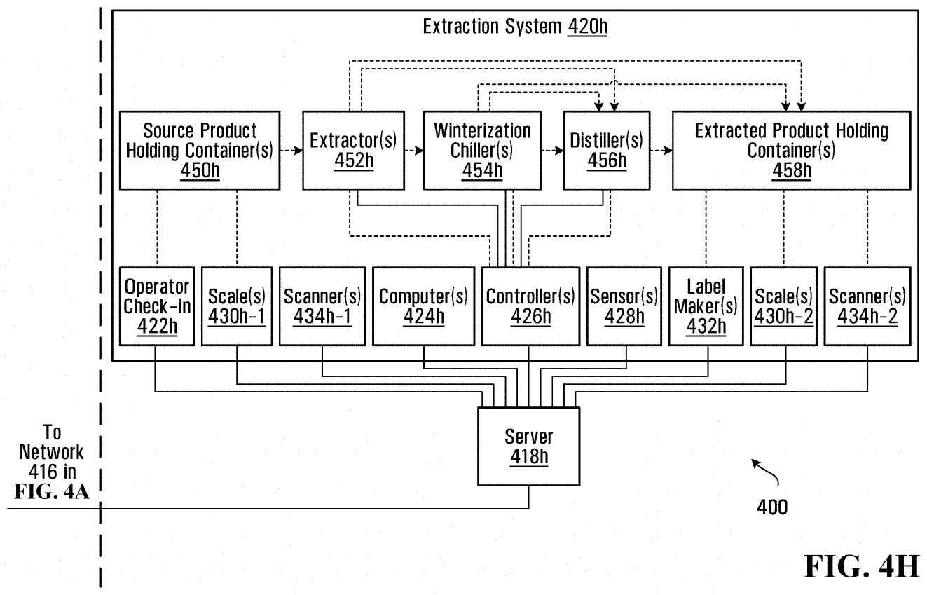

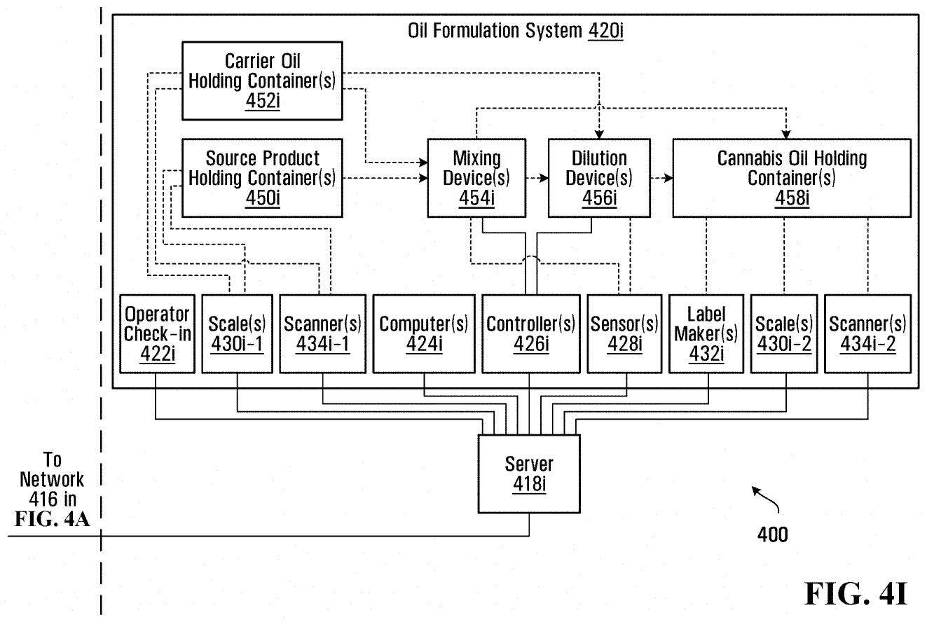

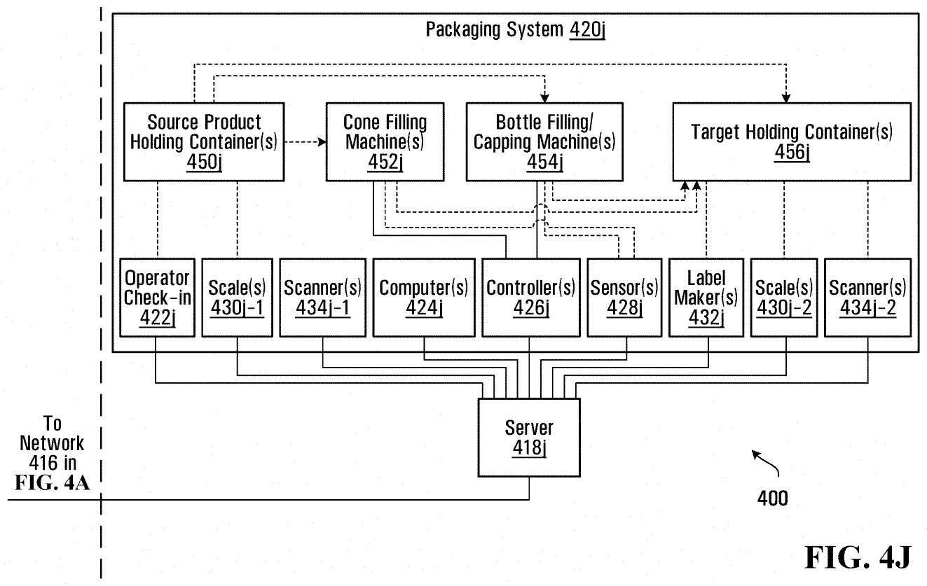

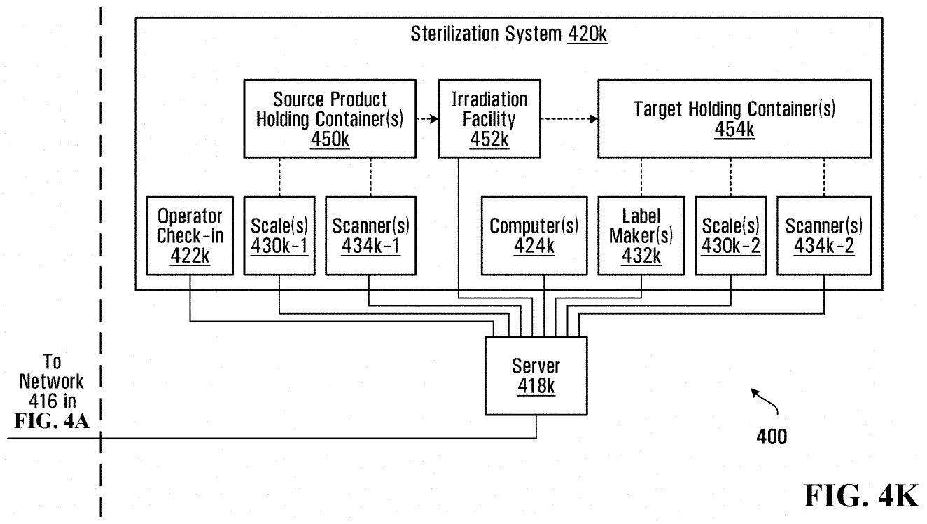

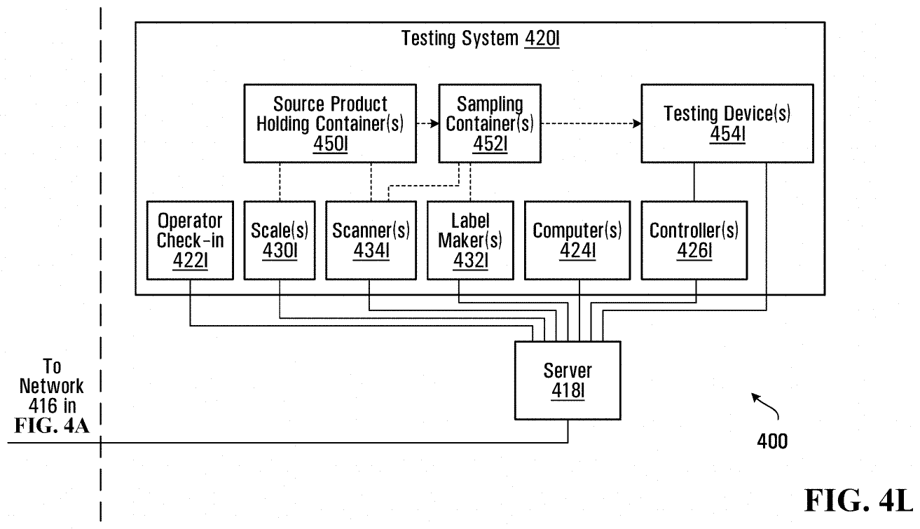

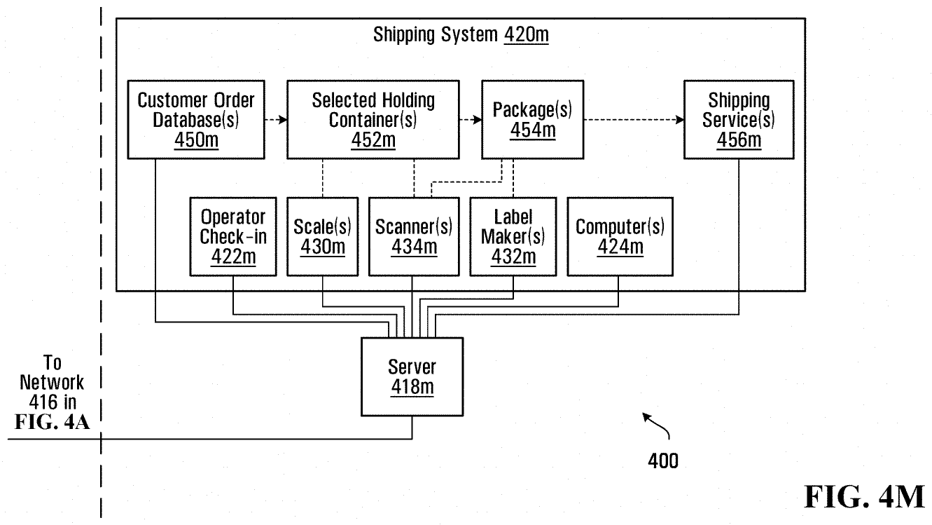

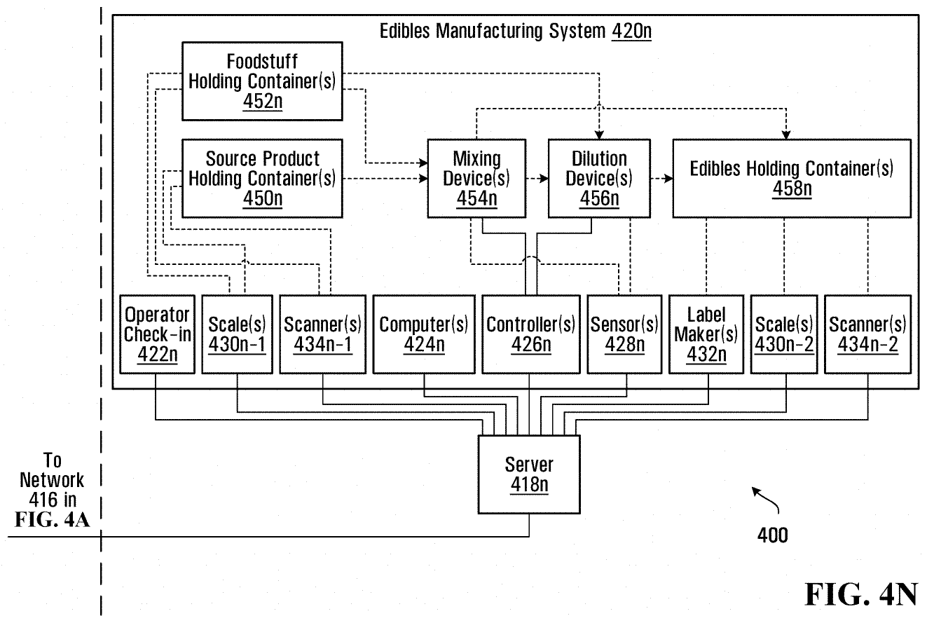

FIGS. 4A-4M are block diagrams illustrating an example system 400 implementing an ICS. The system 400 includes, as shown in FIGS. 4A-4M, respectively, a cultivation and harvest system 420a, a plant part separation system 420b, a waste destruction system 420c, a fresh processing system 420d, a drying system 420e, a milling system 420f, a decarboxylation system 420g, an extraction system 420h, an oil formulation system 420i, a packaging system 420j, a sterilization system 420k, a testing system 420l, and a shipping system 420m. The systems 420a-420m provide illustrative examples of the systems 202, 204, 206, 208, 210, 212, 214, 216, 218, 220, 222, 224, 226 of FIG. 2.

The system 400 also includes a server 402, which could implement, at least in part, an ICS. The server 402 includes a memory 404 storing a database 414, a processor 406, a network interface 408, a display 410, and one or more input/output (I/O) devices 412. In some embodiments, these server components are interconnected to each other by an internal bus and/or other type(s) of connection(s).

The memory 404 could be or include one or more memory devices, such as one or more solid state memory devices, and/or one or more memory devices that use movable or even removable storage media. The database 414 could be formatted or otherwise provided in the memory 404 to store any or all information that is recorded by the ICS. For example, the database 414 could store records, parameters, measurements and/or other information for recording and/or tracking by the ICS.

The processor 406 could be implemented by one or more processors that execute instructions stored in the memory 404. The processor 406 could be implemented, in whole or in part, using dedicated circuitry, such as an application specific integrated circuit (ASIC), a graphics processing unit (GPU), and/or a programmed field programmable gate array (FPGA) for performing any of various operations of the processor, for example.

The network interface 408 is an example of an input-output device, and enables communications between the server 402 and other devices or systems over a network 416. The particular structure of the network interface 408 is implementation-dependent, and may vary between embodiments that support different types of connections and/or communication protocols, for example. The network interface could enable communications over wired and/or wireless connections. In general, a network interface include a physical interface such as a port, connector, or other component to interface with a communication medium, and a receiver and/or transmitter to process received signals and/or transmit signals for transmission. A transceiver is an example of a component that includes both a receiver and a transmitter, and could be implemented in the network interface 408.

The display 410 is another example of an input-output device, to allow users such as system operators to view any or all information stored on the ICS and/or to otherwise interact with the ICS and possibly other components of the system 400. For example, the display 410 could show a record for a cannabis product and/or process. The display 410 could also or instead allow a user to view the current status of any or all systems within the system 400, including information regarding which systems or devices are currently in use, the processes these systems or devices are performing, and/or the operator(s) using the systems or devices, for example. Any of various types of displays could be implemented at 410, including touchscreen displays that also enable user input.

Other I/O devices 412 could also or instead be provided. For example, one or more user input devices that allow a user to manually input information, actions and/or requests could be provided. Examples of user input devices include keyboards, computer mice, touchscreens, buttons, dials and switches. The I/O devices 412 could also or instead include one or more output devices, such as output ports for exporting data stored in the database 414. Other types of I/O devices are also contemplated. An access card scanner, for example, could provide security and access control for the server 402.

In some embodiments, the server 402 itself does not include user I/O devices such as a display 410 or user input devices for receiving inputs from a user. User interaction with the server 402 could be through one or more separate components such as one or more workstations that communicate with the server 402 through local connections with the server and/or network connections through the network 416. Such workstations could be identical to or similar in structure to the server 402, but might not locally store the database 414, for example.

The network 416 could be or include any of various types of network equipment implementing any of various type(s) of network(s). In some embodiments, the network 416 includes a corporate network of a cannabis producer. The network 416 could also or instead include the internet. The particular type(s) of networks(s) in a system such as 400 could be implementation-dependent. The server 402 could be located at a corporate office, and at least some of the systems 420a-420m are located remotely from the server 402. At least the remotely-located systems could connect or otherwise communicate with the server 402 through the internet, whereas co-located systems that are at the same location as the server 402 could connect or otherwise communicate with the server through a local area network (LAN) or other type(s) of local network(s).

The network 416 is connected to or otherwise in communication with multiple servers 418a, 418b, 418c, 418d, 418e, 418f, 418g, 418h, 418i, 418j, 418k, 418l, 418m, 418n shown in FIGS. 4A-4M, respectively. Network/server communications could be provided, for example, using physical connections such as cables and/or wires, and/or using wireless connections or channels, such as WIFI™ connections, Bluetooth™ connection, and/or longer-range wireless communications.

The servers 418a-418n could be generally similar in structure to the sever 402, but there could be at least operational, and/or possibly structural differences between servers. For example, the servers 418a-418n could be involved in maintaining an ICS at the server 402 by sending system-related information through the network 416 to the server 402, but the servers 418a-418n might not locally store a complete copy of the database 414.

In some embodiments, the servers 418a-418n could relay information from other devices to the server 402. Information could also or instead be stored on the servers 418a-418n. Although the servers 418a-418n could be distributed throughout the system 400 as shown, this might not always be the case. Two or more of the servers 418a-418n, for example, could be co-located. Although illustrated separately in FIGS. 4A-4M, in some embodiments two or more of the servers 418a-418n could be implemented using a single server. At least some of the systems 420a-420n could be connected to or otherwise in communication with the network 416 without an intervening server 418a-418n. One or more components of a system 420a-420n could communicate with the network 416 without necessarily traversing a server in connecting to the network. A system component could also or instead communicate with the network 416 through some other type of communication equipment or device that does not necessarily implement a server.

The cultivation and harvest system 420a includes an operator check-in device 422a, one or more computers 424a, one or more controllers 426a, one or more sensors 428a, one or more scales 430a, one or more label makers 432a and one or more scanners 434a. These components are each connected to the server 418a in the example shown. Connections between these components and the server 418 could include wired and/or wireless connections, through any of various types of interfaces. Each component that is connected to or otherwise in communication with the server 418a includes an interface compatible with an interface that is provided at the server. The particular type(s) of interface(s) provided at the system components and the server 418a would be dependent upon the type(s) of connection(s) and/or communication protocol(s) to be supported.

In some embodiments, one or more operator check-in devices such as 422a could be implemented in a cannabis production system to record and track one or more operators involved in a process or using a device. The term operator, as used herein at least with reference to FIGS. 4A-4M, refers to any person involved in operating or using a cannabis production system. Each operator could be assigned a unique ID that is recorded by an operator check-in device when the operator wishes to access secure premises and/or use system equipment or devices. Examples of operator check-in devices include punch card readers to read operator information from an operator's punch card, magnetic card readers to read operator information from a magnetic strip on an operator's identification or access card, and RFID readers to read an RFID tag or chip on an operator's identification or access card. An operator check-in device could also or instead include a computer or controller into which an employee must enter login information, including at least operator information such as the operator's unique ID or username and a password.

Operator information that is read by or otherwise obtained by an operator check-in device 422a could be stored locally at the cultivation and harvest system 420a, and/or transmitted to the server 402 for storage in the database 414. Local storage of operator information could be by the operator check-in device 442a itself, and/or one or more other components of the cultivation and harvest system 420a, such as a computer 424a and/or the server 418a.

Other information could also or instead be recorded. The operator check-in device could record the date and time that an operator enters an area, leaves an area, starts a process or equipment and/or stops a process or equipment, for example.

Any or all information that is obtained or generated by an operator check-in device, such as operator information and/or other information disclosed by way of example above, could be recorded in the ICS. For example, the operator check-in device 422a could transmit information regarding operators and/or their activities in the cultivation and harvest system 420a to the server 418a, which could store this information and/or forward it to the server 402.

In some embodiments, one or more computers such as 424a could be implemented in a cannabis production system, for such purposes as enabling operators to manually enter ICS data, otherwise interact with an ICS system, and/or control system devices. For example, the computer 424a could store entered data and/or transmit entered data to the server 418a, which could store and/or forward the data to the server 402. The computer 424a could also or instead enable an operator to access ICS data, in the database 414 for example, and output an indication of that data on a display screen or other output device. Examples of computers include desktop computers, laptop computers, tablet computers and other electronic devices. In general, the computer 424a could be similar in structure to the server 402, but need not necessarily store the database 414. Depending on implementation, the computer 424a might or might not include a network interface. In a server-based implementation as shown in FIG. 4A, for example, the computer 424a could include an interface that might or might not be a network interface but is compatible with an interface provided at the server 418a.

In some embodiments, one or more controllers such as 426a could be implemented in a cannabis production system, to control any or all of various types of devices or equipment. A controller could be integrated within a controlled device or equipment, or be separate from the controlled device or equipment as shown in FIG. 4A. Controllers could be implemented, for example, using hardware, firmware, one or more components that execute software stored in one or more non-transitory memory devices. Microprocessors, ASICs, FPGAs, and Programmable Logic Devices (PLDs) are examples of processing devices that could be used to execute software.

A controller 426a could store, receive, and/or otherwise obtain control settings, and control one or more devices or equipment to run according to those settings. For example, a controller 426a could be programmable by operators, through a computer 424a and/or through a user interface of the controller for example, and/or by the ICS. An ICS-programmable controller 426a could access, download, or otherwise determine, or be programmed with, control settings from the database 414. In some embodiments, a controller 426a could record control settings and/or other information in the ICS. Information that is used by and/or obtained by a controller 426a could be locally stored, by the controller and/or another component of the cultivation and harvest system 420a for example, and/or transmitted to the server 418a for local storage and/or transmission to the server 402.

In some embodiments, sensors such as 428a could be implemented in a cannabis production system to measure or otherwise determine any or a variety of parameters involved in production. These parameters, and possibly other information such as the time at which measurements were taken, could be recorded in the ICS. Examples of sensors, any one or more of which could be implemented in a cannabis production system, include the following:
carbon dioxide sensor;
nitrogen oxide sensor;
oxygen sensor;
ozone monitor;
pH sensor;
potentiometric sensor;
redox electrode;
smoke detector;
electrical current sensor;
metal detector;
voltage detector;
air pollution sensor;
humidity sensor;
rain sensor;
snow gauge;
soil moisture sensor;
air flow meter;
water meter;
barometer;
pressure sensor;
pressure gauge;
flame detector;
light sensor;
heat flux sensor; and
thermometer.

Sensor readings or measurements could be locally stored, by a sensor 430a and/or another component of the cultivation and harvest system 420a for example, and/or transmitted to the server 418a for local storage and/or transmission to the server 402.

In some embodiments, one or more scales such as 430a could be implemented in a cannabis production system to weigh products, waste material, packages and/or holding containers, for example. Scales could include, for example, electronic scales that are in communication with or otherwise able to access the ICS.

When an electronic scale measures the weight of a cannabis product and/or a holding container, for example, the scale could automatically transmit this weight to the ICS, where it could be recorded. Non-electronic scales could also or instead be used in a cannabis production system, and the weights measured by these scales could be manually entered into the ICS using a computer 424a, for example.

A description of a weight measured by a scale 430a could also be recorded in the ICS. The description could include information regarding the current stage of production of a cannabis product and/or holding container when the cannabis product and/or holding container was weighed. An operator could manually enter this description into an electronic scale 430a or a computer 424, for example, which could then transmit the description to the ICS. A description of a measured weight could also or instead be inferred by the ICS. For example, an electronic scale 430a could be associated with a specific step in a cannabis production process or a specific device or equipment in a cannabis production system, and a description of the weights measured by that scale could therefore be predefined in the ICS. In some embodiments, a certain scale might only be used to measure the weight of holding containers containing extract collected from an extraction process, and the ICS could automatically associate any or all weights measured by the scale with that stage of production.

A record ID and/or other identifier for the cannabis product and/or holding container weighed by a scale 430a could be recorded in the ICS. For example, an electronic scale 430a could transmit a record ID and/or other identifier for a weighed cannabis product and/or holding container to the ICS, along with the measured weight of that cannabis product and/or holding container, allowing the ICS to identify which record the weight should be recorded in. To determine the record ID and/or other identifier, an operator could manually read a label on the holding container and enter information from the label into the ICS using the electronic scale or another device. Also or alternatively, a label on the holding container could be read and recorded in the ICS using a scanner. The scanner could be linked to the scale to automatically associate the label of the holding container with the measured weight.

A scale 430 could also receive control information and/or other information. A scale could be controlled, for example, to record a weight only when a cannabis product or holding container is in proper position for weighing. In some embodiments, a controller sends a control signal to a scale 430a to trigger a measurement. Such a controller could be integrated with a scale 430a, or be separate from the scale. Measurement could also or instead be manually initiated or triggered by an operator, through a user interface of the scale or another component that is connected to or otherwise in communication with the scale.

Weight measurements, and possibly other information that is determined or otherwise obtained by or from a scale 430a could be locally stored, by the scale and/or another component of the cultivation and harvest system 420a for example, and/or transmitted to the server 418a for local storage and/or transmission to the server 402.

In some embodiments, one or more label makers 432a could be implemented in a cannabis production system to generate labels that are applied to holding containers, for example. A label maker 432a could be in communication with or otherwise have access to the ICS. In some embodiments, the ICS could control a label maker 432a, and thereby control the particular labels that are applied to holding containers. For example, the ICS could transmit information and/or machine-readable code for a label to a label maker 432a, and the label maker could generate a label based on the information and/or a machine-readable code that encodes the information. The ICS could also or instead send an image of a label to a label maker 432a, which could print the image onto an adhesive label and/or directly onto a holding container. In some embodiments, a label maker 432a could generate information and/or machine-readable code for a label, produce a label based on the information and/or machine-readable code, and apply the label to one or more holding containers.

A label maker 432a could record each label in the ICS. Label information could be locally stored, by a label maker 432a and/or another component of the cultivation and harvest system 420a for example, and/or transmitted to the server 418a for local storage and/or transmission to the server 402.

A label maker 432a is an example of a labelling or marking system or station, which could include a printing or marking device to print or mark on a label and/or directly on a holding container or package. In some embodiments, a single printing or marking device is suitable for printing or marking, with ink for example, on multiple substrates such as labels and holding containers, labels and packages, or labels, holding containers, and packages. In embodiments that involve printing or marking labels, a labelling system or marking station could also include a label applicator to affix labels to holding containers and/or packages. A controller for a labelling system or marking station could be integrated with the labelling system or marking station, or be a separate component.

In some embodiments, one or more scanners 434a could be implemented in a cannabis production system to read, record, and/or decode markings, which could be directly printed on holding containers and/or packages, and/or on labels that are affixed to the holding containers and/or packages. For example, a scanner could be used to read, record and/or decode machine-readable code(s) on a label.

Examples of scanners 434a include barcode scanners, image scanners and RFID readers. A scanner 434a could be provided in the form of a handheld scanner, a mobile electronic device, a scanner mounted to a structure (a table or counter, for example), a scanner embedded in a structure, a scanner integrated into equipment in a cannabis production system and/or a wearable scanner. Scanners could be wired or wireless. Multiple scanners, of the same type or different types, could be implemented.

When a marking, on a label affixed to a holding container for example, is scanned by a scanner 434a, any of a variety of information could be recorded in the ICS. For example, a scanner 434a could be specific to a certain location, device, equipment, and/or process in a cannabis production system. Scanning a label using that scanner indicates that the holding container or package associated with the label is at that specific location, device, equipment, and/or process.

A scanner could also receive information. In some embodiments, a scanner could receive a search or control parameter and generate an alert or other output when the search or control parameter is found or satisfied. A search parameter could be a lot identifier, for example, and a scanner 434a could generate an alert when a marking consistent with the lot identifier is scanned. A count is an example of a control parameter, and a scanner 434a could generate an alert or other output when a certain number of markings have been scanned and/or provide an output indicating a count of scanned markings. A scanner 434a could also or instead confirm a change in lot and/or batch number at a correct time during a production run.

The example cultivation and harvest system 420a in FIG. 4A also includes one or more watering systems 450a, one or more lighting systems 452a, and one or more ventilation systems 454a. The watering system(s) 450a, the lighting system(s) 452a and the ventilation system(s) 454a are connected to or otherwise in communication with, and are controlled by, one or more of the controllers 426a. Control of a watering system 450a could involve controlling one or more valves, for example, to control water flow to an irrigation system and/or particular components such as sprinkler heads. A lighting system 452a could be controlled by controlling power to lights and/or shades, for example. In some embodiments, control of the ventilation system(s) 454a could involve controlling one or more air inlets, one or more air outlets, one or more heaters, one or more coolers, and/or one or more airflow components such as fans.

Control settings for any or all of the watering system(s) 450a, the lighting system(s) 452a, and the ventilation system(s) 454a could be provided to, determined by, or otherwise obtained by the controller(s) 426a. Watering, lighting, ventilation, and/or temperature programs or schedules could be downloaded to one or more controllers 426a and used to control any or all of the watering system(s) 450a, the lighting system(s) 452a, and the ventilation system(s) 454a. In some embodiments, one or more controller(s) 426a dynamically control any or all of the watering system(s) 450a, the lighting system(s) 452a, and the ventilation system(s) 454a, based on sensor readings, for example. Combinations of predetermined and dynamic control are also contemplated. For example, any or all of the watering system(s) 450a, the lighting system(s) 452a, and the ventilation system(s) 454a could be controlled according to a predetermined program or schedule as long as one or more monitored parameters are maintained within target ranges, and dynamic control of one or more of the systems 450a, 452a, 454a could be initiated in response to an out-of-range parameter.

The cultivation and harvest system 420a also includes one or more grow areas 456a, used to cultivate cannabis plants. The watering system(s) 450a, the lighting system(s) 452a and the ventilation system(s) 454a interact with, and in that sense could be considered to be associated with, the grow area(s) 456a. The interactions between the watering system(s) 450a, the lighting system(s) 452a, the ventilation system(s) 454a, and the grow areas 456a are illustrated using dashed lines in FIG. 4A.

In FIGS. 4A-4M, solid lines are intended to represent wired or wireless connections for communications between components. Dashed lines are intended to indicate that components interact or are related or associated in some way, but are not necessarily in communication with or coupled to each other. By way of example, the watering system(s) 450a could provide water to the grow area(s) 456a, but this does not necessarily mean that the watering system(s) would be in communication with the grow area(s), or that the watering system(s) would necessarily be in any way physically coupled to the grow area(s). Similarly, the lighting system(s) 452a and the ventilation system(s) 454a provide light and air flow to the grow area(s) 456a, but are not necessarily coupled to the grow area(s).

FIG. 4A similarly shows the grow area(s) 456a as being associated with the sensors 428a, which could measure, record and/or track any of a variety of parameters and/or growing conditions in the grow area(s). In some embodiments, the sensor(s) 428a could provide measurements or readings to one or more controller(s) 426a, and the controller(s) could control one or more of the watering system(s) 450a, the lighting system(s) 452a, and the ventilation system(s) 454a based on the measurements or readings from the sensor(s).

During and/or after a harvest, cannabis plants from the grow area(s) 456a could be transferred to one or more plant holding containers 458a. The plant holding container(s) 458a could be weighed by the scale(s) 430a, labeled by the label maker(s) 432a and/or scanned by the scanner(s) 434a, and therefore FIG. 4A includes dashed lines to represent interactions or associations between these components.

In some embodiments, a production system includes other systems with at least some components that may be identical or similar to those in the example cultivation and harvest system 420a. With reference to FIG. 4B, for example, a plant part separation system 420b could include one or more operator check-in devices 422b, one or more computers 424b, one or more controllers 426b, one or more scales at 430b-1 and/or 430b-2, one or more label makers 432b and one or more scanners at 434b-1 and/or 434b-2. These components are connected to or otherwise in communication with the server 418b. Implementation options for all of these components are described herein, at least above with reference to FIG. 4A. Although two sets of scale(s) and scanner(s) are shown at 430b-1, 430b-2 and 434b-1, 434b-2, in some embodiments a plant part separation system could include only one set of either or both of these components. Different holding containers could be transferred to the same weighing station with one set of scales, for example. In some embodiments, different holding containers could be scanned with the same set of one or more portable scanners instead of or in addition to equipment-mounted or equipment-specific scanners. Separate sets of scale(s) and scanner(s) are shown at 430b-1, 430b-2 and 434b-1, 434b-2 solely to simplify the illustration of connecting lines in FIG. 4B.

The example plant part separation system 420b further includes one or more plant holding containers 450b, one or more manual plant part separators 452b, one or more automated plant part separators 454b, one or more flower holding containers 456b, one or more trim holding containers 458b, and one or more waste holding containers 460b. The holding containers 450b, 456b 458b, 460b could be any of various types of containers, and different types of containers could be used to hold harvested and separated plants. In some embodiments, the plant holding container(s) 450b are the same holding container(s) as shown at 458a in FIG. 4A. In some embodiments, a manual plant part separator 452b includes one or more sorting trays or tables at which an operator sorts harvested plant material. An automated plant part separator 454b could include a machine vision system or other means to distinguish flower, trim, and waste from each other, and a sorting station to separate plant material that has been identified as flower, trim, and waste from each other. Some embodiments could include both manual and automated plant part separators.

The plant holding container(s) 450b could be weighed and/or scanned using the scale(s) 430b-1 and/or the scanner(s) 434b-1, to quantify and/or identify inputs into plant part separation. The cannabis plants from the plant holding container(s) 450b could be transferred to the manual plant part separator(s) 452b and/or the automated plant part separator(s) 454b for plant part separation. At least the automated plant part separator(s) 454b could be connected to or otherwise in communication with, and controlled by, a controller 426b. The flower, trim and waste produced by the manual plant part separator(s) 452b and/or the automated plant part separator(s) 454b could be transferred to the flower holding container(s) 456b, the trim holding container(s) 458b, and the waste holding container(s) 460b, respectively. The flower holding container(s) 456b, the trim holding container(s) 458b, and the waste holding container(s) 460b could be weighed by the scale(s) 430b-2 and/or labeled by the label maker(s) 432b, and markings on the holding container(s) or label(s) could be scanned by the scanner(s) 434b-2. Weights as measured by the scale(s) 430b-2 could be used to reconcile input plant material with output plant material, to maintain desired and/or required records of plant material during processing.

The example waste destruction system 420c in FIG. 4C includes one or more operator check-in devices 422c, one or more computers 424c, one or more controllers 426c, one or more scales 430c, one or more sensors 428c, and one or more scanners 434c. These components are connected to or otherwise in communication with the server 418c. Implementation options for all of these components are described herein, at least above with reference to FIG. 4A.

The example waste destruction system 420c further includes one or more waste holding containers 450c and one or more incinerators 452c. The waste holding container(s) 452c could include any of various types of containers, and in some embodiments the waste holding container(s) 450c are those shown at 460b in FIG. 4B. The waste holding container(s) 450c could be weighed and/or scanned using the scale(s) 430c and/or the scanner(s) 434c, to quantify and/or identify inputs into waste destruction. The waste from the waste holding container(s) 450c could be transferred to the incinerator(s) 452c for incineration. The incinerator(s) 452c are connected to or otherwise in communication with the sensor(s) 428c to measure operating parameters and/or monitor the process of incineration. The incinerator(s) 452 are also connected to or otherwise in communication with the controller(s) 426c to control the process of incineration.

Referring now to FIG. 4D, an example fresh processing system 420d includes one or more operator check-in devices 422d, one or more computers 424d, one or more scales at 430d-1 and/or 430d-2, one or more label makers 432d and one more scanners at 434d-1 and/or 434d-2. These components are connected to or otherwise in communication with the server 418d. Implementation options for all of these components are described herein, at least above with reference to FIG. 4A. Although two sets of scale(s) and scanner(s) are shown at 430d-1, 430d-2 and 434d-1, 434d-2, in some embodiments a fresh processing system could include only one set of either or both of these components. Separate sets of scale(s) and scanner(s) are shown at 430d-1, 430d-2 and 434d-1, 434d-2 solely to simplify the illustration of connecting lines in FIG. 4D.

The example fresh processing system 420d further includes one or more source product holding containers 450d and one or more fresh product holding containers 452d. The containers 450d, 452d could include any of various types of container, and different container types could be used for source product and fresh product. The source product holding container(s) 450d could contain cannabis flower and/or trim from plant part separation, for example. In some embodiments, the source product holding container(s) 450d are the same holding container(s) as shown at 456b and/or 458b in FIG. 4B.

The source product holding container(s) 450d could be weighed and/or scanned using the scale(s) 430d-1 and/or the scanner(s) 434d-1, to quantify and/or identify inputs to the fresh processing system 420d. The source product(s) in the source product holding container(s) 450d could then be transferred to the fresh product holding container(s) 452d and sealed. The fresh product holding container(s) 452d could be weighed by the scale(s) 430d-2 and/or labeled by the label maker(s) 432d. Markings on the holding container(s) on the fresh product holding container(s) 452d or label(s) could be scanned by the scanner(s) 434d-2. Weights as measured by the scale(s) 430d-2 could be used to reconcile input source product with total output fresh product, to maintain desired and/or required records of source product during processing.

An example drying system 420e as shown in FIG. 4E includes one or more operator check-in devices 422e, one or more computers 424e, one or more controllers 426e, one or more sensors 428e, one or more scales at 430e-1 and/or 430e-2, one or more label makers 432e and one or more scanners 434e-1 and/or 434e-2. These components are connected to or otherwise in communication with the server 418e. Implementation options for all of these components are described herein, at least above with reference to FIG. 4A. Although two sets of scale(s) and scanner(s) are shown in at 430e-1, 430e-2 and 434e-1, 434e-2, in some embodiments a drying system could include only one set of either or both of these components. Separate sets of scale(s) and scanner(s) are shown at 430e-1, 430e-2 and 434e-1, 434e-2 solely to simplify the illustration of connecting lines in FIG. 4E.

The drying system 420e further includes one or more source product holding containers 450e, one or more dryers 452e, and one or more dried product holding containers 454e. The containers 450e, 454e could include any of various types of container, and different container types could be used for source product and dried product. The source product holding container(s) 450e could contain cannabis flower and/or trim from plant part separation, for example, and could include any of various types of containers. In some embodiments, the source product holding container(s) 450e are the same holding container(s) as shown at 456b and/or 458b in FIG. 4B. The dryer(s) 452e could be or include any of various types of dryers, such as one or more commercial dehydrator systems. Although FIG. 4E illustrates only dryer(s) 452e, a drying system could also or instead provide curing. Curing could be provided, for example, using a curing vessel in which a curing solution is applied to source product to extract moisture from the source product. One or more of the controller(s) 426e could control a curing or parameters such as supply of curing solution(s) from one or more solution holding container(s) to the curing vessel by controlling one or more valves, curing temperature by controlling one or more heaters or coolers to heat or cool the vessel and/or curing solution(s), and/or curing pressure by controlling a vacuum system or compression system to pressurize or depressurize the curing vessel, for example.

The source product holding container(s) 450e could be weighed and/or scanned using the scale(s) 430e-1 and/or the scanner(s) 434e-1, to quantify and/or identify inputs to the drying system. The source product(s) in the source product holding container(s) 450e could then be transferred to the dryer(s) 452e, to dry the source product(s). The controller(s) 426e could be connected to or otherwise in communication with the dryer(s) 452e, to control the dryer(s). The sensors 428e could similarly be connected to or otherwise in communication with the dryer(s) 452e, to measure one or more parameters and/or otherwise monitor one or more properties of a drying process or equipment. Dried product could then be transferred to the dried product holding container(s) 454e. The dried product holding container(s) 454e could be weighed by the scale(s) 430e-2 and/or labeled by the label maker(s) 432e. Markings on the dried product holding container(s) 454e or label(s) could be scanned by the scanner(s) 434e-2. Weights as measured by the scale(s) 430e-2 could be used to reconcile input source product with total output dried product, to maintain desired and/or required records of source product during processing.

FIG. 4F illustrates an example milling system 420f, which includes one or more operator check-in devices 422f, one or more computers 424f, one or more controllers 426f, one or more sensors 428f, one or more scales at 430f-1 and/or 430f-2, one or more label makers 432f and one or more scanners at 434f-1 and/or 434f-2. These components are connected to or otherwise in communication with the server 418f. Implementation options for all of these components are described herein, at least above with reference to FIG. 4F. Although two sets of scale(s) and scanner(s) are shown at 430f-1, 430f-2 and 434f-1, 434f-2, in some embodiments a milling system could include only one set of either or both of these components. Separate sets of scale(s) and scanner(s) are shown at 430f-1, 430f-2 and 434f-1, 434f-2 solely to simplify the illustration of connecting lines in FIG. 4F.

The milling system 420f further includes one or more source product holding containers 450f, one or more milling machines 452f, one or more sifters 454f and one or more dried product holding containers 456f. The containers 450f, 456f could include any of various types of container, and different container types could be used for source product and dried product. The source product holding container(s) 450f could contain cannabis flower and/or trim from plant part separation, and/or dried cannabis product from a drying process, for example. In some embodiments, the source product holding container(s) 450f are the same holding container(s) as shown at 456b, 458b, and/or 454e in FIGS. 4B and 4E.

The source product holding container(s) 450f could be weighed and/or scanned using the scale(s) 430f-1 and/or the scanner(s) 434f-1, to quantify and/or identify inputs to the milling system 420f. The source product(s) in the source product holding container(s) 450f could then be transferred to the milling machine(s) 452f, which could be implemented as milling equipment to mill the source product(s) and/or one or more grinders to grind the source product. One or more of the controller(s) 426f could be connected to or otherwise in communication with the milling machine(s) 452f to control the milling machine(s). The sensor(s) 428f could similarly be connected to or otherwise in communication with the milling machine(s) 452f, to measure one or more parameters and/or otherwise monitor one or more properties of a milling process or equipment.

Milled product could then be transferred to one or more sifters 454f and/or to the dried product holding container(s) 456f. The sifter(s) 454f could include one or more filters or screens to sift the milled cannabis product and separate it based on particle size. The outputs from the sifter(s) 454f could also or instead be transferred to the milled product holding container(s) 456f. The milled product holding container(s) 456f could be weighed by the scale(s) 430f-2 and/or labeled by the label maker(s) 432f. Markings on the milled product holding container(s) or label(s) could be scanned by the scanner(s) 434f-2. Weights as measured by the scale(s) 430f-2 could be used to reconcile input source product with total output milled product, to maintain desired and/or required records of source product during processing.

Referring now to FIG. 4G, an example decarboxylation system 420g includes one or more operator check-in devices 422g, one or more computers 424g, one or more controllers 426g, one or more sensors 428g, one or more scales at 430g-1 and/or 430g-2, one or more label makers 432g and one or more scanners at 434g-1 and/or 434g-2. These components are connected to or otherwise in communication with the server 418g. Implementation options for all of these components are described herein, at least above with reference to FIG. 4A. Although two sets of scale(s) and scanner(s) are shown in at 430g-1, 430g-2 and 434g-1, 434g-2, in some embodiments a decarboxylation system could include only one set of either or both of these components. Separate sets of scale(s) and scanner(s) are shown at 430g-1, 430g-2 and 434g-1, 434g-2 solely to simplify the illustration of connecting lines in FIG. 4G.

The decarboxylation system 420g further includes one or more source product holding containers 450g, one or more decarboxylation ovens 452g and one or more decarboxylated product holding containers 454g. The containers 450g, 454g could include any of various types of container, and different container types could be used for source product and decarboxylated product. The source product holding container(s) 450g could contain cannabis flower and/or trim from plant part separation, dried cannabis product from a drying process, and/or milled cannabis from a milling process, for example. In some embodiments, the source product holding container(s) 450g are the same holding container(s) as shown at 456b, 458b, 454e, and/or 450f in FIGS. 4B, 4E, and 4F.

The source product holding container(s) 450g could be weighed and/or scanned using the scale(s) 430g-1 and the scanner(s) 434g-1, to quantify and/or identify inputs to the decarboxylation system 420g. The source product(s) in the source product holding container(s) 450g could then be transferred to the decarboxylation oven(s) 452g, to heat the source product(s) as described elsewhere herein. One or more of the controller(s) 426g could be connected to or otherwise in communication with the decarboxylation oven(s) 452g, to control the decarboxylation oven(s). The sensor(s) 428g could similarly be connected to or otherwise in communication with the decarboxylation oven(s) 452g, to measure one or more parameters and/or otherwise monitor one or more properties of a decarboxylation process or equipment. Decarboxylated product could then be transferred to the decarboxylated product holding container(s) 454g. The decarboxylated product holding container(s) 454g could be weighed by the scale(s) 430g-2 and/or labeled by the label maker(s) 432g. Markings on the milled product holding container(s) 454g or label(s) could be scanned by the scanner(s) 434g-2. Weights as measured by the scale(s) 430g-2 could be used to reconcile input source product with total output extracted product, to maintain desired and/or required records of source product during processing.

An example extraction system 420h is shown in FIG. 4H, and includes one or more operator check-in device 422h, one or more computers 424h, one or more controllers 426h, one or more sensors 428h, one or more scales at 430h-1 and/or 430h-2, one or more label makers 432h and one or more scanners at 434h-1 and/or 434h-2. These components are connected to or otherwise in communication with the server 418g.

Implementation options for all of these components are described herein, at least above with reference to FIG. 4A. Although two sets of scale(s) and scanner(s) are shown in at 430h-1, 430h-2 and 434h-1, 434h-2, in some embodiments an extraction system could include only one set of either or both of these components.

Separate sets of scale(s) and scanner(s) are shown at 430h-1, 430h-2 and 434h-1, 434h-2 solely to simplify the illustration of connecting lines in FIG. 4H.

The extraction system 420h further includes one or more source product holding containers 450h, one or more extractors 452h, one or more winterization chillers 454h, one or more distillers 456h and one or more extracted product holding containers 458h. The containers 450h, 458h could include any of various types of container, and different container types could be used for source product and extracted product. The source product holding container(s) 450h could contain decarboxylated cannabis products, for example. In some embodiments, the source product holding container(s) 450h are the same holding container(s) as shown at 454g in FIG. 4G.

The source product holding container(s) 450h could be weighed and/or scanned using the scale(s) 430h-1 and the scanner(s) 434h-1, to quantify and/or identify inputs to the extraction system 420h. The source product(s) in the source product holding container(s) 450h could then be transferred to the extractor(s) 452h, which could implement any of various extraction processes to produce one or more extracts from the source product(s). Examples of extraction processes and extracts are disclosed elsewhere herein.

The produced extract(s) could be transferred to the winterization chiller(s) 454h, the distiller(s) 456h and/or the extract product holding container(s) 458h. The winterization chillers 454h could include a refrigerator, for example. In some embodiments, the winterization chiller(s) 454h are provided to cool a mixture of extract and polar solvent(s) to a temperature at which waxes and/or lipids separate from the extract. One or more outputs of the winterization chiller(s) 454h could also or instead be transferred to the distiller(s) 456h and/or the extract product holding container(s) 458h.

The distiller(s) 456h could include a distillation column, for example, to separate one or more cannabinoids and/or terpenes from extract(s). One or more outputs of the distiller(s) 456h could also or instead be transferred to the extract product holding container(s) 458h.

One or more of the controller(s) 426h could be connected to or otherwise in communication with the extractor(s) 452h, the winterization chiller(s) 454h and/or the distiller(s) 456h, to control these components.

The sensor(s) 428h could similarly be connected to or otherwise in communication with the extractor(s) 452h, the winterization chiller(s) 454h and/or the distiller(s) 456h, to measure one or more parameters and/or otherwise monitor one or more properties of an extraction process or equipment.

The extracted product holding container(s) 458h could be weighed by the scale(s) 430h-2. The label maker(s) 432h could generate and/or apply labels to the extracted product holding container(s) 458h. Markings on the extracted product holding container(s) 458h could be scanned by the scanner(s) 434h-2. Weights as measured by the scale(s) 430h-2 could be used to reconcile input source product with total output decarboxylated product, to maintain desired and/or required records of source product during processing. In some embodiments, two or more extracted product holding container(s) 458h could be mixed and marked accordingly.

FIG. 4I illustrates an example oil formulation system 420i, which includes one or more operator check-in devices 422i, one or more computers 424i, one or more controllers 426i, one or more sensors 428i, one or more scales at 430i-1 and/or 430i-2, one or more label makers 432i and one or more scanners at 434i-1 and/or 434i-2. These components are connected to or otherwise in communication with the server 418l. Implementation options for all of these components are described herein, at least above with reference to FIG. 4A. Although two sets of scale(s) and scanner(s) are shown in at 430i-1, 430i-2 and 434i-1, 434i-2, in some embodiments an oil formulation system could include only one set of either or both of these components. Separate sets of scale(s) and scanner(s) are shown at 430i-1, 430i-2 and 434i-1, 434i-2 solely to simplify the illustration of connecting lines in FIG. 4I.

The oil formulation system 420i further includes one or more source product holding containers 450i, one or more carrier oil holding containers 452i, one or more mixing devices 454i, one or more dilution devices 456i and one or more cannabis oil holding containers 458i. The containers 450i, 452i, 458i could include any of various types of container, and different container types could be used for source product, carrier oil, and cannabis oil. The source product holding container(s) 450i could contain cannabis extract-based products, for example, and in some embodiments could include one or more containers as shown at 458h in FIG. 4H. The carrier oil holding container(s) 452i could include carrier oils that, when mixed with a cannabis extract, produce a cannabis oil and/or concentrate. Carrier oils are discussed in greater detail elsewhere herein.

The source product holding container(s) 450i and/or the carrier oil holding container(s) 452i could be weighed and/or scanned using the scale(s) 430i-1 and/or the scanner(s) 434l-1, to quantify and/or identify inputs to the oil formulation system 420i. Moreover, in some embodiments, the carrier oil and/or the source product can be the subject of testing prior to the mixing stage. In some embodiments, such testing is part of a Preventable Control Plan (PCP). In some embodiments, such testing can include allergen testing, label validation testing, microbiological testing, mycotoxins testing, nutritional analysis, organoleptic testing, testing for heavy metals, foreign materials, toxins and/or other contaminants. The results of such tests can be recorded by the ICS in, for example, the database 414 on server 402.

The source product(s) in the source product holding container(s) 450i and the carrier oil(s) in the carrier oil holding container(s) 452i could then be transferred to the mixing device(s) 454i to be mixed. The carrier oil(s) could also be transferred to the dilution devices 456i. The mixing device(s) 454i could dissolve the source product(s) in the carrier oil(s) to produce a homogeneous mixture. The dilution device(s) 456i could add additional carrier oil(s) to the mixture to decrease concentration of cannabinoids in the mixture, for example. A diluted mixture could be further mixed at 456i, returned to the mixer(s) 454i for further mixing. Examples of mixing devices and/or dilution devices that could be implemented at 454i, 456i include containers, vessels and/or tools for mixing, heated water baths, ultrasonic water baths, heated stir plates and heat guns.

One or more of the controller(s) 426i could be connected to or otherwise in communication with the mixing device(s) 454i and/or the dilution device(s) 456i to control these components. The sensor(s) 428i could similarly be connected to or otherwise in communication with the mixing device(s) 454i and/or the dilution device(s) 456i, to measure one or more parameters and/or otherwise monitor one or more properties of an oil formulation process or equipment.

The produced cannabis oil(s) could be transferred from the mixing device(s) 454i and/or dilution device(s) 456i to the cannabis oil holding container(s) 458i. The cannabis oil holding container(s) 458i could be weighed by the scale(s) 430i-2. The label maker(s) 432i could generate and/or apply labels to the cannabis oil holding container(s) 458i, and/or the scanner(s) 434i-2 could scan markings on the container(s) or the label(s). Weights as measured by the scale(s) 430i-2 could be used to reconcile input source product with total output cannabis oil, to maintain desired and/or required records of source product during processing.

In some embodiments, further testing can be carried out after mixing by mixing device(s) 454i and dilution by dilution device(s) 456i. Such testing can be carried out in the holding container(s) 458i, or once the product has been packaged or partially packaged. In some embodiments, such further testing is part of a Preventable Control Plan (PCP). In some embodiments, such further testing can include allergen testing, label validation testing, microbiological testing, mycotoxins testing, nutritional analysis, organoleptic testing, testing for heavy metals, foreign materials, toxins and/or other contaminants. The results of such further tests can be recorded by the ICS in, for example, the database 414 on server 402.

FIG. 4N illustrates an example edibles formulation system 420n. Cannabis-infused edibles include, but are not limited to, cakes, brownies, other baked goods, chocolates, gelatin-based chewable sweets (such as gummy or jelly candies) and other confectionaries, butters, cooking oils, tinctures, dairy-based liquid edibles (such as bhang lassi or bhang thandai), capsules containing one or more cannabinoids, etc. The edibles formulation system 420n includes one or more operator check-in devices 422n, one or more computers 424n, one or more controllers 426n, one or more sensors 428n, one or more scales at 430n-1 and/or 430n-2, one or more label makers 432n and one or more scanners at 434n-1 and/or 434n-2. These components are connected to or otherwise in communication with the server 418n. Implementation options for all of these components are described herein, at least above with reference to FIG. 4A. Although two sets of scale(s) and scanner(s) are shown in at 430n-1, 430n-2 and 434n-1, 434n-2, in some embodiments an edibles formulation system could include only one set of either or both of these components. Separate sets of scale(s) and scanner(s) are shown at 430n-1, 430n-2 and 434n-1, 434n-2 solely to simplify the illustration of connecting lines in FIG. 4N.

The edibles formulation system 420n further includes one or more source product holding containers 450n, one or more base foodstuff holding containers 452n, one or more mixing devices 454n, one or more dilution devices 456n and one or more cannabis edible holding containers 458n. The containers 450n, 452n, 458n could include any of various types of container, and different container types could be used for source product, base foodstuff, and cannabis edibles. The source product holding container(s) 450n could contain cannabis extract-based products (such as a distillate or an emulsified cannabinoid mixture), for example, and in some embodiments could include one or more containers as shown at 458n in FIG. 4N. The base foodstuff holding container(s) 452n could include foodstuffs that, when mixed with a cannabis extract, produce a cannabis edible. Suitable foodstuffs include, but are not limited to, chocolate, gelatin-based chewable sweets, and any other foodstuff suitable for being infused with cannabis or a cannabis-based emulsion.

The source product holding container(s) 450n and/or the base foodstuff holding container(s) 452n could be weighed and/or scanned using the scale(s) 430n-1 and/or the scanner(s) 434n-1, to quantify and/or identify inputs to the edibles formulation system 420n. Moreover, in some embodiments, the base foodstuffs and/or the source product can be the subject of testing prior to the mixing stage. In some embodiments, such testing is part of a Preventable Control Plan (PCP). In some embodiments, such testing can include allergen testing, label validation testing, microbiological testing, mycotoxins testing, nutritional analysis, organoleptic testing, testing for heavy metals, foreign materials, toxins and/or other contaminants. The results of such tests can be recorded by the ICS in, for example, the database 414 on server 402.

The source product(s) in the source product holding container(s) 450n and the food stuff(s) in the foodstuff holding container(s) 452n could then be transferred to the mixing device(s) 454n to be mixed. The foodstuff(s) could also be transferred to the dilution devices 456n. The mixing device(s) 454n could dissolve the source product(s) in the foodstuff(s) to produce a homogeneous mixture. The dilution device(s) 456n could add additional foodstuff(s) to the mixture to decrease concentration of cannabinoids in the mixture, for example. A diluted mixture could be further mixed at 456n, returned to the mixer(s) 454n for further mixing. Examples of mixing devices and/or dilution devices that could be implemented at 454n, 456n include containers, vessels and/or tools for mixing, such as industrial food mixers, industrial blenders, industrial powder mixers, industrial drum/powder mixers, industrial ring-layer mixers, industrial pelletizers, industrial granulators, heated water baths, ultrasonic water baths, heated stir plates and heat guns.

One or more of the controller(s) 426n could be connected to or otherwise in communication with the mixing device(s) 454n and/or the dilution device(s) 456n to control these components. The sensor(s) 428n could similarly be connected to or otherwise in communication with the mixing device(s) 454n and/or the dilution device(s) 456n, to measure one or more parameters and/or otherwise monitor one or more properties of an edibles formulation process or equipment.

The produced cannabis edible(s) could be transferred from the mixing device(s) 454n and/or dilution device(s) 456n to the cannabis edibles holding container(s) 458n. The cannabis edibles holding container(s) 458n could be weighed by the scale(s) 430n-2. The label maker(s) 432n could generate and/or apply labels to the cannabis edibles holding container(s) 458n, and/or the scanner(s) 434n-2 could scan markings on the container(s) or the label(s). Weights as measured by the scale(s) 430n-2 could be used to reconcile input source product with total output cannabis edible, to maintain desired and/or required records of source product during processing.

In some embodiments, further testing can be carried out after mixing by mixing device(s) 454n and dilution by dilution device(s) 456n. Such testing can be carried out in the holding container(s) 458n, or once the product has been packaged or partially packaged. In some embodiments, such further testing is part of a Preventable Control Plan (PCP). In some embodiments, such further testing can include allergen testing, label validation testing, microbiological testing, mycotoxins testing, nutritional analysis, organoleptic testing, testing for heavy metals, foreign materials, toxins and/or other contaminants. The results of such further tests can be recorded by the ICS in, for example, the database 414 on server 402.

Referring now to FIG. 4J, an embodiment of a packaging system 420j includes one or more operator check-in devices 422j, one or more computers 424j, one or more controllers 426j, one or more sensors 428j, one or more scales at 430j-1 and/or 430j-2, one or more label makers 432j and one or more scanners at 434j-1 and/or 434j-2. These components are connected to or otherwise in communication with the server 418j. Implementation options for all of these components are described herein, at least above with reference to FIG. 4A. Although two sets of scale(s) and scanner(s) are shown in at 430j-1, 430j-2 and 434j-1, 434j-2, in some embodiments a packaging system could include only one set of either or both of these components. Separate sets of scale(s) and scanner(s) are shown at 430j-1, 430j-2 and 434j-1, 434j-2 solely to simplify the illustration of connecting lines in FIG. 4J.

The packaging system 420j further includes one or more source product holding containers 450j, one or more cone filling machines 452j, one or more bottle filling and/or capping machines 454j and one or more target holding containers 456j. The containers 450j, 456j could include any of various types of container, and different container types could be used as these containers. The source product holding container(s) 450j could contain cannabis flower and/or trim, dried cannabis, milled cannabis, decarboxylated cannabis, cannabis extracts and/or cannabis oils, for example. In some embodiments, the source product holding container(s) 450j could include containers that hold outputs from any one or more of the examples systems 420a, 420b, 420d, 420e, 420f, 420g, 420h and/or 420i.

The source product holding container(s) 450j could be weighed and/or scanned using the scale(s) 430j-1 and/or the scanner(s) 434j-1, to quantify and/or identify inputs to the packaging system 420j. Milled cannabis source product(s) could be transferred to the cone filling machine(s) 452j, which is provided to fill paper cones with cannabis product to produce cannabis cigarettes. Cannabis oil source product(s) could be transferred into bottles using the bottle filling and/or capping machine(s) 454j. Examples of cone filling machines, bottling filling machines and capping machines are discussed in further detail elsewhere herein.

One or more of the controller(s) 426j could be connected to or otherwise in communication with the cone filling machine(s) 452j and/or the bottle filling and/or capping machine(s) 454j, to control the cone filling machine(s) and/or the bottle filling and/or capping machine(s). The sensor(s) 428j could similarly be connected to or otherwise in communication with the cone filling machine(s) 452j and/or the bottle filling and/or capping machine(s) 454j, to measure one or more parameters and/or otherwise monitor one or more properties of a packaging process or equipment such as the cone filling machine(s) and/or the bottle filling and/or capping machine(s).

Some types of source product could also or instead be transferred from the source product holding container(s) 450j to the target holding container(s) 456j, which could include transferring cannabis product into holding containers that are intended for sale to customers. For example, target holding containers 456j could contain smaller quantities of cannabis product than source product holding container(s) 450j. Target holding containers 456j could also include packages that store multiple holding containers of cannabis product. Cannabis cigarettes produced by the cone filling machine(s) 452j and bottles produced by the bottle filling and/or capping machine(s) 454j could also or instead be transferred to target holding container(s) 456j, but this might not always be the case. For example, bottles of cannabis oil produced by the bottle filling and/or capping machine(s) 454j could be considered to be a holding container that is intended for sale to customers.

Cannabis cigarettes, bottles of cannabis oil, and/or target holding container(s) 456j could be weighed by the scale(s) 430j-2. The label maker(s) 432j could generate and/or apply labels to the cannabis cigarettes, bottles of cannabis oil and/or target holding container(s) 456j, and/or the scanner(s) 434j-2 could scan markings on these cannabis products, container(s), or label(s). Weights as measured by the scale(s) 430j-2 could be used to reconcile input source product with total output product, to maintain desired and/or required records of source product during processing.

The example sterilization system 420k in FIG. 4K includes one or more operator check-in devices 422k, one or more computers 424k, one or more scales and 430k-1 and/or 430k-2, one or more label makers 432k and one or more scanners at 434k-1 and/or 434k-2. These components are connected to or otherwise in communication with the server 418k. Implementation options for all of these components are described herein, at least above with reference to FIG. 4A. Although two sets of scale(s) and scanner(s) are shown in at 430k-1, 430k-2 and 434k-1, 434k-2, in some embodiments a sterilization system could include only one set of either or both of these components. Separate sets of scale(s) and scanner(s) are shown at 430k-1, 430k-2 and 434k-1, 434k-2 solely to simplify the illustration of connecting lines in FIG. 4K.

The sterilization system 420k further includes one or more source product holding containers 450k, an irradiation facility 452k and one or more sterilized product holding containers 454k. The containers 450k, 454k could include any of various types of container, and different container types could be used as these containers. In some embodiments, the source product holding container(s) 450k could include containers that hold outputs from any one or more of the examples systems 420a, 420b, 420d, 420e, 420f, 420g, 420h, 420i and/or 420j.

The source product holding container(s) 450k could be weighed and/or scanned using the scale(s) 430k-1 and the scanner(s) 434k-1, to quantify and/or identify inputs to the sterilization system 420k. Source product(s) could be transferred from the source product holding container(s) 450k, to irradiation facility 452k, and then to the sterilized product holding container(s) 454k. The irradiation facility 452k could include equipment, with one or more internal and/or or external controllers (not shown), to sterilize the source product(s) by irradiation. Other examples of sterilization processes that could also or instead be implemented by equipment in a sterilization system are also disclosed elsewhere herein.

One or more internal or external sensor(s) (not shown) could also or instead be incorporated into, connected to, or otherwise in communication with the irradiation facility 452k and/or other sterilization equipment, to measure one or more parameters of sterilization equipment and/or otherwise monitor one or more properties of a sterilization process or equipment.

The sterilized product holding container(s) 454k could be weighed using the scale(s) 430k-2. The label maker(s) 432k could label using the sterilized product holding container(s) 454k. Markings on the sterilized product holding container(s) 454k or label(s) could be scanned using the scanner(s) 434k-2. Weights as measured by the scale(s) 430k-2 could be used to reconcile input source product with total output product, to maintain desired and/or required records of source product during processing.

An example of a testing system 420l is shown in FIG. 4L, and includes one or more operator check-in devices 422l, one or more computers 424l, one or more controllers 426l, one or more scales 430l, one or more label makers 432l and one or more scanners 434l. These components are connected to or otherwise in communication with the server 418l. Implementation options for all of these components are described herein, at least above with reference to FIG. 4A.

The testing system 420l further includes one or more source product holding containers 450l, one or more sampling containers 452l and one or more testing devices 454l. The containers 450l, 452l could include any of various types of container, and different container types could be used as these containers. In some embodiments, the source product holding container(s) 450l could include containers that hold outputs from any one or more of the examples systems 420a, 420b, 420d, 420e, 420f, 420g, 420h, 420i, 420j and/or 420k.

At least a portion of a source product in each source product holding container(s) 450l could be transferred to a sampling container 452l. Each sampling container 452l could store one or more samples for testing.

The source product holding container(s) 450l and/or the sampling container(s) 452l could be weighed and/or scanned using the scale(s) 430l and/or the scanner(s) 434l, to quantify and/or identify inputs to the testing system 420l. Labels could be applied to the sampling container(s) 452l using the label maker(s) 432l. Markings on either or both of source product holding container(s) 450l and the sampling container(s) 452l, or label(s) thereon, could be scanned by the scanner(s) 434l to track the particular source product(s) being sampled and tested.

The sample(s) in the sampling container(s) 452l could be tested by the testing device(s) 454l. Examples of testing devices 454l include, but are not limited two, devices configured to test for mold and/or the presence of pesticides or other chemicals. The testing device(s) 454l are connected to or otherwise in communication with the server 418l, and could transmit test results to the ICS through the server. Also or alternatively, test results could be recorded manually using the computer(s) 424l, for example.

One or more of the controller(s) 426l could be connected to or otherwise in communication with the testing device(s) 454l, to control the testing device(s).

One or more internal or external sensor(s) (not shown) could also or instead be incorporated into, connected to, or otherwise in communication with the testing device(s) 454l, to measure one or more parameters of sterilization equipment and/or otherwise monitor one or more properties of a testing process or equipment.

Although not explicitly shown in FIG. 4L, the source product holding container(s) 450l could be weighed using the scale(s) 430l after any samples have been taken, to reconcile source product input, remaining source product after testing, and source product samples. In some embodiments, source product(s) samples are taken and stored to maintain archived source product samples. Archived source product samples could be taken in addition to source product samples that are tested by the testing device(s) 454l. In some embodiments, archived samples could be weighed using the scale(s) 430l, possibly labelled using the label maker(s), and have markings scanned by the scanner(s) 434l to enable archived sample recording and tracking.

In the example shown in FIG. 4M, a shipping system 420m includes one or more operator check-in devices 422m, one or more computers 424m, one or more scales 430m, one or more label makers 432m and one or more scanners 434m. These components are connected to or otherwise in communication with the server 418m. Implementation options for all of these components are described herein, at least above with reference to FIG. 4A.

The shipping system 420m further includes one or more customer order databases 450m stored in one or more memory devices, one or more selected holding containers 452m, one or more packages 454m and one or more shipping services 456m. The customer order database(s) 450m store customer orders for cannabis products. The memory device(s) in which the customer order database(s) 450m are stored are connected to or otherwise in communication with the server 418m, and in communication with the ICS in some embodiments. Although FIG. 4M illustrates the customer order database(s) 450m as a separate component, in some embodiments the customer order database(s) 450m could be stored within the ICS and/or a computer 424m.

The selected holding container(s) 452m are intended to represent one or more holding containers of cannabis product(s) that have been selected to meet one or more customer orders stored in the customer order database(s) 450m, and could include containers that hold outputs from any one or more of the examples systems 420a, 420b, 420d, 420e, 420f, 420g, 420h, 420i, 420j, 420k and/or 420m. The selected holding container(s) 452m, or contents such as individual units therein, are transferred to one or more packages 454m. Each package could include all of the holding containers or units that are selected to meet one customer order. Selection of holding container(s) and/or packaging into package(s) could include manual selection and packing and/or automated selection and packaging by “picking” machines.

The package(s) 454m could then be transferred to the shipping service(s) 456m, which ship packages to customers. Shipping service(s) 456m could include, for example, courier services.

The selected holding container(s) 452m and/or the package(s) 452m could be weighed and/or scanned using the scale(s) 430m and the scanner(s) 434m. The selected holding container(s) 452m could be weighed before and after order fulfilment if some but not all contents of a holding container are used to fill an order.

Labels could also or instead be applied to the package(s) 454m using the label maker(s) 432m. The shipping service(s) 456m are connected to or otherwise in communication with the server 418m. A shipping service 456m could be a separate entity from a producer of cannabis products, and connect to server 418m through a different type of connection than other shipping system components, using the internet for example. Tracking numbers provided by the shipping service(s) 456m could then be stored transmitted to the server 418m and stored by the server and/or transmitted to the ICS for storage. Shipping system tracking numbers could be useful, for example, to track order fulfilment, to confirm order shipping, to monitor the location of the package(s) 454m after shipping, and/or to confirm order delivery, for example.

The example system 400 shown in FIGS. 4A-4M and described in detail above represents one illustrative embodiment. Other embodiments are also contemplated. For example, although various components are shown separately in these drawings, multiple components could be implemented in a single component. In some embodiments, any two or more of operator check-in device(s), computer(s), controller(s), sensor(s), scale(s), label maker(s), and scanner(s) could be implemented using a single device. In one example, plant cultivation and harvest and plant part separation are within one facility, and operator check-in devices 422a, 422b are implemented using a single operator check-in device. In another example, a label maker 432a and a scanner 434a are implemented using a single device. Other combinations are also contemplated.

Various implementations, as well as applications of information, equipment, and functions, in an ICS are possible. Illustrative examples are disclosed herein, and others may be or become apparent to those skilled in the art.

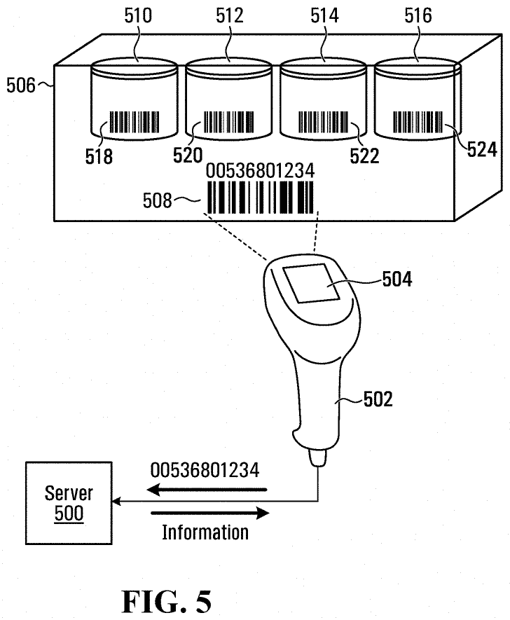

For example, an ICS could use machine-readable code to identify and record cannabis products. FIG. 5 is a block diagram illustrating an example implementation of a barcode scanner 502 in communication with an ICS through a server 500. FIG. 3 includes a barcode scanner 502, which is connected to or otherwise in communication with a server 500. The scanner 502 is illustrated as a portable barcode scanner, which could be wired or wireless, but this is only an example. The server 500 could be the server 418a-m in any of the example systems shown in FIGS. 4A-4M. Various scanner and server implementation examples are disclosed elsewhere herein, at least with reference to FIGS. 4A-4M.

FIG. 5 also includes a package 506, which has a label 508 that includes a barcode encoding a number “00536801234”. In some embodiments, the number could be a unique identifier for the package 508. The package 508 stores multiple holding containers 510, 512, 514, 516. Each of the holding container 510, 512, 514, 516 could contain a respective cannabis product. These cannabis products could be the same type of product, be from the same batch of cannabis plants, and/or be from the same lot of cannabis products. Alternatively, the cannabis products could have no relation to each other. The holding containers 510, 512, 514, 516 each include a respective label 518, 520, 522, 524 with a barcode. In the case that the holding containers 510, 512, 514, 516 contain the same lot of cannabis product, the labels 518, 520, 522, 524 could be identical, or could at least include some common information that is identical across multiple labels.

In some embodiments, the scanner 502 could read the label 508 and determine the number “00536801234”. For example, the scanner 502 could decode the barcode in the label 508. The scanner 502 could then transmit the number to the server 500 for local storage and/or transmission to a central ICS database. Alternatively or additionally, the scanner 502 could send a picture of the label 508 to the server 500, and the label could be decoded at the server or another server that hosts the central ICS database. The scanner 502 could also or instead transmit an action or request to the server 500 with the number. In one example, a user might want to determine the contents of the package 506. In that case, the scanner 502 could send a “look-up” request to the server 500 with the number “00536801234”. Upon receipt of the request, the server 500 could search a local database or request a search of a central ICS database for a record or records associated with the number “00536801234”. Any relevant records or information from those records could be sent to the server 500 and/or to the scanner 502. For example, any or all information in an ICS related to the holding containers 510, 512, 514, 516 could be sent to the server 500 and/or to the scanner 502. The device 502 could display such information to an operator on a screen 504. This could be useful for example, when a customer is unpacking an order and package contents are to be verified.

In another example, the package 506 could have been received at a new location, such as a storage facility. In this case, the scanner 502 could send the number “00536801234” and a “received” action to the server 500. The server 500 could locally store the number and/or update one or more local records to indicate that the package 506 has been received at the new location. The server 500 could also or instead send information to a central ICS database, to enable the central ICS database to be similarly updated with current status of the package 506. In some embodiments, information such as a confirmation or acknowledgement could be sent to the scanner 502 to confirm that package status has been successfully updated.

Other actions and/or requests could also or instead be sent from the scanner 502 to the server 500, from the server 500 to the scanner 502, and/or from the server 500 to other components such as a server that hosts a central ICS database. For example, the scanner 502 could also or instead communicate with the server 500, and the server 500 could communicate with other components when the holding container labels 518, 520, 522, 524 are scanned, to enable traceability of holding containers that were actually placed into and unpacked from the package 506, and/or to detect potential tampering if packed and unpacked holding container information does not match.

The scanner 502 is an illustrative example of an electronic device that could be in communication with a server or other component implementing an ICS. Other electronic devices, such as computers, scales, controllers, and/or sensors, for example, could also or instead be in communication with an ICS component such as a server. These electronic devices could be portable or stationary, and wired or wireless. Examples of these devices are described in further detail elsewhere herein.

More generally, the implementation with a barcode scanner as illustrated in FIG. 5 is provided by way of example. Other ICS implementations could also or instead be used. For example, an ICS could be implemented using physical files, in addition to or instead of electronic files stored in computer memory. In some implementations, recording of products and processes in the ICS could be manual, automated, or a combination of both. In further implementations, authorization levels could be implemented within the ICS, such that the type of actions and/or requests that an operator or device can perform in the ICS is limited by their authorization level.

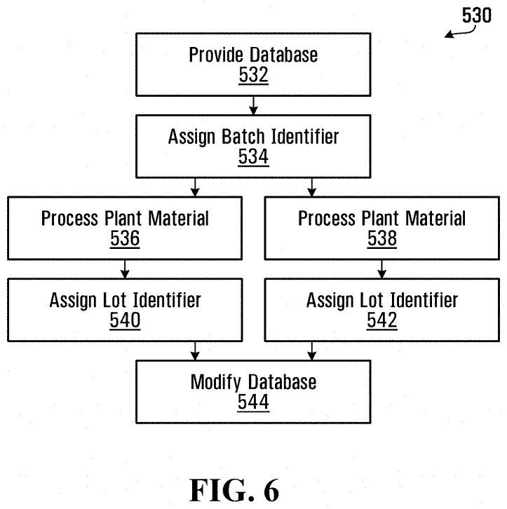

An ICS could be applied or used in any of various ways according to embodiments of the present disclosure. FIG. 6, for example, is a flow chart illustrating an example method according to one embodiment. The example method 530 involves providing, at 532, a database such as the database 414 in FIG. 4A to store information associated with cannabis plants and cannabis products. Such a database could be stored in one or more memory devices, which could include memory devices of different types. Examples of memory devices in which a database could be stored are disclosed elsewhere herein. The database could be populated with any of various types of information. Plant identifiers, such as plant numbers disclosed elsewhere herein, represent an example of information that is associated with cannabis plants and could be stored in a database. Plant information could also or instead include information that conveys such parameters or characteristics as grow area, any of various growing conditions, and/or harvest details, for example. Other examples of plant information are disclosed elsewhere herein. Similarly, any of various types of information associated with cannabis products could be stored in the database, and examples of such information are disclosed elsewhere herein. The example method 530 is not limited to any particular cannabis products. The cannabis product information stored in the database could include different fields and/or different types of information for different types of cannabis products.

A batch identifier is assigned to a batch of the cannabis plants, at 534. A batch identifier could be, for example, a batch number as disclosed elsewhere herein. In some embodiments, batch identifiers are sequential, and a most recently used batch identifier is incremented by a value of one to generate or otherwise determine a next sequential batch identifier when a new batch identifier is to be assigned. Batch identifiers need not be sequential in other embodiments. In general, any batch identifier generation or determination approach that enables different batches to be distinguished from each other could be applied.

Batch identifiers could be generated or determined on an as-needed basis as noted above, but could also or instead be generated in advance and stored to memory, for access or retrieval when a new batch identifier is to be assigned. A central ICS server such as the server 402 in FIG. 4A, the cultivation and harvest system server 418a, the label maker(s) 432a, and/or another component of the cultivation and harvest system 420a could generate or determine batch identifiers.

The actual assignment of a batch identifier at 534 could be accomplished in some embodiments by marking or otherwise recording the batch identifier as assigned, allocated, or reserved in a memory, to indicate that the batch identifier has been assigned to a batch of cannabis plants and therefore should not be assigned to another batch. In some embodiments, batch identifiers are assigned and then incremented or otherwise changed so that a new batch identifier is assigned to a next batch of cannabis plants. Other batch identifier management approaches are also possible.

Plant material from a portion of the cannabis plants in the batch is processed at 536, using a first process, to produce units of a first cannabis product. Plant material from another portion of the cannabis plants in the batch is processed at 538, using a second process, to produce units of a second cannabis product. The first and second processes could, but need not necessarily, be performed concurrently. Examples of processes that could be used to produce different cannabis products are disclosed elsewhere herein, and any of those processes could be used to process plant material at 536, 538.

For example, the processing at 536, 538 could include any one or more of: separating the plant material; drying the plant material; curing the plant material; and extracting one or more cannabinoids from the plant material. An extraction process for extracting one or more cannabinoids from the plant material could involve performing supercritical CO₂ extraction of cannabinoids from the plant material. In some embodiments, extracting one or more cannabinoids from the plant material further involves producing a cannabis extract and distilling the cannabis extract.

At 540, a first lot identifier is assigned to a lot of the units of the first cannabis product, and a second lot identifier is assigned to a lot of the units of the second cannabis product at 542. The first and second lot identifiers could, but need not necessarily, be assigned concurrently. Examples of lots and lot identifiers are disclosed elsewhere herein, and any of those examples could be applied in delineating lots and assigning lot identifiers at 540, 542. The units of the first and second cannabis products could be delineated into lots in similar or different ways. Lot identifiers for the lots of units of the first and second cannabis products could be of the same type or different types. In some embodiments, lot identifiers could be generated, determined, and/or assigned in a similar manner as described above for batch numbers. For example, lot identifiers could be sequential and generated on an as-needed basis.

Lot identifiers could be managed and/or assigned independently for different cannabis products. For example, lot identifiers could be unique within each type of cannabis product lines to allow lots of each cannabis product to be uniquely identified, but need not necessarily be “globally” unique across all product lines. The same lot identifier could be assigned to lots of units of different cannabis products, because those different cannabis products could be distinguished from each other based on product type, even though the lot numbers are the same. In other embodiments, lot identifiers are unique across all product lines, and a particular lot identifier is assigned to only one lot.

The example method 530 also involves modifying the database, at 544, to include information conveying or indicating the batch identifier, the first lot identifier and the second lot identifier, with the first lot identifier and the second lot identifier each being associated with the batch identifier. In some embodiments, such associations between multiple lot identifiers and a batch identifier are inherent in an arrangement or organization of information in the database. For example, a database record could include multiple fields or entries that are populated with information that conveys associated identifiers.

In some embodiments, modifying the database at 544 involves creating a lot record for each lot of units of a cannabis product, with the lot record including information conveying the lot identifier associated with the lot and information conveying the batch identifier associated with the lot identifier. In this example, information conveying the lot identifier and information conveying the batch identifier are in the same lot record, and the lot identifier—batch identifier association is inherent in the arrangement of information in the lot record. Respective lot records could be created for different lots, such as a first lot record for the lot of units of the first cannabis product and a second lot record for the lot of units of the second cannabis product in an example described above.

A lot record could also include other information. In some embodiment, a lot record includes information indicative of the process or processes used in processing plant material to produce units of a cannabis product that is associated with the lot. Examples of processes that could be conveyed or indicated in information in a lot record are disclosed elsewhere herein. Information conveying or indicating any of various parameters or characteristics of such processes could also or instead be included in a lot record.

A lot record could also or instead include information indicative of the number of units of a cannabis product contained in the lot. This information could be useful, for example, to track production output and/or concentration of active substance(s) in cannabis products.

Another type of information that could be included in a lot record in some embodiments is information indicative of the time, date, and/or other details of the processing that was used to produce units of a cannabis product contained in the lot.

The method 530 is an illustrative example of a method according to one embodiment. Other embodiments could involve performing operations in a different order than shown, and/or performing different operations instead of or in addition to those shown in FIG. 6. For example, units of a cannabis product could be packaged, for storage and/or shipment.

Considering the first cannabis product in FIG. 6, each of the units of that cannabis product could be packaged to produce first product packages, and each product package could be marked with product information indicative of the first lot identifier. A method could also or instead involve packaging each of the units of the second cannabis product to produce second packages, and marking each second package with product information indicative of the second lot identifier. Changes of identifiers between different lots are also described in further detail elsewhere herein.

In some embodiments, the product information that is marked on packages is generated, at least in part, from information retrieved from the database. Product information could be stored in the database, retrieved, and used to marked packages, or information retrieved from the database could be coded or otherwise processed to generate the product information with which packages are marked.

The product information for package marking could include any one or more of the following:
information conveying an identity or contact information of a licensed producer of the cannabis plants;
information conveying an identity or contact information of a licensed processor of the cannabis product;
information conveying a brand name of a cannabis product; information conveying recommended storage conditions of a cannabis product; and
information conveying a packaging date of a cannabis product.

Package marking could involve printing the product information on a package. In some embodiments, marking each product package involves printing a label including the product information, and affixing the label to the package. Information could be retrieved from the database, and the label could then be generated using the information retrieved from the database.

The example method 530 illustrates processing of plant material from cannabis plants in one batch to produce first and second cannabis products. Other plant material could also be processed. For example, the processing at 536 could also involve processing plant material from a portion of the cannabis plants in a second batch of cannabis plants using the first process to produce units of the first cannabis product. The modifying at 544 could then involve modifying the database to include information conveying a second batch identifier assigned to the second batch, and to associate the first lot identifier with the second batch identifier. In this example, lot of units of the first cannabis product are produced from plant material from cannabis plants in multiple batches, and the first lot identifier is associated with multiple batch identifiers. Using the database, the first lot identifier in this example could be traced to two batch identifiers, and the lot of units of the first cannabis product could thus be traced to two batches of cannabis plants from which the units of the first cannabis product originated.

In some embodiments, as initially described above with reference to FIG. 6, the units in one lot of a cannabis product are produced from one batch of cannabis plants, and a lot identifier that is assigned to the product lot is associated with only one batch identifier. Lot identifiers assigned to different lots could be associated with the same batch identifier if cannabis plants from one batch are used to produce the different lots. In other embodiments, cannabis plants from multiple batches are used to produce one lot of units, and a lot identifier for such a lot is associated with multiple batch identifiers.

Other variations of the example method 530 may be or become apparent to those skilled in the art.

A method could be implemented using a processor-readable storage medium, examples of which are disclosed elsewhere herein. Such a storage medium could have processor-executable instructions stored thereon, which, when executed by a processor, cause the processor to perform a method. Execution of the instructions could cause a computing device that includes the processor to implement a system configured to perform various operations. In some embodiments, the instructions, when executed, cause the computing device to implement a system configured to: implement a database configured to store information associated with cannabis plants and cannabis products; assign a batch identifier to a batch of the cannabis plants; receive processing information relating to the processing of plant material from a portion of the cannabis plants in the batch using a first process to produce units of a first cannabis product; receive processing information relating to the processing of plant material from another portion of the cannabis plants in the batch using a second process to produce units of a second cannabis product; assign, using the processing information, a first lot identifier to a lot of the units of the first cannabis product and a second lot identifier to a lot of the units of the second cannabis product; and modify the database to include information relating to the batch identifier, the first lot identifier and the second lot identifier, with the first lot identifier and the second lot identifier each being associated with the batch identifier.

Examples of many of these features are described above with reference to FIG. 530. A system implemented by a computing device could be configured to implement a database in one or more memory devices, for example, to store plant and product information, examples of which are described above and elsewhere herein. Such a system could also be configured to assign a batch identifier and lot identifiers, and to modify the database as described above and elsewhere herein.

FIG. 6 and the description thereof refer to processing plant material to produce units of cannabis products. A production system could include processing equipment to process plant material using processes to produce units of cannabis products. Different processing equipment could apply different processes to plant material, for example. A system that is implemented by a computing device might not itself include such processing equipment, but could be part of a production system, or at least communicate with processing equipment in a production system. A system that is implemented by a computing device could receive processing information from processing equipment, for example. In an embodiment, such a system is configured to receive processing information relating to the processing of plant material from a portion of the cannabis plants in the batch using a first process to produce units of a first cannabis product, and to receive processing information relating to the processing of plant material from another portion of the cannabis plants in the batch using a second process to produce units of a second cannabis product. Any of various types of processing information could be received, and examples of information relating to processing of plant material are disclosed elsewhere herein. Different types of processing could have different types of related information.

The server 402 in FIG. 4A, and other servers and computers in FIGS. 4A-4E, for example, could be configured to receive such information in some embodiments.

Processing information could be used to assign a first lot identifier to a lot of units of the first cannabis product and a second lot identifier to a lot of units of the second cannabis product. For example, each lot identifier could be assigned based on a type of the process, conveyed or indicated in the processing information, that was used to produce the units in that lot.

A system implemented by a computing device could be configured to provide other features disclosed herein.

The example method 530 and the example system described above provide and modify a database that includes various types of information. In some embodiments, a hierarchal dataset could have a tree structure, representative of a process flow to transform a batch of cannabis plants into a range of cannabis products. A method for dynamically generating such a hierarchal dataset could involve recording, on a computer readable storage medium, a batch identifier associated with the batch of cannabis plants. With reference to FIG. 6, for example, the batch identifier could be recorded on the computer readable storage medium by modifying a database as shown at 544. Although FIG. 6 represents modifying the database at the end of the example method 530, in other embodiments information such as the batch identifier could be recorded earlier, such as when it is assigned.

The batch identifier distinguishes the batch of cannabis plants among a plurality of batches of cannabis plants. Examples of batch identifiers are disclosed elsewhere herein. In some embodiments, the batch identifier is a root level of the hierarchal dataset.

A first portion of the batch of cannabis plants is processed using a first process to produce units of first cannabis products, and such processing is disclosed by way of example with reference to 536 in FIG. 6. A first lot number associated with the first cannabis products is recorded on the computer readable storage medium, and this is also represented by way of example at 544 in FIG. 6.

A second portion of the batch of cannabis plants is processed using a second process, to produce units of a second cannabis product, and a second lot number associated with the second cannabis products is recorded on the computer readable storage medium. These operations are consistent with 538, 544 in some embodiments.

Generating a hierarchical dataset could also involve linking the first and second lot numbers to the batch identifier in the hierarchal dataset. In some embodiments, the first lot number forms a first branch of the hierarchal dataset ascending, or descending, from the root node and the second lot number forms a second branch of the hierarchal dataset ascending, or descending, from the root node.

The example method 1720, like other methods disclosed herein, could include fewer, additional, and/or different operations, performed in a similar or different order.

For example, the hierarchal dataset could include additional information, such as any one or more of the following:
information indicative of the process or processes used in the steps of processing the first and second portions of the batch of cannabis plants;
information indicative of the number of units produced of the first and second cannabis products;
information indicative of the time and/or date of the processing used to produce the units of the first and second cannabis products.

Processing of the first portion of the batch of cannabis plants and the processing of the second portion of the batch of cannabis plants could include, for example, any one or more of the following, which are also described elsewhere herein:
separating the plant material;
drying the plant material;
curing the plant material; and
extracting one or more cannabinoids from the plant material.

In some embodiments, extracting one or more cannabinoids from the plant material involves performing supercritical CO₂ extraction of cannabinoids from the plant material.

Extracting cannabinoids from the plant material could also or instead involve such operations as producing a cannabis extract; and distilling the cannabis extract.

In some embodiments, each of the units of the first cannabis product is packaged to produce first product packages, and each first product package is marked with product information indicative of the first lot number. Similarly, each of the units of the second cannabis product could be packaged to produce second product packages and each second product package could be marked with product information indicative of the second lot number.

As in other embodiments disclosed herein, marking could involve marking product packages directly and/or printing a label including the product information and affixing the label to a package.

Product information need not be limited only to information indicative of lot number. Product information could also or instead include at least one of: information conveying an identity or contact information of a licensed producer of the cannabis plants; information conveying an identity or contact information of a licensed processor of the cannabis product; information conveying a brand name of the cannabis product; information conveying recommended storage conditions of the cannabis product; and information conveying a packaging date of the cannabis product.

Two portions of a batch of cannabis plants are referenced above. Some embodiments involve processing plant material from a portion of the cannabis plants in a further batch of cannabis plants associated with a further batch identifier, using the first process, to produce the units of the first cannabis product; and linking the first lot number to the further batch identifier in the hierarchal dataset. Such linking or associations between could be accomplished in any of various ways, examples of which are disclosed elsewhere herein.

Embodiments described above with reference to FIG. 6 involve associating various identifiers with each other, and could involve aspects of labelling. For example, units of first and second cannabis products could be marked with product information that is indicative of different lot numbers. At least these features could impact product labelling.

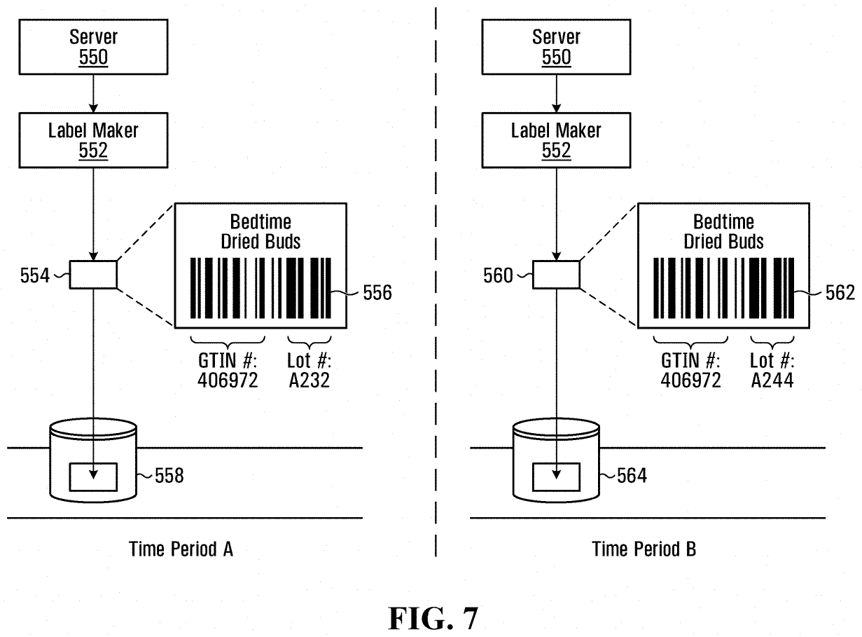

FIG. 7 illustrates operation of a machine, in particular a label maker 552 by way of example, for generating labels, according to one embodiment. In the example shown, the label maker 554 is connected to or otherwise in communication with a server 550. In some embodiments, the server 550 could be the central ICS server 402 in FIG. 4A or any one of the other servers in FIGS. 4A-4M. Similarly, the label maker 552 could be a label maker as shown in any of FIGS. 4A-4M. Examples of a server and a label maker are provided elsewhere herein.

In FIG. 7, during time period A, the label maker 552 generates labels, for units of a particular cannabis product originating from a particular batch of cannabis plants for example. Each label 554 in this example includes a machine-readable code that encodes a number specific to the cannabis product and a number that maps back to an identity of the batch. In the illustrated example, the number specific to the cannabis product is a GTIN and the number that maps back to the identity of the batch is the lot number. For example, in FIG. 7 the label 554 is generated for the cannabis product “Bedtime” dried buds, having a linear barcode 556 that encodes GTIN 406972 and the lot number A232. All labels for “Bedtime” dried buds belonging to the same lot A232 have the same linear barcode 556, or at least include the same GTIN and lot number. Later, during time period B, when units of the cannabis product from a different batch are being labelled for example, then the label maker 552 updates the machine-readable code to at least update the number that maps back to the identity of the batch. For example, in FIG. 7 the label 560 is generated for the cannabis product “Bedtime” dried buds, having a linear barcode 562 that still encodes GTIN 406972, but the lot number is changed to A244. All labels for “Bedtime” dried buds belonging to the same lot A244 have the same linear barcode 562, or at least include the same GTIN and lot number.

The switch in lot numbers in this example is controlled by the server 550, but in other embodiments the label maker could count labelled units of cannabis product and switch lot numbers based on the number of units in a lot. Other control mechanisms are also contemplated.

In another embodiment, the label maker 552 is instead replaced with a machine that generates cannabis product packaging having the machine-readable code. In another embodiment, the label maker 552 is instead replaced with a machine that generates anything in association with cannabis products that is to have the machine-readable code included thereon.

In view of the above, in some embodiments there is provided a cannabis product including packaging (e.g. a container in which the cannabis product is contained) and a machine-readable code included on or as part of the packaging (e.g. on a label affixed to the packaging). In some embodiments, the machine-readable code specifically conveys information that links the cannabis product back to a particular batch of cannabis plants from which cannabis in the cannabis product originates (e.g. the information may be a lot number and/or the batch number).

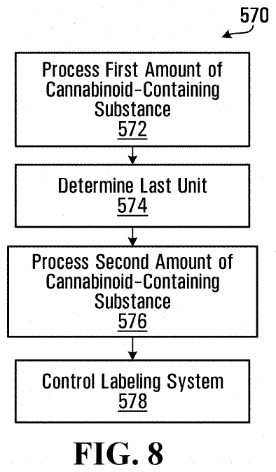

FIG. 8 is a flow diagram illustrating an example method of labelling cannabis products in an automated manufacturing process, and involves controlling a labelling system to label cannabis products with information related to different lot identifiers. The example method 570 involves, at 572, processing a portion of a first amount of cannabinoid-containing substance to sequentially produce first units of a cannabis product. The first amount of cannabinoid-containing substance is associated with a first cannabinoid-containing substance identifier. Examples of cannabinoid-containing substances and identifiers are disclosed elsewhere herein.

The example method 570 also involves, at 574, determining a last unit of cannabis product produced in the first units, or in other words determining the last one of the first units. The last unit could be determined, for example, based on an amount of the cannabinoid-containing substance that is used in producing each unit, and how many units can be produced from the first amount of the cannabinoid-containing substance. The units are produced sequentially at 572, and in some embodiments are counted to identify the last unit of cannabis product that is produced from the first amount of the cannabinoid-containing substance.

A portion of a second amount of the same cannabinoid-containing substance or a different cannabinoid-containing substance could then be processed at 576 to sequentially produce second units of the same cannabis product, or possibly a different cannabis product. The second amount of cannabinoid-containing substance is associated with a second cannabinoid-containing substance identifier. Again, it is noted that examples of cannabinoid-containing substances and identifiers are disclosed elsewhere herein.

The first and second units of cannabis product are labelled at 578, by controlling an automated labelling system to label units of cannabis product with label information conveying a first lot identifier associated with the first cannabinoid-containing substance identifier until the last unit of cannabis product has been labelled, and to then label units of cannabis product with label information conveying a second lot identifier associated with the second cannabinoid-containing substance identifier thereafter. This is consistent with the type of labelling illustrated in time period A and time period B in FIG. 7, for example.

The method 570, like other embodiments, represents an illustrative example. Other embodiments could involve performing operations in a different order than shown, and/or performing different operations instead of or in addition to those shown in FIG. 8.

For example, although shown in sequence in FIG. 8, the illustrated operations need not necessarily be completed in the order shown. A last unit of cannabis product could be determined at 574 before processing of the first amount of cannabinoid-containing substance at 572 is complete. Processing of the second amount of cannabinoid-containing substance at 576 could also or instead begin before the last unit has been determined at 574. In some embodiments, labelling at 578 could begin before other operations have been completed.

As an example of an additional operation that could be performed in some embodiments, the units of cannabis product could be packaged into product packages, and the labelling at 578 could then involve affixing labels to the product packages. Either or both of product units and product packages could be labelled at 578.

Regarding the operation at 574, in a sequential production process in which units are produced sequentially, determining a last unit of cannabis product produced in the first units is equivalent to, and could involve, determining the first unit of cannabis product produced in the second units.

Processing a portion of a first amount of cannabinoid-containing substance at 572, and/or processing a portion of a second amount of cannabinoid-containing substance at 576, could involve one or more of the following, and examples of these types of processing are disclosed elsewhere herein:
metering out amounts of cannabinoid-containing substance;
diluting cannabinoid-containing substance;
emulsifying cannabinoid-containing substance to produce a concentrated cannabinoid emulsion;
distilling cannabinoid-containing substance to produce distillate;
metering out amounts of distillate;
diluting distillate; and
emulsifying distillate to produce a concentrated cannabinoid emulsion.

Label information with which the first units and/or the second units are labelled could include, in addition to information conveying the first or second lot identifier, at least one of: information conveying an identity or contact information of a licensed producer of the cannabinoid-containing substance; information conveying an identity or contact information of a licensed processor of the cannabis product; information conveying a brand name of a cannabis product; information conveying recommended storage conditions of a cannabis product; and information conveying a packaging date of a cannabis product.

A processor-readable storage medium could be used in implementing a method that is consistent with FIG. 8, with processor-executable instructions being stored on such a medium. The instructions, when executed by a processor, cause the processor to perform a method. Execution of the instructions could cause a computing device that includes the processor to implement a system configured to, in some embodiments: receive processing information related to processing of cannabinoid-containing substance as shown at 572 and described above, determine a last unit as shown at 574 and described above, receive processing information related to processing of cannabinoid-containing substance as shown at 576 and described above, and control an automated labelling system receive processing information related to processing of cannabinoid-containing substance as shown at 578 and described above.

An automated production system could include such a computing device, as well as processing equipment and an automated labelling system. The processing shown at 572 and 574 could be performed in one installation of processing equipment, or in separate processing equipment. The first amount of cannabinoid-containing substance could be supplied to processing equipment until either it runs out or the last unit has been produced from the first amount, and then the cannabinoid-containing substance supply could be switched to the second amount to continue production.

Various embodiments of an ICS and example systems, methods, and processor-readable storage media are discussed above. Particular parts of a production system or process, and potential implications from an ICS point of view, are discussed in further detail below.

Harvesting and Plant Part Separation Processes

For the harvest of cannabis plants, any of a variety of information could be recorded in an ICS. For example, information related to a batch of cannabis plants that is harvested in operation 102 of FIG. 1 could be recorded in the ICS. Batch information could be recorded when the plants are harvested, for example. Batch information could also or instead be recorded during cultivation, and then updated when the plants are harvested.

In some embodiments, batch information could be recorded in the ICS in the form of a batch record that includes or is otherwise associated with a batch identifier. Harvest information related to a harvest process could also or instead be recorded in the ICS, as part of a batch record and/or in a separate harvest record. A harvest record could include or otherwise be associated with a harvest identifier, which could be similar in form to other identifiers disclosed herein. A batch record could include a harvest identifier or otherwise be associated with a harvest record for a harvest, or multiple harvest records if the batch was harvested over multiple days or in different ways for example. Similarly, a harvest record could include a batch identifier or otherwise be associated with a batch record, or multiple batch records if multiple batches are harvested in one harvest.

The following is a non-exhaustive list of information that could be recorded in the ICS for a batch of cannabis plants. A batch record and/or a harvest record could include any one or more of the following:
origin of seeds and/or cuttings used to grow a batch;
storage location of the seeds and/or cuttings;
quantity of plants in the batch, possibly recorded at different points in time (for example, number of plants at planting versus number of plants at harvesting);
cannabis plant strain in the batch;
cultivation period prior to harvesting (for example, harvesting performed after 8 weeks of cultivation);
quantity or percentage of plants that perished during the growing and/or harvesting process, possibly along with a record of when the plant perished and/or why/how it perished;
type, quantity, and/or composition of a growing medium used during cultivation;
type, quantity, composition, and/or application schedule of nutrients (for example, fertilizer) during cultivation;
type, quantity, and/or schedule of lighting during cultivation;
schedule of temperature and/or humidity during cultivation;
type, quantity, and/or schedule of air ventilation during cultivation;
quantity of watering and/or watering cycle during cultivation;
quantity and/or percentage of plants that required special attention, possibly along with the details of the attention needed;
treatments performed on the plants during cultivation;
root pH levels during cultivation; and
plant nutrients levels during cultivation.

However, not every item listed above is necessarily applicable to every batch, and not all items would necessarily be recorded even if applicable to a particular batch.

Information could be recorded and/or updated in the ICS during plant part separation. For example, during operation 104 of FIG. 1, the weight and/or volume of flower and trim 106 and waste 108 could be recorded in the ICS for each plant and/or batch. The time, date and/or location of plant part separation could also or instead be recorded in the ICS. Other information related to plant part separation process could also or instead be recorded.

In some embodiments, plant part separation information could be recorded in the ICS in the form of a plant part separation record, which includes or is otherwise associated with a plant part separation record identifier. A plant part separation record identifier could be similar in form to other identifiers disclosed herein.

Plant part separation information related to a plant part separation process could also or instead be recorded in the ICS as part of another record such as a batch record associated with a batch of plants undergoing plant part separation. A batch record could include a plant part separation identifier or otherwise be associated with a plant part separation record, or multiple plant part separation record if the batch was processed through multiple plant part separation processes or equipment or in different ways for example. Similarly, a plant part separation record could include a batch identifier or otherwise be associated with a batch record, or multiple batch records if multiple batches are processed through plant part separation.

In the example cultivation and harvest system 420a in FIG. 4A, any one or more components such as the operator check-in device(s) 422a, the computer(s) 424a, the controller(s) 426a, the sensor(s) 428a, the scale(s) 430a, the label maker(s) 432a, and the scanner(s) 434a could be involved in populating and/or updating the ICS. For example, any one or more of these components could be configured to generate, collect, and/or otherwise obtain batch and/or harvest information and transmit that information to the server 402, through the server 418a in some embodiments, for populating and/or updating the database 414 or particular records therein.

In some embodiments, one or more components of the example plant part separation system 420b in FIG. 4B could be involved in populating and/or updating the ICS. For example, any one or more of the operator check-in device(s) 422b, the computer(s) 424b, the controller(s) 426b, the sensor(s) 428b, the scale(s) 430b-1 and/or 430b-2, the label maker(s) 432b, and the scanner(s) 434b-1 and/or 434b-2 could be configured to generate, collect, and/or otherwise obtain plant part separation information and transmit that information to the server 402 (FIG. 4A), through the server 418b in some embodiments, for populating and/or updating the database 414 or particular records therein. In some embodiments, the difference between the weight of plant material input into the process (e.g. measured by scale(s) 430b-1) and the weight of plant material output from the process (e.g. measured by scale(s) 430b-2) is compared against the amount of waste output from the waste holding container(s) 460b in order to assess lost and/or theft of material. This information can then be recorded by the ICS in, for example, the database 414 on server 402.

Fresh Cannabis Processing

Information relating to fresh cannabis products could be recorded in an ICS. In some embodiments, lot numbers are assigned to fresh cannabis products when fresh cannabis plant material is sent for packaging. For example, a “new lot” action could be automatically or manually initiated in the ICS to assign a lot number to each different fresh cannabis product originating from a batch of cannabis plants. Lot number generation and/or assignment could occur before, during or after packaging the fresh cannabis material into holding containers. All of the holding containers that contain the fresh cannabis plant material from a single batch could be associated with and/or identified by the same lot number.

Examples of fresh product information that could be recorded in an ICS include the following, any one or more of which could be included in a lot record, for example:
plant number;
batch number;
category (for example, flower or trim);
brand name;
source (for example, in-house production or external production);
weight of fresh cannabis product;
volume of fresh cannabis product.

The weight of a fresh cannabis product could be recorded, for example, using a scale that is connected to or otherwise able to communicate with the ICS. The scale could weigh an empty holding container and record this weight in the ICS. An operator or equipment in a production system could then add a fresh cannabis product to the holding container and weigh the full container, using the same scale or another scale that is able to communicate with the ICS. The scale could record the new weight in the ICS, and the ICS could compare this weight to the weight of the empty container to determine and/or confirm the weight of fresh cannabis product that is stored in the holding container.

In some embodiments, fresh cannabis product information could be recorded in the ICS in a lot record that includes or is otherwise associated with a lot identifier. A batch record could include the lot identifier or otherwise be associated with the lot record for a lot of fresh cannabis product, or multiple lot records if the batch was used to produce multiple lots of fresh cannabis product. Similarly, a lot record could include a batch identifier or otherwise be associated with a batch record, or multiple batch records if multiple batches were used to produce fresh cannabis product in one lot.

In the example fresh processing system 420d in FIG. 4D, any one or more components such as the operator check-in device(s) 422d, the computer(s) 424d, the scale(s) 430d-1 and/or 430d-2, the label maker(s) 432d, and the scanner(s) 434d-1 and/or 434d-2 could be involved in populating and/or updating the ICS. For example, any one or more of these components could be configured to generate, collect, and/or otherwise obtain source product and/or fresh product information and transmit that information to the server 402, through the server 418d in some embodiments, for populating and/or updating the database 414 or particular records therein.

In some embodiments, the difference between the weight of source input into the process (e.g. measured by scale(s) 430f-1) and the weight of milling product output from the process (e.g. measured by scale(s) 430f-2) is compared in order to assess lost and/or theft of material. This information can then be recorded by the ICS in, for example, the database 414 on server 402.

Dried Cannabis Manufacturing

Any of various information relating to a drying process and/or dried cannabis products could be recorded in an ICS, in the form of a drying record in some embodiments. A drying record could include or otherwise be associated with a drying record identifier, which could be similar in form to other identifiers disclosed herein. Drying information related to a drying process and/or dried cannabis products could also or instead be recorded in another type of record such as a lot record.

A lot record could include a drying record identifier or otherwise be associated with a drying record for a drying process that was used to produce a lot of dried cannabis product, or multiple drying records if the lot was dried in different equipment, using different drying processes, and/or over multiple days for example. Similarly, a drying record could include a lot identifier or otherwise be associated with a lot record, or multiple lot records if a drying process or equipment produced multiple lots of dried cannabis product.

The following is a non-exhaustive list of information that could be recorded in an ICS for a drying process, and/or curing process if a curing process is also or instead used in producing dried cannabis product:
drying and/or curing process(es) used;
drying and/or curing time;
type, quantity, and/or schedule of lighting during drying and/or curing;
temperature(s) during drying and/or curing;
humidity during drying and/or curing;
type, quantity, and/or schedule of air ventilation during drying and/or curing;
type and/or quantity of any other ingredient(s) added during drying and/or curing.

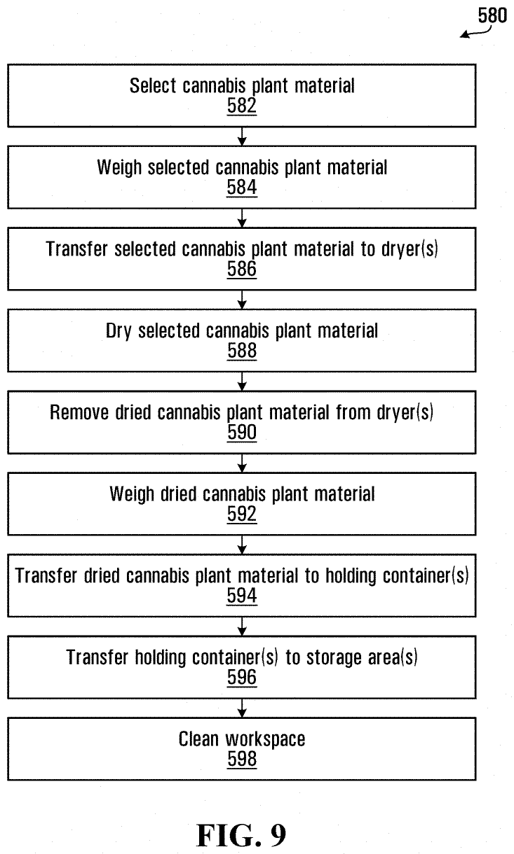

FIG. 9 is a flow diagram illustrating an example method 580 for drying and/or curing a cannabis material, such as the drying performed at operation 112 of FIG. 1.

At step 582, cannabis plant material is selected for the drying process. In some embodiments, the cannabis plant material is selected from harvested cannabis plant material such as flower and trim. The selection could be performed manually based on the weight and/or size of the cannabis plant material, for example.

The selection could also or instead be performed automatically, using one or more sorting machines, for example.

Step 584 includes weighing the cannabis plant material that is selected at step 582. Alternatively, a holding container containing the cannabis plant material could be weighed. Weighing the cannabis plant material at step 584 could be performed using, for example, a laboratory scale that is connected to or otherwise able to communicate with the ICS, such as the scale(s) 430e-1 in FIG. 4E. The measured weight could be recorded in the ICS for the drying process, along with the batch number, lot number, and/or any other information associated with the cannabis plant material in some embodiments.

At step 586, the selected cannabis material is transferred to one or more dryer(s), such as the dryer(s) 452e in FIG. 4E. In some embodiments, a dryer could be or include a commercial dehydrator. In general, a dryer could include such components as a lamp and/or other form of a heater, a fan, and a controller, for example. The controller could control the heater and/or the fan according to settings of the dryer. Step 586 could include transferring the cannabis material onto carriers such as racks or trays and loading the carriers onto shelves inside of the dryer(s). In some embodiments, step 586 could include adding other ingredients or materials to the dryer. For example, ingredients could be added to adjust the flavour, fragrance, look, and/or texture of the dried cannabis product.

At step 588, the cannabis material is dried in the dryer(s). Dryer settings could be controlled manually, be predefined in a dryer controller, and/or received or otherwise obtained or determined by the controller. Examples of dryer settings include temperature, fan speed, and drying time. In some embodiments, the drying temperature is 60° C. and the drying time is at least 1.5 hours. Other dryer settings are possible. The cannabis material could also or instead be actively monitored by an operator, and/or one or more sensors such as the sensor(s) 428e in FIG. 4E, to determine or adjust dryer settings. A controller and/or one or more sensors could be connected to or have access to the ICS to record the dryer settings and/or one or more properties of the cannabis material during the drying process. Alternatively, information could be manually recorded in the ICS for a drying process, using a computer such as 424e in FIG. 4E, for example.

At step 590, the dried cannabis material is removed from the dryer(s). Step 590 could be performed when a predetermined drying time has been reached, or when an operator or sensor determines that the drying is complete.

Step 592 includes weighing the dried cannabis material, by a scale at 430e-2 in FIG. 4E for example. The measured weight could be recorded in the ICS for the drying process.

At step 594, the dried cannabis material is transferred to one or more holding container(s), shown by way of example at 454e in FIG. 4E. A label could be applied to a holding container using the ICS, or a pre-existing label on a holding container could be recorded in the ICS to indicate that the holding container now contains the dried cannabis product. The label maker(s) 432e and the scanner(s) 434e-2 in FIG. 4E are examples of system components that could be configured for marking holding containers and scanning markings on holding containers, respectively. Either or both of these components could transmit label information to other components for storage in or updating of an ICS, in the database 414 in FIG. 4A for example.

Steps 592 and 594 could be reversed in order in some embodiments, such that the dried cannabis material is weighed after being transfer to the holding container(s).

At step 596 the holding container(s) containing the dried cannabis material is transferred to one or more storage areas. Any such transfer could be recorded in an ICS to help track the location of a holding container. A storage area could be an area where holding containers await further processing, such as irradiation, testing and/or final packaging. A storage area could also or instead be an area where holding containers are stored until they are released for sale. In some embodiments, a storage area is a vault and access is restricted to selected users.

Step 598 includes cleaning the workspace, dryer(s) and/or carrier(s) for the drying process. Other components or equipment such as any source product holding container(s) 450e in FIG. 4E could also or instead be cleaned.

Any of various components of a drying system, such as the example drying system 420e in FIG. 4E, could be configured to generate, collect, and/or otherwise obtain drying information and transmit that information to the server 402 in FIG. 4A, through the server 418e in some embodiments, for populating and/or updating the database 414 or particular records therein. This includes the components which are referenced by way of example above in the description of FIG. 9, and/or possibly other components.

The foregoing description of FIG. 9 refers primarily to drying, but could also or instead be applied to curing. Instead of or in addition to one or more dryers, a curing process could involve curing equipment to cure selected cannabis plant material.

For some cannabis products that use or include dried cannabis, smaller particle size and/or finer granularity of dried cannabis might be desired. For example, dried cannabis with a fine granularity could be desired for rolling cannabis cigarettes. Milling could be used to grind or shred cannabis material, such as the dried cannabis produced by method 580, to produce a finer granularity.

Any of various information relating to a milling process and/or milled cannabis products could be recorded in an ICS, in the form of a milling record in some embodiments. A milling record could include or otherwise be associated with a milling record identifier, which could be similar in form to other identifiers disclosed herein. Milling information related to a milling process and/or milled cannabis products could also or instead be recorded in another type of record such as a lot record.

A lot record could include a milling record identifier or otherwise be associated with a milling record for a milling process that was used to produce a lot of milled cannabis product, or multiple milling records if the lot was milled in different equipment, using different milling processes, and/or over multiple days for example. Similarly, a milling record could include a lot identifier or otherwise be associated with a lot record, or multiple lot records if a milling process or equipment produced multiple lots of milled cannabis product.

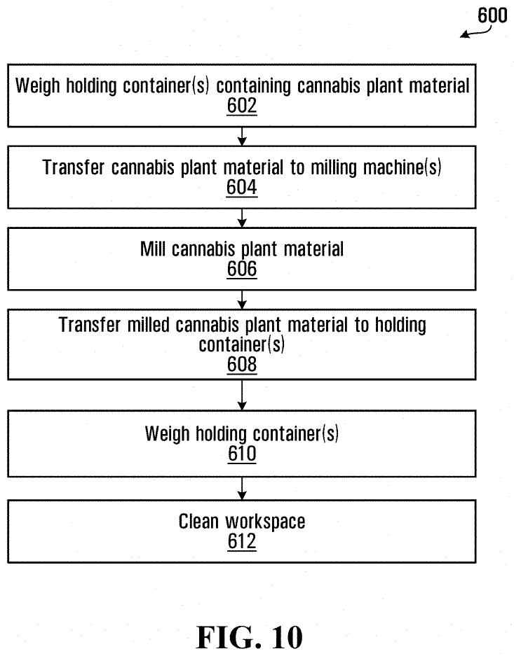

FIG. 10 is a flow diagram illustrating an example method 600 for milling cannabis plant material.

Step 602 includes weighing one or more holding container(s) that contain the cannabis plant material that is to be milled. By way of example, FIG. 4F illustrates source product holding container(s) 450f that could contain source material in the form of cannabis plant material, and weight could be measured by the scale(s) at 430f-1. Alternatively, the cannabis plant material could be removed from the holding container(s) and weighed. Measured weight, batch number, lot number, and/or any other information associated with the cannabis plant material could be recorded in the ICS for the milling process. The weight of the cannabis plant material could be recorded as “pre-mill” weight in the ICS, for example.

At step 604, the cannabis plant material is transferred to one or more milling machines, such as the milling machine(s) 452f in FIG. 4F. In some embodiments, a milling machine could include a rotating blade driven by a motor. An external or integrated controller could be used to control the milling machine. FIG. 4F illustrates an external controller embodiment as an example, in which the controller(s) 426f are connected to or otherwise in communication with the milling machine(s) 452f to control the milling machine(s).

At step 606, the cannabis plant material is milled using the milling machine(s). Milling settings for the milling machine(s) could be controlled manually, be predefined in a milling controller, and/or received or otherwise obtained or determined by the controller. Examples of milling settings include milling time and motor speed. The cannabis plant material could also or instead be actively monitored by an operator, and/or one or more sensors such as the sensor(s) 428f in FIG. 4F, to determine or adjust milling settings. A controller and/or one or more sensors could be connected to or have access to the ICS to record the milling settings and/or one or more properties of the cannabis plant material during the milling process. Alternatively, information could be manually recorded in the ICS for a milling process, using a computer such as 424f in FIG. 4F, for example.

At step 608, the milled cannabis plant material is transferred to one or more holding container(s), shown by way of example at 456f in FIG. 4F. The holding container(s) could include the same holding container(s) that contained un-milled cannabis plant material, and/or one or more different holding containers. In some embodiments, the milled cannabis material could be sifted, using a sifter or sieve for example, before being transferred to a holding container. Sifting could separate the milled cannabis material into different size categories. For example, the milled cannabis product could be separated into fine particles, “ideal mill” particles, and coarse particles. Each size category of milled cannabis material could then be transferred to a respective holding container. The example milling system 420f in FIG. 4F includes one or more sifters 454f.

Any transfer of milled cannabis material to a holding container could be recorded in the ICS. If the material in a holding container was sifted, then this could also or instead be recorded in the ICS.

A label could be applied to a holding container using the ICS, or a pre-existing label on a holding container could be recorded in the ICS to indicate that the holding container now contains milled cannabis material. The label maker(s) 432f and the scanner(s) 434f-2 in FIG. 4F are examples of system components that could be configured for marking holding containers and scanning markings on holding containers, respectively.

Either or both of these components could transmit label information to other components for storage in or updating of an ICS, in the database 414 in FIG. 4A for example.

Step 610 includes weighing the holding container(s) containing the milled cannabis material, using the scale(s) 430f-2 in FIG. 4F for example. The measured weight could be recorded in the ICS as a “post-mill” weight. If sifting was also performed to separate the milled cannabis material, then the weight of a holding container could be recorded as a “post-mill/sift” weight.

The holding container(s) could then be moved to one or more storage areas. Any such transfer could be recorded in an ICS to help track the location of a holding container. Examples of storage areas are provided elsewhere herein.

At step 612, the workspace is cleaned, and this could involve cleaning any milling machines and/or sifting machines that were used. Waste that is produced in a milling process could be weighed and/or otherwise recorded in the ICS before being destroyed.

Any of various components of a milling system, such as the example milling system 420f in FIG. 4F, could be configured to generate, collect, and/or otherwise obtain milling information and transmit that information to the server 402 in FIG. 4A, through the server 418f in some embodiments, for populating and/or updating the database 414 or particular records therein. This includes the components which are referenced by way of example above in the description of FIG. 10, and/or possibly other components.

Milled and/or dried cannabis could be used to produce “pre-rolled” cannabis cigarettes, for example. Pre-rolled cigarettes could be rolled by a producer during lot packaging, as opposed to being rolled by a user. Pre-rolled cannabis cigarettes could be produced manually, or produced with the aid of a cone filling machine.

Any of various information relating to a pre-rolling process and/or pre-rolled cannabis products could be recorded in an ICS, in the form of a pre-rolling record in some embodiments. A pre-rolling record could include or otherwise be associated with a pre-rolling record identifier, which could be similar in form to other identifiers disclosed herein. Pre-rolling information related to a pre-rolling process and/or pre-rolled cannabis products could also or instead be recorded in another type of record such as a lot record.

A lot record could include a pre-rolling record identifier or otherwise be associated with a pre-rolling record for a pre-rolling process that was used to produce a lot of pre-rolled cannabis product, or multiple pre-rolling records if the lot was pre-rolling in different equipment, using different pre-rolling processes, and/or over multiple days for example. Similarly, a pre-rolling record could include a lot identifier or otherwise be associated with a lot record, or multiple lot records if a pre-rolling process or equipment produced multiple lots of pre-rolled cannabis product.

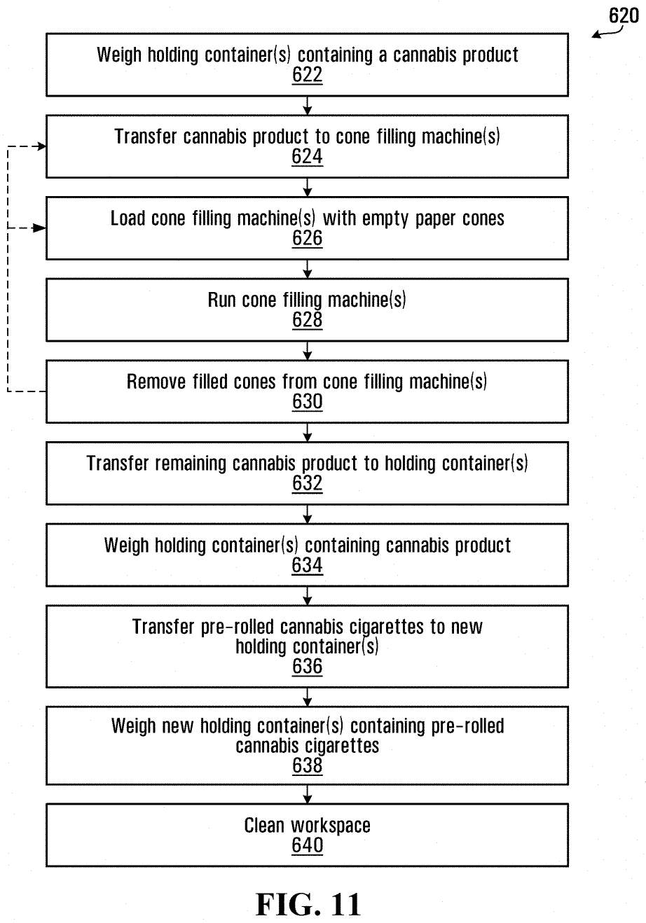

FIG. 11 is a flow diagram illustrating an example method 620 for producing pre-rolled cannabis cigarettes with a cone filling machine. In some embodiments, the method 620 could be performed during the operation 118 of FIG. 1.

Step 602 includes weighing one or more holding containers containing a cannabis product. This cannabis product could include dried and/or milled cannabis plant material, for example. By way of example, FIG. 4J illustrates source product holding container(s) 450j that could contain pre-rolling cannabis product, and weight could be measured by the scale(s) at 430j-1. Alternatively, the source product could be removed from the holding container(s) and weighed. Measured weight, batch number, lot number, and/or any other information associated with the source product could be recorded in the ICS for the pre-rolling process. Measured weight could be recorded in the ICS as a “pre pre-roll” weight, for example.

At step 624, the cannabis product is transferred from the holding container(s) to one or more cone filling machine(s), shown by way of example at 452j in FIG. 4J. The cannabis product could be loaded onto trays and/or other supply carriers for loading the cone filling machine(s), for example.

Step 626 involves loading the cone filling machine(s) with empty paper cones. In some embodiments, empty paper cones are placed onto or into trays or other carriers, which are loaded into the cone filling machine(s). Paper cones could be available in multiple sizes, and cone size determines the cannabis product capacity per pre-rolled cigarette and the size of the pre-rolled cigarettes that are produced. The cone filling machine(s) could be loaded with cones of one size at a time, but this need not be the case in all embodiments. There could also or instead be multiple pre-rolling mechanisms in a machine to handle cones of respective different sizes, and/or multiple machines to handle cones of respective different sizes. In some embodiments, a cone-filling machine is not size-specific, and is configured to handle cones of multiple sizes. A multi-size cone filling machine could dynamically detect cone size and handle multiple different cone sizes at a time, or be configurable to handle different cone sizes but only one cone size at a time.

Step 628 involves running or operating the cone filling machine(s), to fill the paper cones with the cannabis product. One or more settings for the cone filling machine(s) could be adjusted before or during each run. For example, the weight and/or volume of cannabis to be added to each cone could be adjusted manually, or automatically by a controller in a cone filling machine based on the size and/or type of cones currently loaded. In general, settings for a cone filling machine could be controlled manually, be predefined in a controller, and/or received or otherwise obtained or determined by the controller. Filling weight and/or volume, noted above, are examples of such settings. A cone filling machine could also or instead be actively monitored by an operator, and/or one or more sensors such as the sensor(s) 428j in FIG. 4J, to determine or adjust settings.

A controller and/or one or more sensors could be connected to or have access to the ICS to record settings and/or one or more properties of the cone filling machine(s), cannabis product, empty paper cones, and/or machine output(s) during the pre-rolling process. Alternatively, information could be manually recorded in the ICS for a pre-rolling process, using a computer such as 424j in FIG. 4J, for example.

Step 630 includes removing the filled paper cones from the cone filling machine(s). Step 630 could be performed manually by an operator, or be automated by one or more machines. In some embodiments, filled paper cones are ejected from the machine(s), and could drop or otherwise be transferred to one or more holding container(s).

Open ends of filled cones, through which the cones were filled, could be closed by the cone filling machine(s), or could be closed by folding or twisting after the cones are removed from the cone filling machine(s) at 630. Closing the ends of the filled cones could reduce or prevent the cannabis product from falling out of the filled cones, and forms pre-rolled cannabis cigarettes.

Some of the filled cones that are removed might be damaged or otherwise unsuitable for sale. Any cannabis product in damaged cones could be recycled back into the cone filling machine(s) or the original holding container(s). The cone filling machine(s) could be run multiple times by loading the machine with additional empty cones and/or with additional cannabis product. For example, steps 624, 626, 628, 630, could be repeated multiple times, as indicated using dashed lines in FIG. 11. Producing pre-rolled cigarettes could stop when, for example, a pre-defined number of cigarettes have been produced, or the amount of cannabis product remaining is less than the amount needed for a run of the cone filling machine(s).

At step 632, cannabis product that remains after pre-rolling has finished is removed from the cone filling machine(s) and returned to one or more holding container(s), which could be the original holding container(s) from which the cone filling machine(s) were loaded.

Step 634 involves weighing the holding container(s). If remaining cannabis product is transferred back to the original holding container(s) at 632, then the original holding container(s) could be weighed again at 634. Otherwise, the holding container(s) in which the remaining cannabis product is transferred at 632 are also or instead weighed at 634. Both the original and remaining cannabis product holding containers could be weighed unless the original holding container was completely emptied, for example, so that a total remaining “post pre-roll” weight can be measured or otherwise determined, and could be recorded in the ICS. The difference between pre pre-roll and post pre-roll weights should indicate the weight of cannabis in the pre-rolled cannabis cigarettes, provided any remaining cannabis product, including contents of any damaged filled cones, has been returned to one or more holding containers before measurement of post pre-roll weight(s).

At step 636, the pre-rolled cannabis cigarettes that were removed from the cone filling machine at step 630 are transferred to one or more new holding containers, shown by way of example as the target holding container(s) 4456j in FIG. 4J. Step 636 could also include weighing each pre-rolled cannabis cigarette to confirm that it does exceed a maximum weight. In one example, if a pre-rolled cigarette weighs over 1.0 g, then it could be destroyed or recycled. In another example, if the weight and/or volume of a pre-rolled cigarette deviates from a target weight/volume by more than a pre-defined tolerance, then the cigarette could be destroyed or recycled. The pre-defined tolerance could be 5% or 10%, for example.

Step 638 includes weighing the new holding container(s) containing the pre-rolled cannabis cigarettes, using the scale(s) 430j-2 in FIG. 4J. Holding container weight in the case of pre-rolled cigarettes includes the weight of the paper cones and the cannabis product in the pre-rolled cigarettes. This weight could be entered, manually or automatically, into the ICS.

A label could be applied to a holding container using the ICS, or a pre-existing label on a holding container could be recorded in the ICS to indicate that the holding container now contains pre-rolled cannabis cigarettes. The label maker(s) 432j and the scanner(s) 434j-2 in FIG. 4J are examples of system components that could be configured for marking holding containers and scanning markings on holding containers, respectively. Either or both of these components could transmit label information to other components for storage in or updating of an ICS, in the database 414 in FIG. 4A for example.

The holding container(s) could then be moved to one or more storage areas. Any such transfer could be recorded in an ICS to help track the location of a holding container. Examples of storage areas are provided elsewhere herein.

At step 640, the workspace is cleaned, and this could involve cleaning the cone filling machine(s) are cleaned. Waste that is produced in a milling process could be weighed and/or otherwise recorded in the ICS before being destroyed.

Any of various components of a packaging system, such as the example packaging system 420j in FIG. 4J, could be configured to generate, collect, and/or otherwise obtain pre-rolling information and transmit that information to the server 402 in FIG. 4A, through the server 418j in some embodiments, for populating and/or updating the database 414 or particular records therein. This includes the components which are referenced by way of example above in the description of FIG. 11, and/or possibly other components.

Cannabis Extract Manufacturing

Another example of a cannabis product is a cannabis extract, which could be or include oils, and non-oils such as resins. Cannabis extracts could be further processed to produce other cannabis products.

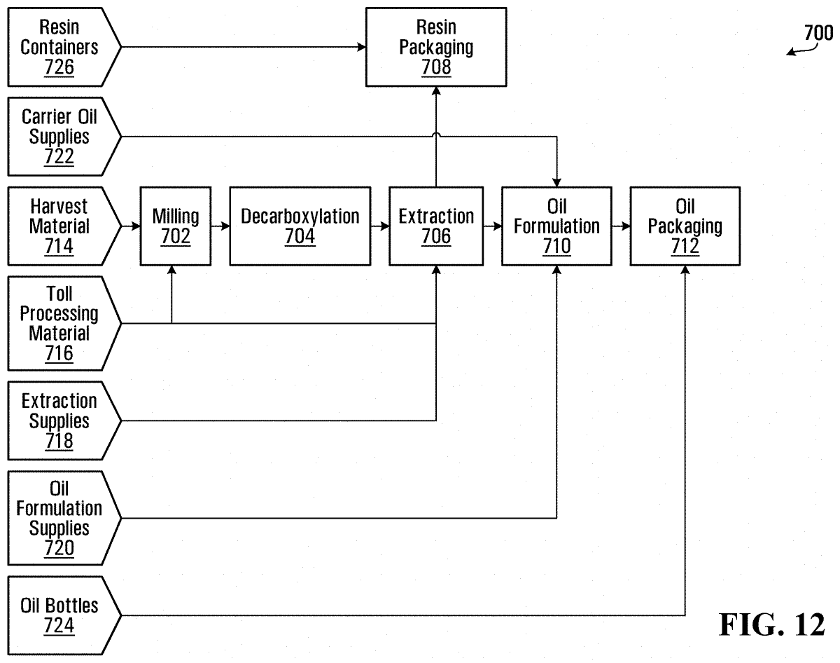

FIG. 12 is a flow diagram illustrating an example process 700 for producing cannabis extracts and other cannabis products. The process 700 includes an operation 702 of milling, an operation 704 of decarboxylation, an operation 706 of extraction, an operation 708 of resin packaging, an operation 710 of oil formulation, and an operation 712 of oil packaging. These operations are discussed in greater detail below, in some instances with additional reference to other drawings. Any or all of the operations 702, 704, 706, 708, 710, 712 could be similar to one or more of the processes performed in operations 114, 118 of FIG. 1. For example, operations 702, 704, 706 of FIG. 12 could be similar to the extraction performed in operation 114 of FIG. 1. Operations 708, 710, 712 could also or instead be similar to the lot packaging performed in operation 118 of FIG. 1.

Harvest material 714 could be a source of cannabis plant material for process 700, and could include plant material output from a plant part separation process, such as the plant part separation process performed in operation 104 of FIG. 1 and/or by a plant part separation system such as the example system 420b in FIG. 4B. Harvest material 714 could include a single batch or lot of cannabis plant material. Alternatively, harvest material 714 could include multiple batches or lots of cannabis plant material.

Toll processing material 716 could also or instead be a source of cannabis material for process 700. Toll processing refers to a situation in which a company or entity processes cannabis material or products for another company or entity, and returns the resultant product(s) the other company or entity for a fee. For example, a company performing process 700 could receive cannabis material from an external company and process this plant material to produce extracts that are returned to the external company. Important considerations for handling toll processing material 716 could include reducing cross-contamination, preventing the addition of any extraneous substance, preserving product integrity, and keeping accurate records of all products to enable identification and traceability.

When toll processing material 716 is received, information such as the name of the individual or organization from which it was received, the address of the site at which it was received, the date on which is was received, the quantity of material received, the intended use of the material received, and/or the brand name of the material received could be recorded in an ICS. Each holding container and/or package of toll processing material 716 could be weighed, and this weight could be recorded in the ICS. The measured weight could be compared to a weight listed in an order request, to confirm that the received weight matches what was ordered and/or what was shipped to the receiver. The received product could then be placed in new holding containers, which could be labelled and recorded in the ICS. Alternatively, the original packages or holding containers of the toll processing material 716 could be labelled and/or recorded in the ICS. The holding containers could then be stored before they are processed, tested and/or allocated a lot number.

In some embodiments, toll processing material 716 could be handled in the same or substantially the same manner as source material or products in example methods disclosed herein. Toll processing material 716 might originate from a different source than the harvest material 714, but need not necessarily be handled in a substantially different way or by substantially different systems or components because of its different origin.

The example process 700 begins with cannabis milling at operation 702, to grind or mill cannabis material for extraction. Examples of milling processes, and potential implications for an ICS, are disclosed elsewhere herein. The milling at 702 could reduce the cannabis plant particle size, which could increase the efficiency of other processing such as extraction. Harvest material 714 could be sent for milling at operation 702. In the case of toll processing material 716 that includes un-milled flower, trim or waste, for example, the toll processing material could also or instead be sent for milling at operation 702. In some embodiments, only harvest material 714 or only toll processing material 716 are processed in any operation at one time.

Milled cannabis plant material that is produced at operation 702 could be sent to operation 704 for decarboxylation in some embodiments. Decarboxylation is a process in which acid forms of cannabinoids are converted to their neutral forms. More specifically, decarboxylation involves a chemical reaction that removes a carboxyl group from cannabinoids and releases CO₂. It should be noted that decarboxylation is shown solely for illustrative purposes in FIG. 12, and need not be performed in all embodiments.

By way of background in relation to decarboxylation, the term “Cannabis plant(s)” encompasses wild type Cannabis and also variants thereof, including cannabis chemovars which naturally contain different amounts of the individual cannabinoids. For example, some Cannabis strains have been bred to produce minimal levels of THC, the principal psychoactive constituent responsible for the high associated with it and other strains have been selectively bred to produce high levels of THC and other psychoactive cannabinoids.

Cannabis plants produce a unique family of terpeno-phenolic compounds called cannabinoids, which produce the cannabis-effect one experiences from consuming marijuana. There are 483 identifiable chemical constituents known to exist in the cannabis plant, and at least 85 different cannabinoids have been isolated from the plant. The two cannabinoids usually produced in greatest abundance are cannabidiol (CBD) and/or Δ9-tetrahydrocannabinol (THC), but only THC is psychoactive. Cannabis plants are categorized by their chemical phenotype or “chemotype,” based on the overall amount of THC produced, and on the ratio of THC to CBD. Although overall cannabinoid production is influenced by environmental factors, the THC/CBD ratio is genetically determined and remains fixed throughout the life of a plant. Non-drug plants produce relatively low levels of THC and high levels of CBD, while drug plants produce high levels of THC and low levels of CBD.

The best studied cannabinoids include tetrahydrocannabinol (THC), cannabidiol (CBD) and cannabinol (CBN). Other cannabinoids include for example, cannabichromene (CBC), cannabigerol (CBG) cannabinidiol (CBND), Cannabicyclol (CBL), Cannabivarin (CBV), Tetrahydrocannabivarin (THCV), Cannabidivarin (CBDV), Cannabichromevarin (CBCV) Cannabigerovarin (CBGV), Cannabigerol Monomethyl Ether (CBGM).

Cannabinoids are derived from their respective 2-carboxylic acids (2-COON) by decarboxylation (catalyzed by heat, light, or alkaline conditions). As a general rule, the carboxylic acids form of the cannabinoid have the function of a biosynthetic precursor.

As used herein THC, CBD, CBN, CBC, CBG, CBND, CBL, CBV, THCV, CBDV, CBCV, CBGV and CBGM refer to the decarboxylated form of the cannabinoid. Whereas, THCa, CBDa, CBNa, CBCa, CBGa, CBNDa, CBLa, CBVa, THCVa, CBDVa, CBCVa and CBGVa refer to the acid form of the cannabinoid.

Tetrahydrocannabinol (THC) is the primary psychoactive component of the Cannabis plant. THC is only psychoactive in is decarboxylated state. The carboxylic acid form (THCa) is non-psychoactive.

Delta-9-tetrahydrocannabinol (Δ9-THC, THC) and delta-8-tetrahydrocannabinol (ΔΔ8-THC), mimic the action of anandamide, a neurotransmitter produced naturally in the body. These two THCs produce the effects associated with cannabis by binding to the CB1 cannabinoid receptors in the brain. THC appears to ease moderate pain (analgesic) and to be neuroprotective, while also offering the potential to reduce neuroinflammation and to stimulate neurogenesis.

The term “Cannabis plant” encompasses wild type Cannabis sativa, Cannabis indica, Cannabis afghanica, and other variants thereof, including cannabis species which naturally contain different amounts of the individual cannabinoids. Also included are Cannabis subspecies and plants which are the result of genetic crosses, self-crosses or hybrids thereof. Also included are hemp plants. The term “Cannabis extract” is to be interpreted accordingly as encompassing material extracted from one or more cannabis plants.

THC and CBD are the main medicinally active constituents in Cannabis. However, these constituents are present as the biologically inactive carboxylic acids in Cannabis plants. When extracting THC or CBD from cannabis plants, it has been the practice to convert the storage precursor compounds of THCA and CBDA into their more readily extractable and pharmacologically active forms. THC and CBD acids slowly decarboxylate over time, and applying heat increases the rate of decarboxylation.

Decarboxylation of cannabinoid acids is a function of time and temperature, thus, at higher temperatures a shorter period of time will be taken for complete decarboxylation of a given amount of cannabinoid acid. In selecting appropriate conditions for decarboxylation consideration must, however, be given to minimising thermal degradation of the desirable, pharmacological cannabinoids into undesirable degradation products, particularly thermal degradation of THC to cannabinol (CBN).

Any of various information relating to a decarboxylation process and/or decarboxylated cannabis products could be recorded in an ICS, in the form of a decarboxylation record in some embodiments. A decarboxylation record could include or otherwise be associated with a decarboxylation record identifier, which could be similar in form to other identifiers disclosed herein. Decarboxylation information related to a decarboxylation process and/or decarboxylated cannabis products could also or instead be recorded in another type of record such as a lot record.

A lot record could include a decarboxylation record identifier or otherwise be associated with a decarboxylation record for a decarboxylation process that was used to produce a lot of decarboxylated cannabis product, or multiple decarboxylation records if the lot was decarboxylated in different equipment, using different decarboxylation processes, and/or over multiple days for example. Similarly, a decarboxylation record could include a lot identifier or otherwise be associated with a lot record, or multiple lot records if a decarboxylation process or equipment produced multiple lots of decarboxylated cannabis product.

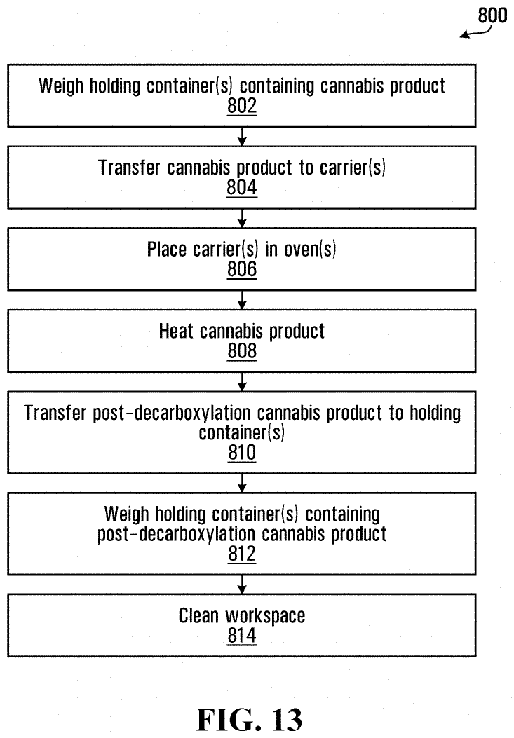

FIG. 13 is a flow diagram illustrating an example method 800 for decarboxylation of a cannabis product, such as the decarboxylation performed at operation 704 of FIG. 12 and/or in a decarboxylation system such as the example system 420g in FIG. 4G.

Step 802 involves weighing one or more holding containers containing a pre-decarboxylation cannabis product. One or more scales and one or more holding containers are shown by way of example at 430g-1 and 450g, respectively, in FIG. 4G. The cannabis product could include plant material that was milled at operation 702 of FIG. 12, for example. The measured weight could be recorded in an ICS, along with the batch number, lot number, or any other information associated with the cannabis product and/or the holding container(s) in some embodiments. This weight could be recorded in the ICS as a “pre-decarboxylation” weight, for example.

At step 804, cannabis product is transferred from the holding container(s) to one or more carriers, such as trays. In some embodiments, a carrier is an aluminum tray. Before transferring the cannabis product to a carrier, the carrier could be cleaned using food grade ethanol, for example.

Step 806 involves placing the carrier(s) into one or more ovens, such as the decarboxylation oven(s) 452g in FIG. 4G. Removable carriers such as trays might not necessarily be used in all embodiments. For example, cannabis product could instead be loaded into one or more ovens without necessarily using a carrier.

An oven could be preheated to a particular temperature before cannabis product is added. In some embodiments, an oven could be set to a temperature of 150° C., and cannabis product might not be transferred to the oven until it has reached a minimum temperature of 120° C. A temperature probe or thermometer could be inserted into the cannabis product to monitor the temperature of the cannabis product during decarboxylation. This temperature probe could be connected to or have access to the ICS to record and track the temperature of the cannabis product. A temperature probe is an example of a sensor shown at 428g in FIG. 4G. An oven could also or instead have access to the ICS, to record its actual and/or set point temperatures.

Oven settings could be controlled manually, be predefined in an oven controller, and/or received or otherwise obtained or determined by the controller. Examples of oven settings include temperature and decarboxylation time. Other settings are possible. The cannabis material could also or instead be actively monitored by an operator, and/or one or more sensors such as the sensor(s) 428g in FIG. 4G, to determine or adjust oven settings. A controller and/or one or more sensors could be connected to or have access to the ICS to record the oven settings and/or one or more properties of the cannabis material during the decarboxylation process. Alternatively, information could be manually recorded in the ICS for a decarboxylation process, using a computer such as 424g in FIG. 4G, for example.

At step 808, the cannabis product is heated. Heating could continue until the cannabis product reaches a predefined temperature. This temperature could be the temperature at which the decarboxylation process occurs. In some embodiments, the predefined temperature could be 120° C. It could be desirable to maintain the temperature of the cannabis product within a certain range of the predefined temperature. For example, a cannabis product could be maintained within 4° C. of 120° C. Heating the cannabis product to temperatures that exceed this range of the predefined temperature might be undesirable. Such temperatures could induce other reactions, such as vaporization of cannabinoids and terpenes, which might affect the properties of the final cannabis product. In some embodiments, if the cannabis product reaches temperatures greater than 125° C., the set point temperature of the oven could be decreased. A damper and/or oven door could also or instead be opened to decrease the temperature of the oven.

At step 810, the cannabis product is be taken out of the oven(s) and transferred to one or more holding container(s). The cannabis product might be taken out of the oven(s) once a particular temperature has been maintained or exceeded for a particular amount of time. This temperature and amount of time could depend, for example, on the temperature-dependent rate of the decarboxylation process for that particular cannabis product. In some embodiments, a cannabis product might be removed from an oven if its temperature exceeds 90° C. for at least 100 minutes. The actual temperature of the cannabis product and/or the time the cannabis product is at a temperature above a particular temperature could be recorded in the ICS. Once out of the oven(s), a carrier containing the cannabis product could be allowed to cool in the ambient atmosphere. The cannabis product could then be transferred to the original holding container(s). Decarboxylated cannabis product could also or instead be transferred to one or more different holding container(s), shown by way of example at 454g in FIG. 4G.

A label could be applied to a holding container using the ICS, or a pre-existing label on a holding container could be recorded in the ICS to indicate that the holding container now contains the dried cannabis product. The label maker(s) 432g and the scanner(s) 434g-2 in FIG. 4G are examples of system components that could be configured for marking holding containers and scanning markings on holding containers, respectively. Either or both of these components could transmit label information to other components for storage in or updating of an ICS, in the database 414 in FIG. 4A for example.

Step 812 includes weighing the holding container(s) containing the decarboxylated (post-decarboxylation) cannabis products material, by a scale at 430g-2 in FIG. 4G for example. The measured weight could be recorded as a “post-decarboxylation” weight in the ICS, for example.

The holding container(s) could then be moved to one or more storage areas. Any such transfer could be recorded in an ICS to help track the location of a holding container. Examples of storage areas are provided elsewhere herein.

At step 814, the workspace is cleaned, and this could involve cleaning the oven(s) and/or any carrier(s) used for decarboxylation. Waste that is produced in a decarboxylation process could be weighed and/or otherwise recorded in the ICS before being destroyed.

The decarboxylation method 800 illustrated in FIG. 13 could be used to produce cannabis material for extraction processes. It should be noted, however, that cannabis products other than decarboxylated cannabis products could be provided as inputs to an extraction process. A cannabis product need not necessarily undergo decarboxylation before extraction.

Referring again to FIG. 12, operation 706 includes an extraction process to produce one or more cannabis extracts. The post-decarboxylation cannabis product from operation 704, or another cannabis material or product, could be used as a source material for the extraction process at operation 706. Harvest material 714 and/or toll processing material 716 could also or instead be used as source material for the extraction process at operation 706. For example, the toll processing material 716 could have undergone milling and/or decarboxylation before being received, or decarboxylation might not be performed before extraction.

Extraction supplies 718 are provided to support the extraction at operation 706. Extraction supplies 718 could include extraction solvents and extract collection vessels, for example. An extraction solvent is used in solvent extraction processes, which separate compounds from a source material based on their relative solubility in the extraction solvent. An extract collection vessel is a container for holding an extract produced by extraction. In some embodiments, an extract collection vessel could be a collection flask or other form of receptacle. However, other extract collection vessels could also or instead be used.

In some embodiments, operation 706 includes supercritical fluid extraction with CO₂. Supercritical fluid extraction with CO₂ is the process of separating an extract from a matrix using supercritical CO₂ as the extraction solvent. When cannabis material is used as the matrix, supercritical fluid extraction with CO₂ could separate cannabinoids and terpenes from the cannabis material. These cannabinoids and terpenes could be captured in the form of a cannabis extract. The remaining cannabis material could be considered to be waste.

Any of various information relating to an extraction process and/or cannabis extracts could be recorded in an ICS, in the form of an extraction record in some embodiments. An extraction record could include or otherwise be associated with an extraction record identifier, which could be similar in form to other identifiers disclosed herein. Extraction information related to an extraction process and/or cannabis extracts could also or instead be recorded in another type of record such as a lot record.

A lot record could include an extraction record identifier or otherwise be associated with an extraction record for an extraction process that was used to produce a lot of cannabis extract, or multiple extraction records if the lot was produced in different extraction equipment, using different extraction processes, and/or over multiple days for example. Similarly, an extraction record could include a lot identifier or otherwise be associated with a lot record, or multiple lot records if an extraction process or equipment produced multiple lots of cannabis extract.

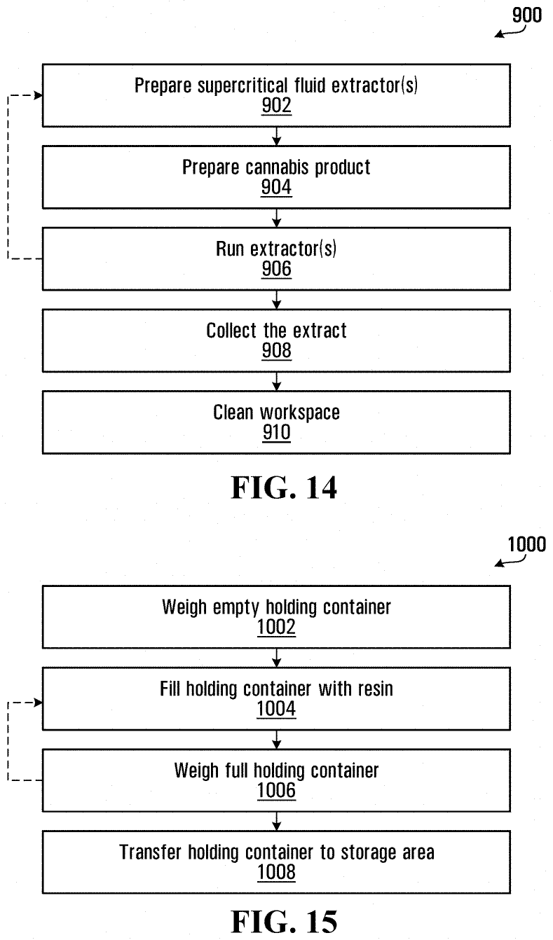

FIG. 14 is a flow diagram illustrating an example method 900 for supercritical fluid extraction with CO₂. The example method 900 represents one possible option for an extraction process at 706 in FIG. 12, and/or could be performed by the extractor(s) 452h in FIG. 4H.

Step 902 includes preparing one or more supercritical fluid extractors. In some embodiments, a supercritical fluid extractor includes a source of compressed CO₂, an extraction chamber, one or more heaters to heat the extraction chamber, one or more collection chambers connected to the extraction chamber to collect extract, a CO₂ monitor, an inlet regulating valve to control the flow of CO₂ into the extraction chamber, an outlet regulating valve to control the flow of CO₂ out of the extraction chamber, a venting valve such as a needle valve to controllably vent the extraction chamber, and a controller. Preparing a supercritical fluid extractor could include venting and opening the extraction chamber, for example. To vent an extraction chamber, inlet and outlet regulating valves could be closed and a venting valve could then be opened to release any CO₂ in the extraction chamber. Once the extraction chamber is vented, the extraction chamber could be opened. This could include dismantling a portion of the extraction chamber, such as the top of the extraction chamber.

At step 904, a cannabis product is prepared for extraction. Step 904 could include transferring the cannabis product into an extraction bag. In some embodiments, a charge of approximately 5 grams of cannabis product is transferred to the extraction bag. Transferring cannabis product into an extraction bag could include placing and securing the extraction bag inside of the extraction chamber, adding cannabis product into the bag using a funnel or other guide if needed, and tying the top of the bag with the cannabis product inside. The extraction chamber could then be closed and sealed. For example, the top of the extraction chamber could be reassembled. A pressure check could be performed to test for any leaks and ensure that all of the seals and fittings in the extraction chamber are operating correctly. Parameters or information such as the source material used for the extraction process could be recorded in the ICS. To record the source material, a batch number, lot number, and/or label on the holding container(s) of the source material could be recorded in the ICS. The ICS could also or instead record the weight of the source material transferred to the extraction bag.

Step 906 involves running the extractor(s). Once an extraction chamber is closed without any leaks, its inlet and outlet regulating valves could be adjusted to allow the extraction chamber to fill up with CO₂. The CO₂ monitor, which is an example of a sensor 428h in FIG. 4H, could be used to monitor the amount of CO₂ in the extraction chamber. After the extraction chamber is filled with CO₂ and has reached a stable pressure, the chamber heater could be started. The chamber could be left for a predefined time, such as 30 minutes, to allow the chamber to reach a stable temperature. Chamber temperature and/or pressure could be measured by other sensor(s) 428h.

With stable temperature and pressure, and extractor could then be run to produce extract from the source material. Running an extractor could include adjusting heat and/or pressure in the extractor to convert gaseous CO₂ into a super critical fluid. In some embodiments, running the extractor is an automated process. For example, an operating program for the extractor could define parameters for the extraction run, including one or more of time duration, CO₂ flow rate, temperatures and pressures. The operation program could be stored on a controller of the extractor. The controller could control one or more of the valves, heater, and/or other components of the extractor during a run. The parameters of the extraction run could be recorded in the ICS. For example, the controller could be in communication with or have access to the ICS to record extraction parameters for the extraction process record. Extraction parameters could also or instead be recorded in the ICS manually, using a computer 424h in FIG. 4H for example. Extraction information could also or instead be collected and/or provided by other components such as one or more sensor(s) 428h.

The ICS could allow a user to view and monitor the status of an extraction run via a computer or other electronic device through which the ICS is accessible.

In some embodiments, steps 902, 904, 906 could be repeated multiple times to produce larger quantities of extract. This repetition is indicated using a dashed line in FIG. 12. In some cases, 8 to 12 extractions could be performed before a collection. Each extraction run at step 906 could be recorded using the ICS, using the same extraction record or different extraction records for example.

At step 908, the extract produced at step 906 is collected, using a collection vessel in some embodiments. Step 908 could include connecting a collection chamber on the extractor to a collection vessel. Purging the collection chamber with CO₂ could help to push the extract from the collection chamber into the collection vessel. The extract could be collected in the form of a resin. In some embodiments, an “extract” record could be created in the ICS to record and track the extract that is collected. Alternatively, the collected extract could be added to an existing extract record in the ICS. Extract records could be identified as, for example, “EXTR-1”, “EXTR-2” and “EXTR-3”. An extract record could be associated with an extraction record in the ICS.

Before collection of the extract at step 908, an empty extract collection vessel could be weighed and recorded in the ICS. A label could be generated by the ICS and applied to the collection vessel, or a pre-existing label on the collection vessel could be recorded in the ICS to indicate that the holding container now contains the cannabis extract. After collection of the extract in the collection vessel, the weight and/or volume of the extract in the collection vessel could be recorded in the ICS. The weight of the extract could be determined, for example, by comparing the weight of the collection vessel before and after it is filled. Either or both of these weights could be measured by one or more scales, such as the scale(s) 430h-2 in FIG. 4H. The volume of the extract could be determined using volume markings on the collection vessel. At least a portion of the extract that is collected at step 908 could be sampled and sent for testing to determine, for example, the cannabinoid concentration in the extract.

A collection vessel is an example of an extracted product holding container 458h in FIG. 4H. The label maker(s) 432h and the scanner(s) 434h-2 in FIG. 4H are examples of system components that could be configured for marking holding containers and scanning markings on holding containers, respectively. Either or both of these components could transmit label information to other components for storage in or updating of an ICS, in the database 414 in FIG. 4A for example.

The holding container(s) could then be moved to one or more storage areas. Any such transfer could be recorded in an ICS to help track the location of a holding container. Examples of storage areas are provided elsewhere herein.

At step 910, the workspace is cleaned, and this could involve cleaning the extractor(s). Waste material and/or residual extract could be removed from the extractor(s). Waste material could include any cannabis product that remains in the extraction bag after the extraction process at step 906. The weight of the waste material produced by the extraction run could be recorded in the ICS for the extraction process record. Comparing the weight of the source material to the weight of the waste material could determine the amount of material used in the extraction run. Water and/or disinfectant could be sprayed inside of the extractor(s) to remove residual extract. Cleaning the extractor could be particularly important if different batches or lots of cannabis products are used for subsequent extraction runs in the same extractor, as residual extract in the extractor could lead to cross-contamination of these subsequent extraction runs.

In some embodiments, other processing such as winterization and/or distillation could be applied to extracts. FIG. 4H illustrates winterization chiller(s) 454h and distiller(s) 456h that could be used to perform these processes.

Any of various information relating to a winterization process and/or winterized cannabis products could be recorded in an ICS, in the form of a winterization record in some embodiments. A winterization record could include or otherwise be associated with a winterization record identifier, which could be similar in form to other identifiers disclosed herein. Winterization information related to a winterization process and/or winterized cannabis products could also or instead be recorded in another type of record such as a lot record.

A lot record could include a winterization record identifier or otherwise be associated with a winterization record for a winterization process that was used to produce a lot of winterized cannabis product, or multiple winterization records if the lot was winterized in different equipment, using different winterization processes, and/or over multiple days for example. Similarly, a winterization record could include a lot identifier or otherwise be associated with a lot record, or multiple lot records if a winterization process or equipment produced multiple lots of winterized cannabis product.

Similarly, any of various information relating to a distillation process and/or distilled cannabis products could be recorded in an ICS, in the form of a distillation record in some embodiments. A distillation record could include or otherwise be associated with a distillation record identifier, which could be similar in form to other identifiers disclosed herein. Distillation information related to a distillation process and/or distilled cannabis products could also or instead be recorded in another type of record such as a lot record.

A lot record could include a distillation record identifier or otherwise be associated with a distillation record for a distillation process that was used to produce a lot of distilled cannabis product, or multiple distillation records if the lot was distilled in different equipment, using different distillation processes, and/or over multiple days for example. Similarly, a distillation record could include a lot identifier or otherwise be associated with a lot record, or multiple lot records if a distillation process or equipment produced multiple lots of distilled cannabis product.

A winterization or distillation method could be substantially similar to the example method 900 in FIG. 14. For example, winterization or distillation equipment (such as the winterization chiller(s) 454h or distiller(s) 456h in FIG. 4H) could be prepared for operation, source cannabis product could be prepared for winterization or distillation, the winterization or distillation equipment could then be operated for one or more runs, and resultant output winterized or distilled extract could then be collected. A workspace and/or equipment could then be cleaned. Winterization or distillation methods could include any of various information collection, recording and/or reporting features as well. Any of such parameters as weights of holding containers that contain source cannabis products and/or output cannabis products, winterization or distillation settings and/or conditions, and/or label information could be measured or otherwise collected, recorded, and/or transmitted to populate or update an ICS. Other features could also or instead be provided in conjunction with winterization or distillation. Examples of winterization and distillation processes are also provided below.

Further, typically, supercritical CO₂ extraction of cannabinoids involves a step of winterization after the CO₂ extraction so as to retain the more polar cannabinoid molecules while ridding the crude extract of most other waxes, which is often referred to as waxy ballast. The secondary extraction or “winterization” is an ethanolic-precipitation for removing waxy ballast and purifying the crude Cannabis extract of wax esters, glycerides, and unsaturated fatty acids, which hinder the extract from a refined liquid state. “Winterization” releases any trapped solvents from the initial extraction from the extremely viscous crude extracts.

The process of removing waxy ballast from crude cannabis extract using “winterization”, involves chilling the crude Cannabis extract to a temperature less than or equal to about 0° C., alternatively less than or equal to below about −10° C., alternatively less than or equal to below about 20° C. for a time period. The time period may be at least 1 hour, alternatively at least about 24 hours, alternatively at least about 48 hours, alternatively at least about 50 hours, alternatively at least about 72 hours. After the chilling freezing period, the crude Cannabis extract can be cold-filtered to remove waxy ballast. For example, a Whatman #1 lab filter with vacuum assist is initially used to remove the material that is insoluble, and secondly the crude extract is run through syringe filters (for example, 0.45 or 0.2 micron filters), which takes out any remaining plant material, as well as any bacteria present.

Optionally, the method for obtaining the cannabis concentrate may further include purification steps such as a distillation step in order to further purify, isolate or crystallize one or more cannabinoids. A cannabis concentrate obtained by distillation may be further cut with one or more terpenes (i.e., chemicals made and stored in the trichomes of the cannabis plant, with the cannabinoids. Terpenes give cannabis its distinctive smell. Alternatively, terpenes can be extracted and obtained from other plants).

At least a portion of resin that is collected in the example method 900 could be sent for packaging, which could include transferring the resin from an extract collection vessel to one or more other holding containers, for example. In some embodiments, the holding container(s) could be recorded in the ICS and assigned a lot number. The packaged resin could then be released for sale to consumers. Packaged resin could also or instead be transferred to other cannabis producers. For example, a cannabis producer could purchase resin in bulk from another producer, and use this resin to create their own brand of cannabis oil. Operation 708 of FIG. 12 illustrates an example of resin packaging. Operation 708 receives an extract from the extraction at operation 706. Operation 708 also receives resin containers 726, which are examples of holding containers for non-oil extracts. Resin containers 726 could include stainless steel containers, for example.

FIG. 15 is a flow diagram illustrating an example method 1000 for resin packaging. The example method 1000 could be performed in a vertical laminar flow hood, for example, to help isolate an operator from any fumes produced by the resin. More generally, any or all processes that involve cannabis extracts could be performed in a laminar flow hood or other protective structure. Protective equipment, such as facemasks, could also or instead be used.

Step 1002 includes weighing an empty holding container. The measured weight could be recorded in the ICS. At step 1004, the holding container is filled with resin. Step 1004 could include transferring the resin from an extract collection vessel into the holding container, for example. Step 1004 could be performed manually and/or be automated by one or more devices.

After the holding container is filled, the holding container is weighed again at step 1006. This weight could be recorded in the ICS, and could be compared to the weight of the empty holding container to determine the weight of resin in the holding container. The weight of the resin in one or more holding containers could be compared to the weight of the resin in the collection vessel to help ensure consistency. A label could be generated for the holding container by the ICS, or a pre-existing label on the holding container could be recorded by the ICS. In some embodiments, the holding container could be associated with an extract record in the ICS, and the label on the holding container could include the extract record's identification number. Steps 1004, 1006 could be repeated multiple times, which is indicated using a dashed line in FIG. 15. For example, step 1004 could be performed twice, where in each instance a resin from a different extraction process is transferred to the same holding container. At step 1006, the holding container could be weighed after each transfer of resin to determine the weight of the respective resin that was added.

The holding container of resin is transferred to a storage area at step 1008. This transfer could be recorded in the ICS to help track the location of the holding container. In some embodiments, the storage area could be a cool, dry and/or dark area, such as a refrigerator, to help preserve the resin in the holding container.

The example packaging system 420j in FIG. 4J could be used in performing the example method 1000. The scale(s) 430j-1 and/or 430j-2 could be used to weight empty and full holding containers 456j, which could be filled and closed by the bottle filling/capping machine(s) 454j. Labelling and/or scanning could be performed by the label maker(s) 432j and/or scanners at 434j-1 and/or 434j-2. Information related to resin packaging could be transmitted from the packaging system 420j or components therein to the server 400 in FIG. 4A, through the server 418j in some embodiments, to populate or otherwise update the database 414 or particular records therein.

At least a portion of the resin that is collected during the extraction in method 900 of FIG. 14 could be used oil formulation. Oil formulation could be performed in addition to or instead of resin packaging. Oil formulation is the process of producing cannabis oils from cannabis extracts. In some embodiments, cannabis oils are produced by adding carrier oils to cannabis resin. The cannabinoid(s) in the resin could be infused into the carrier oil, which becomes a carrier for the cannabinoid(s). Referring again to FIG. 12, oil formulation is performed at operation 710. Operation 710 receives cannabis extracts from the extraction process at operation 706. Operation 710 also receives oil formulation supplies 720 and carrier oil supplies 722. Oil formulation supplies 720 could include, for example, holding containers, flasks, protective equipment, cleaning solutions and mixers. Carrier oil supplies 722 could include any of a variety of food grade oils, such as peppermint oil, fractionated coconut oil (also known as MCT oil), palm oil, olive oil, sunflower oil, canola oil, avocado oil, hemp seed oil and grape seed oil, for example. Carrier oil supplies 722 could include a mixture of two or more different carrier oils.

Any of various information relating to an oil formulation process and/or oil formulation cannabis products could be recorded in an ICS, in the form of an oil formulation record in some embodiments. An oil formulation record could include or otherwise be associated with an oil formulation record identifier, which could be similar in form to other identifiers disclosed herein. Oil formulation information related to an oil formulation process and/or oil formulation cannabis products could also or instead be recorded in another type of record such as a lot record.

A lot record could include an oil formulation record identifier or otherwise be associated with an oil formulation record for an oil formulation process that was used to produce a lot of oil formulation cannabis product, or multiple oil formulation records if the lot was dried in different equipment, using different oil formulation processes, and/or over multiple days for example. Similarly, an oil formulation record could include a lot identifier or otherwise be associated with a lot record, or multiple lot records if an oil formulation process or equipment produced multiple lots of oil formulation cannabis product.

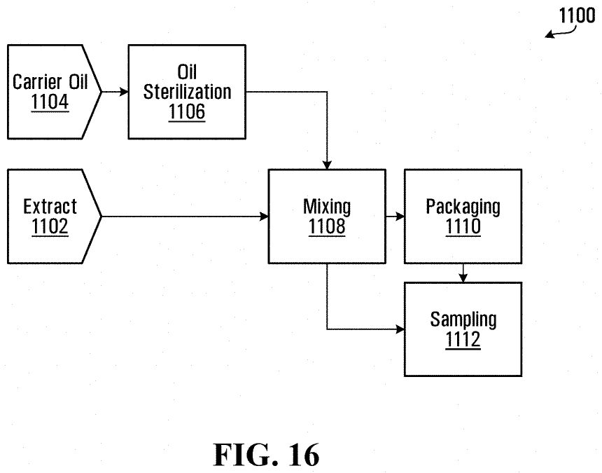

FIG. 16 is a flow diagram illustrating an example process 1100 for oil formulation. An extract 1102 and a carrier oil 1104 are inputs to the example process 1100. In some embodiments, extract 1102 is a cannabis resin produced by an extraction process. The resin could be received in a holding container or in an extract collection vessel, for example. Carrier oil 1104 could be provided by a supplier, and could include a single type of carrier oil or a mixture of multiple types of carrier oils, examples of which are provided elsewhere herein.

In some embodiments, at operation 1106, the carrier oil 1104 is sterilized. Operation 1106 could include transferring at least a portion of the carrier oil 1104 into a clean flask and measuring the volume and/or weight of the carrier oil. The mouth of the flask could then be covered, with aluminum foil for example. The filled flask could be transferred to a sterilization device or system, such as a dry heat sterilization (DHS) oven. In some embodiments, the oven is operated at 180° C. for 2.5 hours for sterilization. The flask could then be removed from the oven and allowed to cool. Sterilization indicator tape could be affixed to the flask before the flask enters the oven. At least a portion of the indictor tape could change colors if a particular temperature has been reached by the flask, which could indicate that sterilization was successful. If sterilization was successful, then the flask could be sealed, with a cap for example. Information relating to the sterilization and/or the flask could be labelled on the flask and/or recorded in the ICS. For example, any one or more of the time of sterilization, the date of sterilization, one or more parameters of the sterilization process, the carrier oil volume, and the carrier oil weight could be recorded on a label and/or in the ICS. Following sterilization, the flask could be stored, in a cool and dark location in some embodiments.

By way of example, FIG. 4K illustrates a sterilization system 420k. Such a system could be used in sterilization of carrier oil and not only for sterilization of cannabis products. In FIG. 4K, sterilization is through irradiation in the irradiation facility 452k. Carrier oil sterilization could also or instead involve heating using an oven instead of or in addition to the irradiation facility 452k. Recording of sterilization information could involve one or more scales such as the scale(s) 430k-1 and/or 431k-2, and/or one or more scanners such as the scanner(s) 434k-1 and/or 434k-2. Labelling of the source product holding container(s) 450k holding carrier oil and/or the target holding container(s) holding sterilized carrier oil, which could be the same containers in the case of carrier oil sterilization, could involve one or more label makers such as the label maker(s) 432k.

Sterilization of carrier oils might not be performed in all oil formulation processes. For example, the carrier oil 1104 could be sent directly to operation 1108 without first being sterilized.

One or more other initial treatments of the carrier oil 1104 could be performed, instead of or in addition to sterilization, before the carrier oil is used in operation 1108. For example, operation 1106 could include testing the carrier oil 1104 before and/or after sterilization, or testing could be performed independently of sterilization. A holding container containing untested carrier oil could be marked as “untested” or “quality hold” on a label and/or in the ICS, to indicate that the carrier oil has not yet been tested and approved for use. To perform testing, a sample of the carrier oil 1104 could be drawn from the holding container, using a dip tube, dipper or pipette for example, and transferred to a sample container such as a glass jar. The holding container could then be marked as “sampled” on the label and/or in the ICS. In some embodiments, the sample is tested for a United States Pharmacopeia (USP) monograph that is specific to a type of carrier oil. USP monographs provide standards for identity, quality, purity and/or strength for certain substances. USP monographs could confirm that the type of carrier oil being tested matches what is indicated on the label and/or what was ordered. By way of example, for testing olive oil, the USP monograph USP29-NF24 could be used. The carrier oil sample could also or instead be screened for heavy metal contaminants.

If the test of the sample returns satisfactory results, then the holding container that was sampled could be marked as “cleared for use” on the label and/or in the ICS. If the sample failed one or more tests, then a second sample could be drawn from the holding container and tested. If the second sample also fails the test, then the holding container could be marked as “not for use” on the label and/or in the ICS, and returned to the supplier of the carrier oil.

By way of example, FIG. 4L illustrates a testing system 420l. Such a system could be used in testing carrier oil and not only for testing cannabis products. Source product holding container(s) 450l, sampling container(s) 452l, and testing device(s) 454l are all shown in FIG. 4I, and could be used to hold and test carrier oil.

Recording of testing information could involve one or more scales such as the scale(s) 430l and/or one or more scanners such as the scanner(s) 434l. Labelling of the source product holding container(s) 450l holding carrier oil, sampled carrier oil, and/or tested carrier oil, and/or labelling of the sampling container(s) 452l, could involve one or more label makers such as the label maker(s) 432l.

For each of the manufacturing examples described above, a lot release process can be implemented. In some embodiments, a lot of cannabis product can be tested in order to ensure that the batch of cannabis product is within a certain cannabinoid concentration range (e.g. milligrams of THC per milliliter of cannabis product, or milligrams of THC per gram of cannabis product). In some embodiments, such testing can include Quality Assurance (QA) testing, and may be part of a Preventable Control Plan (PCP). In some embodiments, such QA testing can include allergen testing, label validation testing, microbiological testing, mycotoxins testing, nutritional analysis, organoleptic testing, testing for heavy metals, foreign materials toxins and/or other contaminants. The results of such concentration and QA tests can be recorded by the ICS in, for example, the database 414 on server 402.

In some embodiments, the aforementioned testing can be performed prior to packaging or bottling. In some embodiments, the aforementioned testing can be performed once the cannabis product has been bottled or packaged. In such an embodiment, the testing can be performed on a representative sample of the bottles or packages. Once the lot release testing is complete and a lot has passed any concentration and QA tests, the lot is released.

Any of various information relating to carrier oil testing could be recorded in an ICS, in the form of a carrier oil testing record in some embodiments. A carrier oil testing record could include or otherwise be associated with a carrier oil testing record identifier, which could be similar in form to other identifiers disclosed herein. Carrier oil testing information related to testing of a carrier oil could also or instead be recorded in another type of record such as a sterilization record or a carrier oil lot record.

A sterilization record or a lot record could include a carrier oil testing record identifier or otherwise be associated with a carrier oil testing record, or multiple carrier oil testing records if carrier oil the lot was tested in different equipment, using different testing processes, and/or over multiple days for example. Similarly, a carrier oil testing record could include a sterilization identifier and/or a lot identifier or otherwise be associated with a sterilization record and/or a lot record, or multiple sterilization and/or lot records if multiple carrier oil samples were tested at the same time and under the same testing conditions, for example.

With reference again to FIG. 16, at operation 1108 carrier oil 1104, which may have been sterilized, tested, and/or otherwise processed, is mixed with extract 1102. This mixing could include dissolving and/or suspending the extract 1102 in the carrier oil. In some embodiments, operation 1108 is intended to prepare a super saturated solution of cannabis resin and carrier oil, which could also be referred to as a cannabinoid concentrate. Operation 1108 could include weighing a holding container containing cannabis resin, and recording this weight in the ICS. Carrier oil could be transferred to a beaker or other vessel, and the weight and/or volume of the carrier oil in the beaker could also be recorded in the ICS. The carrier oil could then be transferred from the beaker to the holding container containing the cannabis resin.

In some embodiments, the carrier oil is transferred incrementally. For example, if the resin in the holding container weighs 50-100 g, then the carrier oil could be added in 10 mL increments until all of the resin is dissolved in the oil. If the resin weighs 100-300 g then the carrier oil could be added in 50 mL increments, and if the resign weighs over 300 g then the carrier oil could be added in 100 mL increments. Alternatively, a weight ratio of 3:2 for carrier oil to resin could be used. Other extract and carrier oil ranges, increments, and/or ratios are also possible, and such parameters could be determined and/or controlled dynamically in some embodiments.

Resin could be dissolved and/or suspended by a carrier oil without any additional actions to encourage mixing, however this might not always be the case. For example, a resin with a high wax content might not be dissolved by a carrier oil without performing additional actions. The following is a non-limiting list of actions that could be used to encourage dissolution of resin in carrier oil, and any one or more of these actions could be performed at 1108:
submerging at least a portion of the holding container containing the resin and the carrier oil in a heated water bath (for example, the temperature of the water bath could be 40° C.);
submerging at least a portion of the holding container containing the resin and the carrier oil in an ultrasonic water bath (for example, the holding container could be sonicated in 5 minute intervals);
placing the holding container on a heated stir plate (for example, the temperature of the stir plate could be set to 65° C.);
directing a heat gun at the holding container to dissolve resin that is adhered to the walls of the holding container;
sonication.

Adding carrier oil to the holding container and/or performing any additional actions to dissolve the resin could be repeated multiple times until the resin is substantially dissolved by the carrier oil. Two or more actions could be performed, possibly at the same time, to dissolve the resin in the carrier oil. For example, sonication and heating in a water bath could be performed simultaneously. Carrier oil could also or instead be added to a holding container while performing additional actions to dissolve the resin. Any or all of the actions taken to dissolve the resin could be recorded in the ICS. When the resin is substantially dissolved by the carrier oil, the solution could appear homogenous. After mixing, the weight of the beaker of carrier oil and/or the weight of the holding container containing the produced cannabis oil could be measured and recorded in the ICS. Comparing one or more of these weights to their initial weights could determine the weight of carrier oil that was added to the holding container. Using the weight/volume of the resin in the holding container, the weight/volume of the carrier oil that was added to the holding container, and the cannabinoid concentration in the resin (determined by prior testing, for example), the cannabinoid concentration in the produced cannabis oil could be determined. In some embodiments, multiple different resins and/or multiple different carrier oils could be mixed in operation 1108. Multiple different cannabis oils could also or instead be mixed together in operation 1108.

The operation 1108 could be recorded in the ICS using a “suspend” action, for example. For example, a suspension process could be recorded in the ICS the form of a suspension record, which could be assigned a suspension record ID. The suspend action could modify an extract record that is associated with extract 1102 to indicate that at least a portion of this extract is now suspended in a carrier oil. Using the suspend action, the volume of the cannabis oil produced in operation 1108 could be recorded in the ICS, and the extract record in the ICS could be updated to have that volume. The suspend action could record the holding container storing the produced cannabis oil as an “oil container”. The oil container record could be assigned an ID such as “OC-1”, “OC-2” or “OC-3”.

In some embodiments, operation 1108 could further include diluting cannabis oils with additional sterilized carrier oil. Dilution could be performed to achieve a particular cannabinoid concentration in the final cannabis oil. For example, a cannabis oil could be diluted such that the concentration of THC does not exceed 30 mg/mL. The volume and/or weight of carrier oil that should be added to the cannabis oil to reach a particular cannabinoid concentration could be calculated beforehand. Prior testing of the cannabinoid concentrations in the extract used to produce a cannabis oil, and/or in the cannabis oil itself, could help determine the amount of additional carrier oil that should be added during dilution. In the case that a specific weight of additional carrier oil is calculated, a flask of un-diluted cannabis oil could be placed on a scale and the weight of the flask could be monitored as carrier oil is added until the calculated weight is reached. Heated water baths, ultrasonic water baths and/or heated stir plates could be used to help homogenize the diluted solutions. In some embodiments, carrier oil could be added to the cannabis oil at a pre-defined dilution rate.

Dilution of a cannabis oil could be recorded in the ICS using a “dilute” action. Using the dilute action, an extract record and/or an oil container record could be selected for dilution. The weight and/or volume of cannabis oil that is being diluted could be measured and recorded in the ICS. The weight, volume and/or type of carrier oil added during dilution, as well as the final weight and/or volume of the diluted cannabis oil, could also be measured and recorded in the ICS. If the diluted cannabis oil is transferred to a different holding container, a new oil container record could be created by the dilute action.

FIG. 4I illustrates an example oil formulation system 420i. Holding containers for carrier oil, source cannabis product, and output cannabis oil(s) are shown at 452i, 450i, 458i, respectively. Recording of information related to an oil formulation process could involve such components as the scale(s) 430i-1 and/or 430i-2, the sensor(s) 428i, and/or the scanner(s) 434i-1 and/or 434i-2. Other components could also or instead be used to collect and/or enter oil formulation process information for recording in an ICS. The label maker(s) 432a could be used in labelling of any or all of the holding containers 452i, 450i, 458i.

As referred to herein, cannabis vaping oils have a viscosity and a flash point which are suitable for use in a vaping device where the vaping device is configured for using a vaping oil having a viscosity at room temperature below a threshold, and where the vaping device is further configured to heat the vaping oil at a vaporization temperature at which one or more cannabinoids in the vaping oil vaporize.

Generally speaking, several options exist to obtain cannabis vaping oil having the herein described desired viscosity and flash point for use in a vaping device.

A first option is to dilute a cannabis concentrate having a viscosity at room temperature which is above the threshold to the point of obtaining the desired viscosity with an additive having a flash point equal to or above the vaporization temperature. The dilution creates a mixture that has a sufficiently lower viscosity than the cannabis concentrate without the additive, while maintaining a flash point equal to or above the vaporization temperature for safely vaporizing one or more cannabinoids contained in the cannabis concentrate. Furthermore, when the mixture is loaded into a cartridge component of a vaping device with a pipette at room temperature, the mixture flows in and out of the pipette into the cartridge without much difficulty. In other words, the mixture behaves like a liquid.

A second option is to dilute a cannabis concentrate having a viscosity at room temperature which is above the threshold to the point of obtaining the desired viscosity with an additive having a flash point below the vaporization temperature. In this option, the cannabis concentrate has a flash point equal to or above the vaporization temperature such that the dilution creates a mixture that has a flash point equal to or above the vaporization temperature for safely vaporizing one or more cannabinoids contained in the cannabis concentrate. In this option, the proportions of cannabis concentrate and additive are selected such that the mixture has a viscosity below the threshold while maintaining a flash point equal to or above the vaporization temperature.

In a practical implementation, the additive includes a compound which operates to lower the viscosity of the cannabis concentrate. The additive can be a single material or a blend of different materials. Optionally, the rate of addition of the additive to the cannabis concentrate can be adjusted according to expected storage or the vaping device's operational parameters.

In one embodiment, the additive used in the present disclosure does not significantly alter the organoleptic properties of the cannabis concentrate; in other words, the taste, smell and touch of the cannabis concentrate is not significantly altered by the addition of the additive.

In an advantageous non-limiting embodiment, a single additive is added to the cannabis concentrate. This simplifies the manufacturing of the cannabis vaping oil and may increase regulatory approval likelihood by local regulatory bodies. However, it is also conceivable for two or more different additives to be added to the cannabis concentrate, especially when particular further advantageous properties are to be obtained. For example, a first additive having a flash point equal to or above the vaporization temperature may be used together with a second additive having a flash point below the vaporization temperature. In such situation, the overall proportion of cannabis concentrate required to obtain a suitable flash point for the whole mixture may not be as high compared to the situation where the additive(s) has (have) a flash point below the vaporization temperature. Accordingly, less cannabis concentrate may be required to have a cannabis oil with suitable flash point, although the person of skill may still wish to include higher proportion of cannabis concentrate in other to increase potency of the cannabis oil, i.e., increase the concentration of cannabinoid(s) in the cannabis vaping oil.

In one non-limiting embodiment, the cannabis vaping oil of the present disclosure includes a mixture of the cannabis concentrate and the additive, where the cannabis concentrate is present in a proportion of 40 wt. % relative to the weight of the additive. Preferably, the proportion of cannabis concentrate is 70 wt. % relative to the weight of the additive, such that the cannabis vaping oil retains sufficient free-flowing liquid properties to afford ease of use with the vaping device.

Examples of additives that typically have a flash point above the vaporization temperature include Vegetable Glycerin (VG), Polyethylene Glycol (PEG), and Propylene Glycol (PG). Objectively, those compounds are less desirable than other examples provided in this disclosure because they are known to potentially produce toxic and carcinogenic impurities as a result of the thermal decomposition of VG, PEG and PG.

In one non-limiting embodiment, the additive includes one or more carrier oil(s). In one non-limiting embodiment, the one or more carrier oil(s) is (are) of plant origin. For example, but without being limited to, terpenes, essential oils, and the like, such as for example, d-limonene, Orange sweet (Citrus sinensis), b-myrcene, Pine (Pinus sylvestris), Fir (Abies siberica or Abies balsamea), Juniper Berry (Juniperus communis), lemon Lime Flavor, peppermint oil, and the like.

In one non-limiting embodiment, the additive includes a medium chain triglyceride (MCI) or a mixture of MCT and another additive. For example, the additive can include a mixture of peppermint oil and MCT in proportions such that the typical taste of peppermint oil is tamed down with the MCT.

The cannabis concentrate of the present disclosure may be obtained with any known method in the art. For example, the cannabis concentrate may be obtained by a process including an extraction step from plant materials using heat decarboxylation to convert cannabinoids in their acid forms to neutral forms followed by or after CO₂ extraction (under sub-critical or super-critical conditions), and then, optionally, followed by ethanol winterization to remove waxes. Optionally, the method for obtaining the cannabis concentrate may further include purification steps such as a distillation step in order to further purify, isolate or crystallize one or more cannabinoids. A cannabis concentrate obtained by distillation may be further cut with one or more terpenes.

The cannabis concentrate includes one or more cannabinoid(s). Examples of cannabinoids include, but are not limited to, cannabigerolic acid (CBGA), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C4 (CBD-C4), cannabidivarin (CBDV), cannabidiorcol (CBD-C1), delta-9-tetrahydrocannabinol (Δ9-THC), delta-9-tetrahydrocannabinolic acid A (THCA-A), delta-9-tetrahydrocannabionolic acid B (THCA-B), delta-9-tetrahydrocannabinolic acid-C4 (THCA-C4), delta-9-tetrahydrocannabinol-C4, delta-9-tetrahydrocannabivarin (THCV), delta-9-tetrahydrocannabiorcol (THC-C1), delta-7-cis-iso tetra hydrocannabivarin, delta-8-tetrahydrocannabinol (Δ8-THC), cannabicyclol (CBL), cannabicyclovarin (CBLV), cannabielsoin (CBE), cannabinol (CBN), cannabinol methylether (CBNM), cannabinol-C4 (CBN-C4), cannabivarin (CBV), cannabinol-C2 (CBN-C2), cannabiorcol (CBN-C1), cannabinodiol (CBND), cannabinodivarin (CBVD), cannabitriol (CBT), 10-ethoxy-9hydroxy-delta-6a-tetrahydrocannabinol, 8,9-dihydroxy-delta-6a-tetrahydrocannabinol, cannabitriolvarin (CBTV), ethoxy-cannabitriolvarin (CBTVE), dehydrocannabifuran (DCBF), cannabifuran (CBF), cannabichromanon (CBCN), cannabicitran (CBT), 10-oxo-delta-6a-tetrahydrocannabionol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-H HCV), cannabiripsol (CBR), trihydroxy-delta-9-tetrahydrocannabinol (triOH-THC), cannabinol propyl variant (CBNV), and derivatives thereof.

In some embodiments, the cannabinoid is tetrahydrocannabinol (THC). THC is only psychoactive in its decarboxylated state. The carboxylic acid form (THCA) is non-psychoactive. Delta-9-tetrahydrocannabinol (Δ9-THC) and delta-8-tetrahydrocannabinol (Δ8-THC) produce the effects associated with cannabis by binding to the CB1 cannabinoid receptors in the brain.

In some embodiments, the cannabinoid is cannabidiol (CBD). The terms “cannabidiol” or “CBD” are generally understood to refer to one or more of the following compounds, and, unless a particular other stereoisomer or stereoisomers are specified, includes the compound “Δ2-cannabidiol.” These compounds are: (1) Δ5-cannabidiol (2-(6-isopropenyl-3-methyl-5-cyclohexen-I-yl)-5-pentyl-I,3-benzenediol); (2) Δ4-cannabidiol (2-(6-isopropenyl-3-methyl-4-cyclohexen-I-yl)-5-pentyl-I,3-benzenediol); (3) Δ3-cannabidiol (2-(6-isopropenyl-3-methyl-3-cyclohexen-I-yl)-5-pentyl-I,3-benzenediol); (4) Δ3,7-cannabidiol (2-(6-isopropenyl-3-methylenecyclohex-I-yl)-5-pentyl-I,3-benzenediol); (5) Δ2-cannabidiol (2-(6-isopropenyl-3-methyl-2-cyclohexen-I-yl)-5-pentyl-I,3-benzenediol); (6) Δ1-cannabidiol (2-(6-isopropenyl-3-methyl-I-cyclohexen-I-yl)-5-pentyl-I,3-benzenediol); and (7) Δ6-cannabidiol (2-(6-isopropenyl-3-methyl-6-cyclohexen-1-yl)-5-pentyl-I,3-benzenediol).

In one embodiment, the cannabis oil of the present disclosure includes ≥300 mg/ml of CBD, for example, ≥650 mg/ml, ≥550 mg/ml, ≥550 mg/ml, ≥460 mg/ml, ≥450 mg/ml, ≥400 mg/ml, and the like.

In one embodiment, the cannabis oil of the present disclosure includes ≥300 mg/ml of THC, for example, ≥650 mg/ml, ≥550 mg/ml, ≥550 mg/ml, ≥460 mg/ml, ≥450 mg/ml, ≥400 mg/ml, and the like.

In one embodiment, the cannabis oil of the present disclosure includes ≥300 mg/ml of CBD and ≤30 mg/ml THC, for example, 30 mg/ml, ≤25 mg/ml, ≤20 mg/ml, and the like.

The cannabis oil of the present disclosure can be used in any suitable cartridge component of a vaping device.

Packaging of cannabis oils is illustrated at operation 1110 in FIG. 16. Operation 1110 includes packaging diluted and/or un-diluted cannabis oils in holding containers such as bottles. Packaging could include assigning a lot number to an oil container record using a new lot action, for example. All of the holding containers that contain cannabis oil from that oil container record could be labelled with or otherwise identified by that lot number.

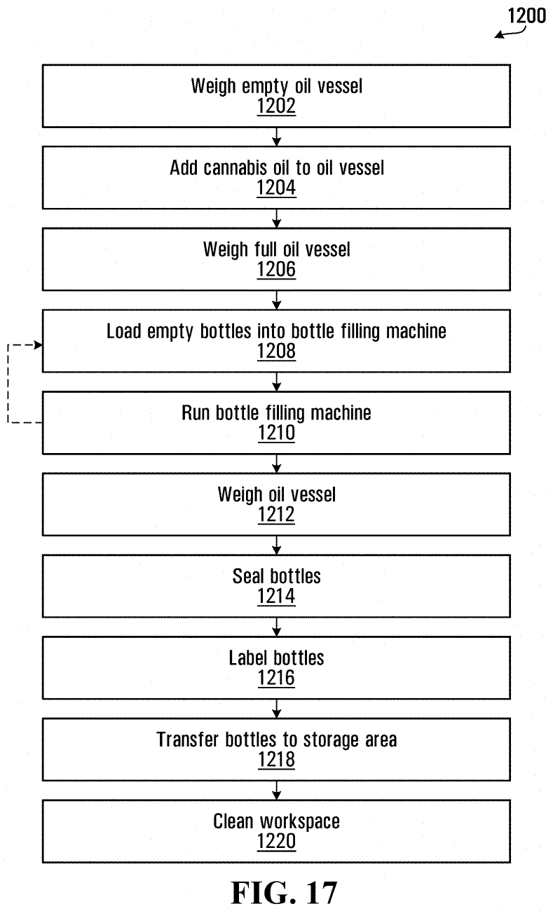

In some embodiments, cannabis oils are packaged into bottles using an automated bottle filling machine, such as the bottle filling/capping machine(s) 454J in FIG. 4J. FIG. 17 is a flow diagram illustrating an example method 1200 for oil packaging using a bottle filling machine.

At step 1202, an empty oil vessel from which cannabis oil is supplied to the bottle filling machine during a production run is weighed. In some embodiments, this oil vessel is a pressure vessel. Weighing the oil vessel could include removing and/or decoupling the oil vessel from the bottle filling machine and placing the oil vessel on a scale. Alternatively, the oil vessel could be weighed while coupled to the bottle filling machine. In this case, the oil vessel and/or bottle filling machine could have an integrated scale to measure the weight of the oil vessel.

At step 1204, cannabis oil is added to the oil vessel. The cannabis oil, and/or any holding container(s) in which the cannabis oil was stored, could be recorded in the ICS. By way of example, cannabis oil could be stored in one or more of the source product holding container(s) 450j in FIG. 4J.

At step 1206, the oil vessel is weighed again, to determine the weight of cannabis oil that was added to the oil vessel. Any or all of the weights measured in steps 1202, 1206 could be recorded in the ICS, and/or these weights could be used to determine cannabis oil weight for recording in the ICS. Cannabis oil could be otherwise measured, by volume, for example. In some embodiments, volume of oil added to the oil vessel from a holding container could be metered as it is added.

If appropriate, the oil vessel could then be re-coupled to the bottle filling machine. For example, an input hose of the bottle filling machine could be placed into or otherwise fluidly connected to the contents of the oil vessel.

Step 1208 includes loading empty bottles into the bottle filling machine. In some embodiments, the bottles could be added to a tray that is loaded into the bottle filling machine. These bottles could be cleaned, dried and/or sterilized to prevent contamination of the cannabis oil. In some embodiments, the bottle filling machine is part of a production line, and is supplied with bottles as they are needed, by a conveyor system for example. Bottles used in a bottle filling machine represent an example of the target holding container(s) 456j in FIG. 4J.

The bottle filling machine is then run at step 1210. Before the run, any pneumatic connections, electrical connections, valves and/or tubing on the bottle filling machine could be cleaned, inspected, aligned and/or tested to confirm that they are installed and/or operating correctly. During a run, the bottle filling machine draws cannabis oil from the oil vessel and adds a pre-defined volume of cannabis oil to each of the empty bottles. The bottle filling machine could include a controller to adjust the volume of cannabis oil that is added to each bottle, a rate at which the cannabis oil is added and/or a number of bottles filled.

Some settings of the bottling filling machine could be adjusted based on properties of the cannabis oil. For example, a pressure that is applied to the cannabis oil during a run could be adjusted based on viscosity of the oil. The controller could be connected or otherwise have access to the ICS to receive and/or record any or all settings of the bottle filling machine.

Settings for a bottle filling machine could be controlled manually, be predefined in a controller, and/or received or otherwise obtained or determined by the controller. Bottle filling machine operation could also or instead be actively monitored by an operator, and/or one or more sensors such as the sensor(s) 428j in FIG. 4J, to determine or adjust settings. A controller and/or one or more sensors could be connected to or have access to the ICS to record the bottle filling machine settings and/or one or more parameters of the production run. Alternatively, information could be manually recorded in the ICS for a drying process, using a computer such as 424j in FIG. 4J, for example.

In some embodiments, the bottle filling machine could be run multiple times before the oil vessel is emptied. For example, a full oil vessel could contain enough cannabis oil to supply two or more runs of the bottling filling machine. In these embodiments, steps 1208, 1210 could be performed multiple times, as indicated using a dashed line in FIG. 17.

At step 1212, the oil vessel is weighed again. This weight could be recorded in the ICS and/or used to determine and/or confirm the weight of cannabis oil that was added to the bottles in step 1210. Any unused cannabis oil in the oil vessel could be returned to the original holding container, or transferred to a new holding container. This transfer could be recorded in the ICS.

At step 1214, the filled bottles are sealed. This could include removing the bottles from the bottle filling machine and loading them into a capping machine to apply caps to the bottles, for example. Capping could be performed, entirely or in part, manually by one or more operators. In some embodiments, capping could be performed in the same equipment as bottling, as in the example packaging system 420j in FIG. 4J. Each bottle could be weighed, by one or more scales such as the scale(s) 430j-2 in FIG. 4J, to determine whether the correct amount of cannabis oil was added. If the weight of a bottle differs from a target weight by more than a threshold, such as 5% for example, then the bottle could be rejected, and its contents could be recycled into another production run or destroyed.

At step 1216, labels are applied to the bottles. These labels could be generated by the ICS, and could include any of various types of information regarding the cannabis oil that they contain. The label could also or instead include the volume of cannabis oil in the bottle, the date on which the bottle was packaged, and/or any other information regarding the bottle contents. The label maker(s) 432j and the scanner(s) 434j-2 in FIG. 4J are examples of system components that could be configured for marking holding containers and scanning markings on holding containers, respectively. Either or both of these components could transmit label information to other components for storage in or updating of an ICS, in the database 414 in FIG. 4A for example.

At step 1218, the bottles are transferred to a storage area, examples of which are provided elsewhere herein. Any such transfer could be recorded in an ICS to help track the location of a holding container.

At step 1220, the workspace, bottle filling machine(s) and/or oil vessel(s) are cleaned. Step 1220 could include washing or flushing certain components of the bottle filling machine with solvents (for example, water and/or ethanol) and/or compressed air.

Any of various components of a packaging system, such as the example packaging system 420j in FIG. 4J, could be configured to generate, collect, and/or otherwise obtain information and transmit that information to the server 402 in FIG. 4A, through the server 418j in some embodiments, for populating and/or updating the database 414 or particular records therein. This includes the components which are referenced by way of example above in the description of FIG. 17, and/or possibly other components.

In some embodiments, a holding container that contains cannabis oil could be recorded in the ICS as an “oil jar record”. By way of example, each bottle that was filled with cannabis oil in the example method 1200 could be recorded as an oil jar record. Oil jar records could be identified as “JAR-1”, “JAR-2” and “JAR-3”. These records could be created using a “new oil jar” action, for example. The new oil jar action could record, for example, any one or more of the following information:
the label(s) on the holding container;
the volume of cannabis oil in the holding container;
the weight of the holding container; and
the location of the holding container.

Multiple holding containers of cannabis oil could also or instead be recorded in the ICS as an oil jar record. In this case, a “new bulk” action could be used to create the oil jar record, for example. The new bulk action could record, for example, any one or more of the following information:
an oil container record associated with the holding containers;
the number of holding containers;
the volume of cannabis oil in each holding container;
the weight of cannabis oil in each holding container; and
the weight of each empty holding container.

Referring again to FIG. 16, at least a portion the cannabis oil produced at operation 1108, and/or a portion of the cannabis oil packaged at operation 1110, could be sent for sampling at operation 1112. Sampling could be performed to collect cannabis oil for testing and/or archiving for future testing.

A holding container of cannabis oil that has been selected for sampling could be weighed, using a scale 430l in FIG. 4L for example, and this weight could be recorded in the ICS as a “pre-sample weight”. A sample container such as a glass vial, shown by way of example in FIG. 4L at 452l, could be used to hold the sample of cannabis oil. The weight of the empty sample container could be measured, using a scale 430l in FIG. 4L for example, and recorded in the ICS. Cannabis oil could then be transferred from the holding container into the sample container, using a pipette for example. The amount of cannabis oil that is transferred could be pre-determined. For example, a desired sample volume could be 50 mL. The sample container could then be closed, using a cap and/or an induction seal for example. The filled sample container and/or the holding container that contained the cannabis oil could be reweighed, using a scale 430l in FIG. 4L for example. Any or all measured weights could be recorded in the ICS. The weight of the holding container after sampling could be recorded as a “post-sample weight”, which might be used to determine or confirm the weight of cannabis oil that was transferred to the sample container. The sample container could then be labelled, using a label maker(s) 432l in FIG. 4L for example, and/or stored in a storage area to await testing. In some embodiments, samples are tested when they are taken.

A sampling process could be recorded in the ICS using a “create sample” action, for example. This action could create a “lab sample” record in the ICS to record any of various information regarding a given sample of cannabis oil. Lab sample records could be assigned specific identifiers, such “LS-1”, “LS-2” and “LS-3”, for example. When a lab sample is sent for testing, the associated lab sample record could be labelled as “sent to lab” in the ICS.

Although the sampling process described above primarily relates to cannabis oils, a similar process could be performed to collect samples of other cannabis products, such as resin and/or plant material. Similar processes could also or instead be applied to sampling cannabis plants during cultivation, harvest and/or plant part separation. In some embodiments, samples of cannabis products could be archived and possibly tested at a later date.

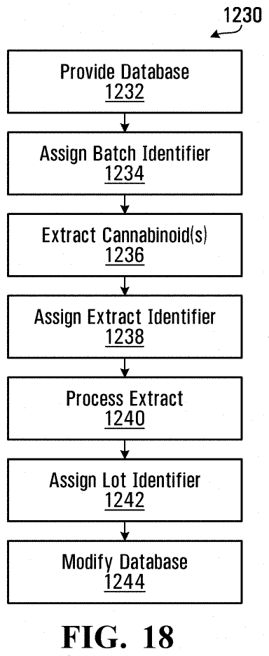

FIG. 18 is a flow diagram illustrating an example method according to another embodiment relating to cannabis extracts. The example method 1230 involves providing, at 1232, a database to store information associated with cannabis plants and cannabis products, and assigning, at 1234, a batch identifier to a batch of the cannabis plants. These operations are described by way of example above, with reference to FIG. 6, for example. FIG. 6 and the description thereof refer to processing plant material using first and second processes. The example method 1230 relates to a process, at 1236, of extracting one or more cannabinoids from the plant material of a portion of the cannabis plants in the batch using an extraction method to produce a cannabis extract. In some embodiments, extracting cannabinoids from the plant material at 1236 involves performing supercritical CO₂ extraction of cannabinoids. Extracting cannabinoids from the plant material at 1236 could also or instead involve distilling the cannabis extract, and/or other operations as disclosed elsewhere herein.

An extract identifier is assigned to the cannabis extract at 1238. An extract identifier could include alphanumeric characters and/or other symbols, and could be managed and/or assigned in the same way as lot identifiers or batch identifiers, for example.

An amount of the cannabis extract is processed at 1240 to produce units of a cannabis product. The processing at 1240 could involve, for example, any one or more of:
metering out amounts of the cannabis extract, illustratively by weight and/or by volume;
diluting the cannabis extract with one or more diluents such as a carrier oil;
emulsifying the cannabis extract to create a cannabinoid emulsion;
distilling the cannabis extract to produce a distillate;
metering out amounts of the distillate, illustratively by weight and/or by volume;
diluting the distillate with one or more diluents such as water and/or oil;
emulsifying the distillate to create a cannabinoid emulsion.

Examples of these processes are disclosed elsewhere herein.

At 1242, a lot identifier is assigned to a lot of the units of the cannabis product, and the database is modified at 1244 to include information relating to the batch identifier, the extract identifier and the lot identifier, with the lot identifier being associated with the extract identifier and the extract identifier being associated with the batch identifier. Lot delineation, lot identifiers, modifying a database, and identifier associations are all disclosed by way of example elsewhere herein.

For instance, similar to an example described above, modifying the database at 1244 could involve creating a lot record for the lot of units of a cannabis product. The lot record could include information conveying or indicating the lot identifier associated with the lot and information conveying or indicating at least one of the batch identifier and the extract identifier associated with the lot identifier.

A lot record could include other information, such as information indicative of the process or processes used at 1240 to produce the units of cannabis product.

In some embodiments, the lot record further includes information indicative of the number of units of a cannabis product contained in the lot. This type of information could be useful in confirming that the cannabis extract was used to produce an expected number of units of a cannabis product, for example.

Other examples of information that could be part of a lot record include information indicative of at least one of:
the time of the extracting that was used to produce the cannabis extract;
the date of the extracting that was used to produce the cannabis extract;
the time of the processing that was used to produce the units of cannabis product contained in the lot;
the date of the processing that was used to produce the units of cannabis product contained in the lot.

The method 1230, like other methods disclosed herein, is an illustrative example. Other embodiments could involve performing operations in a different order than shown, and/or performing different operations instead of or in addition to those shown in FIG. 18. For example, units of a cannabis product could be packaged, for storage and/or shipment, and a method could involve packaging each of the units of the cannabis product to produce product packages. Each product package could be marked with product information indicative of the lot identifier. A product package could be marked with other information in some embodiments.

Product information could be generated, at least in part, from information retrieved from the database, and examples of product information generation are disclosed elsewhere herein.

Marking each product package could involve marking the product package directly, and/or printing a label including the product information and affixing the label to the package. In some embodiments, a method involves retrieving information from the database, and generating the label using information retrieved from the database.

The product information with which a product package is marked could include at least one of the following:
information conveying or indicating an identity or contact information of a licensed producer of the cannabis product;
information conveying or indicating an identity or contact information of a licensed processor of the cannabis product;
information conveying a brand name of a cannabis product;
information conveying recommended storage conditions of a cannabis product;
information conveying a packaging date of a cannabis product.

The example method 1230 has been described above in the context of extracting one or more cannabinoids from plant material of a portion of cannabis plants in one batch. It should be appreciated, however, that extracting cannabinoid(s) at 1236 could also include extracting cannabinoids from plant material of a portion of cannabis plants in a second batch of cannabis plants using an extraction method to produce the same or a different cannabis extract, with the second batch of cannabis plants having a second batch identifier. The modifying at 1244 could then involve modifying the database to include information conveying or indicating the second batch identifier, and to associate the extract identifier with the second batch identifier.

Other variations of the example method 1230 may be or become apparent to those skilled in the art.

A processor-readable storage medium could be used in implementing a method, with processor-executable instructions being stored on such a medium. The instructions, when executed by a processor, cause the processor to perform a method. Execution of the instructions could cause a computing device that includes the processor to implement a system configured to, in some embodiments: implement a database configured to store information associated with cannabis plants and cannabis products; assign a batch identifier to a batch of the cannabis plants; receive extraction information relating to the extraction of one or more cannabinoids from the plant material of a portion of the cannabis plants in the batch using an extraction method to produce cannabis extract; assign an extract identifier to the cannabis extract; receive processing information related to the processing of an amount of the cannabis extract to produce units of a cannabis product; assign a lot identifier to a lot of the units of the cannabis product; and modify the database to include information relating to the batch identifier, the extract identifier and the lot identifier, with the lot identifier being associated with the extract identifier and the extract identifier is associated with the batch identifier.

Examples of many of these features are described above with reference to FIG. 18. A system implemented by a computing device could be configured to implement a database in one or more memory devices, for example, to store plant and product information, examples of which are described above and elsewhere herein. Such a system could also be configured to assign a batch identifier, an extract identifier, and a lot identifier, and to modify the database as described above and elsewhere herein.

FIG. 18 and the description thereof refer to extracting one or more cannabinoids from plant material and processing cannabis extract to produce units of a cannabis product. A production system could include equipment such as extraction equipment to extract cannabinoids from plant material and processing equipment to process cannabis extract. A system that is implemented by a computing device might not itself include such equipment, but could be part of a production system, or at least communicate with equipment in a production system. A system that is implemented by a computing device could receive information from production system equipment, for example. In an embodiment, such a system is configured to receive extraction information relating to the extraction of one or more cannabinoids from the plant material using an extraction method to produce cannabis extract, and to receive processing information related to the processing of an amount of the cannabis extract to produce units of a cannabis product. Any of various types of processing information could be received, and examples of information relating to extraction and processing are disclosed elsewhere herein. Different types of operations, such as extraction and processing, could have different types of related information.

Extraction information could be used to assign an extract identifier, and processing information could be used to assign a lot identifier to cannabis product units in a lot. For example, an extract identifier could be assigned based on a type of extraction as conveyed or indicated in the extraction information, and/or a lot identifier could be assigned based on a type of process, conveyed or indicated in the processing information, that was used to produce the units in a lot.

A system implemented by a computing device could be configured to provide other features disclosed herein.

Other implementations are also contemplated. For example, the features described above with reference to FIG. 18 could involve various components of the example system 400 illustrated in FIGS. 4A-4M.

Sterilization is shown at 1106 in FIG. 16 for carrier oil, but could also or instead be performed on cannabis material and/or cannabis products at any of various stages of production. For example, sterilization could be performed during or following harvest, plant part separation, drying, milling, decarboxylation, pre-rolling, extraction and/or packaging. Sterilization could be performed before and/or after cannabis products are assigned lot numbers. A sterilization process could be recorded in the ICS, in the form of a sterilization record for example, which could be assigned a sterilization record ID.

Irradiation is one method for sterilizing cannabis products, as in the example sterilization system 420k in FIG. 4K. During an irradiation process, ionizing radiation could be directed towards a cannabis product to kill bacteria and/or other organic material that is present on and/or in the cannabis product. Examples of ionizing radiation include gamma rays, X-rays and electron beams. In some embodiments, ionizing radiation could penetrate the walls of the holding containers that contain a cannabis product, such as the source product holding container(s) 450k in FIG. 4K, and therefore a cannabis product might not be removed from a holding container during irradiation. Irradiation processes could be performed by a cannabis producer, but this might not always be the case. For example, cannabis products could be sent to another company for irradiation.

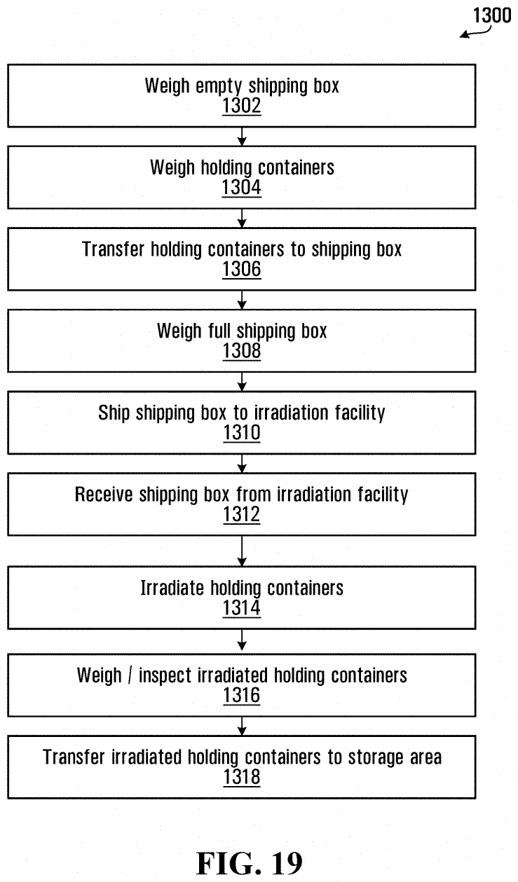

FIG. 19 is a flow diagram illustrating an example method 1300 for cannabis product irradiation. In the example method 1300, step 1302 includes weighing an empty shipping box that is used to ship cannabis products to an external facility for irradiation. This irradiation facility could be owned and/or managed by the producer of the cannabis product, or it could be owned and/or managed by another company. The weight of the empty shipping box could be recorded in the ICS. Although the example shipping system 420m in FIG. 4M is described above primarily in the context of customer order fulfillment, a similar system could be used to ship cannabis product for irradiation. For example, empty shipping boxes could be weighed by one or more scales such as the scale(s) 430m.

At step 1304, the holding containers that contain the cannabis product to be irradiated are weighed, by one or more scales such as the scale(s) 430m in FIG. 4M. These weights could be recorded in the ICS as “pre-irradiation” weights. The holding containers are then transferred to the shipping box at step 1306, and the full shipping box is weighed at step 1308, by one or more scales such as the scale(s) 430m in FIG. 4M. The weight of the full shipping box could be recorded in the ICS, and compared to the combined weights of the holding containers and the empty shipping box to confirm that the full shipping box weight is consistent with the total weights of the empty shipping box and the holding containers. A label for the shipping box could be generated by the ICS and/or recorded by the ICS. Labelling and/or recording could involve components such as one or more label makers and/or one or more scanners, examples of which are shown at 432m and 434m in FIG. 4M.

One or more tamper evidence seals could be provided on the shipping box. Such seals could be incorporated into labels or separate from labels.

At step 1310 the shipping box is shipped to the irradiation facility, and at step the 1312 the shipping box is received from the irradiation facility sometime later. Receiving the shipping box could include recording the label on the shipping box in the ICS. The ICS could then be updated to indicate that the shipping box has been received. The shipping box could also be weighed and compared to the weight that was recorded at step 1308 to confirm that no cannabis products were lost or added. A received shipping box and the holding containers inside the shipping box represent examples of the source product holding container(s) 450k in FIG. 4K. Weight and/or label recording could involve one or more scales 430k-1 and/or one or more scanners 434k-1, for example.

At 1314, the holding containers are irradiated, in the irradiation facility 452k in FIG. 4K for example. The holding containers could be removed from the shipping box before irradiation, or irradiated without being removed from the shipping box.

Step 1316 includes weighing the holding containers, individually and/or in the received shipping box, and recording these weights as “post-irradiation” weights in the ICS. The weights measured at step 1314, using the scale(s) 430k-2 for example, could be compared to the weights that were measured at step 1304 and/or step 1308, to confirm or re-confirm that no cannabis products were lost or added. Step 1316 could include inspecting the irradiated holding containers. For example, tamper detection devices on the holding containers could be inspected to detect any evidence of tampering. Step 1316 could further include sampling one or more of the irradiated holding containers to test the effectiveness of the irradiation process, for example.

At step 1318, the holding containers are transferred to one or more storage areas, to await further processing and/or shipping for example. Any such transfer could be recorded in the ICS.

Any of various components of a shipping system and/or a sterilization system, such as the example systems 420m, 420k in FIGS. 4M and 4J, could be configured to generate, collect, and/or otherwise obtain information and transmit that information to the server 402 in FIG. 4A, through the servers 418m, 418j in some embodiments, for populating and/or updating the database 414 or particular records therein. This includes the components which are referenced by way of example above in the description of FIG. 19, and/or possibly other components.

Testing of cannabis products is disclosed herein by way of example, and could be performed on cannabis material and/or cannabis products at any or all stages of production. For example, testing could be performed before and/or after sterilization. In some embodiments, sampling might first be performed to collect a representative sample of a cannabis material and/or cannabis product for testing. For example, the number n of holding containers to be selected for testing, from a lot of cannabis product, could be defined as n=1+\sqrt{N}, where N is the total number of holding containers in the lot. Other selection criteria could also or instead be used. Some the samples could be tested when taken, and/or some samples could be stored as archived samples for future testing.

Testing could be performed by a cannabis producer, and/or by another entity. A test could be recorded in the ICS, in the form of a test record for example, which could be assigned a test record ID. In some embodiments, a lot number is not assigned to a cannabis product until after at least one sample of the cannabis product has passed one or more quality assurance tests. The result(s) of the test(s) could be recorded in the ICS and/or on a label of the cannabis product. For example, a cannabinoid concentration that is determined for a cannabis product through testing could be recorded in the ICS using a new lot action, and/or added to a label.

Testing could also or instead be used to determine safety and/or effectiveness of the holding containers that contain a cannabis product. For example, a sample of holding containers for cannabis oil could be tested for leakage before and/or after the holding containers are filled with cannabis oil.

Testing for leaks could be performed in any of a variety of ways. In some embodiments, one or more holding containers could be filled with a test liquid and positioned over a clean piece of blotting paper or any other material that stains or otherwise changes appearance upon contact with a liquid. In one example of leak testing, the holding containers could be positioned at an inverted angle of 45° below horizontal with a container closure in the lowest position and free of any obstruction. After a particular amount of time, such as one hour, the blotting paper could be examined for any evidence of leakage. If a visual examination of the blotting paper discloses any trace of the test liquid, then the holding container has failed the leakage test and a holding container of the same type might not be used for packaging cannabis products. If no trace of the test liquid is found on the paper, the sample has passed the leakage test and a holding container of the same type could be used for cannabis oil. The result(s) of the leak test(s) could be recorded in the ICS. Other tests of holding containers could include testing tamper detection seals and/or child-resistant features, for example.

A testing system 420l is shown by way of example in FIG. 4L. Such a testing system could be used to test cannabis material and/or cannabis products, and holding container testing could be implemented in the same or a similar manner.

In each of the aforementioned systems, the difference between the weight of material input into a process (or series of processes) and the weight of material output from the process (or series of processes) can be compared against any amount of waste output from the process (or series of processes) in order to assess lost and/or theft of material. This information can then be recorded by the ICS in, for example, the database 414 on server 402.

Packaging and Shipping

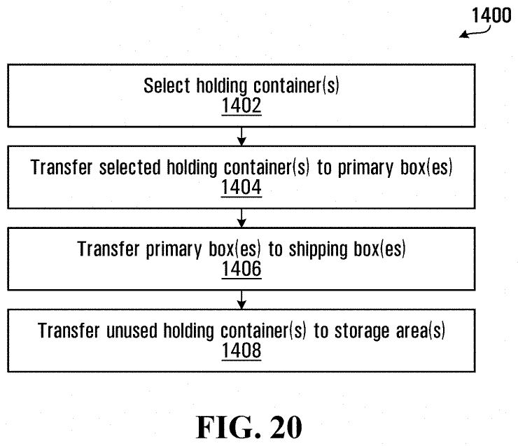

Final packaging and shipping could be managed by an ICS. The ICS could include a database to store information related to final packaging and shipping, for example. FIG. 20 is a flow diagram illustrating an example method 1400 for final packaging. Method 1400 could be similar to the final packaging performed at operation 122 in FIG. 1.

At step 1402, one or more holding containers containing the same or different cannabis products are selected for final packaging. By way of example, FIG. 4M illustrates one or more selected holding containers 452m.

The number of holding containers selected at step 1402, and/or the type(s) of cannabis product(s) stored in these holding containers, could be based on a customer order. The selection could be performed automatically by the ICS, and/or manually by an operator. Labels on the holding containers could be used to help identify the desired holding container(s) for selection. Step 1402 could include selecting and removing the holding containers from a storage area, for example. Step 1402 could also or instead include selecting and transferring holding containers directly from a production system or process. In some embodiments, cannabis products from one or more holding containers could be transferred to one or more different holding containers at step 1402. For example, a lot of cannabis product could be stored in a large holding container, and then transferred to multiple smaller holding containers during final packaging.

The holding containers selected at step 1402 could be transferred to a packaging and/or shipping area or equipment, and the weight of and/or other information related to each selected holding container could be recorded in the ICS. One or more scales such as the scale(s) 430m in FIG. 4M could measure weight(s) of selected holding container(s) 452m. The scanner(s) 434m in FIG. 4M are illustrative of devices that could collect other information related to each selected holding container 452m.

Step 1404 includes transferring the selected holding container(s) to one or more primary boxes. In some embodiments, a primary box could store all of the holding containers that have been selected to meet a customer order. The selected holding container(s) could be compared to a customer order as they are transferred to the primary box to confirm that the order is being met. Protective materials, such as bubble wrap and/or Styrofoam for example, could be added to the primary box(es) to help protect the holding container(s) during shipping. Insulation could also or instead be added to protect the holding container(s) from hot or cold environments during shipping.

Step 1404 could include adding or updating labels on the holding container(s) before they are added to the primary box(es). Labels could also or instead be added to and/or updated on the primary box(es). In some embodiments, a label associated with the customer could be added to the primary box(es) and/or the holding container(s), using one or more label makers such as the label maker(s) 432m in FIG. 4M.

The ICS could record all of the holding container(s) that are transferred to the primary box(es) by scanning their labels, for example. The ICS could also record any or all labels on the primary box(es). One or more scanners such as the scanner(s) 434m in FIG. 4M could be used for container, primary box and/or label scanning.

In some embodiments, as shown at step 1406, the primary box(es) are transferred into one or more shipping boxes. The shipping box(es) could include protective and/or insulating materials to help protect the cannabis products. In some embodiments, these shipping boxes could be specific to a courier service that is used to ship cannabis products. Similar to the primary box(es), the shipping box(es) could be labelled and/or recorded in an ICS, using the label maker(s) 432m and/or scanner(s) 434m in FIG. 4M for example. The weight of the shipping box(es), and/or the primary box(es), could also or instead be recorded in the ICS, using the scale(s) 430m in FIG. 4M for example. In some embodiments, orders are placed directly into shipping boxes, and no separate primary boxes are used.

Labels on shipping boxes could include shipping information, such as the name and address of the customer. Step 1406 could include transferring the shipping boxes to a pick-up location for a courier service, and/or actually shipping the shipping box(es). Any such transfer and/or shipping could be recorded in the ICS. Other information such as the date, time and/or location of final packaging and/or shipping could also or instead be recorded in the ICS.

Step 1408 includes transferring any unused holding containers to one or more storage areas. Any such transfer could be recorded in the ICS. For example, a number of containers could be retrieved from storage, but only some of those containers might be selected for order fulfillment. The remaining containers could then be returned to storage.

The following example describes a specific example implementation of the method 1400 for final packaging of dried cannabis according to a customer order. At step 1402, a holding container of dried cannabis is selected by the ICS to meet the customer order. The ICS indicates that the holding container is stored in a particular storage area. Using a barcode scanner, an operator could locate the holding container and scan the label on the holding container to confirm that it is the holding container selected by the ICS. The holding container could then be transferred to a final packaging area. Step 1402 also includes transferring the dried cannabis from the holding container into multiple bags, which are recorded in the ICS. The number of bags and the weight of dried cannabis in each bag could be specified by the customer order and/or the ICS. These bags are then transferred to a primary box at step 1404, which is also recorded in the ICS. The primary box is then transferred to a courier shipping box at step 1406, and the shipping box is placed in a courier pick-up location. At step 1408, the dried cannabis that remains the original holding container could be sealed, and the holding container could be transferred back to the storage area.

Shipping could be performed by a courier service and/or postal service, however other means for shipping are also possible. Cannabis products could be shipped to a store or a private residence. Cannabis products could also or instead be shipped to another cannabis producer, in a bulk transaction for example. Further, cannabis products could be shipped to another producer for further processing, as discussed elsewhere herein.

The ICS could record and track any or all aspects of final packaging and shipping. For example, the weight, volume and/or type of any or all products that undergo final packaging and shipping could be recorded in the ICS. Information related to shipping destination(s) could also or instead be recorded in the ICS. A tracking number that is assigned to a shipped package could be recorded in the ICS. The ICS could have access to a package tracking system provided by a courier/postal service to actively track the location of a shipped package. Proof of delivery could also or instead be recorded in the ICS. In some embodiments, the amount of cannabis products shipped to each customer could be recorded to ensure that any allowance and/or shipping limits are not exceeded. The ICS could convert the shipped cannabis products into an equivalent amount of cannabis plants for the purposes of the recording.

Any of various components of a shipping system such as the example shipping system 420m in FIG. 4M, could be configured to generate, collect, and/or otherwise obtain information and transmit that information to the server 402 in FIG. 4A, through the server 418m in some embodiments, for populating and/or updating the database 414 or particular records therein. This includes the components which are referenced by way of example above in the description of FIG. 20, and/or possibly other components.

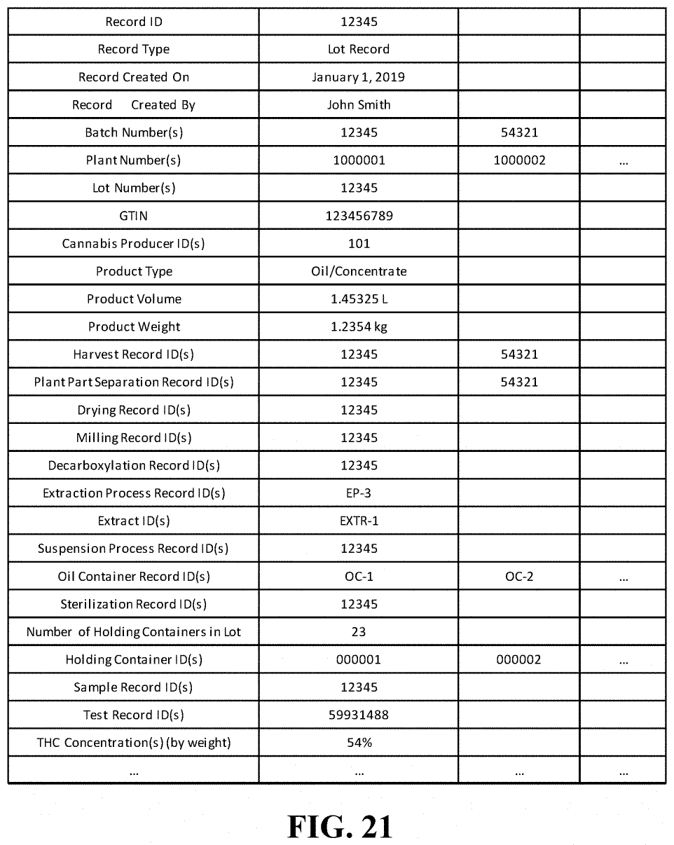

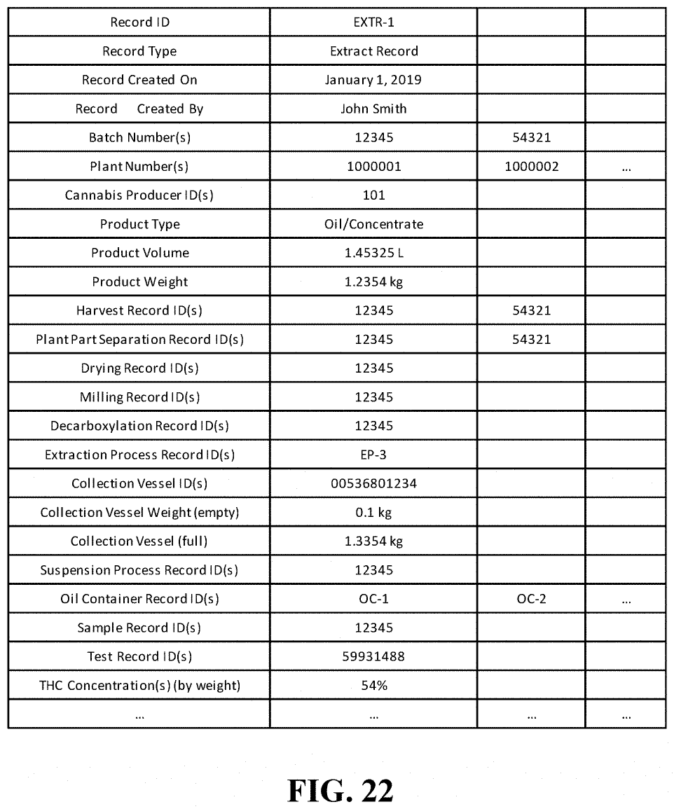

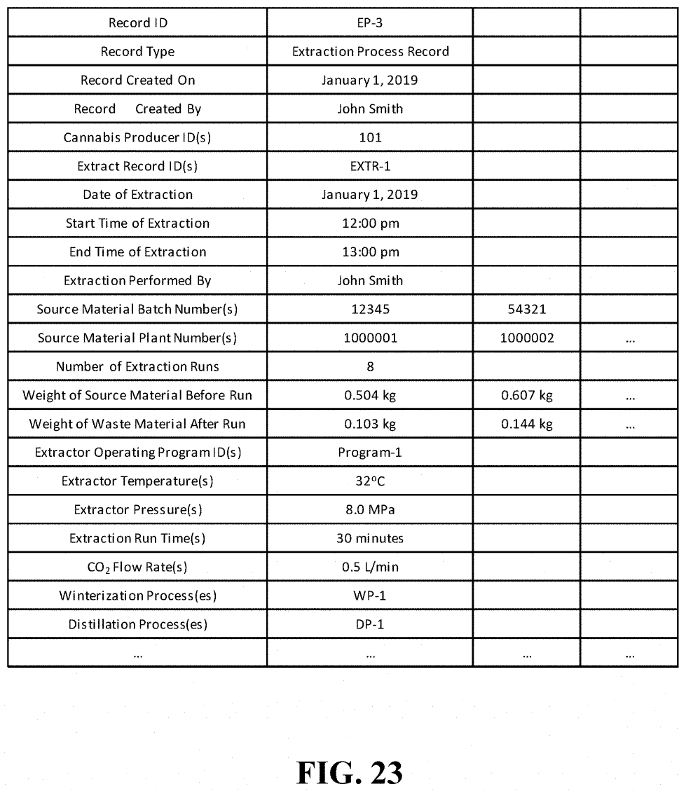

Various types of records that could be stored in an ICS are referenced herein. Several detailed examples are shown in FIGS. 21-23. FIG. 21 illustrates an example of a lot record, FIG. 22 illustrates an example of an extract record, and FIG. 23 illustrates an example of an extraction process record.

The example lot record in FIG. 21 includes a Record ID and a Record Type. Date of creation and creator of the record are also included, in Record Created On and Record Created By fields in this example.

The Batch Number(s) field in this example illustrates one way in which batch(es) and lot(s), and/or identifiers thereof, could be associated with each other. The batch numbers in the Batch Number(s) field are explicitly associated with the lot to which the lot record corresponds, by including the batch numbers in the lot record. Plant numbers, if specified in the Plant Number(s) field, similarly associate plants and/or plant numbers with a lot and/or lot number, and possibly with one or more batches and/or batch numbers as well.

In FIG. 21, the lot record also includes a Lot Number(s) field, which could be useful if a lot record has a different Record ID that does not match the lot number. For a lot record, it may be preferred to use the lot number as the Record ID, but this might not always be the case.

Other information regarding the lot is also included in the example lot record, in the following fields: GTIN, Cannabis Producer ID(s), Product Type, Product Volume, Product Weight, Number of Holding Containers in Lot, and THC Concentration(s) (by weight).

Other explicit associations are included in the example lot record as well, in the fields Harvest Record ID(s), Plant Part Separation Record ID(s), Drying Record ID(s), Milling Record ID(s), Decarboxylation Record ID(s), Extraction Process Record ID(s), Extract ID(s), Suspension (i.e. Mixing/Dilution) Process Record ID(s), Oil Container Record ID(s), Sterilization Record ID(s), Holding Container ID(s), Sample Record ID(s) and Test Record IDs.

Lot information, whether stored in a lot record or otherwise, could be searchable. Searchable lot information could be especially useful in facilitating traceability. A search for a batch number in an ICS database using a computer, for example, could identify any associated lots much more quickly and reliably than a manual search of lot information by an operator. Speed and reliability could be crucial in such applications as identifying lots for product recalls for example.

Explicit associations as shown in the example lot record in FIG. 21 could also impact search speed and reliability.

The example lot record in FIG. 21 represents one embodiment. All fields are populated in FIG. 21, but this might not be the case for every lot. Also, in other embodiments, a lot record could include further, fewer, and/or different fields, arranged in a similar or different order. Lot records might not even be used in other embodiments in which information related to lots is instead stored in some other way.

With reference now to the example extract record in FIG. 22, this example record, like the example lot record in FIG. 21, also includes a Record ID field, a Record Type field, a Record Created On field, and a Record Created By field. In FIG. 22, the example record includes a Record ID without a separate extract identifier, and this illustrates an embodiment in which a cannabis product identifier, in this case an extract identifier, is used as a Record ID and need not be separately specified in a record.

Inclusion of the Batch Number(s) field in an extract record is one way in which batch(es) and extract(s), and/or identifiers thereof, could be associated with each other. The batch numbers in the Batch Number(s) field are explicitly associated with the extract and/or extract identifier to which the extract record corresponds. Plant numbers, if specified in the Plant Number(s) field, similarly associate plants and/or plant numbers with an extract and/or extract number, and possibly with one or more batches and/or batch numbers as well.

In some embodiments, an extract record could include a Lot Number(s) field in addition to or instead of the Batch Number(s) field. A Lot Number(s) field, without a Batch Number(s) field, could provide for “indirect” associations between extracts and batches. For example, one or more lot numbers could be specified in a Lot Number(s) field, to associate the lot number(s) with an extract number, and extract-batch association could then be determined from a lot record that includes one or more associated batch numbers in a Batch Number(s) field. In the examples shown in FIGS. 21 and 22, batch numbers are explicitly associated with a lot number (FIG. 21) and an extract number (FIG. 22), and from these associations a lot-extract association could be determined. There is also an explicit association between lot and extract as well, in that the lot record in FIG. 21 includes the Record ID of the extract record in FIG. 22, in the Extract ID(s) field of the lot record.

Other information regarding the extract is included in the example extract record, in the following fields: Cannabis Producer ID(s), Product Type, Product Volume, Product Weight, and THC Concentration(s) (by weight). The Collection Vessel (full) and Collection Vessel (empty) fields in the example extract record represent an example of fields that could be used to determine or verify values in other fields. As shown in FIG. 22, the Collection Vessel (full) weight minus the Collection Vessel (empty) weight is consistent with the Product Weight entry. Product Weight entry could be calculated from the Collection Vessel (full) weight and the Collection Vessel (empty) weight, or could be measured and verified using the Collection Vessel (full) weight and the Collection Vessel (empty) weight.

Other explicit associations are also included in the example extract record as well, in the fields Harvest Record ID(s), Plant Part Separation Record ID(s), Drying Record ID(s), Milling Record ID(s), Decarboxylation Record ID(s), Extraction Process Record ID(s), Collection Vessel ID(s), Suspension (i.e. Mixing/Dilution) Process Record ID(s), Oil Container Record ID(s), Sample Record ID(s), and Test Record ID(s).

Extract information, whether stored in an extract record or otherwise, could be searchable. Searchable extract information could be especially useful in facilitating traceability. A search for a batch number in an ICS database using a computer, for example, could identify any associated extracts much more quickly and reliably than a manual search of extract information by an operator. Speed and reliability could be crucial in such applications as identifying lots for product recalls for example.

Explicit associations as shown in the example extract record in FIG. 22 could also impact search speed and reliability.

The example extract record in FIG. 22, like the example lot record in FIG. 21, represents one embodiment. All fields are populated in FIG. 22, but this might not be the case for every extract. Also, in other embodiments, an extract record could include further, fewer, and/or different fields, arranged in a similar or different order. Extract records might not even be used in other embodiments in which information related to extracts is instead stored in some other way.

Turning to FIG. 23, the example extraction process record, like the example records in FIGS. 21 and 22, also includes a Record ID field, a Record Type field, a Record Created On field, and a Record Created By field. In FIG. 23, as in FIG. 22, the example record includes a Record ID without a separate extraction process identifier. This illustrates another embodiment in which a process identifier, in this case an extraction process identifier, is used as a Record ID and need not be separately specified in a record.

Other extraction process details are specified in the following fields: Cannabis Producer ID(s), Date of Extraction, Start Time of Extraction, End Time of Extraction, Extraction Performed By, Number of Extraction Runs, Weight of Source Material Before Run, Weight of Source Material After Run, Extractor Operating Program ID(s), Extractor Temperature(s), Extractor Pressure(s), Extraction Run Time(s), CO₂ Flow Rate(s), Winterization Process(es), and Distillation Process(es).

Several associations are also explicitly specified in the example extraction record, in the Extract Record ID(s) field, the Source Material Batch Number(s) field, and the Source Material Plant Number(s) field. The entry in the Extract Record ID(s) field references the extract record in FIG. 22, which cross-references the extraction process record in its Extraction Process Record ID(s) field. The entries in the Source Material Batch Number(s) field and the Source Material Plant Number(s) field in FIG. 23 include the same batch and plant numbers as the entries in the Batch Number(s) fields and Plant Number(s) fields in the example lot record in FIG. 21 and the example extract record in FIG. 22. The example lot record in FIG. 21 also cross-references the extraction process record in its Extraction Process Record ID(s) field.

Such associations and cross-references could enable a much higher level of accessibility to various types of information in an ICS database, relative to manually maintained records and/or even electronic records that are not as highly organized or cross-referenced as in the examples shown. Common information that appears in multiple records and/or explicit associations between related records could vastly improve search speed and reliability. Automated collection of information, creation of records, and/or population of fields in records, could be especially preferred to maintain record integrity and accuracy.

Extraction process information, whether stored in an extraction process record or otherwise, could be searchable. Searchable extraction process information could be especially useful in facilitating traceability. A search for a batch number in an ICS database using a computer, for example, could identify any associated extraction processes much more quickly and reliably than a manual search of extraction process information by an operator. As noted at least above for the example records in FIGS. 21 and 22, speed and reliability could be crucial in such applications as identifying lots for product recalls for example, and explicit associations as shown in the example extract record in FIG. 23 could also impact search speed and reliability.

The example extraction process record in FIG. 23, like the example lot record in FIG. 21 and the example extract record in FIG. 23, represents one embodiment. All fields are populated in FIG. 23, but this might not be the case for every extraction process. Also, in other embodiments, an extraction process record could include further, fewer, and/or different fields, arranged in a similar or different order. Extraction process records might not even be used in other embodiments in which information related to extraction processes is instead stored in some other way.

FIGS. 21-23 provide illustrative examples of records that could be used to store information relating to lots, extracts, and extraction processes, respectively, in an ICS. Similar or different records could be used to store other types of information. Examples of information that could be stored for cannabis plant material, other cannabis products, and/or other processes are disclosed by way of example elsewhere herein.

Some embodiments disclosed herein relate to a hierarchical dataset in which a batch identifier is a root node and lot numbers form branches of the hierarchical dataset from the root node. Consider, for example, two lot records of the form shown in FIG. 21. Two lots of cannabis product could be produced from the same batch of plants, and each lot could have a corresponding lot record including the same batch number. In this sense, the lot numbers could be considered branches from the same batch number in a hierarchical dataset. Other branches in such a dataset are also possible. For example, units of a cannabis product with a certain lot number could be further processed to produce multiple different cannabis products, such as beverages and edibles, to which further identifiers could be assigned. Those further identifiers form branches from their originating lot number, which in turn branches from a batch number or possibly multiple batch numbers as in the example shown in FIG. 21.

A hierarchical dataset is not restricted to only two levels (batch level and lot level), or even to levels associated with batches and/or lots.

Cannabis-Infused Consumer Product Manufacturing

In some embodiments, a cannabis producer processes cannabis plants from a batch of cannabis plants in order to produce one or more units of a cannabinoid-containing substance. A cannabinoid-containing substance is any substance that contains cannabinoids. A cannabinoid-containing substance is itself a cannabis product. However, a subsequent product produced using a cannabinoid-containing substance may also be called a cannabis product. Also, a cannabinoid-containing substance may sometimes instead be called a cannabis-containing substance.

In some embodiments, the tracking and traceability system disclosed herein is also used to track other materials which can be used in the manufacture of cannabis products and/or materials which can form part of cannabis products. For example, in some embodiments, edible ingredients such as chocolate, gelling agents, emulsifiers, etc., can be tracked and/or traced by the traceability system disclosed herein. As described in more detail, above, in some embodiments, the process of tracking such edible ingredients form part of a Preventative Control Plan (PCP), or other such protocol that demonstrates how risks to food and food animals are identified and controlled.

In some embodiments, some or all of a unit of a cannabinoid-containing substance is used as an ingredient in a process to produce one or more cannabis-infused consumer products. The following is a non-exhaustive list of examples of cannabinoid-containing substances that may be used as an ingredient in a process to produce one or more cannabis-infused consumer products:
an extract (e.g. the substance output from an extraction process or machine, e.g. a resin resulting from a CO₂ extraction process);
a distillate (e.g. the substance output from a distillation or fractionation process or machine, e.g., a distilled extract containing almost pure cannabinoid or mixture of cannabinoids, such as at least 90 wt. % pure cannabinoid);
a distillate/extract in an emulsification system (e.g. a substance in which a distillate or an extract has been mixed with one or more emulsifiers, e.g., hydrophobic cannabinoid molecules that have been covered/coated with, or incorporated into, an emulsifier);
a cannabinoid emulsion (e.g. distillate/extract in an emulsification system+aqueous liquid);
a concentrated cannabinoid emulsion (e.g. a cannabinoid emulsion that has a high concentration of cannabinoids, e.g. having at least 3 wt. % of a cannabinoid, taking into account both the acid form of the cannabinoid, such as THC-a, and the decarboxylated form of the cannabinoid, such as THC).

The following is a non-exhaustive list of examples of cannabis-infused consumer products that may be produced using a cannabinoid-containing substance as an ingredient:
Cannabis-infused beverages (beverages incorporating cannabinoid-containing substance(s) and which are intended to be consumed in the same manner as beverage drinks);
Cannabis-infused edibles (products incorporating cannabinoid-containing substance(s) and which are intended to be consumed in the same manner as food);
Cannabis-infused topicals (products that incorporate cannabinoid-containing substance(s) and which are intended to be used on external body surfaces, such as skin, hair, and/or nails);
Cannabis-infused mucoadhesive delivery systems (products that incorporate cannabinoid-containing substance(s) and which are intended to be used on mucosa body surfaces, such as mouth, anal, nasal and vaginal cavities);
Cannabis-infused vaping oil (oil products incorporating cannabinoid-containing substance(s) and which are intended to be consumed in a vaping device, such as an electronic cigarette);
A cartridge containing cannabis-infused vaping oil.

An entity that uses some or all of a unit or amount of a cannabinoid-containing substance as an ingredient to produce one or more cannabis-infused consumer products will be referred to as a cannabis processor. A cannabis processor may sometimes be called a licensed processor. In some embodiments, the cannabis producer and the cannabis processor may be the same entity. However, more generally, the cannabis processor is a separate entity from the cannabis producer, and the cannabis processor receives one or more units of a cannabinoid-containing substance from a cannabis producer, and then uses some or all of the one or more units of the cannabinoid-containing substance to produce one or more units of a cannabis-infused consumer product. A consumer product is sometimes referred to instead as a consumable product.

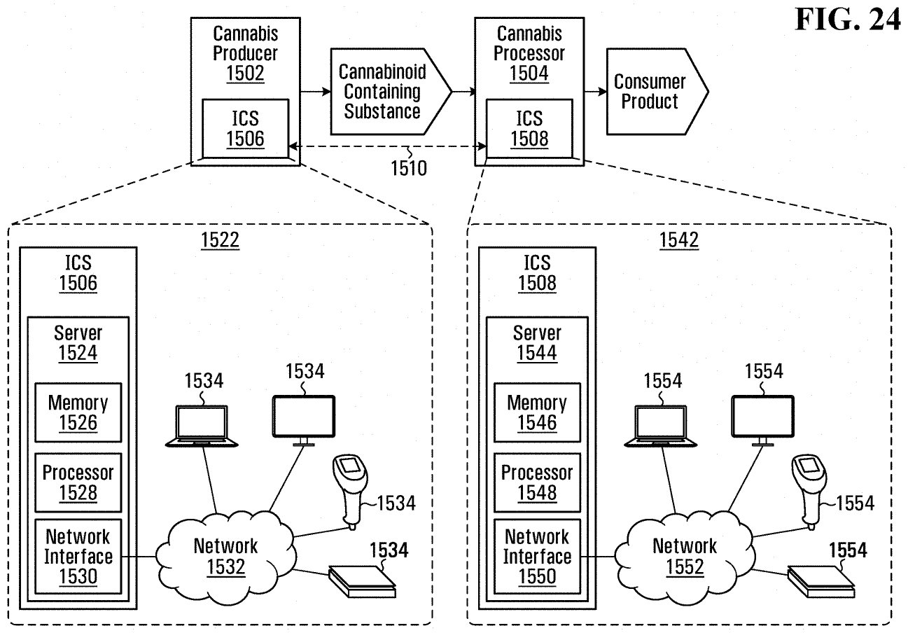

FIG. 24 is a block diagram of a cannabis producer 1502 and a cannabis processor 1504, according to one embodiment. The cannabis producer 1502 utilizes an ICS 1506, e.g. the ICS described earlier in relation to FIGS. 4A-4M. The cannabis processor 1504 also utilizes an ICS 1508. The ICS 1506 and the ICS 1508 may be the same ICS, e.g. if the cannabis producer 1502 and the cannabis processor 1504 are the same entity or related entities. In the description of FIG. 24 below, it will be assumed that the cannabis producer 1502 and the cannabis processor 1504 are different entities, and that the ICS 1506 and ICS 1508 are different ICS's.

An example of ICS 1506 is illustrated in stippled bubble 1522. The ICS 1506 includes a server 1524 having a memory 1526, processor 1528, and network interface 1530. The processor 1528 controls the operations of the ICS 1506. The processor 1528 may be implemented by one or more processors that execute instructions stored in the memory 1526. Alternatively, some or all of the processor 1528 may be implemented using dedicated circuitry, such as an ASIC, GPU, or FPGA for performing the operations of the processor 1528. Several input/output (I/O) devices 1534 are connected to the server 1524 via a network 1532. Examples of I/O devices 1534 include computers, displays, scanners, scales, label makers, etc. which are used as part of the cannabis production process by the cannabis producer 1502. For example, server 1524 may be server 402 in FIG. 4A, and I/O devices 1534 may include items such as:
computer(s) 424a, sensors(s) 428a, scale(s) 430a, label maker(s) 432a, and/or scanner(s) 434a in the cultivation and harvest system 420a; and/or
scale(s) 430b-1 and 430b-2, scanner(s) 434b-1 and 434b-2, computer(s) 424b, and/or label maker(s) 432b in the plant part separation system 420b; and/or
scales(s) 430c, scanner(s) 434c, computer(s) 424c, and/or sensor(s) 428c in the waste destruction system 420c; and/or
scale(s) 430d-1 and 430d-2, scanner(s) 434d-1 and 434d-2, computer(s) 424d, and/or label maker(s) 432d in the fresh processing system 420d; and/or
scale(s) 430e-1 and 430e-2, scanner(s) 434e-1 and 434e-2, computer(s) 424e, sensor(s) 428e and/or label maker(s) 432e in the drying system 420e; and/or
scale(s) 430f-1 and 430f-2, scanner(s) 434f-1 and 434f-2, computer(s) 424f, sensor(s) 428f, and/or label maker(s) 432f in the milling system 420f; and/or
scale(s) 430g-1 and 430g-2, scanner(s) 434g-1 and 434g-2, computer(s) 424g, sensor(s) 428g, and/or label maker(s) 432g in the decarboxylation system 420g; and/or
scale(s) 430h-1 and 430h-2, scanner(s) 434h-1 and 434h-2, computer(s) 424h, sensor(s) 428h, and/or label maker(s) 432h in the extraction system 420h; and/or
scale(s) 430i-1 and 430i-2, scanner(s) 434i-1 and 434i-2, computer(s) 424i, sensor(s) 428i, and/or label maker(s) 432i in the oil formulation system 420i; and/or
scale(s) 430j-1 and 430j-2, scanner(s) 434j-1 and 434j-2, computer(s) 424j, sensor(s) 428j, and/or label maker(s) 432j in the packaging system 420j; and/or
scale(s) 430k-1 and 430k-2, scanner(s) 434k-1 and 434k-2, computer(s) 424k, and/or label maker(s) 432k in the sterilization system 420k; and/or
scale(s) 430l, scanner(s) 434l, label maker(s) 432l, and/or computer(s) 424l in the testing system 420l; and/or
scale(s) 430m, scanner(s) 434m, label maker(s) 432m, and/or computer(s) 424m in the shipping system 420m.

An example of ICS 1508 is illustrated in stippled bubble 1542. The ICS 1508 includes a server 1544 having a memory 1546, processor 1548, and network interface 1550. The processor 1548 controls the operations of the ICS 1508. The processor 1548 may be implemented by one or more processors that execute instructions stored in the memory 1546. Alternatively, some or all of the processor 1548 may be implemented using dedicated circuitry, such as an ASIC, GPU, or FPGA for performing the operations of the processor 1548. Several I/O devices 1554 are connected to the server 1544 via a network 1552. Examples of I/O devices 1554 include computers, displays, scanners, scales, label makers, etc. which are used as part of the processing by the cannabis processor 1504.

In some embodiments, ICS 1506 and ICS 1508 may share information, as shown by stippled line 1510. The shared information may be transferred over a network in some embodiments, and the information may relate to an association between records and/or lots, e.g. linking a lot number on a cannabis-infused consumer product produced by the cannabis processor 1504 back to the lot number of a unit of a cannabinoid-containing substance received from the cannabis producer 1502 and used to make that cannabis-infused consumer product.

In operation, the cannabis producer 1502 produces one or more units of a cannabinoid-containing substance, e.g. a cannabinoid emulsion, which is used as a raw material/ingredient by the cannabis processor 1504 to produce one or more units of a cannabis-infused consumer product, e.g. a cannabis-infused edible, beverage, and/or topical product. The cannabis producer 1502 uses its ICS 1506 to record, log, track and/or monitor production of the cannabinoid-containing substance throughout cultivation, harvesting, processing, sales, shipping, and/or other operations, e.g. as described in detail earlier. The cannabis processor 1504 similarly uses its ICS 1508 to record, log, track and/or monitor its cannabis-infused consumer products, e.g. from receipt of the cannabinoid-containing substance from the cannabis producer 1502 through to production of the cannabis-infused consumer product and shipping and/or sale of the cannabis-infused consumer product.

For example, the ICS 1508 may be used by the cannabis processor 1504 to record information relating to the production of each lot of cannabis-infused consumer product. The ICS 1508 may record any or all transfers of the cannabinoid-containing substance or a product or intermediary product incorporating some of all of the cannabinoid-containing substance within and/or between the systems used by the cannabis processor 1504. The ICS 1508 may enable traceability of any or all cannabis through at least part of a production process, including traceability to lot level and/or batch level. This may include enabling traceability through and back to: a master batch of the cannabis-infused consumer product that was produced using a particular lot of a cannabinoid-containing substance; and/or the cannabinoid-containing substance as received from the cannabis producer 1502; and/or a diluted form of the cannabinoid-containing substance (e.g. if the cannabinoid-containing substance received from the cannabis producer 1502 is diluted or added to a larger volume of other liquid); and/or units of consumer product produced using the cannabinoid-containing substance; and/or units of consumer product in storage; and/or units of consumer product that have been released for sale or sold.

By way of example, in the event of a recall, the ICS 1508 may be used to determine the status and/or location of all cannabis-infused consumer products that fall within the scope of the recall. As another example, the ICS 1508 may be used to trace a particular cannabis-infused consumer product (e.g. an edible, beverage, or topical) to a particular lot of cannabinoid-containing substance received from the cannabis producer 1502. The ICS 1508 may therefore facilitate traceability back through to cannabis producer 1502, e.g. the lot number associated with a particular unit of cannabis-infused consumer product may be traced back to the lot number of a unit of cannabinoid-containing substance received from the cannabis producer 1502. This may allow for both: (1) the cannabis processor 1504 to determine which other cannabis-infused consumer products may be subject to the recall; and (2) the cannabis producer 1502 to use their ICS 1506 to determine which batch of cannabis plants, and hence which other cannabinoid-containing substances produced by the cannabis producer 1502, may be subject to the recall.

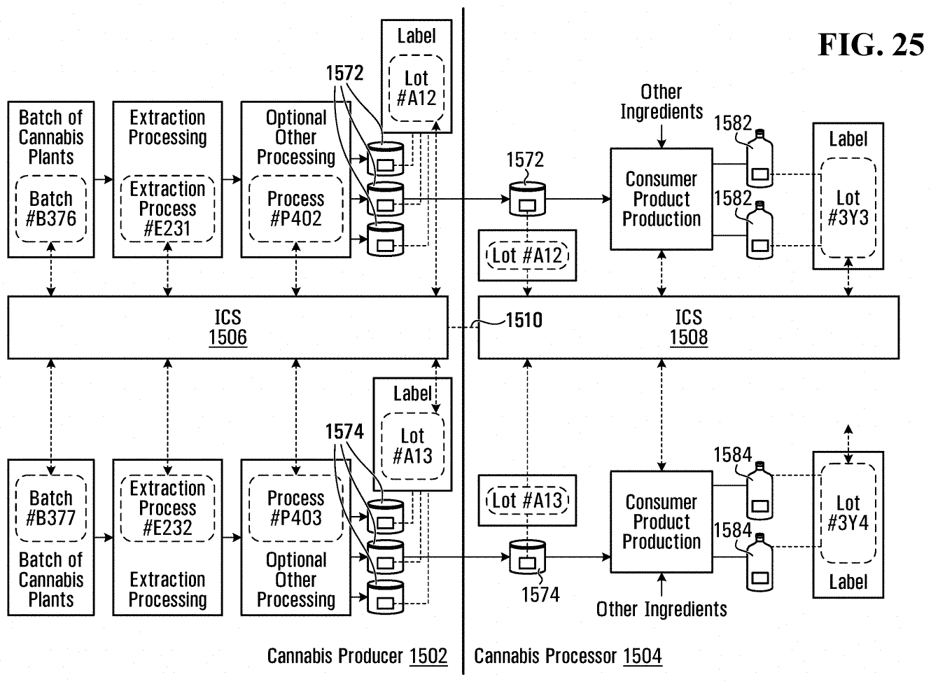

FIG. 25 is a schematic illustrating an example of traceability from a cannabis-infused consumer product back to a batch of cannabis plants. A cannabis producer 1502 cultivates and harvests different batches of cannabis plants, two of which are illustrated and respectively assigned batch numbers B376 and B377. The batches may be cultivated or harvested in parallel or serially. The batch numbers B376 and B377 are stored in the ICS 1506. In this illustrated example, at least some of the plants from batch B376 undergo extraction processing to create an extract, which is optionally subjected to additional processing (e.g. distillation, adding an emulsifier, etc.). The result is a plurality of units 1572 of a cannabinoid-containing substance, each of the units 1572 being cannabis in concentrated form in this particular embodiment. The extraction process is assigned an extraction process number E231, and the additional processing (if performed) is assigned a process number P402. The numbers E231 and P402 are stored in the ICS 1506 in association with/linked to the batch number B376. The plurality of units 1572 of cannabinoid-containing substance are each assigned the same lot number A12, which is marked on the holding container of each one of the plurality of units 1572, e.g. via a label. The lot number A12 is stored in the ICS 1506 in association with/linked to the processing and batch numbers E231, P402, and B376.

Similarly, in this illustrated example, at least some of the plants from batch B377 undergo extraction processing to create an extract, which is optionally subjected to additional processing (e.g. distillation, adding an emulsifier, etc.) to produce a plurality of units 1574 of a cannabinoid-containing substance, each of the units 1574 being cannabis in concentrated form in this particular embodiment. The extraction process is assigned an extraction process number E232, and the additional processing (if performed) is assigned a process number P403. The numbers E232 and P403 are stored in the ICS 1506 in association with/linked to the batch number B377. The plurality of units 1574 of cannabinoid-containing substance are each assigned the same lot number A13, which is marked on the holding container of each one of the plurality of units 1574, e.g. via a label. The lot number A13 is stored in the ICS 1506 in association with/linked to the processing and batch numbers E232, P403, and B377.

Some or all of the batch, process, and lot numbers (e.g. numbers B376, B377, E231, E232, P402, P403, A12, and A13) may be generated by the ICS 1506, or instead generated manually or by local equipment and stored in the ICS 1506.

In the embodiment illustrated in FIG. 25, the lot number A12 assigned to each unit 1572 of cannabinoid-containing substance originating from batch B376 and output from extraction process E231 (and optional additional processing P402) is different from the lot number A13 assigned to each unit 1574 of cannabinoid-containing substance originating from batch B377 and output from extraction process E232 (and optional additional processing P403). The lot number therefore allows, through the ICS 1506, traceability from a unit of cannabinoid-containing substance all the way back to the particular batch of cannabis plants used to produce that unit of cannabinoid-containing substance.

The cannabis processor 1504 receives a unit 1572 of cannabinoid-containing substance having lot number A12. The lot number A12 is stored in the ICS 1508. Some or all of the unit 1572 of cannabinoid-containing substance is used in a consumer product production process, in combination with other ingredients, to produce a plurality of units 1582 of cannabis-infused consumer products for sale, e.g. a plurality of cannabis-infused beverages. The plurality of units 1582 of cannabis-infused consumer product are each assigned the same lot number 3Y3, which is marked on each one of the plurality of units 1582, e.g. via a label. The lot number 3Y3 is stored in the ICS 1508 in association with/linked to the cannabinoid-containing substance lot number A12. Other processing numbers may be assigned during the consumer product production and linked to the lot numbers 3Y3 and A12, e.g. a master consumer product batch number, a processing number, a holding tank number, etc., depending upon the implementation.

Similarly, in this illustrated example, the cannabis processor 1504 receives a unit 1574 of cannabinoid-containing substance having lot number A13. The lot number A13 is stored in the ICS 1508. Some or all of the unit 1574 of cannabinoid-containing substance is used in a consumer product production process, in combination with other ingredients, to produce a plurality of units 1584 of cannabis-infused consumer products for sale, e.g. a plurality of cannabis-infused beverages. The plurality of units 1584 of cannabis-infused consumer product are each assigned the same lot number 3Y4, which is marked on each one of the plurality of units 1584, e.g. via a label. The lot number 3Y4 is stored in the ICS 1508 in association with/linked to the cannabinoid-containing substance lot number A13. Other processing numbers may be assigned during the consumer product production and linked to the lot numbers 3Y4 and A13, e.g. a master consumer product batch number, a processing number, a holding tank number, etc., depending upon the implementation.

In this example, the lot number 3Y4 assigned to each consumer product unit 1584 is the same because units 1584 originate from the same amount (lot) A13 of cannabinoid-containing substance. Similarly, the lot number 3Y3 assigned to each consumer product unit 1582 is the same because units 1582 originate from the same amount (lot) A12 of cannabinoid-containing substance. However, the lot number 3Y4 is different from the lot number 3Y3 because consumer product units 1582 originate from a different amount (lot) of cannabinoid-containing substance than consumer product units 1584.

In the embodiment illustrated in FIG. 25, indicia marked on a unit of cannabis-infused consumer product (e.g. lot number 3Y3) is indicative of (e.g. mapped back to) a particular amount (e.g. particular lot) of a cannabinoid-containing substance. That particular amount of cannabinoid-containing substance was derived from particular cannabis plant material and contains one or more cannabinoids. That particular amount of cannabinoid-containing substance may be a particular lot of cannabinoid-containing substance, which is assigned a particular lot number, and which differs from another particular amount (lot) of cannabinoid-containing substance. For example, in FIG. 25 indicia in the form of lot number 3Y3 is mapped back to (indicative of) lot number A12, and lot number A12 was derived from and ultimately maps back to a particular batch of cannabis plant material from which the cannabis originated (e.g. lot number 3Y3 is mapped back to lot number A12, which is mapped back to cannabis plants batch number B376). In some embodiments, the lot number on a unit of the cannabis-infused consumer product (e.g. lot number 3Y3) may also be used to identify particular processing or other steps in the process of creating that unit of cannabis-infused consumer product from a batch of cannabis plants (e.g. lot number 3Y3 links back to extraction process E231). The ICS 1506 and 1508 facilitate the traceability by storing the processing records and numbers in association with each other.

The exact processing performed by cannabis processor 1504 is implementation specific and depends upon the cannabis-infused consumer product being produced. Some examples will now be described in the context of producing cannabis-infused beverages.

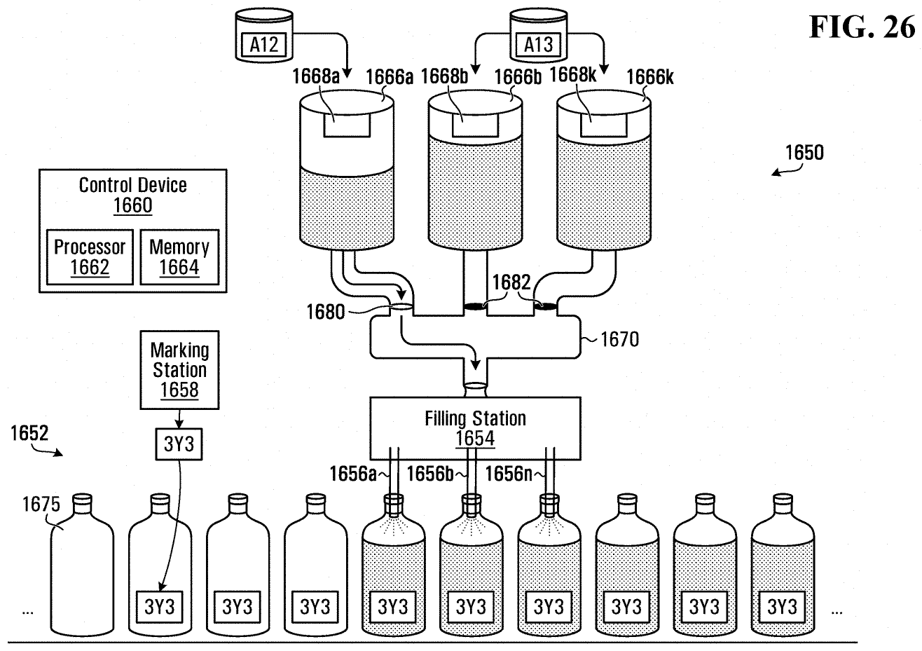

FIG. 26 is a block diagram of a system 1650 for producing cannabis-infused beverages, according to one embodiment. The system 1650 includes: a filling line 1652; a filling station 1654 having a plurality of nozzles 1656a-n; a container marking station 1658 for applying indicia to containers or packaging for the units of cannabis-infused beverages, which in this embodiment is a labeling station that applies a label on each container; a control device 1660 including a processor 1662 and computer readable storage in the form of memory 1664; a plurality of holding tanks 1666a-k, each having a respective liquid level sensor 1668a-k; and a supply selection valve 1670 interposed between and in fluid communication with the holding tanks 1666a-k and the filling station 1654. The processor 1662 may be implemented by one or more processors that execute instructions stored in the memory 1664. Alternatively, some or all of the processor 1662 may be implemented using dedicated circuitry, such as an ASIC, GPU, or FPGA. The processor 1662 implements the operations of the control device 1660. A control device may alternatively be called a controller.

The filling line 1652 comprises a conveyor of containers. In the illustrated embodiment the containers are bottles 1675. In general, any type of container may be used, e.g. a glass, plastic, or aluminum container. Although the bottles 1675 are illustrated in a conveyor line, a conveyor line is only an example. A different configuration may be used instead, e.g. pallets or disks holding bottles that are presented to the filling station 1654 and filled in batches. The filling station 1654 uses nozzles 1656a-n to fill the bottles 1675 with cannabis-infused beverage from holding tanks 1666a-k. Several bottles are filled in parallel, with each bottle being filled by a respective one of the nozzles 1656a-n. The container marking station 1658 prints and applies a label on each bottle. Instead of generating labels, the marking station 1658 may apply indicia to each bottle in another manner instead, e.g. by stamping each bottle or providing indentations in each bottle, etc. The supply selection valve 1670 is capable of selectively acquiring a plurality of supply positions, each supply position associating a respective holding tank with the filling station 1654 to supply the filling station 1654 from the respective holding tank.

Operation of the system 1650 will be explained in the context of the example introduced in relation to FIG. 25. A unit of cannabinoid-containing substance having lot number A12 is received by the cannabis processor 1504 from the cannabis producer 1502. A first master batch of cannabis-infused beverage is prepared by the cannabis processor 1504 using that unit of cannabinoid-containing substance, and the first master batch of cannabis-infused beverage is stored in holding tank 1666a. Preparing the first master batch may include processing steps such as adding additional ingredients (e.g. water, flavourants), and possibly first diluting or otherwise modifying the cannabinoid-containing substance to put it in a form suitable for adding as an ingredient (e.g. forming an emulsion from the cannabinoid-containing substance if the lot of cannabinoid-containing substance as received is not an emulsion). In some embodiments, the mater batch may be tested for cannabinoid concentration levels. In some embodiments, the cannabinoid concentration levels determined as a result of testing can be recorded by way of the ICS 1508. In some embodiments, the cannabinoid concentration levels can be recorded in a master batch record.

The control device 1660 is in communication with the ICS 1508 (not illustrated), and the control device 1660 associates product lot number 3Y3 with each bottle to be filled with cannabis-infused beverage from the first master batch. The control device 1660 controls marking station 1658 to affix a label having lot number 3Y3 to each bottle that is to be filled with cannabis-infused beverage from the first master batch. The label is from a supply of labels used by the marking station 1658.

In some embodiments, the ICS 1508 may also store a holding tank number and/or master batch number and/or other processing number(s) in association with/linked to the product lot number 3Y3 to allow for traceability throughout the process performed by the cannabis processor 1504. As an example, product lot number 3Y3 may also be associated with: (1) master batch number MB35, where “MB35” is a number associated with a particular unit of master batch that was produced using cannabinoid-containing substance lot number A12 and that is held in a particular holding tank 1666a; and (2) holding tank number HT1, where “HT1” is a number assigned to the holding tank 1666a that holds a unit of master batch. This may allow for traceability within the cannabis processor's operations, e.g. if there was a problem with a particular bottle of cannabis-infused beverage, then the lot number 3Y3 on the label of the bottle may be used by the ICS 1508 to identify that the cannabis-infused beverage for that particular bottle was stored in holding tank HT1 and from master batch MB35, and that master batch MB35 is a batch of cannabis-infused beverage made using a unit of cannabinoid-containing substance from lot number A12. The traceability could extend back through the cannabis producer 1502 also: the lot number A12 of cannabinoid-containing substance was generated using an extract from extraction process E231, and the input to that extraction process was cannabis from batch B376 of cultivated/harvested cannabis plants. In this way, in some embodiments it is possible to use to lot number 3Y3 on a bottle 1675 to trace some or all steps of the processes involving the cannabis ingredient, possibly all the way back to the batch of plants cultivated and harvested to produce the cannabis ingredient.

In FIG. 26, a second master batch of the cannabis-infused beverage is prepared using a unit of cannabinoid-containing substance from another lot A13, and that second master batch of cannabis-infused beverage is stored in holding tanks 1666b and 1666k. The second master batch of cannabis-infused beverage is held in the holding tanks until the first master batch of cannabis-infused beverage has depleted. For example, the control device 1660 controls the valve 1670 to open the flowline from holding tank 1666a, as shown at 1680, and to close the flowline from holding tanks 1666a and 1666k, as shown at 1682.

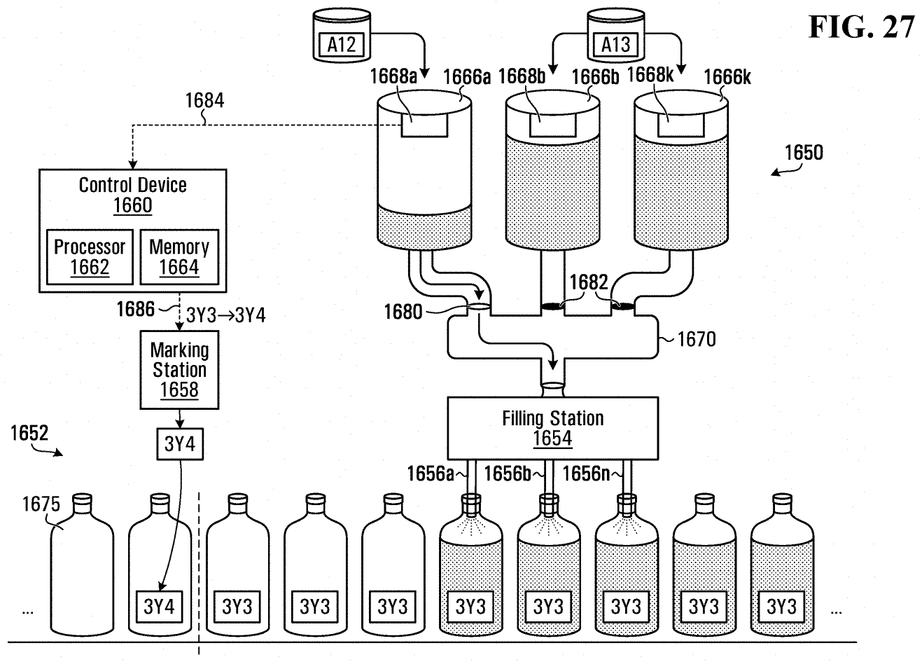

Turning to FIG. 27, in some embodiments a signal 1684 from liquid level sensor 1668a in holding tank 1666a indicates to control device 1660 that the cannabis-infused beverage in holding tank 1666a is becoming depleted, such that the control device 1660 knows the point at which bottles will need to start being filled from holding tank 1668b instead. Alternatively, the number of bottles that can be filled with cannabis-infused beverage in a holding tank may be fixed or known in advance by the control device 1660, such that the control device 1660 may simply count the number of bottles and switch over to the next holding tank when the maximum number of bottles for a holding tank have been filled, in which case the liquid level sensors 1668a-k may not be utilized or present. In some embodiments, the control device 1660 may count and store the counted number, e.g. so that the number of bottles that can filled is known for future master batches and/or known for future lots (amounts) of cannabinoid-containing substance.

The control device 1660 stores information from the ICS 1508 indicating that holding tank 1668b holds cannabis-infused beverage from a second master batch, which is associated with a different product lot number 3Y4. Therefore, the control device 1660 sends a signal 1686 to marking station 1658 to modify the label being affixed to reflect lot number 3Y4, and to begin applying that label to each bottle, starting at the first bottle that is to receive the cannabis-infused beverage from the second master batch.

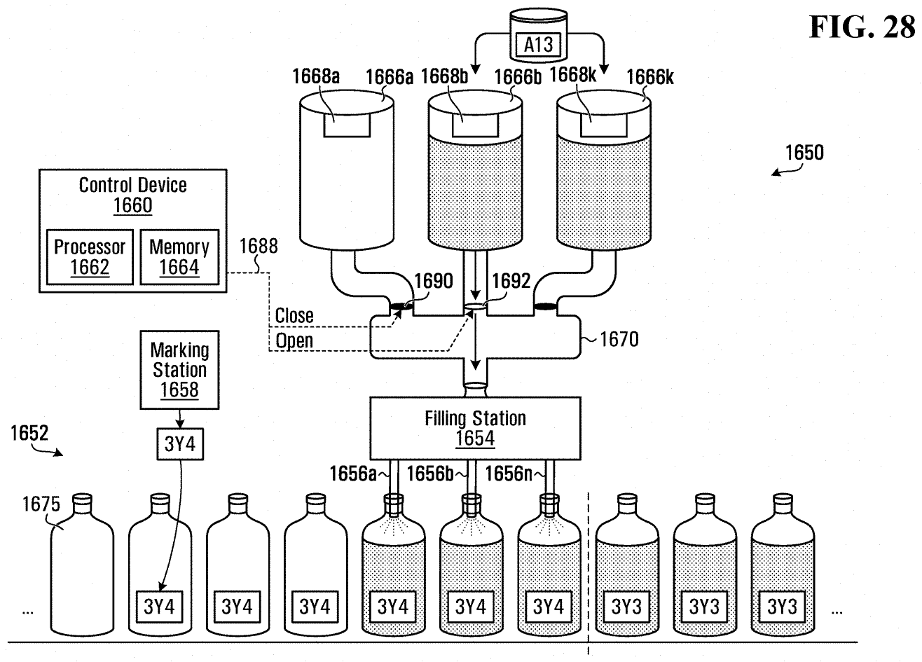

Turning to FIG. 28, at the appropriate switching point, the control device 1660 transmits a signal 1688 to valve 1670 to close the filling line from holding tank 1666a, as shown at 1690, and to open the filling line from holding tank 1666b, as shown at 1692. Although not illustrated, a similar switch occurs to go from holding tank 1666b to holding tank 1666k when holding tank 1666b is depleted of beverage. However, in this embodiment the lot number 3Y4 would not be changed because both holding tanks 1666b and 1666k include beverage from the same second master batch that originates from lot number A13 of the cannabinoid-containing substance. In an alternative embodiment, the lot number 3Y4 may be changed when switching from holding tank 1666b to holding tank 1666k in order to uniquely associate a lot number with a particular holding tank.

In some embodiments, a lot number on a unit of cannabis-infused consumer product (e.g. 3Y3 and 3Y4) and/or a lot number on a unit of cannabinoid-containing substance (e.g. A12 and A13) may be encoded in a machine-readable code, e.g. a barcode. The barcode may be decoded by a computer to obtain the lot number. In some embodiments, the computer may be connected to (or in network communication with) ICS 1506 and/or ICS 1508. In some embodiments, any of the numbers discussed herein, e.g. B376, B377, E231, E232, P402, P403, 3Y3, and 3Y4 may be encoded in a machine-readable code. Also, each number is an identifier, which in general may include alphanumeric characters and/or other symbols.

The bottles 1675 marked with label 3Y3 form one set of containers, and the bottles 1675 marked with label 3Y4 form another set of containers, such that the conveyor provides to the marking station 1658 successive sets of containers, each set having a label unique to that set in that the indicia applied to containers in one set would differ from the indicia applied to containers in another set. For example, each set would at least have its own lot number, and possibly other information listed also (e.g. date of bottling or creation of master batch, cannabis concentration if it differs amongst lots, etc.).

In FIGS. 26-28, the container marking station 1658 is located upstream of (i.e. before) the filling station 1654. However, in other embodiments, the container marking station 1658 may be located downstream of (i.e. after) the filling station 1654 and apply the labels after the bottles are filled, which may be useful in situations in which the controller 1660 cannot determine in advance of filling the exact switching point from one master batch (e.g. one master batch or lot number) to another master batch (e.g. another master batch or lot number). For example, the controller 1660 may receive a signal from sensor 1668a indicating that the liquid in tank 1666a is empty, near-empty, or depleted, perhaps such that there is not enough liquid left for even filling another bottle, at which point the controller 1660 may perform a supply switch to the second master batch in tank 1666b. The controller 1660 would then control the marking station 1658 downstream of the filling station 1654 to change the indicia applied to each bottle to update the lot number 3Y3 to 3Y4 at the point at which the cannabis-infused beverage supply switched from tank 1666a to tank 1666b. In any case, the control device 1660 synchronizes the operation of the marking station 1658 with the order in which the stream of individual containers is arranged such that each individual container receives an indicia (e.g. lot number) associated with the particular lot of cannabinoid-containing substance from which the consumer product in the container is made.

It will be appreciated that the general method and approach described above in relation to FIGS. 26-28 can also apply to non-beverage cannabis-infused consumer products in a similar way, e.g. to cannabis-infused edibles, topicals, mucoadhesive delivery systems, vape oils, vape oil cartridges, etc. For example, a set of units of cannabis-infused consumer product originating from the same lot of cannabinoid-containing substance may have the same marking (e.g. label) on the packaging of each unit, where the marking conveys the same lot number, and that lot number is different from the lot number used for a set of units of consumer product originating from a different lot of cannabinoid-containing substance. The control device 1660 would synchronizes the operation of the marking station 1658 with the order in which the stream of individual packages are arranged such that each individual package receives an indicia (e.g. lot number) associated with the particular lot of cannabinoid-containing substance from which the consumer product in the package is made.

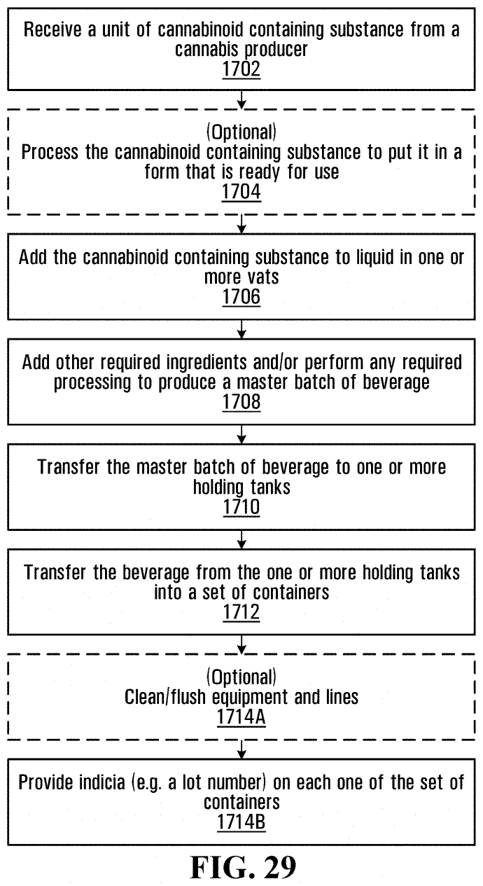

FIG. 29 is a method of producing cannabis-infused beverages, according to one embodiment.

In step 1702, a unit of cannabinoid-containing substance is received from a cannabis producer 1502. The unit of cannabinoid-containing substance has a particular dose and particular lot number. The unit of cannabinoid-containing substance is of or from a particular amount, e.g. of or from a particular lot comprising an amount of cannabinoid-containing substance derived from cannabis plant material to produce the lot, which has a particular lot number. The unit of cannabinoid-containing substance may be associated with a particular extraction process record, and/or an extract record, and/or an oil container record, and/or a lab sample record, and/or an oil jar record, and/or a lot record, which is/are stored in the ICS 1506 of the cannabis producer 1502. However, all of this record information is not necessarily transferred to the ICS 1508 of the cannabis processor 1504. In some embodiments, perhaps only the lot number and other required information (e.g. dose of the cannabinoid-containing substance, name or ID of the cannabis producer 1502, etc.) is provided to the cannabis processor 1504. The lot number and possibly other information (e.g. the dose of the cannabinoid-containing substance and name/ID of cannabis producer 1502) is stored in the ICS 1508, e.g. in a record created and stored in the ICS 1508 in association with the received unit of cannabinoid-containing substance.

Optionally, in step 1704, processing is performed on the cannabinoid-containing substance to put it in a form that is ready for use by the cannabis processor 1504. Whether step 1704 is performed, and if so, the extent of the processing in step 1704, will depend upon the form of the unit of cannabinoid-containing substance as received from the cannabis producer 1502. For example, if the unit of cannabinoid-containing substance is received as a “ready-to-mix” concentrated cannabinoid emulsion, then step 1704 may not need to be performed at all, whereas if the unit of cannabinoid-containing substance is received as a distillate, then step 1704 may be performed and include mixing the distillate with one or more emulsifiers.

In step 1706, the cannabinoid-containing substance is added to liquid in one or more vats. For example, the liquid may be or primarily consist of water. The ICS 1508 may generate a record documenting the transfer of cannabinoid-containing substance, e.g. date and/or time of transfer, amount transferred to each vat, vats receiving the cannabinoid-containing substance, etc.

In step 1708, any other required ingredients are added to the cannabis-infused liquid in the one or more vats, and/or any required processing is performed, in order to produce a master batch of the cannabis-infused beverage. The master batch may sometimes instead be referred to simply as a “batch”. The ICS 1508 may generate a record for the master batch, e.g. assigning a master batch number that is associated with/linked to the record documenting the transfer of the cannabinoid-containing substance and the lot number of the cannabinoid-containing substance.

In step 1710, the master batch is transferred to one or more holding tanks. The ICS 1508 may generate a record documenting the transfer. The record may include assigned holding tank number(s) to which the master batch was transferred.

In step 1712, the master batch of cannabis-infused beverage, which is held in the one or more holding containers, is transferred into a set of containers, e.g. into bottles or cans.

In step 1714A, optionally, the workspace, bottle filling machine(s) and lines and/or holding tanks are cleaned. Step 1714A could include washing or flushing certain components of the bottle filling machine with solvents (for example, water and/or ethanol) and/or compressed air. Cleaning/flushing the equipment helps prevent cross-contamination of toxins/contaminants between lots/batches, and also isolates cannabinoids between lots/batches, which further improves traceability of cannabinoids.

In step 1714B, indicia (e.g. labels having a lot number) are provided, a respective one on each container in the set of containers. In some embodiments, the indicia may be provided prior to filling the set of containers, e.g. as in FIG. 26, which shows a marking station upstream of a filling station. The indicia are markings, and each indicia includes an identification (e.g. text, number, and/or machine-readable code) that is unique to the set of containers filled with the cannabis-infused beverage originating from that particular unit of cannabinoid-containing substance received from the cannabis producer 1502. For example, the indicia may include a consumer product lot number (e.g. lot number 3Y3 in the examples above). In some embodiments, each container in the set of containers has the same indicia (e.g. same lot number and/or same other information) applied because each container in the set of containers has cannabis-infused beverage originating from the same lot of cannabinoid-containing substance. In some embodiments, the indicia includes the dose of cannabis in the beverage, which is labelled as the same for each container in the set, e.g. “Cannabis content: 2.5 milligrams THC per bottle”.

The ICS 1508 may assign the indicia, and/or may store the indicia, and/or may control a marking station, e.g. a label maker, to print the indicia on the set of containers. In some embodiments, the indicia is the same for all containers in the set and is indicative of: the master batch of cannabis-infused beverage used to fill the container, and/or the lot of cannabinoid-containing substance used to produce the cannabis-infused beverage, and/or the particular dose of cannabis, etc. In some embodiments, the indicia links each container in the set back to the same particular cannabinoid-containing substance (e.g. lot number) received from the cannabis producer.

In some embodiments, steps 1702 to 1714B are repeated for each subsequent different lot number of cannabinoid-containing substance received from the cannabis producer 1502. A different record and/or indicia is created in the ICS 1508 for each received lot of cannabinoid-containing substance used to produce a respective batch of cannabis-infused beverage containers. In this way, the indicia on a particular container of cannabis-infused beverage may be used to trace back to the particular cannabinoid-containing substance (e.g. lot number of cannabinoid-containing substance) used by the cannabis processor 1504 to produce that particular container of cannabis-infused beverage, and may also indicate the particular dose, e.g. particular concentration of the cannabinoid(s) in that particular container, which in some embodiments could vary between different lots of cannabinoid-containing substance. The same indicia may be used for containers holding beverage from the same batch (e.g. bottles having cannabis originating from the same lot).

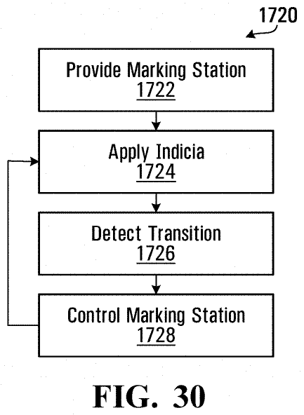

FIGS. 26-28 and the descriptions thereof relate primarily to cannabis-infused beverages. FIG. 30 is a flow diagram illustrating an example method for applying an indicia to containers filled with cannabis-infused beverage, according to another embodiment. The containers could be glass bottles, plastic bottles, or cans, for example. The example method 1720 involves, at 1722, providing a marking station to mark with an indicia containers that are filled with cannabis-infused beverage. Providing a marking station is not intended to imply that a marking station is manufactured as part of the example method 1720. A marking station could be purchased or otherwise acquired, for example.

In some embodiments, the indicia is indicative of a particular amount of a cannabinoid-containing substance derived from cannabis plant material and containing one or more cannabinoids, from which the cannabis-infused beverage is prepared, and the marking station is configured to receive a succession of containers filled with cannabis-infused beverage. The succession of containers is arranged in successive sets, where each set of containers is filled with cannabis-infused beverage made from a respective amount of the cannabinoid-containing substance.

At 1724, a first indicia is applied at each container from a first set. The first indicia is associated with a first amount of cannabinoid-containing substance from which the cannabis-infused beverage dispensed in the first set of containers is made. For completeness, it is noted that the cannabis-infused beverage could be dispensed into containers by any of various types of dispensing or container filling equipment, and be conveyed or otherwise provided to the marking station for marking. Applying the indicia at 1724 could involve, for example, printing the indicia onto the containers, otherwise marking the indicia on the containers, or generating labels that include the indicia and affixing the labels to the containers.

A transition from a first set of containers to a second set of containers in the succession of containers is detected at 1726. The first set of containers is filled with cannabis-infused beverage prepared from a first amount of cannabinoid-containing substance and the second set of containers is filled with cannabis-infused beverage prepared from a second amount of cannabinoid-containing substance. This detection could be made based on a count of a predetermined number of containers, a dynamically determined number of containers that can be filled from an available supply of cannabinoid-containing substance, and/or other production, processing, or control parameters.

The example method 1720 also involves controlling the marking station at 1728, to apply the first indicia to the last container of the first set in the succession and to apply a second indicia to the next container in the succession of containers, which is the first container of the second set. The first indicia is associated with the first amount as noted above, and the second indicia is associated with the second amount.

A beverage production run could include cannabis-infused beverages that are prepared from different amounts, which could be different lots for example, of cannabinoid-containing substance. The description of FIG. 30 above refers to first and second amounts, but there could be additional amounts as well. Subsequent transitions between sets of containers that are filled with cannabis-infused beverage prepared from different amounts of cannabinoid-containing substance could be detected, to initiate marking system control and indicia changes for additional sets of containers. Containers that are filled with cannabis-infused beverage prepared from different amounts of cannabinoid-containing substance can therefore be labelled with respective different indicia, to allow each container to be traced to the amount of cannabinoid-containing substance from which the cannabis-infused beverage that it contains was prepared.

A processor-readable storage medium could be used in implementing at least the operations at 1724, 1726, 1728, with processor-executable instructions being stored on such a medium. The instructions, when executed by a processor, cause the processor to perform a method. Execution of the instructions could cause a computing device that includes the processor to implement a system configured to, in some embodiments: control a marking station to apply indicia as shown at 1724 and described above, detect one or more transitions as shown at 1726 and described above, and control a marking system to change indicia between sets of containers as shown at 1728 and described above.

An automated marking system could include such a computing device, as well as a marking station such as an automated labelling system. These and/or other possible implementation options in respect of a system that could be configured or used to perform a method consistent with FIG. 30 could be or become apparent. FIGS. 26-28, for example, illustrate one possible embodiment of a system in which components could be configured to perform such a method.

In an embodiment, a variation of the example method 1720 relates to a method for bottling a cannabis-infused beverage. In the context of such a method, or a system that implements or performs such a method, the term “bottling” is intended to be generally indicative of filling containers, which could include bottles such as glass bottles and/or plastic bottles, and could also or instead include other types of containers such as cans, for example. In general, containers could be or include one or more of: glass containers, plastic containers, and/or other containers such as aluminum containers.

FIG. 30 illustrates, at 1722, providing a marking station. In some embodiments, a filling line including a filling station, a container marking station and a control device is provided. Providing a filling line is not intended to imply that a filling line is manufactured as part of a bottling method. A filling line or filling line equipment could be purchased or otherwise acquired, and thereby be provided for use in a bottling method, as noted above.

A filling station is operative to fill containers, and various examples of fillings stations will be apparent to those familiar with bottling or filling lines. Although the particular structure of a filling station may vary depending on the type(s) of containers to be filled, a filling station includes a supply or input stage or substation to prepare or receive the beverage(s) to be with which containers are to be filled, a dispensing station or substation including a dispenser or set of dispensers such as nozzles to dispense the beverage(s) into one or more containers at a time, and an output stage through which filled containers are output for further handling. Closing of containers, for example, could be performed by a closing stage or substation of a filling station or by separate equipment on a filling line.

Examples of marking stations are provided elsewhere herein.

The control device of a filling line is configured to control an operation of the container marking station, and could also control other filling line components. A control device could be implemented, for example, as part of a production control system. Examples of control devices, such as controllers, are provided elsewhere herein.

Although not shown in FIG. 30, some embodiments could involve filling containers, at the filling station, with cannabis-infused beverage supplied from a master batch of cannabis-infused beverage. The master batch could be prepared from an amount of cannabis-containing substance derived from cannabis plant material. The cannabis-containing substance contains one or more cannabinoids. The master batch includes a quantity of cannabis-infused beverage to fill a plurality of containers, and the filling station is configured to perform a supply switch from a first master batch to a second master batch of cannabis-infused beverage. The supply switch could switch from one supply source to another, and involve controlling one or more valves for example. A first set of containers is filled with cannabis-infused beverage drawn from the first master batch and, after the supply switch, a second set of containers is filled with cannabis infused beverage drawn from the second master batch.

An indicia could be applied on each container at the marking station, as discussed above with reference to operation 1724 for example. In the present embodiment involving multiple master batches, the indicia is indicative of the master batch of the cannabis-infused beverage supplying the filling station when the container is filled by the filling station. In some embodiments, the indicia is, includes, conveys, or is indicative of a lot number.

The example method 1720 includes controlling the marking station at 1728, and a bottling method could similarly involve controlling, with the control device of the filling line, the operation of the marking station such that when a supply switch is performed from the first master batch to the second master batch, a marking switchover from a first indicia to a second indicia is performed by the marking station. Marking station operation is controlled to perform the marking switchover such that containers filled with cannabis-infused beverage drawn from the first master batch are marked with a first indicia associated with the first master batch, and containers filled with cannabis-infused beverage drawn from the second master batch are marked with a second indicia associated with the second master batch. Examples of indicia and how indicia could be applied to containers, are disclosed elsewhere herein.

A bottling method could also involve other operations, such as preparing a master batch from an amount of cannabis-containing substance. Preparing the master batch could include diluting the cannabis-containing substance with a diluent. In an embodiment, the diluent includes water.

In some embodiments, a bottling method includes adjusting an amount of diluent added to the cannabis-containing substance to achieve a target concentration, which could be a predetermined concentration or a dynamically determined concentration, of the cannabinoid in the master batch. A method could involve holding the prepared master batch, and/or a master batch that was provided in a prepared and filling-ready form, in or into a holding tank. The filling station could then be supplied with cannabis-infused beverage from the holding tank.

As part of a bottling method, multiple holding tanks, each configured to hold a respective master batch of cannabis-infused beverage, could be provided. As noted elsewhere herein for other components such as a marking station and a filling line, providing holding tanks could involve purchasing or otherwise acquiring holding tanks and not necessarily manufacturing holding tanks.

Some embodiments involve providing, by manufacturing or otherwise, a supply selection valve in fluid communication with the holding tanks and with the filling station. The supply selection valve is capable of selectively acquiring any of a number of supply positions, with each supply position associating a respective holding tank with the filling station to supply the filling station from the respective holding tank. Such a supply selection valve could be manually operable. In an automated bottling system, however, the control device of the filling line could control the supply selection valve and direct the supply selection valve to acquire a selected supply position among its supply positions.

For example, in an embodiment, the control device is configured to command the supply selection valve to switch a supply position to perform a supply switch from a first holding tank holding the first master batch to a second holding tank holding the second master batch. The control device could command the supply selection valve to switch the supply position to perform the supply switch from the first holding tank to the second holding tank when sensing that the first holding tank is empty, or is at or below a minimum threshold volume of the first master batch.

In some embodiments, the first holding tank and the second holding tank include respective level sensors generating outputs indicative of the level of cannabis-infused beverage in the respective holding tanks, and the control device receives the outputs of the respective level sensors. The control device is thereby able to determine fill level of a current supply holding tank from which containers are currently being filled, and switch supply to another holding tank when the current supply holding tank runs low or empty. A supply switch could be made when the beverage volume remaining in the current supply holding tank is at or below a volume required to fill a certain number of containers, for example. The number of containers from which a minimum volume threshold is determined could be one, to minimize production loss or waste, or more than one, to potentially reduce the likelihood of a holding tank running dry and interrupting production. Different minimum volumes could be used for different holding tanks and/or different master batches.

Supply switching could alternate between holding tanks, and not switch only from one holding tank to another. For example, a filling line that is operable with either of two holding tanks could switch supply from a first holding tank to a second holding tank when a volume of beverage in the first holding tank is at or below a minimum volume. The first holding tank could then be refilled with a further master batch, a supply switch back to the first holding tank could be performed when a volume of beverage in the second holding tank is at or below a minimum volume, and then the second holding tank could be refilled with another master batch. Filling lines that work in conjunction with more than two holding tanks or supply sources are also contemplated.

In some embodiments, the filling station receives a succession of empty containers and fills the empty containers with cannabis-infused beverage. Containers could be filled in succession, one at a time. In other embodiments, the filling station includes nozzles, or other types of dispensers, so that a set of multiple empty containers can be filled simultaneously.

The marking station could be or include, for example, a labeling station for applying a label on each container, in which case the label bears the indicia that is to be applied to a container. In such an implementation, the marking station could include a supply of labels and be configured to apply to each container a label from the supply of labels.

The marking station could be configured to apply the indicia to a label from the supply of labels and apply the label to the container. In some embodiments, the labels in the supply of labels are pre-printed with indicia. A label supply could include sets of labels that are pre-printed with respective different indicia, and the marking station could then select one of the sets of labels for a container based on the indicia with which the container is to be marked.

Label-based marking is one illustrative embodiment. The marking station could also or instead print the indicia on a container.

Whether label-based marking or another type of marking is applied by the marking station, the marking station could apply the indicia to each container before the container is filled with cannabis-infused beverage. The marking station could apply the indicia to each container after the container is filled with cannabis-infused beverage in some embodiments.

A control device could include, or at least access, a computer readable storage, and be configured to determine a number of containers filled with cannabis-infused beverage from a particular master batch and store in the computer readable storage the determined number. This could be useful, for example, in tracking productivity and/or inventory control.

The control device could include an input to receive an identifier associated with the amount of cannabis-containing substance, and be further operative to link in the computer readable storage the determined number of containers filled with cannabis-infused beverage made from the amount of cannabis-containing substance and the identifier. The control device could also or instead be operative to link, in the computer readable storage, the indicia applied on the containers filled with cannabis-infused beverage made from the amount of cannabis-containing substance and the identifier associated with the predetermined amount of cannabis-containing substance.

A processor-readable storage medium could be used in implementing at least some of the operations in these variations of the example method 1720, with processor-executable instructions being stored on such a medium. The instructions, when executed by a processor, cause the processor to perform a method. Execution of the instructions could cause a computing device that includes the processor to implement a system configured to, in some embodiments: fill containers, apply indicia, and control a marking station as discussed above and/or elsewhere herein.

An automated marking system could include such a computing device, as well as a filling station and a marking station such as an automated labelling system. These and/or other possible implementation options in respect of a system that could be configured or used to perform a method consistent with these variations in the example method illustrated in FIG. 30 could be or become apparent. FIGS. 26-28, for example, illustrate one possible embodiment of a system in which components could be configured to perform such a method.

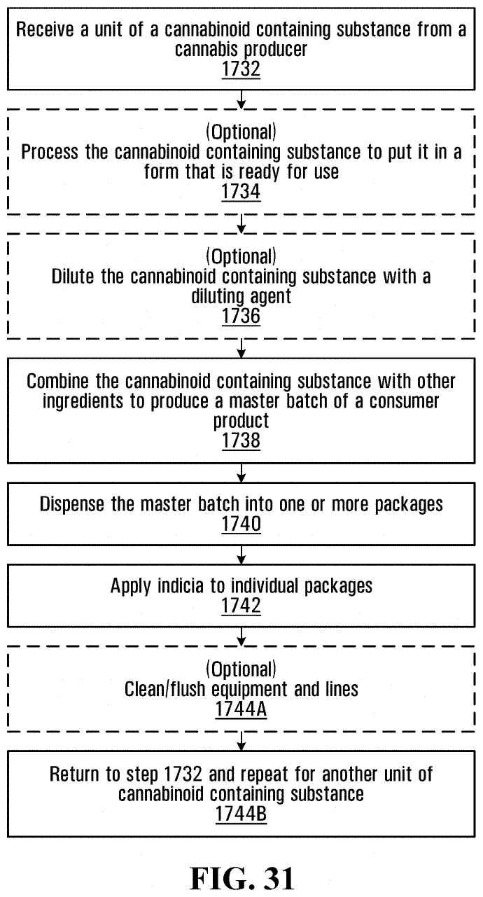

FIG. 31 is a method of producing a cannabis-infused consumer product, according to one embodiment. The cannabis-infused consumer product may be any one of the examples described above, e.g. an edible, beverage, topical, mucoadhesive delivery system, vape oil, vape oil cartridge, etc.

In step 1732, a unit of cannabinoid-containing substance is received from a cannabis producer 1502. The unit of cannabinoid-containing substance has a particular dose and particular lot number. The unit of cannabinoid-containing substance is of or from a particular amount, e.g. of or from a particular lot comprising an amount of cannabinoid-containing substance derived from cannabis plant material to produce the lot, which has a particular lot number. The unit of cannabinoid-containing substance may be associated with a particular extraction process record, and/or an extract record, and/or an oil container record, and/or a lab sample record, and/or an oil jar record, and/or a lot record, which is/are stored in the ICS 1506 of the cannabis producer 1502. However, all of this record information is not necessarily transferred to the ICS 1508 of the cannabis processor 1504. In some embodiments, perhaps only the lot number and other required information (e.g. dose of the cannabinoid-containing substance, name or ID of the cannabis producer 1502, etc.) is provided to the cannabis processor 1504. The lot number and possibly other information (e.g. the dose of the cannabinoid-containing substance and name/ID of cannabis producer 1502) is stored in the ICS 1508, e.g. in a record created and stored in the ICS 1508 in association with the received unit of cannabinoid-containing substance.

Optionally, in step 1734, processing is performed on the cannabinoid-containing substance to put it in a form that is ready for use by the cannabis processor 1504. Whether step 1704 is performed, and if so, the extent of the processing in step 1704, will depend upon the form of the unit of cannabinoid-containing substance as received from the cannabis producer 1502.

Optionally, in step 1736, the cannabinoid-containing substance is diluted with a diluting agent. In some embodiments, the diluting agent may be water and/or oil.

In step 1738, the cannabinoid-containing substance is combined with other ingredients to produce a master batch of a consumer product. The ICS 1508 may generate a record for the master batch, e.g. assigning a master batch number that is associated with/linked to the record documenting the transfer of the cannabinoid-containing substance and the lot number of the cannabinoid-containing substance. An identifier, e.g. a lot number, may be stored in the ICS 1508 and/or in memory in a control device (e.g. control device 1660) in association with the master batch and/or in association with units of consumer product produced from the master batch.

In step 1740, the master batch is dispensed into one or more packages. In some embodiments, the packages are containers or bottles depending upon the consumer product. Each package holds a portion of the master batch.

In step 1742, indicia (e.g. a lot number) is applied on individual packages by feeding a stream of individual packages to a marking station. A marking station is sometimes called a marking unit.

In step 1744A, optionally, the workspace, bottle filling machine(s) and lines and/or holding tanks are cleaned. Step 1744A could include washing or flushing certain components of the bottle filling machine with solvents (for example, water and/or ethanol) and/or compressed air. Cleaning/flushing the equipment helps prevent cross-contamination of toxins/contaminants between lots/batches, and also isolates cannabinoids between lots/batches, which further improves traceability of cannabinoids.

In step 1744B, steps 1732-1742 are repeated for each unit of cannabinoid-containing substance, and in step 1742 a control device (e.g. control device 1660) distinguishes between individual packages holding a unit of consumer product made from different lots of cannabinoid-containing substance. The marking station is controlled by the control device to apply to each individual package an indicia (e.g. lot number) derived from the identifier (e.g. lot number) of the respective lot of cannabinoid-containing substance from which the consumer product in the package was made.

In some embodiments, when a master batch of a consumer product is being dispensed into individual packages, there may result in a residual volume of consumer product from the master batch that is less than the volume of consumer product required to fill the individual package to capacity. A control device, e.g. control device 1660 described earlier, may obtain the number of individual packages that are or can be filled to capacity from that master batch. The number may be stored in machine-readable storage (e.g. memory 1664) accessible by the control device. In some embodiments, the control device controls the marking station to operate the marking station a corresponding number times to apply to each individual package from the master batch an indicia associated with/linked back to the lot of cannabinoid-containing substance from which the master batch (and each consumer product in each package originating from the master batch) was made. In this way, the control device controls the marking station to apply the correct indicia (e.g. lot number) to each package across different master batches originating from different lots of cannabinoid-containing substance.

In some embodiments, the dispensing of the master batch is performed such that the consumer product in each package of a set of packages originates from a same single amount (e.g. same lot) of cannabinoid-containing substance. In some embodiments, the number of packages may be counted by a control device and stored.

In another embodiment, a method for manufacturing and packaging a cannabis-infused consumable product made from a cannabis-containing substance could include at least some operations similar to those in FIG. 30. For example, a manufacturing method could include providing one or more manufacturing inputs, such as multiple amounts of cannabis-containing substance that contains one or more cannabinoids. Each amount of cannabis-containing substance could be derived from cannabis plant material, and be associated with an identifier allowing distinguishing of one amount from another amount. Extract identifiers and lot identifiers as disclosed elsewhere herein are examples of identifiers with which each amount could be associated to enable amounts to be distinguished from each another.

One or more manufacturing line or system components could also be provided. For example, a manufacturing and packaging method could involve providing a control device that has, or at least has access to, a machine-readable storage. A method could then include storing, in the machine-readable storage, identifiers associated with respective ones of the amounts of cannabis-containing substance.

In manufacturing a consumable product, a method could involve, for example diluting each amount of cannabis-containing substance with a diluent or diluting agent, such as water or oil, to produce a master batch of consumable product. The master batch could then be dispensed into a set of packages, with each package holding a portion of the master batch. A marking unit or station could be provided, as shown at 1722 for example, and a method could include applying an indicia on individual packages, as shown by way of example at 1744B. Applying an indicia could include feeding a stream of individual packages to a marking unit or station, and distinguishing, in the stream, between individual packages holding a consumable product made from different amounts of cannabis-containing substance and controlling the marking unit or station, as shown at 1728 for example, with the control device to apply to each individual package an indicia derived from the identifier of the respective amount from which the consumable product in the package was made. The indicia could be, include, convey, or indicate the identifier of the amount.

In some embodiments, the cannabis-infused consumable product is vaping oil. In such embodiments, each package could be a vaping cartridge containing vaping oil.

Another example of a consumable product is a cannabis-infused beverage.

The consumable product is an emulsion in some embodiments.

Dispensing of the master batch could be performed such that the consumable product in each package originates from a single amount of cannabis-containing substance. This could involve the control device controlling filling or dispensing equipment to fill the packages from a single source of diluted concentrate, for example. A different source could subsequently be used to fill another set of packages.

A method could include determining, by counting for example, the number of packages produced from a particular amount of cannabis-containing substance and storing the counted number in the machine-readable storage. The number of packages could be useful for production monitoring and/or inventory control, for example.

Another embodiment of a method for manufacturing and packaging a cannabis-infused consumable product made from a cannabis-containing substance also involves providing multiple amounts of cannabis-containing substance containing one or more cannabinoids, with each dose being derived from cannabis plant material; providing a control device having a machine-readable storage; storing in the machine-readable storage identifiers associated with respective ones of the amounts of cannabis-containing substance to allow distinguishing one amount from another amount; diluting each amount of cannabis-containing substance with a diluting agent to produce respective master batches of consumable product; and dispensing the master batches into respective sets of individual packages, with each package of a given set holding a portion of the respective master batch, as described in an example above. In the present embodiment, a stream of individual packages is fed to a marking unit, and the stream is arranged in an order determined by which master batch is the source of the consumable product held in each individual package. Packages for which one master batch is the source could be fed to the marking unit first, followed by packages for which a different master batch is the source, for example. Other arrangements are also possible.

Under control of the control device, the operation of the marking unit is synchronized with the order in which the stream of individual packages is arranged, such that each individual package receives an indicia associated with the particular dose from which the consumable product in the package is made. Such synchronization could be based on a number of packages for which each amount is the source. For example, if one amount was the source for “x” packages, then the marking unit, the control device, or another component could count packages until “x” packages have received an indicia associated with that amount, and then the control device could control the marking unit to change the indicia to a different indicia associated with a different amount of cannabis-containing substance that is the source for subsequent packages in the package stream.

In another embodiment, a method for manufacturing and packaging a cannabis-infused consumable product made from a cannabis-containing substance involves operations of providing multiple amounts of cannabis-containing substance, providing a control device, and diluting each amount of cannabis-containing substance with a diluent or diluting agent as described above. In an embodiment, each amount is diluted to produce respective master batches of consumable product. For each master batch, the master batch is dispensed into a set of individual packages, with each package holding a portion of the master batch, and a residual volume of consumable product from the master batch that is less than the volume of consumable product required to fill the individual package to capacity is withheld from dispensing into an individual package.

For one or more master batches, the number of individual packages filled to capacity from the master batch in the respective set of individual packages could be determined, and the number could then be stored in the machine-readable storage.

A stream of individual packages could be fed to a marking unit, which is controlled with the control device. Controlling the marking unit could include deriving from the machine readable storage the number of filled packages and operating the marking unit a corresponding number of times to apply to each individual package in the set an indicia linked to the particular amount of cannabis-containing substance from which the consumable product in the package is made. In this manner, sets of packages including consumable product produced from respective different amounts of cannabis-containing substance have an indicia, applied by that marking unit, which is linked to the respective amount.

In some embodiments, residual amounts from multiple cannabis-containing substance amounts could be combined in order to collect sufficient volume to fill one or more packages. An indicia, or multiple indicia, linked to each of the multiple amounts of cannabis-containing substance, could then be applied to such packages by the marking unit under control of the control device.

Residual amounts could instead be designated as waste and collected for destruction. The residual amounts could be measured and recorded, and used in production monitoring and/or inventory control, for example.

Features disclosed elsewhere herein could be implemented in conjunction with such a manufacturing and packaging method. For example, such a method could be employed in manufacturing and packaging a cannabis-infused consumable product in the form of vaping oil. In some embodiments, each package for vaping oil could be a vaping cartridge containing vaping oil. Oil is one example of a diluting agent that could be used in manufacturing vaping oil.

A cannabis-infused beverage is another example of a consumable product. In some embodiments, the diluting agent for manufacturing a cannabis-infused beverage is water.

Yet another example of a consumable product is an emulsion.

In a manufacturing method, the step of dispensing the master batch could be performed such that the consumable product in each package originates from a single amount of cannabis-containing substance.

Some embodiments could include counting the number of packages produced from a particular amount of cannabis-containing substance and storing the counted number in machine-readable storage.

The cannabis-containing substance could be a cannabis extract in some embodiments.

As noted at least above in respect of other embodiments, a processor-readable storage medium could be used in implementing at least some of the operations in these example methods relating to cannabis-infused consumer products, with processor-executable instructions being stored on such a medium. The instructions, when executed by a processor, cause the processor to perform a method. Execution of the instructions could cause a computing device that includes the processor to implement a system configured to, in some embodiments, perform at least some of the method operations discussed above and/or elsewhere herein.

A production system could include such a computing device, as well as other components involved in producing a cannabis-infused consumer product. These and/or other possible implementation options in respect of a system that could be configured or used to perform a method consistent with these example methods disclosed herein could be or become apparent. FIGS. 26-28, for example, illustrate one possible embodiment of a system in which components could be configured to perform such methods.

In some embodiments, the cannabis-containing substance is a food additive. A food additive provided herein comprising an emulsion or nanoemulsion microencapsulation system may be formed using any of the techniques available to fabricate emulsions and nanoemulsions. The techniques available are commonly classified as either high or low energy approaches.

High energy approaches use mechanical devices known as “homogenizers” that generate intense disruptive forces that mix the oil and water phases together, as well as break larger droplets into smaller ones. O/W emulsions are usually prepared by homogenizing an oil phase and a watery phase together in the presence of a water-soluble hydrophilic emulsifier. A variety of specialized homogenization equipment is available for fabricating emulsions and nanoemulsions that include, but are not limited to, high shear mixers, high pressure valve homogenizers, microfluidizers, colloid mills, ultrasonic homogenizers, and membrane and microchannel homogenizers.

High shear mixers are a type of rotor-stator device that homogenizes oil, water, and other ingredients in a batch process. Typically, the droplets produced by a high shear mixer range between about 1 and about 10 μm in diameter. A suitable vessel may contain as a few cm³ or as large as several m³. The rapid rotation of the mixing head generates a combination of longitudinal, rotational, and radial velocity gradients in the fluids, which disrupts the interfaces between the oil and water phases, causing the liquids to become intermingled, and breaks the larger droplets into smaller ones. Efficient homogenization is achieved when the horizontal and vertical flow profiles distribute the liquids evenly throughout the vessel, which can be facilitated by having baffles fixed to the inside walls of the vessel. The design of the mixing head determines the efficiency of the homogenization process, and a number of different types are available for different situations, for example, blades, propellers, and turbines.

High-pressure valve homogenizers are used to produce fine emulsions from pre-existing emulsions (“coarse emulsion”), with emulsion droplets as small as 0.1 μm. The homogenizer has a pump that pulls the coarse emulsion into a chamber on its backstroke and then forces it through a narrow valve at the end of the chamber and on its forwards stroke it experiences a combination of intense disruptive forces that cause the larger droplets to be broken down to smaller ones. The flow regime that is responsible for disrupting the droplets in a particular high pressure valve homogenizer depends on the characteristics of the material being homogenized, the size of the homogenizer, and the design of the homogenization nozzle.

Microfluidization creates emulsions with very fine droplets whose diameter can be less than 0.1 μm. This type of homogenizer typically consists of a fluid inlet (single or double), some kind of pumping device, and an interaction chamber containing two channels. Fluids are introduced into the homogenizer, accelerated to a high velocity and then made to simultaneously impinge with each other on a solid surface, which causes the fluids to intermingle and disrupt larger droplets.

Colloid mills are used to homogenize medium and high viscosity liquids. A colloid mill typically contains two disks: a rotor (a rotating disk) and a stator (a static disk). The liquids and other ingredients to be homogenized are usually fed into the center of the colloid mill in the form of a pre-existing emulsion. The intensity of the shear stresses (and therefore the droplet disruption forces) can be altered by varying the rotation speed, gap thickness, rotor/stator type, and throughput to reduce droplet sizes. Typically, colloid mills can be used to produce emulsions with droplet diameters in the range between about 1 and about 5 μm.

Ultrasonic homogenizers use high-intensity ultrasonic waves that generate intense shear and pressure gradients within a material that disrupt droplets mainly through cavitation and turbulent effects. The present invention can use any of the available methods that are available for generating high-intensity ultrasonic waves including, but not limited to, piezoelectric transducers and liquid jet generators.

Membrane homogenizers can be used in two main ways to process emulsions, direct homogenization and premix homogenization. Direct homogenization involves forming an emulsion directly from the separate oil and water phases in the presence of a suitable emulsifier. Premix homogenization involves reducing the size of the droplets present within an existing coarse emulsion. The droplet size attained depends on the membrane pore size, the oil-water interfacial tension, the applied pressure, the flow profile of the continuous phase, and the type and amount of emulsifier used.

Low energy approaches to produce emulsions and nanoemulsions rely on the spontaneous formation of oil droplets in surfactant-oil-water mixtures which either their composition or environment is altered in a controlled way. Examples of low energy methods include, but are not limited to, spontaneous emulsification methods, emulsion inversion point methods, and phase inversion temperature methods.

Spontaneous emulsification involves titrating a mixture of oil and water-soluble surfactant into a water phase with continuous stirring. Small oil droplets are spontaneously formed at the oil-water boundary as the surfactant molecules move from the oil phase to the water phase. The spontaneous emulsification method has been used widely within the pharmaceutical industry to encapsulate and deliver lipophilic drugs. Such systems are known as either self-emulsifying drug delivery systems (SEDDS) or self-nanoemulsifying drug delivery systems (SNEDDS) depending on the droplet size produced. Self-emulsifying formulations are readily dispersed in the gastrointestinal tract, where the motility of the stomach and small intestine provides the agitation necessary for emulsification.

Emulsion inversion point methods involve titrating water into a mixture of oil and water-soluble surfactant with continuous stirring. As increasing amounts of water are added, a W/O emulsion is initially formed, then an O/W/O emulsion, and then an O/W emulsion.

Phase inversion temperature (PIT) methods rely on heating a surfactant-oil-water mixture around or slightly above its PIT and the quench cooling with continuous stirring. When the emulsion passes through the PIT, the optimum curvature tends towards unity, thereby leading to an ultralow interfacial tension and a highly dynamic interface. For a general overview of emulsification technology, see, e.g., McClements, David J., Food Emulsions: Principles, Practices, and Techniques, 3^{rd} ed (Boca Raton, Fla.: CRC Press, 2016).

In some embodiments, the herein described emulsion of cannabinoids may include, for example, per total volume of emulsion up to 1 g/ml, up to 750 mg/ml, up to 700 mg/ml, up to 650 mg/ml, up to 600 mg/ml, up to 550 mg/ml, up to 500 mg/ml, up to 450 mg/ml, up to 400 mg/ml, up to 350 mg/ml, up to 300 mg/ml, up to 250 mg/ml, up to 200 mg/ml, up to 150 mg/ml, up to 100 mg/ml, up to 50 mg/ml, up to 40 mg/ml, up to 35 mg/ml, up to 30 mg/ml, up to 25 mg/ml, up to 20 mg/ml, or up to 15 mg/ml of a specific cannabis extract such as THC, CBD, terpene (e.g., D-limonene) or any mixtures thereof, and the like.

In some embodiments, once a suitable emulsion of the cannabinoid has been produced, the emulsion is dehydrated to form a powder, typically using spray drying. For example, the emulsion may be dried to obtain a water activity (a_{w}) of less than 0.6, for example 0.04≤a_{w}≤0.3. Water activity may be measured using an Aqualab Water Activity Meter 4TE (Decagon Devices, Inc., U.S.A.). For extra protection, the resulting powder can be atomized and coated with a secondary layer, typically a high melting fat or starch.

In some embodiments, the food additive is a beverage additive which includes the herein described emulsion of cannabinoid. Dilution or infusion of the beverage additive in a cannabinoid-less beverage or blending with a beverage base results in a beverage product comprising at least 0.002 mg/ml of cannabinoid in total volume of the beverage product. For example, the beverage product may include from 0.002 mg/ml to about 1 mg/ml of cannabinoid in volume of the beverage product.

In some embodiments, the food additive is a beverage additive which includes the herein described emulsion of cannabinoid. Dilution or infusion of the beverage additive in a cannabinoid-less beverage or blending with a beverage base results in a beverage product comprising at least 0.002 mg/ml of cannabinoid in volume of the beverage product, the beverage product having a turbidity of less than 0.05 cm⁻¹ at 600 nm.

In some embodiments, the food additive is a beverage additive which includes the herein described emulsion of cannabinoid. Dilution or infusion of the beverage additive in a cannabinoid-less beverage or blending with a beverage base results in a beverage product comprising at least 0.002 mg/ml of cannabinoid in volume of the beverage product, the beverage product having a viscosity selected in the range of from 50 mPas (for juice-like beverages) to 1500 mPas (for more honey-like beverages, such as fruit juice concentrates) measured at room temperature. In some embodiments, the beverage product may have a viscosity which is substantially the same as that one of the cannabinoid-less beverage.

In some embodiments, the food additive is a beverage additive which includes the herein described emulsion of cannabinoid. Dilution or infusion of the beverage additive in a cannabinoid-less beverage or blending with a beverage base results in a beverage product comprising at least 0.002 mg/ml of cannabinoid in volume of the beverage product, the beverage product having an odor index which is substantially the same as that one of the cannabinoid-less beverage. Odor index can be determined based on odor intensity index measuring method known in the art with which practical odor intensity can be objectively and easily measured, for example but without being limited thereto, as described in Somchai Rice and Jacek Koziel, PLOS ONE 10(12): e0144160.

In some embodiments, the food additive is a beverage additive which includes the herein described emulsion of cannabinoid. Dilution or infusion of the beverage additive in a cannabinoid-less liquid beverage results in a beverage comprising at least 0.002 mg/ml of cannabinoid in total volume of the liquid beverage and having a taste index which is substantially the same as that one of the cannabinoid-less beverage. Testing methods for assessing taste index are known in the art, and some of which are described in McDaniel, ACS Symposium Series, Vol. 289, chapter 1, p. 1-10.

In one embodiment, the expression “substantially the same” as used herein when referring to a tested parameter of a cannabinoid-containing beverage when compared to the same parameter tested in the cannabinoid-less beverage generally refers to the value resulting from the test being more or less 20%, identical, or more or less 15% identical, or more or less 10% identical. Typically, such will occur when a sensory evaluation (by a subject, e.g., tasting, smelling, looking, touching) will not detect any significant variations and yet, depending on the instrumentation used, may result in slight measured variations, e.g., more or less 20%, identical, or more or less 15% identical, or more or less 10% identical. However, because it is the sensory evaluation which likely has a more significant effect on the user experience and/or derived commercial benefit, even such slight variations will be deemed to be “substantially the same” for the purposes of the user's perspective, i.e., the consumer.

In some embodiments, a cannabinoid may be microencapsulated in micelles. Micelles consist of small clusters of surfactant molecules that self-assemble into a structure where the hydrophobic tails are located in the interior and the hydrophilic heads are located at the exterior. Micelles are thermodynamically stable systems under a particular range of compositional and environmental conditions, and should therefore form spontaneously. Nevertheless, some form of energy often has to be applied during their formation (such as simple mixing) to overcome kinetic energy barriers to the self-assembly of the surfactant molecules. Micelles are one of the smallest colloidal particles that are widely used as delivery systems, with diameters typically in the range from about 5 to 20 nm. Nonpolar active agents can be solubilized within the hydrophobic interior of micelles, whereas amphiphilic active agents can be incorporated at their exterior, with the loading capacity depending on the molecular dimensions of the active agents and the optimum curvature of the surfactant monolayer. Larger thermodynamically stable micelles (e.g., diameters up to 100 nm) may also contain an oil phase and possibly a co-surfactant. Termed “microemulsions” by IUPAC, larger thermodynamically stable micelles can solubilize higher levels of nonpolar active agents. They are usually fabricated from one or more small-molecule surfactants, but amphiphilic block copolymers can also be used.

In some embodiments, a cannabinoid may be microencapsulated in solid lipid nanoparticles or nanostructured lipid carriers. Solid lipid nanoparticles (SLNs) have similar structures to nanoemulsions (or emulsions), but the oil phase is crystallized rather than liquid. SLNs are typically fabricated by preparing an oil-in-water nanoemulsion at a temperature above the melting point (Tₘ) of the oil phase, and then cooling the system well below Tₘ to promote droplet crystallization. In principle, the crystallization of the lipid phase slows down molecular diffusion processes inside the particles, which may help to protect an encapsulated active agent from chemical degradation. SLNs have proven to be useful delivery systems for many applications in the pharmaceutical industry, where they are mainly used to encapsulate hydrophobic drugs. However, if the lipid phase is not carefully selected there can be appreciable challenges to their utilization for this purpose. Lipids that form highly regular crystalline structures (such as pure triacylglycerols) have a tendency to expel other nonpolar substances when they undergo a liquid-to-solid transition. Moreover, there may be an appreciable change in the morphology of the lipid nanoparticles, from spherical to irregular, when the lipid phase crystallizes or undergoes a polymorphic transition. As a result of the increase in particle surface area, there may be insufficient emulsifier to coat the particles, which leads to extensive aggregation. These problems can be overcome by using nanostructured lipid carriers (NLCs). In this case, a lipid phase is selected that forms more irregular crystals when it solidifies, which leads to less expulsion of encapsulated active agents and less particle aggregation.

In some embodiments, a cannabinoid may be microencapsulated in liposomes, nanoliposomes, or niosomes. Liposomes (diameter >100 nm) and nanoliposomes (diameter <100 nm) are colloidal systems that are composed of particles made up of concentric layers of phospholipid bilayers. Niosomes are formed when non-ionic surfactants assemble into similar structures. The bilayers form due to the hydrophobic effect, that is, the tendency for the system to reduce the contact area between the nonpolar phospholipid or surfactant tails and water. These systems may contain one (unilamellar) or numerous (multilamellar) phospholipid bilayers depending on the preparation method and ingredients used. Hydrophilic functional ingredients can be trapped inside the aqueous interior of liposomes and nanoliposomes, whereas amphiphilic and lipophilic active agents can be trapped in the bilayer region. Liposomes and nanoliposomes can be fabricated from natural components, such as phospholipids. Cholesterol is often added to the formulation as it increases rigidity strength of the membrane and confers steric stability. Egg yolk- and soy-derived phosphatidylcholines are commonly used to form liposomes, whereas Tween® 80, Span® 80 and sucrose laurate have been used to form niosomes.

In some embodiments, a cannabinoid may be microencapsulated in polymer or hydrogel particles. Polymer microparticles (diameter>100 nm) and nanoparticles (diameter<100 nm) are fabricated from either synthetic or natural polymers, such as proteins and polysaccharides. Commonly, they are produced from antisolvent precipitation methods where a polymer dissolved in a good solvent is injected into a poor solvent, which promotes spontaneous particle formation. Hydrogel particles (sometimes called nanogels or microgels) may also be fabricated from synthetic or natural polymers, but they contain higher levels of water (typically >80% to 90%). A wide variety of different methods are available for producing hydrogel particles including injection, templating, emulsion, and phase separation methods. The composition and porosity of hydrogel particles must be carefully controlled to ensure appropriate loading, retention, and release properties.

In some embodiments, a food additive provided herein may further comprise a terpene or terpenoid. The term “terpene” is generally understood to include any organic compound derived biosynthetically from units of isoprene, and the term “terpenoid” generally refers to a chemically modified terpene (e.g., by oxidation). As used herein, terpenes include terpenoids. Terpenes may be classified in various ways, such as by their sizes. For example, suitable terpenes may include monoterpenes, sesquiterpenes, or triterpenes. At least some terpenes are expected to interact with, and potentiate the activity of, cannabinoids.

Examples of terpenes known to be extractable from cannabis include aromadendrene, bergamottin, bergamotol, bisabolene, borneol, 4-3-carene, caryophyllene, cineole/eucalyptol, p-cymene, dihydrojasmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpineol, 4-terpineol, terpinolene, and derivatives thereof.

Additional examples of terpenes include nerolidol, phytol, geraniol, alpha-bisabolol, thymol, genipin, astragaloside, asiaticoside, camphene, beta-amyrin, thujone, citronellol, 1,8-cineole, cycloartenol, and derivatives thereof. Further examples of terpenes are discussed in US Patent Application Pub. No. US2016/0250270.

In some embodiments, an edible product provided herein may further comprise other additives. Examples of suitable other additives include, but are not limited to, carbonation, pH control agents, vitamins, minerals, chelating agents, antioxidants, antimicrobial agents, flavors, sweeteners, colorants, weighting agents, fat replacers, and mixtures thereof.

In some embodiments, a food additive provided herein may be made using a method comprising the steps of: (1) adding cannabis oil, powder, distillate, or isolate to water; (2) adding an emulsifier; (3) subjecting the mixture to a high shear mixer as described in the Internet site of Proscientific which can be found on https://proscientific.com/cannabis on Jul. 31, 2018.

In some embodiments, a food additive provided herein may be made using a method comprising the steps of: (1) gently warming a cannabis extract by water bath; (2) addition of a starch-based powder, such as maltodextrin, to the warm cannabis extract; (3) mix the cannabis extract and starch-based powder together to create a uniform concentrated cannabis extract powder, and (4) addition of powder to hot water to dissolve the powder and emulsify the extract, as disclosed in U.S. Pat. No. 9,629,886 B2. The preferred temperature of the water bath is between 80 and 100 degrees Fahrenheit and more preferably between 84 and 90 degrees Fahrenheit. The ratio of the starch-based powder to the cannabis extract may be at least 24:1 w/w. The mixing step may be performed using an industrial blender to ensure even absorption of the powder by the extract. Other types of powders fit for human consumption may be used in place of the starch-based powder, including but not limited to, whey protein isolate (both dairy-based and plant-based), xanthan gum, guar gum (guaran), mono- and diglycerides, and carboxymethylcellulose (cellulose gum) so long as they absorb the oil when blended together, dissolve when added to a liquid, remain dissolved in that liquid and have no post-mixing separation of the powder and the oil.

In some embodiments, a food additive provided herein may be made using a method comprising the steps of: (1) heating an oil; (2) addition of a cannabis extract to the heated mixture; (3) addition of water or an aqueous solution to the heated mixture; (4) addition of at least one emulsifying agent to the heated mixture; and (5) mixing the heated mixture and added ingredients, as disclosed in WO 2017/180948A1. The oil is preferably in the range of 0.1% to 40% of the liquid formulation. The preferred oil temperature is between 120 to 220 degrees Fahrenheit. The amount of cannabis extract will be in the range of about 5 mg to 30 mg per 2 ounces of liquid formulation. The water or aqueous solution will be present in the range of 60% to 99.9% of the liquid formulation. The emulsifying agent(s) will be added in the amount of 0.15% and 2% of the total volume of the edible product and may be selected from the group consisting of xanthan gum, guar gum, cyclodextrin, lecithin, carrageen, monoglycerides, natural emulsifiers and organic emulsifiers that are safe for ingestion by humans. The mixing step may be performed using a high speed blender (or similar machine). The blender is run at high speed for between 30 seconds and 2 minutes. In some embodiments, caffeine (or anhydrous caffeine) may be added after the mixing step in the amounts ranging from 10-300 mg per 2 ounces of the emulsification. Alternatively, the caffeine can be added prior to adding the emulsifying agent or at the same time.

In some embodiments, a food additive provided herein may be made using a method comprising the steps of: (1) adding water; (2) adding one or more surfactant; (3) mixing water and one or more emulsifier using a magnetic stirring plate or stick; (4) adding cannabis oil to the mixture; (5) subjecting the mixture to a low shear mixer; (6) subjecting the mixture to a high shear mixer as described in the Internet site of Analytic company in the UK which can be found at https://analytik.co.uk/wp-content/uploads/2017/03/application-note-use-of-microfluidizer-technology-for-cannabis-products.pdf on Jul. 31, 2018. The low-shear mixer may be a rotor-stator mixer. The high shear mixer may be a microfluidizer. The mixture may be passed through each mixer one or more times. Pressure, number of passes, and temperature of the process may be adjusted.

In some embodiments, a food additive provided herein may be made using a method comprising the steps of: (1) mixing cannabis oil and a first emulsifier; (2) adding baicalein; (3) adding ethanol; (4) heating the mixture to 50° C. until all ingredients melt to form the oil phase mixture; (5) mixing a second emulsifier with water to form an aqueous phase mixture; (6) mixing the aqueous and oil phase mixtures; (7) subjecting the mixture to a high shear mixer for 5 minutes; (8) subjecting the mixture to a microfluidizer as described in Juntao Yin et al, “Biocompatible nanoemulsions based on hemp oil and less surfactants for oral delivery of baicalein with enhanced bioavailability” (2017) Int J Nanomedicine, 12, 2923. A particle size of 90.6 nm can be achieved using a formulation comprising 40 mg of baicalein, 1,000 mg of hemp oil, 50 mg of poly(ethylene glycol) monooleate as the first emulsifier, and 50 mg of sodium oleate as the second emulsifier mixed with 20 mL of water. The ratio of the first emulsifier to the second emulsifier may be about 1:1.

In some embodiments, a food additive provided herein may be made using a method comprising the steps of: (1) dilution of the cannabis extract with an oil; (2) addition of an emulsifier; (3) sonication to produce an oil-cannabis mixture; and (4) emulsification of the oil-cannabis mixture with water, as described at the Internet site of the hielscher company at https://www.hielscher.com/ultrasonic-cannabis-oil-emulsion.htm on Jul. 29, 2018. The oil may be vegetable oil such as olive oil or coconut oil. The ratio of the extract to the oil may be about 1:40 v/v. The emulsifier may be lecithin, arabic gum, or a starch-based emulsifier. The ratio of the extract to the emulsifier may be between 1:10 and 1:15 w/v. The sonication step may be performed using an ultrasonic homogenizer. The ratio of cannabis-oil mixture to water may be about 2:5 v/v. The emulsification step may be performed using an ultrasonic homogenizer.

In some embodiments, a food additive provided herein may be made using a method comprising the steps of: (1) adding cannabis oil, distillate, or isolate; (2) adding a carrier oil; (3) adding a mixture of emulsifiers; (4) adding of distilled water; (5) subjecting the mixture to sonication to produce a nanoemulsion with droplet sizes of about 20 to 40 nm, as described in the Internet site of Sonomechanics which can be retrieved at http://blog.sonomechanics.com/blog/stabilizer-package-for-producing-water-soluble-cannabis-extracts on Jul. 31, 2018. The sonication may be performed using an ultrasonic homogenizer.

In some embodiments, a food additive provided herein may be made using a method comprising the steps of: (1) adding cannabis oil, distillate, or isolate; (2) adding a carrier oil; (3) adding a first emulsifier; (4) heating the mixture up to 110° C.; (5) cooling the mixture for 24 hours; (6) mixing water and a second emulsifier and heating the mixture up to 45° C., then allowing the mixture to cool for 24 hours; (7) mixing the two mixtures using a magnetic stirrer at room temperature; (8) subjecting the mixture to sonication as described in https://leherbe.com/knowledge-center/experiment/emulsification on Jul. 31, 2018. The first emulsifier may be Span 80. The second emulsifier may be Tween 80. Preferably, the oil volume fraction is at ϕₒ=0.10 and the total emulsifier volume fraction is at ϕₛ=0.08. Preferably, the sonication time is between 5 and 7.5 minutes.

In some embodiments, a food additive provided herein may be made using a method comprising the steps of: (1) adding cannabis oil; (2) adding a suitable pair of emulsifiers 5 or 10 wt % with a hydrophilic-lipophilic balance (HLB) ranging from 6 to 10; (3) adding distilled water; (4); heating the mixture to 70° C.; (5) subjecting the mixture immediately to sonication for 15 minutes as described in Mikulcová et al., “Formulation, Characterization and Properties of Hemp Seed Oil and Its Emulsions”, Molecules (2017) 22, 700. The use of Tween 85 and Span 85 at 10 wt % as emulsifiers produced particles ranging in diameter from 84 nm to 122 nm.

In some embodiments, a food additive provided herein may be made using a method comprising the steps of: (1) spreading cannabis oil on a thin film of parchment paper or PTFE sheets; (2) subjecting oil to a 100 hour purge in a vacuum oven until cannabis shatter forms; (3) flipping the extracted solution during the process on a 12-hour schedule or twice daily; (4) allowing the cannabis shatter to cool; (5) heating the chatter to 50-60° C. to a semi-smooth texture; (5) adding 190/200 proof ethanol; (6) heating the mixture and reducing it to nearly the starting weight; (7) cooling the mixture in an ice bath; (8) subjecting the mixture to slow clockwise sonication, pausing the sonicator on one minute intervals for two minutes and stirring in between; (9) subjecting the mixture to sonication at a 30000J output for five to eight minutes; (10) subjecting the mixture to magnetic stirring hotplate [at a temperature of 60 C, 300-320 rpm, and 72 hours of continuous mixing] as described in https://cdn.shopify.com/s/files/1/1726/3473/files/A Methodology for the Preparation of Liquid Textured Cannabinoids.pdf?14822043847272496341 on Jul. 31, 2018. A preferred catalyst for enthalpy of vaporization may be added to step (6). A ratio of the oil to the catalyst may be 1:1.

In some embodiments, a food additive provided herein may be made using a method comprising the steps of: (1) adding a water-soluble surfactant to distilled water to form the aqueous phase; (2) heating the aqueous phase mixture to 70° C.; (3) mixing an oil-soluble surfactant and cannabis oil to form the oil phase; (4) heating the oil phase mixture to 70° C.; (5) adding the aqueous phase drop-by-drop to the oil phase; (6) stirring the mixture at a constant rate for 30 minutes; (7) maintaining the temperature of the process at 70° C. as described in Mikulcova et al., “Formulation, Characterization and Properties of Hemp Seed Oil and Its Emulsions”, Molecules (2017) 22, 700. The water-soluble surfactant may be a Tween surfactant. The oil-soluble surfactant may be a Span surfactant. The use of Tween 80 and Span 80 at 5 wt % as emulsifiers produced particles ranging in diameter from 502 nm to 1050 nm.

In some embodiments, a food additive provided herein may be made using a method comprising the steps of: (1) preparing a mixture comprised of triglyceride, polyoxyl 40-hydroxy castor oil, Tween 20, and Span 80; (2) preparing a separate mixture comprised of amphiphilic co-solvent with soy phospholipid and heating the mixture to 40° C. until complete dissolution; (3) mixing the mixtures in steps (1) and (2); (4) stirring gently; (5) heating the mixture to 40° C. until homogenous pre-concentrate solution is formed; (6) adding a cannabinoid to the pre-concentrate; (7) stirring the mixture gently, where upon gentle agitation of the cannabinoid in the aqueous phase, the pre-concentrate spontaneously forms drug encapsulated O/W nano-dispersion; (8) heating the mixture to 40° C. until homogenous solution is formed, as described in WO2013/108254 A1.

The ratio of triglyceride to polyoxyl 40-hydroxy castor oil to Tween 20 to Span 80 may be about 1:1:1:1. The amphiphilic co-solvent may be ethyl lactate. The ratio of amphiphilic co-solvent to lechitin may be about 4:1. The mixture of emulsifiers in step (1) may be comprised of polysorbate 20 at 14.1% w/w, sorbitan monoleate at 14.1% w/w, lechitin at 8.3% w/w, tricaprine at 14.1% w/w, polyoxyl 40-hydroxy castor oil at 14.1% w/w, and ethyl lactate at 35.4% w/w. The mixture comprised of an amphiphilic co-solvent with soy phospholipid may be heated in a scintillation tube. In some embodiments, the cannabinoid may be tetrahydrocannabinol or cannabidiol. The cannabinoid may be added at 3% w/w.

In some embodiments, a food additive provided herein may be made using a method comprising the steps of: (1) preparing a water and a lipid source mixture in a flask; (2) heating the water-lipid source mixture to boiling; (3) removing the boiling water-lipid source from heat; (3) immediately adding cannabis material enclosed in a tea bag (or similar porous enclosure) to the boiling water-lipid source; and (4) steeping the cannabis mixture. The lipid source may include, but is not limited to, milk such as 10% milk, or butter, or combinations thereof. The ratio of the water to the lipid source may be about 4:1. The cannabis material may be the bud or the trim. The cannabis material may be processed using a hand miller, such as a handheld food processor, or an industrial miller. The heating step may be performed using an electric water heater or a microwave (e.g., set to a length of time of 2 minutes). The steeping step may last from about 3 minutes to about 10 minutes.

In some embodiments, an edible product provided herein further comprises an antidote to the cannabinoid. The person of skill will readily understand that in one embodiment, the antidote may be included in the food additive comprising the cannabinoid. In an alternate embodiment, the person of skill will readily understand that the antidote may be included in the edible product, separate from the food additive containing the cannabinoid.

As used herein, the term “antidote” means any compound capable of reducing or neutralizing the effects of a cannabinoid.

In some embodiments, the cannabinoid is psychoactive. In the context of the present disclosure, a cannabinoid is psychoactive if it affects mood, perception, consciousness, cognition or behaviour of a subject when consumed, as a result of changes in the functioning of the nervous system. Psychoactive effects of a cannabinoid may include euphoria, enhanced well-being, easy laughter, relaxation, fatigue, sleepiness, dysphoria, anxiety, panic, paranoia, depersonalisation, increased sensory perception, feeling of the body floating or sinking, heightened sexual experience, hallucinations, alteration of time perception, aggravation of psychotic states, fragmented thinking, enhanced creativity, disturbed memory, difficulty in concentration, headache, unsteady gait, ataxia, slurred speech, weakness, deterioration or amelioration of motor coordination, impaired learning, analgesia, muscle relaxation, improved taste responsiveness, appetite stimulation, cravings for cannabis, nausea, vomiting, and antiemetic effects. An antidote to a psychoactive cannabinoid is a compound capable of reducing or neutralizing the psychoactive effects of a cannabinoid.

In some embodiments, the psychoactive cannabinoid provided herein is THC, and the antidote is CBD; acorus calamus or extracts thereof; black pepper or extracts thereof; citrus or extracts thereof; pine nuts or extracts thereof; pistachio nuts or extracts thereof; fruits of Pistacia terebinthus or extracts thereof; piperine; or terpenes, such as β-caryophyllene, limonene, myrcene, or α-pinene. The antidote may be encapsulated in a microencapsulation system that is different from the microencapsulation system of THC.

Complaint, Recall, Return and Feedback Handling

The ICS discussed herein (e.g. the ICS implemented via system 400 and/or ICS 1506 and/or ICS 1508) may be used to manage and record complaints, recalls, returns and/or feedback for cannabis products. Complaints could be recorded in the ICS using a “create complaint” action. Complaints could originate from a customer due to an adverse reaction to a cannabis product and/or a dislike for a cannabis product, for example. However, complaints might not always relate directly to cannabis products. For example, issues with the holding containers that contain cannabis products could also or instead result in a complaint. Complaints could be received in the form of phone calls, emails or written letters, for example. Any or all of the information regarding a complaint could be recorded in the ICS. A non-limiting list of complaint information includes:
type and/or brand of product that initiated the complaint;
any or all identification numbers for the product (for example, lot number(s), batch number(s) and/or plant number(s));
quantity of the product used by the customer;
quantity of the product remaining in the customer's possession;
time and date the complaint was received;
name and contact information of the customer providing the complaint; and
the customer's explanation for the complaint.

A recall could be initiated in the ICS using a “new recall” action. For example, a recall could be initiated upon receipt of a complaint from a customer. A test of a product that returned an undesirable result could also or instead initiate a recall of a product. For example, a customer complaint could lead to an archived sample of a cannabis product being tested or re-tested, which might produce a failed result leading to a recall of that cannabis product. The new recall action could record the complaint, test results and/or other reason for initiating the recall. The product to be recalled could be identified in the ICS by a batch number, plant number, lot number, or any other form of product identifier. When the recall is created, the ICS could be automatically updated to reflect the recall. The products that are affected by the recall and still held by the cannabis producer could be frozen in the ICS such that they are not sold or shipped. In some embodiments, these products could also or instead be labelled to indicate that they have been recalled, transferred to a quarantine area, and/or destroyed.

In the event of a recall, the ICS could generate a list of customers affected by the recall. Customers affected by the recall could include distributors who have received and/or sold the recalled product, cannabis processors, other producers who have used the recalled product to produce other products, and end users who have received the recalled product or a product that includes or incorporates the recalled product. This list could be organized into different regions that the recalled product was distributed to. Any or all customers on the list could be notified of the recall and provided with instructions to return the affected products. For example, a distributor could be instructed to stop the sale of the recalled products, provide an inventory of the recalled products, and/or contact customers who bought the recalled products. Return kits could also or instead be sent to customers to help them safely return the recalled products. The return kits could include labelled packaging for sending a recalled product back to a cannabis producer. The return kits could be packaged, shipped and/or recorded in the ICS using any of the methods described herein. After the recalled products have been returned to the cannabis producer, these products could be weighed and/or recorded in the ICS. Labels could also or instead be added and/or updated on the returned products. At some point, the returned products could be transferred to a quarantine area and/or destroyed. Replacement products could be sent to affected customers at any time during and/or after a recall. In some embodiments, at least some of communication with the customers during a recall could be automated using the ICS.

Products could also be returned without a recall being issued. For example, a customer could file a complaint that does not warrant a recall of a product, and that customer could be provided with instructions to return the relevant products, a return kit, and/or a replacement product. These returns could be recorded in the ICS using a “create return” action. The create return action could record any or all information regarding the complaint, information regarding the product that was returned, and/or information regarding the replacement product that was shipped.

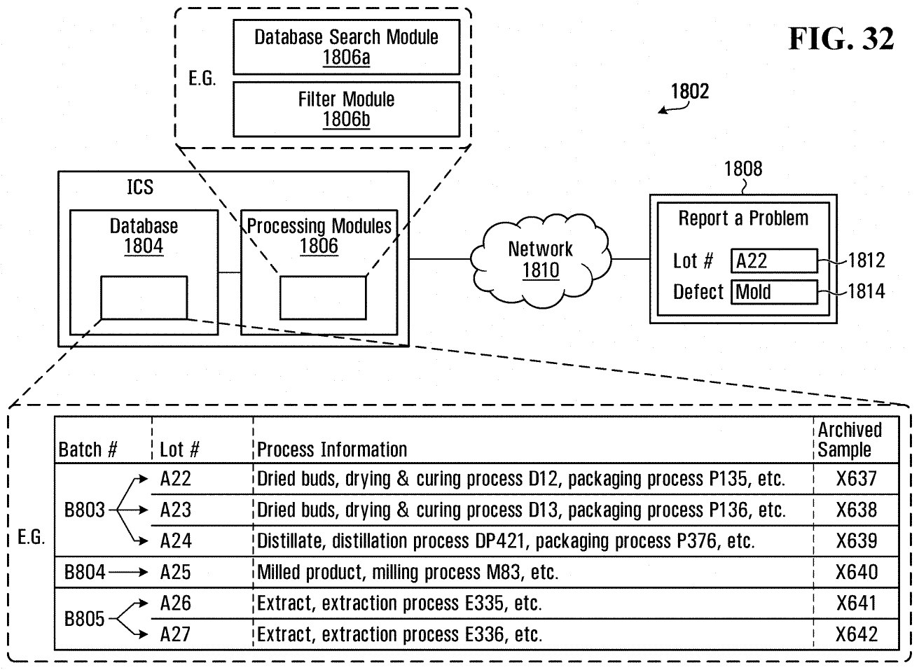

FIG. 32 illustrates a system 1802 for identifying a lot of cannabis products for recall, according to one embodiment. The system 1802 includes memory, e.g. a database 1804, and processing modules 1806. The processing modules 1806 may be implemented by one or more processors that execute instructions stored in the memory 1804. Alternatively, some or all of the processing modules 1806 may be implemented using dedicated circuitry, such as an ASIC, GPU, or FPGA. In FIG. 32, the processing modules 1806 include a database search module 1806a and a filter module 1806b, which operate in the manner described below.

In the example illustrated in FIG. 32, the database 1804 and processing modules 1806 are part of an ICS. However, this is only an example. In other embodiments, the database 1804 and/or processing modules 1806 may be separate from and/or independent of an ICS.

Stored in the database 1804 is information associated with a plurality of batches of cannabis plants. Each batch is associated with a batch identifier, which will be called a batch number. Also stored in the database 1804 is information associated with a plurality of lots of cannabis products. Each lot is associated with a lot identifier, which will be called a lot number. Each batch number is linked to/associated with the lot number for each lot of cannabis product originating from that batch. An example is illustrated in FIG. 32 in which some plants from batch B803 are processed to produce lot A22 of dried buds, other plants from batch B803 are processed to produce lot A23 of dried buds, and other plants from batch B803 are processed to produce lot A24 of a distillate product. The batch number B803 is therefore associated with lot numbers A22, A23, and A24. Also illustrated in the example of FIG. 32 is a single lot A25 produced from batch B804, and two lots A26 and A27 produced from batch B805. Although not illustrated in the example, it could be the case that one or more lots originate from more than one batch (e.g. another lot A28—not illustrated—may be produced using plants from batch B803 and B805).

A user interface, e.g. a graphical user interface (GUI) 1808 in the form of a display, is coupled to the processing modules 1806 and database 1804. In the example of FIG. 32, this is implemented by the GUI 1808 being communicatively coupled to the ICS via a network 1810. The GUI 1808 allows for a user to input information relating to a defective unit of cannabis product, e.g. to input a lot number for the unit of cannabis product, as shown at 1812. In an alternative embodiment, the user interface may not be a GUI, and/or it may include other components. For example, the user interface may be or include a barcode scanner that reads the lot number encoded in a machine-readable code on a unit of cannabis product.

The particular GUI 1808 illustrated in FIG. 32 also allows for the user to enter defect information indicative of the nature of the defect resulting in the defective unit of cannabis product, as shown at 1814. However, other embodiments may not support this functionality.

The lot number of a defective unit of cannabis product, which is provided by the user, e.g. via GUI 1808, will be referred to as a “suspect lot number”. It is a lot number of a lot suspected to be defective. In some embodiments, one or more of the processing modules 1806, e.g. the database search module 1806a, queries the database 1804 to identify any batch number associated with the suspect lot number. An associated batch number will be referred to as a “suspect batch number”. The database search module 1806a may then query the database 1804 to determine all of the lot numbers associated with each suspect batch number. For example, if the suspect lot number is A22, then there is one suspect batch number (B803), and the associated lot numbers are A22, A23, and A24. Each lot number associated with a suspect batch number will be referred to as a “recall lot number” (or recall lot identifier) because it is a lot number that is possibly subject to a recall. For example, if the suspect batch number is B803, then the recall lot numbers are A22, A23, and A24.

In some embodiments, there may be one or more units of archived cannabis material associated with each batch and/or lot, and information used to identify the archived material may be stored in database 1804. For example, in FIG. 32, an archived sample exists for each lot, and is identified by a respective number, which is stored in database 1804. For example, lot A22 is associated with the archived sample identified as X637, lot A23 is associated with archived sample X638, and lot A24 is associated with archived sample X639, such that batch number B803 is associated with three archived samples X637, X638, and X639. In some embodiments, any archived cannabis material sample that is associated with a suspect batch identifier is examined or tested to determine whether it is defective. If a tested archived cannabis material sample is found to be defective, then the associated lot number(s) in the database 1804 are identified, and a recall of the affected lot(s) may be triggered.

In some embodiments, each lot number may have process information stored in the database 1804 and associated with the lot. The process information may be associated with a manufacturing process used to manufacture the lot of cannabis product. An example is illustrated in FIG. 32 in which process information is included in database 1804. For example, the process information for lot A22 identifies that the product of lot A22 is dried buds, which was produced using drying and curing process D12, and was packaged into containers using packaging process P135, etc. Examples of manufacturing processes may include processes such as: separating the plant material; and/or drying the plant material; and/or curing the plant material; and/or extracting cannabinoids from the plant material to produce a cannabis extract; and/or distilling cannabis extract to produce a distillate.

In some embodiments, the GUI 1808 enables a user to input defect information indicative of the nature of the defect resulting in the defective cannabis product. The filter module 1806b then obtains the defect information from the GUI 1808. The filter module 1806b also obtains, from the database 1804, the process information associated with the recall lot identifiers that are associated with the at least one suspect batch identifier. The recall lot identifiers may then be filtered by the filter module 1806b using the process information and the defect information, e.g. to identify which lots may need to be recalled (and which lots would perhaps be exempt from the recall) based on the defect information and the process information.

As an example: The defect information entered on the GUI 1808 is that a unit of lot A22 of cannabis product contains mold, as shown in GUI 1808 as illustrated. The suspect batch number is therefore B803, and so the recall lot identifiers are A22, A23, and A24. The filter module 1806b retrieves process information for each of the recall lot identifiers. The process information associated with recall lot number A24 indicates that the manufacturing process used in the production of lot A24 included extracting cannabinoids from the plant material to produce a cannabis extract and distilling a cannabis extract to produce a distillate. The act of distillation is known to eliminate the possibility of mold, i.e. distillation remedies the defect of mold, and so lot A24 should not need to be recalled. The filter module 1806b therefore exempts lot A24 from recall by filtering out recall lot number A24. Only products from lot numbers A22 and A23 are identified as being subject to recall.

Recalls represent an example of an application of various assigned and recorded identifiers disclosed herein. These identifiers could be used to trace cannabis products through at least part of a processing or production chain, and potentially to plant batch or even individual plant, depending on the depth or granularity of identifiers.

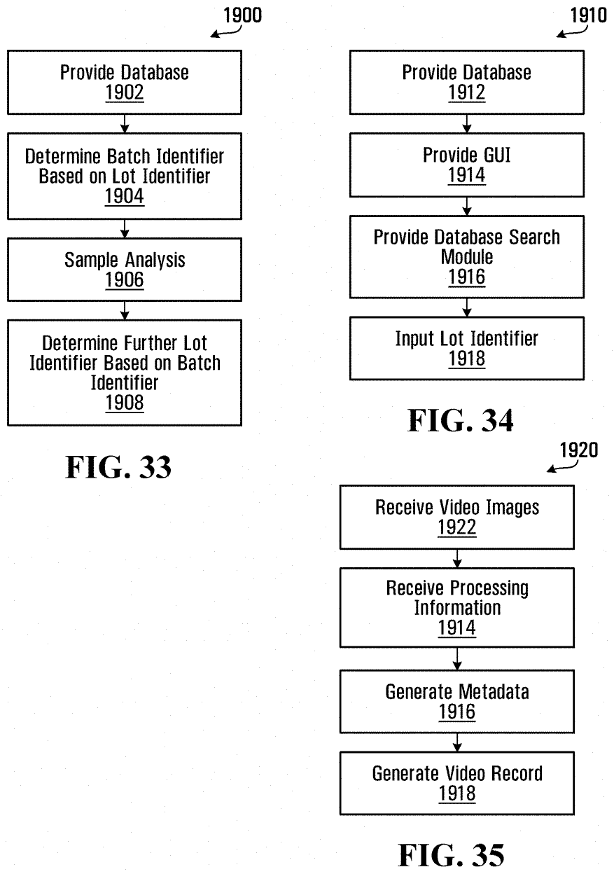

FIG. 33 is a flow diagram illustrating an example method of identifying a lot of cannabis products for recall. The example method 1900 involves, at 1902, providing a database in which information associated with batches of cannabis plants is stored. Each batch is associated with a batch identifier. Information associated with lots of cannabis products is also stored in the database. Each lot is associated with a lot identifier. Each batch identifier in the database is also associated with at least one lot identifier.

Provision of a database at 1902 does not necessarily involve populating the database. The database could have been previously populated with information during harvest of cannabis plants, processing of those plants into any of various cannabis products, and/or packaging of those products, for example. Therefore, providing a database at 1902 could, but need not necessarily, involve populating or otherwise generating the database. For the purposes of identifying a lot of cannabis products for recall, and/or possibly other embodiments that involve using information in a database, providing a database could entail providing access to an existing database.

The example method 1900 also involves, at 1904, determining, using a lot identifier associated with a defective cannabis product, at least one suspect batch identifier associated with the lot identifier. As described above, each batch identifier in the database is also associated with at least one lot identifier, and accordingly the lot identifier associated with the defective cannabis product can be used to determine a batch identifier associated with the lot identifier, or each associated batch identifier if there is more than one batch identifier associated with the lot identifier. Multiple batch identifiers could be associated with the same lot identifier if plant material from multiple batches of cannabis plants is used in producing the defective cannabis product. A determined batch identifier could be considered a “suspect” batch identifier in the sense that it has an association with the lot identifier of a defective cannabis product.

The lot identifier associated with a defective cannabis product could be received or entered into a recall process in any of various ways. Markings on a product container, a product package, a product container label, and/or a product package label, for example, could be scanned and at least information conveying or indicating the lot identifier could be transmitted to or otherwise entered into a recall process. Manual entry of a lot identifier or other information that enables determination of the lot identifier is also contemplated.

In some embodiments, one or more cannabis material samples are archived for each batch of cannabis plants. The sample analysis at 1906 represents determining, for each archived cannabis material sample associated with the at least one suspect batch identifier, whether the archived cannabis material sample is defective. Sample analysis could involve any of various analysis processes. In some embodiments, sample analysis could involve consulting records of testing that was previously conducted during processing or production, at 120 in FIG. 1, for example. Sample analysis could also or instead involve repeating previous testing and/or conducting different testing or analysis on one or more archived samples. The type(s) of testing or analysis performed at 1906 could be predetermined and/or selected based on one or more factors such as the type of cannabis product with which the lot identifier is associated, the manner in which the cannabis product is defective, parameters or characteristics associated with the batch(es) associated with the at least one suspect batch identifier, and/or other factors.

The sample analysis at 1906 could find that one or more archived materials samples are defective. A method could include, as shown at 1908, determining all lot identifiers in the database associated with each archived cannabis material sample that is found to be defective. A batch of cannabis plants could have been processed to produce multiple lots of one or more cannabis products, in which case multiple lot identifiers could be associated with the same batch identifier. The determining at 1906 could involve determining such lot identifiers, possibly including further lot identifiers in addition to the lot identifier associated with the defective cannabis product, using a batch identifier with which each defective archived cannabis material sample is associated. For example, lot records could be searched for each batch identifier that is associated with a defective archived cannabis material sample, and the lot identifier associated with each lot record that includes any searched batch identifier can then be determined.

The example method 1900 could therefore involve “bi-directional” searching or tracing in the database. At 1904, searching or tracing is from lot to batch, and then at 1908 the searching or tracing is in the opposite direction, from batch to lot.

FIG. 33 helps demonstrate not only the potential importance of traceability for the purpose of recalls, but also how depth or granularity of identifiers could impact functions or tasks for which it is necessary or desirable to determine batch or plant origin of cannabis products. Larger plant batches and/or smaller lot sizes, for example, could result in a larger number of product lots being associated with a batch. This could in turn lead to more extensive recalls if any lot from a batch is determined to be defective. Smaller plant batches and/or larger lot sizes might result in fewer associated lots for each batch, but it may be necessary to use plant material from multiple batches to produce enough product for a lot, in which case a recall for a defective lot could extend to multiple batches and potentially all lots associated with any one of those multiple batches. Any of these and/or other factors could be taken into account in determining manageable batch and/or lot sizes.

A recall process could also include other features as well. One or more of any lot identifiers that are determined at 1908 could be included in a product recall, for example. Not all determined lot identifiers might necessarily be included in a recall. For example, a defect could be related to a particular substance that is used only in certain production processes and not in others. A defect could be associated with a processing or treatment residue that only affects particular types of products. Only lots of those particular types of products could be recalled, even if other products were also produced from the same batch(es). Other defects could affect the same and/or other product types, or all products.

Another example of a method of identifying a lot of cannabis products for recall is illustrated in the flow diagram in FIG. 34. The example method 1910 involves, at 1912, providing a database in which information associated with a plurality of batches of cannabis plants and a plurality of lots of cannabis products is stored. Each batch is associated with a batch identifier, each lot is associated with a lot identifier, and each batch identifier in the database is associated with at least one lot identifier. Providing a database is discussed elsewhere herein, such as above with reference to FIG. 33.

A GUI implemented on a computer system is provided at 1914, to enable a user to input a suspect lot identifier associated with a defective cannabis product. A database search module implemented on the computer system is also provided, at 1916. The database search module is configured to determine, in response to a user inputting a suspect lot identifier, at least one suspect batch identifier associated with the suspect lot identifier in the database and all lot identifiers associated with the at least one suspect batch identifier in the database. Providing the GUI and the database search module could involve, for example, accessing the computer system in which the GUI and the database search module are implemented. In some embodiments, these features are implemented at least in part using software and one or more components of the computer system, such as a processor, that execute the software. Software could also or instead configure the database search module to determine the at least one suspect batch identifier and all lot identifiers associated with the at least one suspect batch identifier in the database. Such lot-batch-lot searching or tracing is disclosed elsewhere herein, such as above with reference to FIG. 33.

The example method 1910 also involves inputting a suspect lot identifier into the graphical user interface, at 1918. In response to this input of a suspect lot identifier, at least one suspect batch identifier associated with the suspect lot identifier in the database and all lot identifiers associated with the at least one suspect batch identifier in the database are determined by the database search module. The database search module could provide an output indicative of any one or more of the suspect lot identifier, the at least one suspect batch identifier, and the lot identifiers associated with the at least one suspect batch identifier in the database. The output could be provided to a user and/or to other components of a system, and could be used to generate recall notices or orders for one or more lots, for example.

A processor-readable storage medium could be used in implementing at least some of the operations in the example recall-related methods 1900 and/or 1910, with processor-executable instructions being stored on such a medium. The instructions, when executed by a processor, cause the processor to perform a method. Execution of the instructions could cause a computing device that includes the processor to implement a system configured to perform such a method.

A system for identifying a lot of cannabis products for recall, whether implemented using a processor-readable storage medium and a processor or in some other way, could include in some embodiments a database, a graphical user interface, and a database search module. In the database, information associated with a plurality of batches of cannabis plants and a plurality of lots of cannabis products is stored. As in other embodiments disclosed herein, such as with reference to FIGS. 29 and 30, each batch is associated with a batch identifier, each lot is associated with a lot identifier, and each batch identifier in the database is associated with at least one lot identifier. The graphical user interface is implemented on a computer system, to enable a user to input a suspect lot identifier associated with a defective cannabis product, and the database search module is also implemented on the computer system. The database search module is configured to determine, in response to a user inputting a suspect lot identifier through the graphical user interface, at least one suspect batch identifier associated with the suspect lot identifier in the database and recall lot identifiers associated with the at least one suspect batch identifier in the database. These operations are discussed elsewhere herein, for example above with reference to FIG. 34.

In some embodiments, the database further includes, for each lot identifier, process information associated with the manufacturing process(es) used to manufacture the associated lot of cannabis products from plant material of one or more batches of cannabis plants. The graphical user interface could be further configured to enable a user to input defect information indicative of the nature of the defect resulting in the defective cannabis product.

A filter module could also be implemented on the computer system, using software and a component of the computer system such as a processor to execute the software. The filter module could be configured to: receive the defect information that is input by the user; receive the process information associated with the recall lot identifiers associated with the at least one suspect batch identifier in the database; and filter the recall lot identifiers using the process information and the defect information. Such filtering represents an example of how only lot identifiers associated with product lots that are potentially affected by a defect could be distinguished from unaffected product lots, for at least certain defects that do not necessarily affect all products or all product types that originated from an affected batch of plants.

Examples of processing or manufacturing processes are provided elsewhere herein. The manufacturing processes used to manufacture the lot of cannabis product from plant material of one or more batches of cannabis plants could include one or more of: separating the plant material; drying the plant material; curing the plant material; extracting cannabinoids from the plant material to produce a cannabis extract; distilling cannabis extract to produce a distillate, and/or others disclosed herein.

In some embodiments, the filter module is configured to filter out the recall lot identifiers associated with manufacturing processes that are known to result in a remediation of the defect resulting in the defective cannabis product. A defect could relate to plant bacteria that would be killed by certain types of manufacturing processes such as extraction, for example. Recall lot identifiers associated with extraction could be filtered out by the filter module in this example.

As another example, if the defect information conveys that the nature of the defect resulting in the defective cannabis product relates to the presence of mold, then the filter module could be configured to filter out the recall lot identifiers associated with process information that conveys that the manufacturing processes used in the production of the lot included one of extracting cannabinoids from the plant material to produce a cannabis extract and distilling a cannabis extract to produce a distillate.

The recall-related methods and systems described above are intended solely for illustrative purposes. Other embodiments could include fewer, further, and/or different features, performed or arranged in a similar or different order than described. For example, features described in the context of a method could be provided in a system embodiment, and features described in the context of a system could be provided in a method embodiment.

Furthermore, recall-related features need not necessarily be specific only to recalls. The same or similar features could also or instead be used in other applications in which it may be necessary or useful to determine batch or plant origin of cannabis products. For example, it might be desirable to enable a cannabis product that has high customer ratings to be traced back through a production stream. This could enable growth, harvest, and/or processing parameters or conditions to be determined, and potentially replicated in an effort to reproduce highly rated cannabis products that are expected to be well-received by customers.

As noted at least above in respect of other embodiments, a processor-readable storage medium could be used in implementing at least some of the operations in these example methods relating to recalls, with processor-executable instructions being stored on such a medium. The instructions, when executed by a processor, cause the processor to perform a method. Execution of the instructions could cause a computing device that includes the processor to implement a system configured to, in some embodiments, perform at least some of the method operations discussed above and/or elsewhere herein.

A system could include such a computing device, as well as other components involved in producing a cannabis-infused consumer product. These and/or other possible implementation options in respect of a system that could be configured or used to perform a method consistent with these example methods disclosed herein could be or become apparent. FIG. 32, for example, illustrate one possible embodiment of a system in which components could be configured to perform such methods.

Manufacturing Area Surveillance

In some embodiments, video cameras are installed on-site to record activities relating to the handling and/or processing of cannabis, e.g. for security and/or regulatory purposes. For example, a video camera may record images of a cannabis operations area in which cannabis material is being processed.

In addition to the video recordings, processing information associated with the processing of the cannabis material may also be recorded and stored, e.g. in the ICS. The processing information may include information such as:
a batch identifier/number identifying a batch of cannabis plants associated with the cannabis material being processed in the operations area; and/or
a lot identifier/number identifying a lot of cannabis products associated with the cannabis material being processed in the cannabis operations area; and/or
the identity of the person or people carrying out the processing in the cannabis operations area; and/or
the date and/or time at which the processing is being performed; and/or
the date and/or time at which the video images are recorded; and/or
the location of the cannabis operations area.

As an example, it may be recorded in the ICS that harvested plants from batch B378 are placed into holding container H212 at 2 pm on Apr. 15, 2019. As another example, it may be recorded in the ICS that fifty containers of dried buds are packaged on May 1, 2019 at 4 pm to produce lot number A75. During this time, the video camera(s) is/are recording video images of all such activities.

In some embodiments, the processing information is used as metadata that is tagged to the video record by combining the video images with the metadata. The metadata may then be used, e.g. by the ICS, to automatically retrieve and present to a user interface (e.g. a GUI) the relevant video footage when it is necessary to review video footage to investigate a problem.

As one example, fifty containers of dried buds are packaged on May 1, 2019 at 4 pm to produce lot number A75. The ICS stores in memory (e.g. in a record) that lot number A75 has been created. Meanwhile, a digital video recording of the event is also stored in a database. The ICS then associates: (1) the packaging of containers to produce lot number A75, and (2) the video recording of the event. For example, the start and end times of the packaging may be input by a person or machine into the ICS, or the ICS may select a predefined window of time around the time indicated by the person or machine, e.g. if the packaging happened at 4 pm, then a window of 3:45 pm-4:15 pm may be selected. The video footage of that time and at that location may then be indexed with this processing information. Then, for example, if it is later determined that there is a problem with a container of dried buds from lot number A75, the ICS may automatically retrieve the indexed video footage that recorded the packaging of lot number A75, and present that to a user interface. The user therefore does not have to sort through vast amounts of video footage manually. Instead, the relevant video footage is presented to the user for viewing.

In some embodiments, the processing information metadata may be overlaid onto the video footage. For example, in the scenario described above, when the video footage of lot packaging around 4 pm on May 1, 2019 is presented to the user, the information “packaging lot number A75” may be overlaid on top of the video images, possibly along with other metadata (e.g. the date/time, the person performing the packaging, etc., as recorded in the ICS).

In some embodiments, the ICS uses the link between batch, processing, and lot identifiers to associate together all video footage relevant to the creation of a particular lot of cannabis product. For example, if a problem is identified with a unit of cannabis product belonging to lot number A75, then the ICS may retrieve and present (for user selection) all video footage relating to the creation of lot A75, e.g. from the harvesting of the batch of plants from which the cannabis in lot A75 originated, to the recording of movement/transfer of that cannabis, to the recording of any processing performed on that cannabis, all the way through to the packaging of the containers of lot A75. The ICS is able to automatically retrieve this video because of: (1) the association of records in the ICS relating to harvesting of a particular batch of cannabis plants from which the cannabis in the lot originated, through all steps in the process up to and including creation of the lot; and (2) the association of video images with each step of the processing.

As an example: video footage ‘A’ is associated with the harvesting of batch B378; video footage ‘B’ is associated with transferring the harvested plants from batch B378 into holding container H212; video footage ‘C’ is associated with extraction process E567 performed on the plants in holding container H212; video footage ‘D’ is associated with lot packaging of the output of extraction process E567 to create lot A93. The association between batch B378, holding container H212, extraction process E567, and lot A93 is stored in the ICS to link lot A93 to all previous processing operations and have traceability all the way back to the batch B378. Subsequently, if there is a problem with a unit of cannabis product from lot A93, the ICS may retrieve, for presentation to the user interface, any or all of video footage ‘A’ to ‘D’, depending upon the user's request.

In this way, in some embodiments the ICS associates a unit of cannabis product from a particular lot with a plurality of digital video segments, each digital video segment corresponding to a respective different part of a multi-step process for producing that unit of cannabis product from a particular batch of cannabis plants.

In some embodiments, video images may be tagged with metadata associated with a detected security event. An example of a security event is attempted or actual unauthorized access to the cannabis operations area or illicit conduct within the cannabis operations area. For example, if an alert signal is triggered by a security system, the relevant video images (e.g. the video recording of the location at which the alert was triggered around the time at which the alert was triggered) may be stored in the ICS in association with the alert signal. Metadata indicative of the alert signal may be generated and possibly overlaid on top of the video images.

Method embodiments related to video content are also contemplated. FIG. 35 is a flow diagram illustrating an example method of creating video content, according to one embodiment. The example method 1920 involves receiving, at 1922, video images of a cannabis operations area in which cannabis material is being processed. The video images are captured by one or more video cameras installed to record activities at one or more operations areas, and could be transmitted to a central ICS server that hosts and ICS database, and/or to one or more other components.

For example, referring to FIG. 4A, one or more video cameras could be provided to record activities during cultivation, in the grow area(s) 456a for example, and/or during harvest. A video camera could be connected to or otherwise in communication with the server 418a, and/or to other components of the example cultivation and harvest system 420a such as a computer 424a and/or a controller at 426a, to transmit video images to the server 418a and/or other component(s). Video images could be locally stored by the video camera(s) and/or other component(s) to which video images are transmitted by the video camera(s), and/or further transmitted, to the server 402 for example.

In some embodiments, a video camera is connected to or otherwise in communication with one or more controllers 426a, to control operation of the video camera. A video camera could be configured or controlled to record continuously, or according to a program or schedule. Dynamic video camera control and/or recording are also contemplated. For example, a video camera could be turned on when any operator first checks in, using an operator check-in device 422a, to an empty facility or area in which no other operators are currently checked in, and could record video images until all operators have checked out. A video camera could also or instead be responsive to intrusion detection by a security system. Such dynamic control could be implemented in combination with programmed or scheduled recording. A video camera operate in accordance with a program or schedule unless or until a trigger event such as an operator check-in or intrusion detection occurs. After a trigger event is no longer valid or active, such as after all operators check out of a monitored facility or area or an intrusion detector is reset, a video camera could return to programmed or scheduled recording.

Other video camera settings or parameters could also or instead be predetermined and/or controlled. Examples of such settings or parameters include illumination settings for a camera light or controller, video speed such as frames per second, and/or focus settings.

Camera orientation could also or instead be controlled in some embodiments. This could involve controlling a camera and/or a movable platform or mount on which the camera is mounted, for example.

One or more video cameras could be provided to record activities in any cannabis operations area. Video monitoring of cultivation and harvest, and provision of one or more video cameras in the example cultivation and harvest system 420a, are intended as illustrative example embodiments. One or more video cameras could also or instead be provided to monitor other cannabis operations areas.

At 1914 in FIG. 35, processing information associated with the processing being carried out in in the cannabis operations area is received. Examples of such processing information and how such information could be used with video images are provided elsewhere herein, at least above in the context of creating video content. The processing information could be received from other components, such as one or more of the computer(s) 424a, controller(s) 426a, sensor(s) 428a, scale(s) 430a, label maker(s) 432a, and scanner(s) 434a in FIG. 4A. As noted above, the example cultivation and harvest system 420a is an illustrative example application of video monitoring, which could also or instead be provided for other cannabis operations areas. For other cannabis operations areas, processing information could be received from similar and/or different components.

The example method 1920 in FIG. 35 also includes, at 1916, generating metadata using at least some of the processing information. Metadata could include excerpts from the processing information itself. In some embodiments, a one-way transformation such as a hashing function is applied at least some of the processing information to generate metadata. A resulting transformed value such as a hash value could subsequently be used to verify the part(s) of the processing information to which the transformation was applied. Another example of metadata is a code generated based on at least some of the processing information. Such a code could encode at least some of the processing information and be used to recover the coded part(s) of the processing information. In some embodiments, the code is a machine-readable code. Other types of metadata based on at least some of the processing information are also contemplated.

Referring again to the example cultivation and harvest system 420a in FIG. 4A as an illustrative example, metadata could be generated by any one or more of a computer 424a, another component that generates or collects the processing information, the server 418a, and the server 402. For example, if metadata is generated based only in processing information from a particular sensor or particular equipment, then that sensor or that equipment or its controller could generate the metadata and store and/or transmit the metadata with the processing information.

At 1918, a video record is generating by combining the video images and the metadata. The video images and the metadata could be combined in any of various ways to generate a video record. The metadata could be added, as file metadata for example, to a video file that includes the video images. The metadata and video images could also or instead be stored in the video record. Generating a video record could involve overlaying at least part of the metadata onto the video images. File metadata, storage of the metadata and video images in the video record, and overlaying a part of the metadata onto the video images represent illustrative examples of how metadata and video images could be combined to generate a video record.

A video record could be stored in a database, such as the database 414 in FIG. 4A for example. A video record stored in such a database could be indexed using the processing information. For example, the processing information could include a batch identifier/number and/or a lot identifier/number. As disclosed elsewhere herein, other types of records in an ICS could include such identifiers/numbers, and a video record could similarly include and/or otherwise be indexed by batch and/or lot identifier/number. Examples of records are provided herein at least with reference to FIGS. 21-23, and a video record could include at least some similar record fields in some embodiments.

The method 1920, like other methods disclosed herein, is an illustrative embodiments. Variations of the method are also contemplated. For example, a method could involve receiving an alert signal. An alert signal could be indicative of an attempted or actual unauthorized access to the cannabis operations area or illicit conduct within the cannabis operations area, for example. Metadata indicative of the alert signal could be generated, and combined with the video data in generating a video record. Examples of metadata generation are provided herein, at least above with reference to step 1918 in FIG. 35. In some embodiments, both metadata indicative of an alert signal and metadata that is based on at least some processing information is combined with video data in generating a video record.

Video images as discussed herein represent one example of visual content. Images need not necessarily be continuous in time. For example, a series of images spaced apart by more than a visually perceivable time gap could be sufficient for the purposes of monitoring a cannabis operations area. Such series of images could be considered a form of video images, despite the time gaps. In general, any features disclosed herein in the context of video content or video images could also or instead be applied to still images.

As noted at least above in respect of other method embodiments, a processor-readable storage medium could be used in implementing at least some of the operations in methods relating to video content, with processor-executable instructions being stored on such a medium. The instructions, when executed by a processor, cause the processor to perform a method. Execution of the instructions could cause a computing device that includes the processor to implement a system configured to, in some embodiments, receive video images, receive processing information, generate metadata, and generate a video record.

A system could include such a computing device, and possibly other components. Embodiments that involve video content could be implemented in any of various ways in the example system 400 in FIGS. 4A-4M, for example.

Reporting

In some embodiments, the ICS system described herein can use any of the information collected and/or stored in order to generate regular or ad hoc reports relating to any aspect of cultivation, extraction, processing, manufacturing, testing, packaging, shipping, or any other activity, task or operation described herein. Such reports can be used to feed into integrated systems for managing business processes (e.g. Enterprise Resource Planning (ERP) platforms). The ICS system described herein can also generate compliance, operational and Business Intelligence (BI) reports. Examples of such reports include, but are not limited to:
Regulatory reports, such as monthly reports, annual reports, notifications to regulatory bodies, onsite inspection reporting, including:
   Amount of cannabis reported lost or theft,
   Lists of cannabis products made available for sale,
   Lists of amounts of cannabis produced and types of cannabis classes,
   Number and nature of deviations and corrective actions taken,
   Number of shipments and associated geographic locations,
   List of adverse reactions per batch/lot,
   List of complains per batch/lot,
   Amount of cannabis product recalled (and batch/lot identification),
   Amount of cannabis product produced for research and development,
   Inventory reporting, including:
      lists of items held in inventory (oils, extracts, distillates, terpenes, etc.)
      Physical locations in inventory, current weight/volume;
   Reports relating to whether or not processes are executed in accordance with predetermined Standard Operating Procedures (SOPs), which can include the reviewing of camera footage associated with particular tasks and/or time periods and/or batches/lots; and
   Adverse reaction reports relating to customers having adverse reactions to particular batches/lots;
Financial reports, such as government agency reports relating to taxation and statistics;
Business Intelligence reports relating to the cost for each product line/task, Cost of Manufacturing (COM) reports, evaluation cost; and
Quality Assurance (QA) reports including test results of cannabinoid concentration levels, and the presence of heavy metals, microbiological contaminants and/or pesticides in finished products.

### CONCLUSION

An ICS as disclosed herein could be leveraged in any of various ways, to track, monitor, verify, and/or control any of a multitude of logistical or operational aspects of cannabis production, from cultivation to final sale of cannabis products, and anywhere in between. An ICS could include at least an inventory of assets, including asset locations, status, and/or other information relating to assets. Any or all transfers of cannabis-containing substances between different holding containers and/or different locations could also or instead be recorded. In general, any time a cannabis-containing substance is produced, combined, separated, and/or transferred, an ICS could be updated with any of various types of information.

Whenever “number” is used herein, it encompasses any arrangement of characters or symbols, e.g. it encompasses alphanumeric numbers, characters, and/or symbols also. The word “number” may be used interchangeably with “identifier” or “indicia”.

Although the foregoing has been described with reference to certain specific embodiments, various modifications thereof will be apparent to those skilled in the art without departing from the scope of the invention as defined by the claims.

For example, embodiments disclosed in the context of a cannabis producer or a cannabis processor are not necessarily exclusive to cannabis producer applications or cannabis processor applications. Embodiments could potentially be applied to cannabis producers and/or cannabis processors.

Embodiments are disclosed primarily in terms of collecting and recording information and controlling labelling for the purpose of enabling traceability. Other features could also or instead be provided. Inventory stored in an ICS could be routinely audited to verify that the ICS is accurate. An inventory audit could include taking account of assets, for example by counting and/or weighing all cannabis seeds and plants, counting and/or weighing all cannabis being dried, counting and/or weighing all holding containers for dried and fresh cannabis, counting and/or weighing all holding containers for cannabis oil and resin, and counting and/or weighing all cannabis waste. Alternatively, a random selection of cannabis products could be counted, weighed, and/or otherwise accounted. Results of an inventory audit could be checked against the ICS to determine whether the ICS is consistent with the inventory audit. In the event of a discrepancy between the ICS and the inventory audit is discovered, an investigation could be launched to determine the cause of the discrepancy.

Any module, component, or device exemplified herein that executes instructions may include or otherwise have access to a non-transitory computer/processor readable storage medium or media for storage of information, such as computer/processor readable instructions, data structures, program modules, and/or other data. A non-exhaustive list of examples of non-transitory computer/processor readable storage media includes magnetic cassettes, magnetic tape, magnetic disk storage or other magnetic storage devices, optical disks such as compact disc read-only memory (CD-ROM), digital video discs or digital versatile disc (DVDs), Blu-ray Disc′, or other optical storage, volatile and non-volatile, removable and non-removable media implemented in any method or technology, random-access memory (RAM), read-only memory (ROM), electrically erasable programmable read-only memory (EEPROM), flash memory or other memory technology. Any such non-transitory computer/processor storage media may be part of a device or accessible or connectable thereto. Any application or module herein described may be implemented using computer/processor readable/executable instructions that may be stored or otherwise held by such non-transitory computer/processor readable storage media.

1.-118. (canceled)

119. A method for manufacturing and packaging a cannabis-infused consumable product made from a cannabis-containing substance, the method comprising:
providing multiple amounts of cannabis-containing substance, each amount derived from cannabis plant material, the cannabis-containing substance containing one or more cannabinoids, each amount of cannabis-containing substance being associated with an identifier allowing one amount to be distinguished from another amount;
providing a control device having a machine-readable storage;
storing, in the machine-readable storage, identifiers associated with respective ones of the amounts of cannabis-containing substance;
diluting each amount of cannabis-containing substance with a diluting agent to produce a master batch of consumable product;
dispensing the master batch into a set of packages, each package holding a portion of the master batch; and
applying an indicia on individual packages, including the steps of:
feeding a stream of individual packages to a marking unit; and
distinguishing in the stream between individual packages holding a consumable product made from different amounts of cannabis-containing substance and controlling the marking unit with the control device to apply to each individual package an indicia derived from the identifier of the respective amount from which the consumable product in the package was made.

120. The method as defined in claim 119, wherein the cannabis-infused consumable product is vaping oil.

121. The method as defined in claim 119, wherein the diluting agent is oil or water.

122. The method as defined in claim 119, wherein the consumable product is a cannabis-infused beverage or an emulsion.

123. The method as defined in claim 119, wherein dispensing the master batch is performed such that the consumable product in each package originates from a single amount of cannabis-containing substance.

124. The method as defined in claim 119, including counting a number of packages produced from a particular amount of cannabis-containing substance and storing the counted number in the machine-readable storage.

125. A method for manufacturing and packaging a cannabis-infused consumable product made from a cannabis-containing substance, the method comprising:
providing multiple amounts of a cannabis-containing substance, each amount derived from cannabis plant material, the cannabis-containing substance containing one or more cannabinoids;
providing a control device having a machine-readable storage;
storing, in associated with respective ones of the amounts of cannabis-containing substance, identifiers allowing distinguishing one amount from another amount;
diluting each amount of cannabis-containing substance with a diluting agent to produce respective master batches of consumable product;
dispensing the master batches into respective sets of individual packages, each package of a given set holding a portion of the respective master batch;
feeding a stream of individual packages to a marking unit, the stream being arranged in an order determined by which master batch is a source of the consumable product held in each individual package; and
under control of the control device, synchronizing operation of the marking unit with the order in which the stream of individual packages is arranged such that each individual package receives an indicia associated with a particular amount from which the consumable product in the package is made.

126. The method as defined in claim 125, wherein the cannabis-infused consumable product is vaping oil.

127. The method as defined in claim 125, wherein the diluting agent is oil or water.

128. The method as defined in claim 125, wherein the consumable product is a cannabis-infused beverage or an emulsion.

129. The method as defined in claim 125, wherein dispensing the master batch is performed such that the consumable product in each package originates from a single amount of cannabis-containing substance.

130. The method as defined in claim 125, including counting a number of packages produced from a particular amount of cannabis-containing substance and storing the counted number in the machine-readable storage.

131. A method for manufacturing and packaging a cannabis-infused consumable product made from a cannabis-containing substance, the method comprising:
providing multiple amounts of cannabis-containing substance, each amount derived from cannabis plant material, the cannabis-containing substance containing one or more cannabinoids;
providing a control device having a machine-readable storage;
diluting each amount of cannabis-containing substance with a diluting agent to produce respective master batches of consumable product;
for each master batch:
dispensing the master batch into a set of individual packages, each package holding a portion of the master batch; and
withholding from dispensing into an individual package a residual volume of consumable product from the master batch that is less than the volume of consumable product required to fill the individual package to capacity;
for one or more master batches, determining a number of individual packages filled to capacity from the master batch in the respective set of individual packages;
storing the number in the machine-readable storage;
feeding a stream of individual packages to a marking unit; and
controlling with the control device the marking unit including deriving from the machine-readable storage the number and operating the marking unit a corresponding number of times to apply to each individual package in the set an indicia linked to a particular amount of cannabis-containing substance from which the consumable product in the package is made.

132. The method as defined in claim 131, wherein the cannabis-infused consumable product is vaping oil.

133. The method as defined in claim 131, wherein each package is a vaping cartridge containing vaping oil.

134. The method as defined in claim 131, wherein the diluting agent is oil or water.

135. The method as defined in claim 131, wherein the consumable product is a cannabis-infused beverage or an emulsion.

136. The method as defined in claim 131, wherein dispensing the master batch is performed such that the consumable product in each package originates from a single amount of cannabis-containing substance.

137. The method as defined in claim 131, including counting the number of packages produced from a particular amount of cannabis-containing substance and storing the counted number in the machine-readable storage.
